(19) United States
(12) Patent Application Publication
(10) Pub. No.: US 2013/0184320 A1
(43) Pub. Date: Jul. 18, 2013

(54) Imine Compounds

(71) Applicants: Karsten Koerber, Eppelheim (DE); Florian Kaiser, Mannheim (DE); Wolfgang Von Deyn, Neustadt (DE); Prashant Deshmukh, Mannheim (DE); Arun Narine, Mannheim (DE); Joachim Dickhaut, Heidelberg (DE); Nina Gertud Bandur, Mannheim (DE)

(72) Inventors: Karsten Koerber, Eppelheim (DE); Florian Kaiser, Mannheim (DE); Wolfgang Von Deyn, Neustadt (DE); Prashant Deshmukh, Mannheim (DE); Arun Narine, Mannheim (DE); Joachim Dickhaut, Heidelberg (DE); Nina Gertud Bandur, Mannheim (DE)

(73) Assignee: BASF SE, Ludwigshafen (DE)

(21) Appl. No.: 13/876,322

(22) PCT Filed: Sep. 30, 2011
(86) PCT No.: PCT/EP2011/067087

§ 371 Date: Mar. 27, 2013

Related U.S. Application Data
(60) Provisional application No. 61/388,635, filed on Oct. 1, 2010.

(51) Int. Cl. C07D 263/24; A01N 43/36; C07D 207/09; A01N 43/76

(52) U.S. Cl. CPC C07D 263/24; A01N 43/76; A01N 43/36; C07D 207/09
USPC 514/376; 514/429; 548/231; 548/577

(57) ABSTRACT

The present invention relates to imine compounds which are useful for combating or controlling invertebrate pests, in particular arthropod pests and nematodes. The invention also relates to a method for controlling invertebrate pests by using these compounds and to plant propagation material and to an agricultural and a veterinary composition comprising said compounds.

The present invention relates to imine compounds which are useful for combating or controlling invertebrate pests, in particular arthropod pests and nematodes. The invention also relates to a method for controlling invertebrate pests by using these compounds and to plant propagation material and to an agricultural and a veterinary composition comprising said compounds.

Invertebrate pests and in particular arthropods and nematodes destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating invertebrate pests, in particular insects, arachnids and nematodes.

WO 2010/072781 relates to isoxazoline compounds of formula

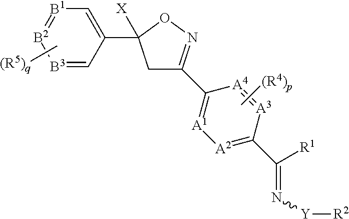

wherein, inter alia, A¹, A², A³, A⁴, B¹, B² and B³ are independently carbon or nitrogen atoms. This document does not disclose imine compounds comprising the azolinyl or azolidinyl rings of the present invention.

It was an object of the present invention to provide compounds that have a good pesticidal activity, in particular insecticidal activity, and show a broad activity spectrum against a large number of different invertebrate pests, especially against difficult to control arthropod pests and/or nematodes.

It has been found that these objectives can be achieved by imine compounds of the formula I below, by their steroisomers and by their salts, in particular their agriculturally or veterinarily acceptable salts.

Therefore, in a first aspect, the invention relates to imine compounds of formula I

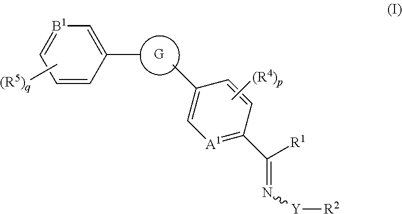

wherein
A¹ is N or CH;
B¹ is N or CH;
G is a bivalent heterocyclic ring selected from the following groups G-1 to G-28

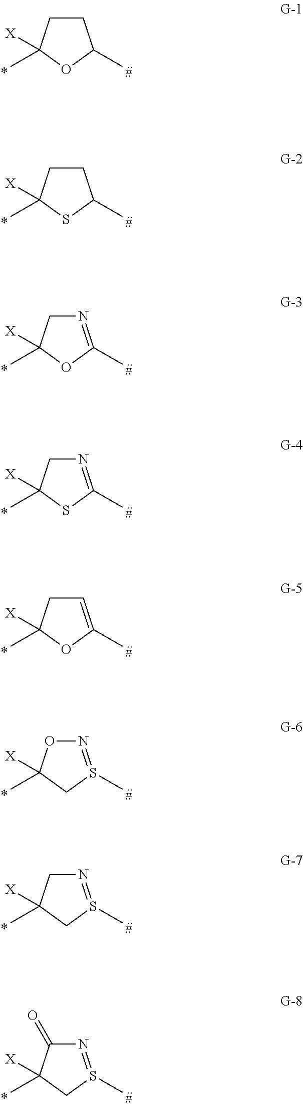

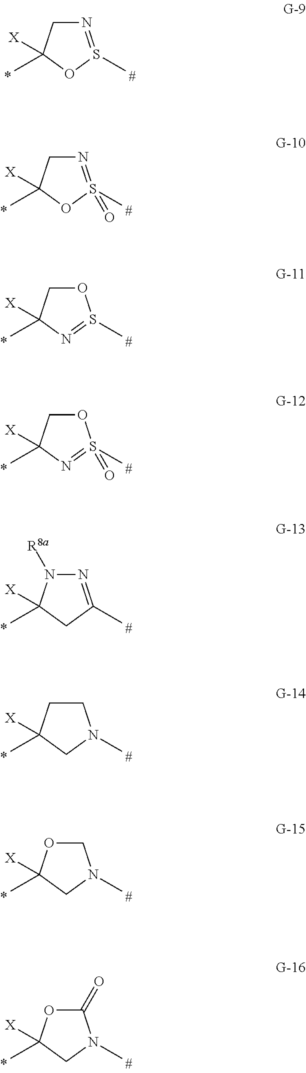

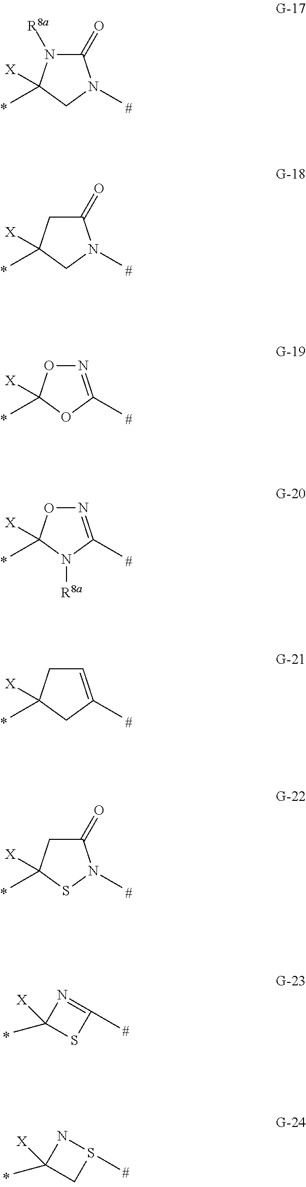

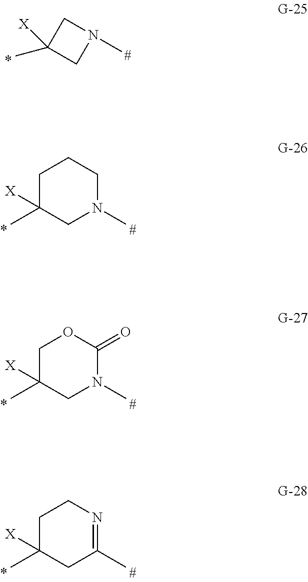

wherein the “*” and “#” in the variables G-1 to G-28 indicate the bonds to the neighbouring phenyl or pyridyl rings;
X is selected from the group consisting of C₁-C₄-alkyl, C₁-C₄-alkoxy-C₁-C₄-alkyl, C₁-C₄-haloalkoxy-C₁-C₄-alkyl, C₂-C₄-alkenyl, C₂-C₄-haloalkenyl, C₂-C₄-alkynyl, C₂-C₄-haloalkynyl, C₃-C₆-cycloalkyl and C₃-C₆-halocycloalkyl;
Y is O, N—R³, S(O)ₙ or a chemical bond;
R¹ is selected from the group consisting of hydrogen; cyano; C₁-C₁₀-alkyl which may be partially or fully halogenated and/or may be substituted by one or more radicals R⁶; C₁-C₁₀-alkoxy; C₁-C₁₀-haloalkoxy; C₁-C₁₀-alkylthio; C₁-C₁₀-haloalkylthio; C₁-C₁₀-alkylsulfinyl; C₁-C₁₀-haloalkylsulfinyl; C₁-C₁₀-alkylsulfonyl; C₁-C₁₀-haloalkylsulfonyl; C₃-C₈-cycloalkyl which may be partially or fully halogenated and/or may be substituted by one or more radicals R⁶; C₂-C₁₀-alkenyl which may be partially or fully halogenated and/or may be substituted by one or more radicals R⁶; C₂-C₁₀-alkynyl which may be partially or fully halogenated and/or may be substituted by one or more radicals R⁶; —C(═O)R⁶; —C(═O)OR⁷; —C(═O)N(R⁸)R⁹; —C(═S)R⁶; —C(═S)OR⁷; —C(═S)N(R⁸)R⁹; phenyl which may be substituted by 1, 2, 3, 4 or 5 radicals R¹⁰; and a C-bound 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
R² is selected from the group consisting of hydrogen; cyano; C₁-C₁₀-alkyl which may be partially or fully halogenated and/or may be substituted by one or more radicals R⁶; C₃-C₈-cycloalkyl which may be partially or fully halogenated and/or may be substituted by one or more radicals R⁶; C₂-C₁₀-alkenyl which may be partially or fully halogenated and/or may be substituted by one or more radicals R⁶; C₂-C₁₀-alkynyl which may be partially or fully halogenated and/or may be substituted by one or more radicals R⁶; —N(R⁸)R⁹; —N(R⁸)C(═O)R⁶; —Si(R¹⁴)₂R¹³; —OR⁷; —SR⁷; —S(O)ₘR⁷; —S(O)ₙN(R⁸)R⁹; —C(═O)R⁶; —C(═O)OR⁷; —C(═O)N(R⁸)R⁹; —C(═S)R⁶; —C(═OS)OR⁷, —C(═OS)N(R⁸)R⁹; —C(═NR⁸)R⁶; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰; with the proviso that R² is not —OR⁷ if Y is O;
R³ is selected from the group consisting of hydrogen; cyano; C₁-C₁₀-alkyl which may be partially or fully halogenated and/or may be substituted by one or more radicats R⁶; C₃-C₈-cycloalkyl which may be partially or fully halogenated and/or may be substituted by one or more radicals R⁶; C₂-C₁₀-alkenyl which may be partially or fully halogenated and/or may be substituted by one or more radicals R⁶; C₂-C₁₀-alkynyl which may be partially or fully halogenated and/or may be substituted by one or more radicals R⁶; —N(R⁸)R⁹; —Si(R¹⁴)₂R¹³; —OR⁷; —SR⁷; —S(O)ₘR⁷; —S(O)ₙN(R⁸)R⁹; —C(═O)R⁶; —C(═O)OR⁷; —C(═O)N(R⁸)R⁹; —C(═S)R⁶; —C(═S)OR⁷; —C(═S)N(R⁸)R⁹; —C(═NR⁸)R⁶; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
   or R² and R³ together form a group ═CR¹¹R¹²; ═S(O)ₘR⁷; ═S(O)ₘN(R⁸)R⁹; ═NR⁸; or ═NOR⁷;
   or R² and R³ together form a C₂-C₇ alkylene chain, thus forming, together with the nitrogen atom to which they are bound, a 3-, 4-, 5-, 6-, 7- or 8-membered ring, where the alkylene chain may be interrupted by 1 or 2 O, S and/or NR¹⁸ and/or 1 or 2 of the CH₂ groups of the alkylene chain may be replaced by a group C═O, C═S and/or C═NR¹⁸; and/or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
each R⁴ is independently selected from the group consisting of halogen; cyano; azido; nitro; —SCN; SF₅; C₁-C₆-alkyl which may be partially or fully halogenated and/or may be substituted by one or more radicals R⁶; C₃-C₈-cycloalkyl which may be partially or fully halogenated and/or may be substituted by one or more radicals R⁶; C₂-C₆-alkenyl which may be partially or fully halogenated and/or may be substituted by one or more radicals R⁶; C₂-C₆-alkynyl which may be partially or fully halogenated and/or may be substituted by one or more radicals R⁶; —Si(R¹⁴)₂R¹³; —OR⁷; —OS(O)ₙR⁷; —SR⁷; —S(O)ₘR⁷; —S(O)ₙN(R⁸)R⁹; —N(R⁸)R⁹; —N(R⁸)C(═O)R⁶; —C(═O)R⁶; —C(═O)OR⁷; —C(═NR⁸)H; —C(═NR⁸)R⁶; —C(═O)N(R⁸)R⁹; C(═S)N(R⁸)R⁹; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
   or two radicals R⁴ bound on adjacent carbon atoms may be together a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR⁸—, —CH₂CH═N—, —CH═CH—NR⁸—, —OCH═N— and —SCH═N—, thus forming, together with the carbon atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group;
each R⁵ is independently selected from the group consisting of halogen, cyano, azido, nitro, —SCN, SF₅, C₁-C₆-alkyl which may be partially or fully halogenated and/or may be substituted by one or more radicals R⁶, C₃-C₈-cycloalkyl which may be partially or fully halogenated and/or may be substituted by one or more radicals R⁶, C₂-C₆-alkenyl which may be partially or fully halogenated and/or may be substituted by one or more radicals R⁶, C₂-C₆-alkynyl which may be partially or fully halogenated and/or may be substituted by one or more radicals R⁶, —Si(R¹⁴)₂R¹³, —OR⁷, —OS(O)ₙR⁷, —S(O)ₘR⁷, —S(O)ₙN(R⁸)R⁹, —N(R⁸)R⁹, N(R⁸)C(═O)R⁶, —C(═O)R⁶, —C(═O)OR⁷, —C(═S)R⁶, —C(═S)OR⁷, —C(═NR⁸)R⁶, —C(═O)N(R⁸)R⁹, —C(═S)N(R⁸)R⁹, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
each R⁶ is independently selected from the group consisting of cyano, azido, nitro, —SCN, SF₅, C₃-C₈-cycloalkyl, C₃-C₈-halocycloalkyl, —Si(R¹⁴)₂R¹³, —OR⁷, —OSO₂R⁷, —SR⁷, —S(O)ₘR⁷, —S(O)ₙN(R⁸)R⁹, —N(R⁸)R⁹, —C(═O)N(R⁸)R⁹, —C(═S)N(R⁸)R⁹, —C(═O)OR⁷, —C(═O)R¹⁹, —C(═NR⁸)R¹⁹, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
   and, in case R⁶ is bound to a cycloalkyl group, R⁶ may additionally be selected from the group consisting of C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, C₂-C₆-haloalkynyl and benzyl which may be substituted by 1, 2, 3, 4 or 5 radicals R¹⁰;
   and in groups —C(═O)R⁶, —C(═S)R⁶, —C(═NR⁸)R⁶ and —N(R⁸)C(═O)R⁶, R⁶ may additionally be selected from hydrogen, halogen, C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, C₂-C₆-haloalkynyl and benzyl which may be substituted by 1, 2, 3, 4 or 5 radicals R¹⁰;
   or two geminally bound radicals R⁶ together form a group selected from ═CR¹¹R¹², ═S(O)ₘR⁷, ═S(O)ₘN(R⁸)R⁹, ═NR⁸, ═NOR⁷ and ═NNR⁸;
   or two radicals R⁶, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members;
each R⁷ is independently selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁴)₂R¹³, —SR⁸, —S(O)ₘR⁷, —S(O)ₙN(R⁸)R⁹, —N(R⁸)R⁹, —N═CR¹⁵R¹⁶, —C(═O)R¹⁷, —C(═O)N(R⁸)R⁹, —C(═S)N(R⁸)R⁹, —C(═O)OR¹⁷, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
   with the proviso that R⁷ is not C₁-C₆-alkoxy or C₁-C₆-haloalkoxy if it is bound to an oxygen atom;
each R⁸ is independently selected from the group consisting of hydrogen, cyano, C₁-C₆-alkyl which may be partially or fully halogenated and/or may be substituted by one or more radicals R¹⁹, C₁-C₆-alkoxy, C₁-C₆-haloalkoxy, C₁-C₆-alkylthio, C₁-C₆-haloalkylthio, where the alkyl moiety in the four last-mentioned radicals may be substituted by one or more radicals R¹⁹, C₃-C₈-cycloalkyl which may be partially or fully halogenated and/or may be substituted by one or more radicals R¹⁹, C₃-C₈-cycloalkyl-C₁-C₄-alkyl where the cycloalkyl moiety may be partially or fully halogenated and/or may be substituted by one or more radicals R¹⁹, C₂-C₆-alkenyl which may be partially or fully halogenated and/or may be substituted by one or more radicals R¹⁹, C₂-C₆-alkynyl which may be partially or fully halogenated and/or may be substituted by one or more radicals R¹⁹, —S(O)ₘR²⁰, —S(O)ₙN(R²¹)R²², phenyl which may be substituted by 1, 2, 3, 4 or 5 radicals R¹⁰, benzyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
each R⁹ is independently selected from the group consisting of hydrogen, cyano, C₁-C₆-alkyl which may be partially or fully halogenated and/or may be substituted by one or more radicals R¹⁹, C₁-C₆-alkoxy, C₁-C₆-haloalkoxy, C₁-C₆-alkylthio, C₁-C₆-haloalkylthio, where the alkyl moiety in the four last-mentioned radicals may be substituted by one or more radicals R¹⁹, C₃-C₈-cycloalkyl which may be partially or fully halogenated and/or may be substituted by one or more radicals R¹⁹, C₃-C₈-cycloalkyl-C₁-C₄-alkyl where the cycloalkyl moiety may be partially or fully halogenated and/or may be substituted by one or more radicals R¹⁹, C₂-C₆-alkenyl which may be partially or fully halogenated and/or may be substituted by one or more radicals R¹⁹, C₂-C₆-alkynyl which may be partially or fully halogenated and/or may be substituted by one or more radicals R¹⁹, —S(O)ₘR²⁰, —S(O)ₙN(R²¹)R²², phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
   or R⁸ and R⁹ together form a group ═CR¹¹R¹²;
   or R⁸ and R⁹, together with the nitrogen atom to which they are bound, may form a 3-, 4-, 5-, 6- or 7-membered saturated, partially unsaturated or aromatic heterocyclic ring which may additionally containing 1 or 2 further heteroatoms or heteroatom groups selected from N, O, S, NO, SO and SO₂, as ring members, where the heterocyclic ring may be substituted by one or more radicals R¹⁰;
each R^{8a} is independently defined like R⁸;
each R¹⁰ is independently selected from the group consisting of halogen, cyano, azido, nitro, —SCN, SF₅, C₁-C₁₀-alkyl which may be partially or fully halogenated and/or may be substituted by one or more radicals R¹⁹, C₃-C₈-cycloalkyl which may be partially or fully halogenated and/or may be substituted by one or more radicals R¹⁹, C₂-C₁₀-alkenyl which may be partially or fully halogenated and/or may be substituted by one or more radicals R¹⁹, C₂-C₁₀-alkynyl which may be partially or fully halogenated and/or may be substituted by one or more radicals R¹⁹, —Si(R¹⁴)₂R¹³, —OR²⁰, —OS(O)ₙR²⁰, —SR²⁰, —S(O)ₘR²⁰, —S(O)ₙN(R²¹)R²², —N(R²¹)R²², C(═O)R¹⁹, —C(═O)OR²⁰, —C(═NR²¹)R²², —C(═O)N(R²¹)R²², —C(═S)N(R²¹)R²², phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more radicals independently selected from halogen, cyano, nitro, C₁-C₆-alkyl, C₁-C₆-haloalkyl, C₁-C₆-alkoxy and C₁-C₆-haloalkoxy;
   or two radicals R¹⁰ bound on adjacent atoms together form a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR²¹—, —CH₂CH═N—, —CH═CH—NR²¹—, —OCH═N— and —SCH═N—, thus forming, together with the atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group;
R¹¹, R¹² are, independently of each other and independently of each occurrence, selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, C₁-C₆-alkoxy, C₁-C₆-haloalkoxy, —C(═O)R¹⁹, —C(═O)OR²⁰, —C(═NR²¹)R²², —C(═O)N(R²¹)R²², —C(═S)N(R²¹)R²², phenyl which may be substituted by 1, 2, 3, 4, or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, which may be substituted by one or more radicals R¹⁰;
R¹³, R¹⁴ are, independently of each other and independently of each occurrence, selected from the group consisting of C₁-C₄-alkyl, C₃-C₆-cycloalkyl, C₁-C₄-alkoxy-C₁-C₄-alkyl, phenyl and benzyl;
R¹⁵, R¹⁶ are, independently of each other and independently of each occurrence, selected from the group consisting of C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, phenyl which may be substituted by 1, 2, 3, 4, or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, which may be substituted by one or more radicals R¹⁰;
each R¹⁷ is independently selected from the group consisting of C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, phenyl and benzyl;
each R¹⁸ is independently defined like R³;
each R¹⁹ is independently selected from the group consisting of cyano, azido, nitro, —SCN, SF_{S}, C₃-C₈-cycloalkyl, C₃-C₈-halocycloalkyl, —Si(R¹⁴)₂R¹³, —OR²⁰, —OSO₂R²⁰, —SR²⁰, —S(O)ₘR²⁰, —S(O)ₙN(R²¹)R²², —N(R²¹)R²², —C(═O)N(R²¹)R²², —C(═S)N(R²¹)R²², —C(═O)OR²⁰, —C(═O)R²⁰, —C(═NR²¹)R²⁰, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
   and, in case R¹⁹ is bound to a cycloalkyl group, R¹⁹ may additionally be selected from the group consisting of C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl and C₂-C₆-haloalkynyl; and in groups —C(═O)R¹⁹, R¹⁹ may additionally be selected from hydrogen, halo gen, C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, and C₂-C₆-haloalkynyl;
   or two geminally bound radicals R¹⁹ together form a group selected from ═cR¹¹R¹², ═S(O)ₘR²⁰, ═S(O)ₘN(R²¹)R²², ═NR²¹, ═NOR²⁰ and ═NNR²¹;
   or two radicals R¹⁹, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members;
each R²⁰ is independently selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁴)₂R¹³, C₁-C₆-alkylaminosulfonyl, amino, C₁-C₆-alkylamino, di-(C₁-C₆-alkyl)-amino, C₁-C₆-alkylcarbonyl, C₁-C₆-haloalkylcarbonyl, aminocarbonyl, C₁-C₆-alkylaminocarbonyl, di-(C₁-C₆-alkyl)-aminocarbonyl, C₁-C₆-alkoxycarbonyl, C₁-C₆-haloalkoxycarbonyl, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, benzyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
   with the proviso that R²⁰ is not C₁-C₆-alkoxy or C₁-C₆-haloalkoxy if it is bound to an oxygen atom;
R²¹ and R²² are independently of each other and independently of each occurrence selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₃-C₈-cycloalkyl-C₁-C₄-alkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, benzyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
   or R²¹ and R²², together with the nitrogen atom to which they are bound, may form a 3-, 4-, 5-, 6- or 7-membered saturated, partially unsaturated or aromatic heterocyclic ring which may additionally containing 1 or 2 further heteroatoms or heteroatom groups selected from N, O, S, NO, SO and SO₂, as ring members, where the heterocyclic ring may be substituted by one or more radicals selected from halogen, C₁-C₆-alkyl, C₁-C₆-haloalkyl, C₁-C₆-alkoxy and C₁-C₆-haloalkoxy;
each m is independently 1 or 2;
each n is independently 0, 1 or 2;
p is 0, 1, 2, 3 or 4; and
q is 0, 1, 2, 3, 4 or 5;
and the stereoisomers and agriculturally or veterinarily acceptable salts thereof.

The present invention also provides an agricultural composition comprising at least one compound of the formula I as defined herein and/or an agriculturally acceptable salt thereof and at least one liquid or solid carrier.

The present invention also provides a veterinary composition comprising at least one compound of the formula I as defined herein and/or a veterinarily acceptable salt thereof and at least one liquid or solid carrier.

The present invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I or a salt thereof as defined herein.

The present invention also relates to plant propagation material, in particular seed, comprising at least one compound of formula I and/or an agriculturally acceptable salt thereof as defined herein.

The present invention further relates to a method for treating or protecting an animal from infestation or infection by parasites which comprises bringing the animal in contact with a parasiticidally effective amount of a compound of the formula I or a veterinarily acceptable salt thereof as defined herein. Bringing the animal in contact with the compound I, its salt or the veterinary composition of the invention means applying or administering it to the animal.

The term “steroisomers” encompasses both optical isomers, such as enantiomers or diastereomers, the latter existing due to more than one center of chirality in the molecule, as well as geometrical isomers (cis/trans isomers).

Depending on the substitution pattern, the compounds of the formula I may have one or more centers of chirality, in which case they are present as mixtures of enantiomers or diastereomers. One center of chirality is the carbon ring atom of the G ring carrying radical X. The invention provides both the pure enantiomers or diastereomers and their mixtures and the use according to the invention of the pure enantiomers or diastereomers of the compound I or its mixtures. Suitable compounds of the formula I also include all possible geometrical stereoisomers (cis/trans isomers) and mixtures thereof. Cis/trans isomers may be present with respect to the imine group. Preference is given to compounds wherein the group —Y—R² is trans with respect to the ring containing A¹ as ring member, i.e. to compounds of formula trans-I

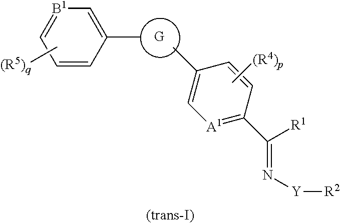

Enantiomers with respect to the position of the group X and the ring containing B¹ as ring member on the ring G may be represented by following simplified formulae

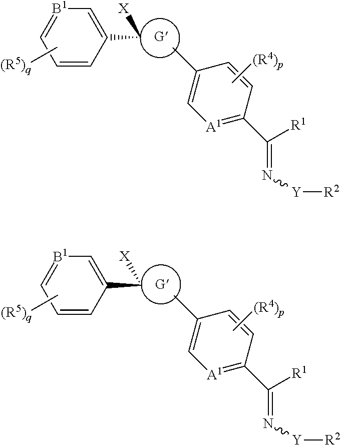

wherein G′ is ring G without group X.

The compounds of the present invention may be amorphous or may exist in one or more different crystalline states (polymorphs) which may have a different macroscopic properties such as stability or show different biological properties such as activities. The present invention includes both amorphous and crystalline compounds of the formula I, mixtures of different crystalline states of the respective compound I, as well as amorphous or crystalline salts thereof.

Salts of the compounds of the formula I are preferably agriculturally and veterinarily acceptable salts. They can be formed in a customary method, e.g. by reacting the compound with an acid of the anion in question if the compound of formula I has a basic functionality or by reacting an acidic compound of formula I with a suitable base.

Suitable agriculturally acceptable salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzl-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I with an acid of the corresponding anion, preferably of hydrochloric acid, hydrobromic acid, sulfuric acid, phosphoric acid or nitric acid.

By the term “veterinarily acceptable salts” is meant salts of those cations or anions which are known and accepted in the art for the formation of salts for veterinary use. Suitable acid addition salts, e.g. formed by compounds of formula I containing a basic nitrogen atom, e.g. an amino group, include salts with inorganic acids, for example hydrochlorids, sulphates, phosphates, and nitrates and salts of organic acids for example acetic acid, maleic acid, dimaleic acid, fumaric acid, difumaric acid, methane sulfenic acid, methane sulfonic acid, and succinic acid.

The term “invertebrate pest” as used herein encompasses animal populations, such as insects, arachnids and nematodes, which may attack plants, thereby causing substantial damage to the plants attacked, as well as ectoparasites which may infest animals, in particular warm blooded animals such as e.g. mammals or birds, or other higher animals such as reptiles, amphibians or fish, thereby causing substantial damage to the animals infested.

The term “plant propagation material” as used herein includes all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “plants” comprises any types of plants including “non-cultivated plants” and in particular “cultivated plants”.

The term “non-cultivated plants” refers to any wild type species or related species or related genera of a cultivated plant.

The term “cultivated plants” as used herein includes plants which have been modified by breeding, mutagenesis or genetic engineering. Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transitional modification of protein(s) (oligo- or polypeptides) poly for example by glycosylation or polymer additions such as prenylated, acetylated or farnesylated moieties or PEG moieties (e.g. as disclosed in Biotechnol Prog. 2001 July-August; 17(4):720-8, Protein Eng Des Sel. 2004 January; 17(1):57-66, Nat. Protoc. 2007; 2(5):1225-35, Curr. Opin. Chem. Biol. 2006 October; 10(5):487-91. Epub 2006 Aug. 28, Biomaterials. 2001 March; 22(5):405-17, Bioconjug Chem. 2005 January-February; 16(1):113-21).

The term “cultivated plants” as used herein further includes plants that have been rendered tolerant to applications of specific classes of herbicides, such as hydroxy-phenylpyruvate dioxygenase (HPPD) inhibitors; acetolactate synthase (ALS) inhibitors, such as sulfonyl ureas (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073); enolpyruvylshikimate-3-phosphate synthase (EPSPS) inhibitors, such as glyphosate (see e.g. WO 92/00377); glutamine synthetase (GS) inhibitors, such as glufosinate (see e.g. EP-A-0242236, EP-A-242246) or oxynil herbicides (see e.g. U.S. Pat. No. 5,559,024) as a result of conventional methods of breeding or genetic engineering. Several cultivated plants have been rendered tolerant to herbicides by conventional methods of breeding (mutagenesis), for example Clearfield® summer rape (Canola) being tolerant to imidazolinones, e.g. imazamox. Genetic engineering methods have been used to render cultivated plants, such as soybean, cotton, corn, beets and rape, tolerant to herbicides, such as glyphosate and glufosinate, some of which are commercially available under the trade names RoundupReady® (glyphosate) and LibertyLink® (glufosinate).

The term “cultivated plants” as used herein further includes plants that are by the use of recombinant DNA techniques capable to synthesize one or more insecticidal proteins, especially those known from the bacterial genus bacillus, particularly from bacillus thuringiensis, such as ä-endotoxins, e.g. CryIA(b), CryIA(c), CryIF, CryIF(a2), CryIIA(b), CryIIIA, CryIIIB(b1) or Cry9c; vegetative insecticidal proteins (VIP), e.g. VIP1, VIP2, VIP3 or VIP3A; insecticidal proteins of bacteria colonizing nematodes, for example Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, for example WO 02/015701). Further examples of such toxins or genetically-modified plants capable of synthesizing such toxins are disclosed, for example, in EP-A 374 753, WO 93/007278, WO 95/34656, EP-A 427 529, EP-A 451 878, WO 03/018810 and WO 03/052073. The methods for producing such genetically modified plants are generally known to the person skilled in the art and are described, for example, in the publications mentioned above. These insecticidal proteins contained in the genetically modified plants impart to the plants producing these proteins protection from harmful pests from certain taxonomic groups of arthropods insects, particularly to beetles (Coleoptera), flies (Diptera), and butterflies and moths (Lepidoptera) and to plant parasitic nematodes (Nematoda).

The term “cultivated plants” as used herein further includes plants that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, for example EP-A 0 392 225), plant disease resistance genes (for example potato cultivars, which express resistance genes acting against Phytophthora infestans derived from the mexican wild potato Solanum bulbocastanum) or T4-lyso-zym (e.g. potato cultivars capable of synthesizing these proteins with increased resistance against bacteria such as Erwinia amylvora). The methods for producing such genetically modified plants are generally known to the person skilled in the art and are described, for example, in the publications mentioned above.

The term “cultivated plants” as used herein further includes plants that are by the use of recombinant DNA techniques capable to synthesize one or more proteins to increase the productivity (e.g. bio mass production, grain yield, starch content, oil content or protein content), tolerance to drought, salinity or other growth-limiting environmental factors or tolerance to pests and fungal, bacterial or viral pathogens of those plants.

The term “cultivated plants” as used herein further includes plants that contain by the use of recombinant DNA techniques a modified amount of substances of content or new substances of content, specifically to improve human or animal nutrition, for ex-ample oil crops that produce health-promoting long-chain omega-3 fatty acids or unsaturated omega-9 fatty acids (e.g. Nexera® rape).

The term “cultivated plants” as used herein further includes plants that contain by the use of recombinant DNA techniques a modified amount of substances of content or new substances of content, specifically to improve raw material production, for example potatoes that produce increased amounts of amylopectin (e.g. Amflora® potato).

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

The term “C₁-C₁₀-alkyl” as used herein and in the alkyl moieties of alkoxy, alkylthio, alkylsulfinyl, alkylsulfonyl, alkylcarbonyl, alkoxycarbonyl and the like refers to saturated straight-chain or branched hydrocarbon radicals having 1 to 2 (“C₁-C₂-alkyl”), 1 to 4 (“C₁-C₄-alkyl”), 1 to 6 (“C₁-C₆-alkyl”), 1 to 8 (“C₁-C₈-alkyl”) or 1 to 10 (“C₁-C₁₀-alkyl”) carbon atoms. C₁-C₂-Alkyl is methyl or ethyl. C₁-C₄-Alkyl is additionally propyl, isopropyl, butyl, 1-methylpropyl(sec-butyl), 2-methylpropyl(isobutyl) or 1,1-dimethylethyl(tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl. C₁-C₈-Alkyl is additionally also, for example, heptyl, octyl, 2-ethylhexyl and positional isomers thereof. C₁-C₁₀-Alkyl is additionally also, for example, nonyl, decyl and positional isomers thereof.

The term “C₁-C₁₀-haloalkyl” as used herein, which is also expressed as “C₁-C₁₀-alkyl which is partially or fully halogenated”, refers to straight-chain or branched alkyl groups having 1 to 2 (“C₁-C₂-haloalkyl”), 1 to 4 (“C₁-C₄-haloalkyl”), 1 to 6 (“C₁-C₆-haloalkyl”), 1 to 8 (“C₁-C₈-haloalkyl”) or 1 to 10 (“C₁-C₁₀-haloalkyl”) carbon atoms (as mentioned above), where some or all of the hydrogen atoms in these groups are replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl.

“Halomethyl” is methyl in which 1, 2 or 3 of the hydrogen atoms are replaced by halogen atoms. Examples are bromomethyl, chloromethyl, fluoromethyl, dichloromethyl, trichloromethyl, difluoromethyl, trifluoromethyl, chlorofluoromethyl, dichlorofluoromethyl, chlorodifluoromethyl and the like.

The term “C₂-C₁₀-alkenyl” as used herein and in the alkenyl moiety of alkenyloxy and the like refers to monounsaturated straight-chain or branched hydrocarbon radicals having 2 to 4 (“C₂-C₄-alkenyl”), 2 to 6 (“C₂-C₆-alkenyl”), 2 to 8 (“C₂-C₈-alkenyl”), 3 to 8 (“C₃-C₈-alkenyl”), 2 to 10 (“C₂-C₁₀-alkenyl”) or 3 to 10 (“C₃-C₁₀-alkenyl”) carbon atoms and a double bond in any position, for example C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₈-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like, or C₂-C₁₀-alkenyl, such as the radicals mentioned for C₂-C₆-alkenyl and additionally 1-heptenyl, 2-heptenyl, 3-heptenyl, 1-octenyl, 2-octenyl, 3-octenyl, 4-octenyl, 1-nonenyl, 2-nonenyl, 3-nonenyl, 4-nonenyl, 1-decenyl, 2-decenyl, 3-decenyl, 4-decenyl, 5-decenyl and the positional isomers thereof.

The term “C₂-C₁₀-haloalkenyl” as used herein, which is also expressed as “C₁-C₁₀-alkenyl which is partially or fully halogenated”, and the haloalkenyl moieties in haloalkenyloxy, haloalkenylcarbonyl and the like refers to unsaturated straight-chain or branched hydrocarbon radicals having 2 to 4 (“C₂-C₄-haloalkenyl”), 2 to 6 (“C₂-C₆-haloalkenyl”), 2 to 8 (“C₂-C₆-haloalkenyl”) or 2 to 10 (“C₂-C₁₀-haloalkenyl”) carbon atoms and a double bond in any position (as mentioned above), where some or all of the hydrogen atoms in these groups are replaced by halogen atoms as mentioned above, in particular fluorine, chlorine and bromine, for example chlorovinyl, chloroallyl and the like.

The term “C₂-C₁₀-alkynyl” as used herein and the alkynyl moieties in alkynyloxy, alkynylcarbonyl and the like refers to straight-chain or branched hydrocarbon groups having 2 to 4 (“C₂-C₄-alkynyl”), 2 to 6 (“C₂-C₆-alkynyl”), 2 to 8 (“C₂-C₈-alkynyl”), 3 to 8 (“C₃-C₈-alkynyl”), 2 to 10 (“C₂-C₁₀-alkynyl”) or 3 to 10 (“C₃-C₈-alkynyl”) carbon atoms and one or two triple bonds in any position, for example C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

The term “C₂-C₁₀-haloalkynyl” as used herein, which is also expressed as “C₁-C₁₀-alkynyl which is partially or fully halogenated”, and the haloalkynyl moieties in haloalkynyloxy, haloalkynylcarbonyl and the like refers to unsaturated straight-chain or branched hydrocarbon radicals having 2 to 4 (“C₂-C₄-haloalkynyl”), 3 to 4 (“C₃-C₄-haloalkynyl”), 2 to 6 (“C₂-C₆-haloalkynyl”), 3 to 6 (“C₃-C₆-haloalkynyl”), 2 to 8 (“C₂-C₈-haloalkynyl”), 3 to 8 (“C₃-C₈-haloalkynyl”), 2 to 10 (“C₂-C₁₀-haloalkynyl”) or 3 to 10 (“C₃-C₁₀-haloalkynyl”) carbon atoms and one or two triple bonds in any position (as mentioned above), where some or all of the hydrogen atoms in these groups are replaced by halogen atoms as mentioned above, in particular fluorine, chlorine and bromine;

The term “C₃-C₈-cycloalkyl” as used herein refers to mono- or bi- or polycyclic saturated hydrocarbon radicals having 3 to 8, in particular 3 to 6 carbon atoms (“C₃-C₆-cycloalkyl”). Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.2.1]heptyl, bicyclo[3.1.1]heptyl, bicyclo[2.2.2]octyl and bicyclo[3.2.1]octyl.

The term “C₃-C₈-halocycloalkyl” as used herein, which is also expressed as “C₃-C₈-cycloalkyl which is partially or fully halogenated”, and the halocycloalkyl moieties in halocycloalkoxy, halocycloalkylcarbonyl and the like refers to mono- or bi- or polycyclic saturated hydrocarbon groups having 3 to 8 (“C₃-C₈-halocycloalkyl”) or preferably 3 to 6 (“C₃-C₆-halocycloalkyl”) carbon ring members (as mentioned above) in which some or all of the hydrogen atoms are replaced by halogen atoms as mentioned above, in particular fluorine, chlorine and bromine.

The term “C₃-C₈-cycloalkyl-C₁-C₄-alkyl” refers to a C₃-C₈-cycloalkyl group as defined above which is bound to the remainder of the molecule via a C₁-C₄-alkyl group, as defined above. Examples are cyclopropylmethyl, cyclopropylethyl, cyclopropylpropyl, cyclobutylmethyl, cyclobutylethyl, cyclobutylpropyl, cyclopentylmethyl, cycloppentylethyl, cyclopentylpropyl, cyclohexylmethyl, cyclohexylethyl, cyclohexylpropyl, and the like. The term “C₁-C₂-alkoxy” is a C₁-C₂-alkyl group, as defined above, attached via an oxygen atom. The term “C₁-C₄-alkoxy” is a C₁-C₄-alkyl group, as defined above, attached via an oxygen atom. The term “C₁-C₆-alkoxy” is a C₁-C₆-alkyl group, as defined above, attached via an oxygen atom. The term “C₁-C₁₀-alkoxy” is a C₁-C₁₀-alkyl group, as defined above, attached via an oxygen atom. C₁-C₂-Alkoxy is methoxy or ethoxy. C₁-C₄-Alkoxy is additionally, for example, n-propoxy, 1-methylethoxy(isopropoxy), butoxy, 1-methylpropoxy(sec-butoxy), 2-methylpropoxy(isobutoxy) or 1,1-dimethylethoxy(tertbutoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “C₁-C₂-haloalkoxy” is a C₁-C₂-haloalkyl group, as defined above, attached via an oxygen atom. The term “C₁-C₄-haloalkoxy” is a C₁-C₄-haloalkyl group, as defined above, attached via an oxygen atom. The term “C₁-C₆-haloalkoxy” is a C₁-C₆-haloalkyl group, as defined above, attached via an oxygen atom. The term “C₁-C₁₀-haloalkoxy” is a C₁-C₁₀-haloalkyl group, as defined above, attached via an oxygen atom. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₄-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

The term “C₁-C₂-alkylthio” is a C₁-C₂-alkyl group, as defined above, attached via a sulfur atom. The term “C₁-C₄-alkylthio” is a C₁-C₄-alkyl group, as defined above, attached via a sulfur atom. The term “C₁-C₆-alkylthio” is a C₁-C₆-alkyl group, as defined above, attached via a sulfur atom. The term “C₁-C₁₀-alkylthio” is a C₁-C₁₀-alkyl group, as defined above, attached via a sulfur atom. C₁-C₂-Alkylthio is methylthio or ethylthio. C₁-C₄-Alkylthio is additionally, for example, n-propylthio, 1-methylethylthio(isopropylthio), butylthio, 1-methylpropylthio(sec-butylthio), 2-methylpropylthio(isobutylthio) or 1,1-dimethylethylthio(tert-butylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio. C₁-C₈-Alkylthio is additionally, for example, heptylthio, octylthio, 2-ethylhexylthio and positional isomers thereof. C₁-C₁₀-Alkylthio is additionally, for example, nonylthio, decylthio and positional isomers thereof.

The term “C₁-C₂-haloalkylthio” is a C₁-C₂-haloalkyl group, as defined above, attached via a sulfur atom. The term “C₁-C₄-haloalkylthio” is a C₁-C₄-haloalkyl group, as defined above, attached via a sulfur atom. The term “C₁-C₆-haloalkylthio” is a C₁-C₆-haloalkyl group, as defined above, attached via a sulfur atom. The term “C₁-C₁₀-haloalkylthio” is a C₁-C₁₀-haloalkyl group, as defined above, attached via a sulfur atom. C₁-C₂-Haloalkylthio is, for example, SCH₂F, SCHF₂, SCF₃, SCH₂Cl, SCHCl₂, SCCl₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio or SC₂F₅. C₁-C₄-Haloalkylthio is additionally, for example, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio, 1-(CH₂Br)-2-bromoethylthio, 4-fluorobutylthio, 4-chlorobutylthio, 4-bromobutylthio or nonafluorobutylthio. C₁-C₆-Haloalkylthio is additionally, for example, 5-fluoropentylthio, 5-chloropentylthio, 5-brompentylthio, 5-iodopentylthio, undecafluoropentylthio, 6-fluorohexylthio, 6-chlorohexylthio, 6-bromohexylthio, 6-iodohexylthio or dodecafluorohexylthio.

The term “C₁-C₂-alkylsulfinyl” is a C₁-C₂-alkyl group, as defined above, attached via a sulfinyl [S(O)] group. The term “C₁-C₄-alkylsulfinyl” is a C₁-C₄-alkyl group, as defined above, attached via a sulfinyl [S(O)] group. The term “C₁-C₆-alkylsulfinyl” is a C₁-C₆-alkyl group, as defined above, attached via a sulfinyl [S(O)] group. The term “C₁-C₁₀-alkylsulfinyl” is a C₁-C₁₀-alkyl group, as defined above, attached via a sulfinyl [S(O)] group. C₁-C₂-Alkylsulfinyl is methylsulfinyl or ethylsulfinyl. C₁-C₄-Alkylsulfinyl is additionally, for example, n-propylsulfinyl, 1-methylethylsulfinyl(isopropylsulfinyl), butylsulfinyl, 1-methylpropylsulfinyl(sec-butylsulfinyl), 2-methylpropylsulfinyl(isobutylsulfinyl) or 1,1-dimethylethylsulfinyl(tert-butylsulfinyl). C₁-C₆-Alkylsulfinyl is additionally, for example, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl. C₁-C₈-Alkylsulfinyl is additionally, for example, heptylsulfinyl, octylsulfinyl, 2-ethylhexylsulfinyl and positional isomers thereof. C₁-C₁₀-Alkylsulfinyl is additionally, for example, nonylsulfinyl, decylsulfinyl and positional isomers thereof.

The term “C₁-C₂-haloalkylsulfinyl” is a C₁-C₂-haloalkyl group, as defined above, attached via a sulfinyl [S(O)] group. The term “C₁-C₄-haloalkylsulfinyl” is a C₁-C₄-haloalkyl group, as defined above, attached via a sulfinyl [S(O)] group. The term “C₁-C₆-haloalkylsulfinyl” is a C₁-C₆-haloalkyl group, as defined above, attached via a sulfinyl [S(O)] group. The term “C₁-C₁₀-haloalkylsulfinyl” is a C₁-C₁₀-haloalkyl group, as defined above, attached via a sulfinyl [S(O)] group. C₁-C₂-Haloalkylsulfinyl is, for example, S(O)CH₂F, S(O)CHF₂, S(O)CF₃, S(O)CH₂Cl, S(O)CHCl₂, S(O)CCl₃, chlorofluoromethylsulfinyl, dichlorofluoromethylsulfinyl, chlorodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2-chloro-2-fluoroethylsulfinyl, 2-chloro-2,2-difluoroethylsulfinyl, 2,2-dichloro-2-fluoroethylsulfinyl, 2,2,2-trichloroethylsulfinyl or S(O)C₂F₅. C₁-C₄-Haloalkylsulfinyl is additionally, for example, 2-fluoropropylsulfinyl, 3-fluoropropylsulfinyl, 2,2-difluoropropylsulfinyl, 2,3-difluoropropylsulfinyl, 2-chloropropylsulfinyl, 3-chloropropylsulfinyl, 2,3-dichloropropylsulfinyl, 2-bromopropylsulfinyl, 3-bromopropylsulfinyl, 3,3,3-trifluoropropylsulfinyl, 3,3,3-trichloropropylsulfinyl, S(O)CH₂—C₂F₅, S(O)CF₂—C₂F₅, 1-(CH₂F)-2-fluoroethylsulfinyl, 1-(CH₂Cl)-2-chloroethylsulfinyl, 1-(CH₂Br)-2-bromoethylsulfinyl, 4-fluorobutylsulfinyl, 4-chlorobutylsulfinyl, 4-bromobutylsulfinyl or nonafluorobutylsulfinyl. C₁-C₆-Haloalkylsulfinyl is additionally, for example, 5-fluoropentylsulfinyl, 5-chloropentylsulfinyl, 5-brompentylsulfinyl, 5-iodopentylsulfinyl, undecafluoropentylsulfinyl, 6-fluorohexylsulfinyl, 6-chlorohexylsulfinyl, 6-bromohexylsulfinyl, 6-iodohexylsulfinyl or dodecafluorohexylsulfinyl.

The term “C₁-C₂-alkylsulfonyl” is a C₁-C₂-alkyl group, as defined above, attached via a sulfonyl [S(O)₂] group. The term “C₁-C₄-alkylsulfonyl” is a C₁-C₄-alkyl group, as defined above, attached via a sulfonyl [S(O)₂] group. The term “C₁-C₆-alkylsulfonyl” is a C₁-C₆-alkyl group, as defined above, attached via a sulfonyl [S(O)₂] group. The term “C₁-C₁₀-alkylsulfonyl” is a C₁-C₁₀-alkyl group, as defined above, attached via a sulfonyl [S(O)₂] group. C₁-C₂-Alkylsulfonyl is methylsulfonyl or ethylsulfonyl. C₁-C₄-Alkylsulfonyl is additionally, for example, n-propylsulfonyl, 1-methylethylsulfonyl(isopropylsulfonyl), butylsulfonyl, 1-methylpropylsulfonyl(sec-butylsulfonyl), 2-methylpropylsulfonyl(isobutylsulfonyl) or 1,1-dimethylethylsulfonyl(tert-butylsulfonyl). C₁-C₆-Alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl. C₁-C₈-Alkylsulfonyl is additionally, for example, heptylsulfonyl, octylsulfonyl, 2-ethylhexylsulfonyl and positional isomers thereof. C₁-C₁₀-Alkylsulfonyl is additionally, for example, nonylsulfonyl, decylsulfonyl and positional isomers thereof.

The term “C₁-C₂-haloalkylsulfonyl” is a C₁-C₂-haloalkyl group, as defined above, attached via a sulfonyl [S(O)₂] group. The term “C₁-C₄-haloalkylsulfonyl” is a C₁-C₄-haloalkyl group, as defined above, attached via a sulfonyl [S(O)₂] group. The term “C₁-C₆-haloalkylsulfonyl” is a C₁-C₆-haloalkyl group, as defined above, attached via a sulfonyl [S(O)₂] group. The term “C₁-C₁₀-haloalkylsulfonyl” is a C₁-C₁₀-haloalkyl group, as defined above, attached via a sulfonyl [S(O)₂] group. C₁-C₂-Haloalkylsulfonyl is, for example, S(O)₂CH₂F, S(O)₂CHF₂, S(O)₂CF₃, S(O)₂CH₂Cl, S(O)₂CHCl₂, S(O)₂CCl₃, chlorofluoromethylsulfonyl, dichlorofluoromethylsulfonyl, chlorodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl or S(O)₂C₂F₅. C₁-C₄-Haloalkylsulfonyl is additionally, for example, 2-fluoropropylsulfonyl, 3-fluoropropylsulfonyl, 2,2-difluoropropylsulfonyl, 2,3-difluoropropylsulfonyl, 2-chloropropylsulfonyl, 3-chloropropylsulfonyl, 2,3-dichloropropylsulfonyl, 2-bromopropylsulfonyl, 3-bromopropylsulfonyl, 3,3,3-trifluoropropylsulfonyl, 3,3,3-trichloropropylsulfonyl, S(O)₂CH₂—C₂F₅, S(O)₂CF₂—C₂F₅, 1-(CH₂F)-2-fluoroethylsulfonyl, 1-(CH₂Cl)-2-chloroethylsulfonyl, 1-(CH₂Br)-2-bromoethylsulfonyl, 4-fluorobutylsulfonyl, 4-chlorobutylsulfonyl, 4-bromobutylsulfonyl or nonafluorobutylsulfonyl. C₁-C₆-Haloalkylsulfonyl is additionally, for example, 5-fluoropentylsulfonyl, 5-chloropentylsulfonyl, 5-brompentylsulfonyl, 5-iodopentylsulfonyl, undecafluoropentylsulfonyl, 6-fluorohexylsulfonyl, 6-chlorohexylsulfonyl, 6-bromohexylsulfonyl, 6-iodohexylsulfonyl or dodecafluorohexylsulfonyl.

The term “3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members” as used herein refers to monocyclic radicals, the monocyclic radicals being saturated, partially unsaturated or aromatic. The heterocyclic radical may be attached to the remainder of the molecule via a carbon ring member or via a nitrogen ring member.

Examples of 3-, 4-, 5-, 6- or 7-membered saturated heterocyclyl include: Oxiranyl, aziridinyl, oxetidinyl (radical of trimethylene oxide), thietidinyl (radical of trimethylene sulfide), azetidinyl, 2-tetrahydrofuranyl, 3-tetrahydrofuranyl, 1,3-dioxolane-2-yl, 1,3-dioxolane-4-yl, 2-tetrahydrothienyl, 3-tetrahydrothienyl, 1,3-thiolane-2-yl, 1,3-dithiolane-4-yl, 1-thia-3-oxolan-2-yl, 1-thia-3-oxolan-4-yl, 1-thia-3-oxolan-5-yl, 2-pyrrolidinyl, 3-pyrrolidinyl, 3-pyrazolidinyl, 4-pyrazolidinyl, 5-pyrazolidinyl, 2-imidazolidinyl, 4-imidazolidinyl, 2-oxazolidinyl, 4-oxazolidinyl, 5-oxazolidinyl, 3-isoxazolidinyl, 4-isoxazolidinyl, 5-isoxazolidinyl, 2-thiazolidinyl, 4-thiazolidinyl, 5-thiazolidinyl, 3-isothiazolidinyl, 4-isothiazolidinyl, 5-isothiazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2-tetrahydropyranyl, 3-tetrahydropyranyl, 4-tetrahydropyranyl, 1,3-dioxan-2-yl, 1,3-dioxan-4-yl, 1,3-dioxan-5-yl, 1,4-dioxan-2-yl, 2-thianyl, 3-thianyl, 4-thianyl, 1,3-dithian-2-yl, 1,3-dithian-4-yl, 1,3-dithian-5-yl, 1,4-dithian-2-yl, 1-oxa-3-thian-2-yl, 1-oxa-3-thian-4-yl, 1-oxa-3-thian-5-yl, 1-oxa-3-thian-6-yl, 1-oxa-4-thian-2-yl, 1-oxa-4-thian-3-yl, 2-piperidinyl, 3-piperidinyl, 4-piperidinyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl, 2-morpholinyl, 3-morpholinyl, 2-thiomorpholinyl, 3-thiomorpholinyl, 1-oxothiomorpholin-2-yl, 1-oxothiomorpholin-3-yl, 1,1-dioxothiomorpholin-2-yl, 1,1-dioxothiomorpholin-3-yl, hexahydroazepin-1-, -2-, -3- or -4-yl, hexahydrooxepinyl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of 3-, 4-, 5-, 6- or 7-membered partially unsaturated heterocyclyl include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.

3-, 4-, 5-, 6- or 7-membered aromatic heterocyclyl is 5- or 6-membered aromatic heterocyclyl (hetaryl). Examples are: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

C₂-C₇-alkylene is divalent branched or preferably unbranched saturated aliphatic chain having 2 to 7 carbon atoms, for example CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂, CH₂CH₂CH₂CH₂CH₂CH₂, and CH₂CH₂CH₂CH₂CH₂CH₂CH₂

The remarks made below concerning preferred embodiments of the variables of the compounds of formula I, especially with respect to their substituents X, Y, G, A¹, B¹, R¹, R², R³, R⁴, R⁵, R⁶, R⁷, R⁸, R^{8a}, R⁹, R¹⁰, R¹¹, R¹², R¹³, R¹⁴, R¹⁵, R¹⁶, R¹⁷, R¹⁸, R¹⁹, R²⁰, R²¹, R²², m, n, p and q, the features of the use and method according to the invention and of the composition of the invention are valid both on their own and, in particular, in every possible combination with each other.

As a matter of course, the q radicals R⁵ replace a hydrogen atom on a carbon ring atom. For instance, if B¹ is defined to be CH and if this position is to be substituted by a radical R⁵, then B¹ is of course C—R⁵. If there is more than one radical R⁵, these can be the same or different.

As a matter of course, the p radicals R⁴ replace a hydrogen atom on a carbon ring atom. For instance, if A¹ is defined to be CH and if this position is to be substituted by a radical R⁴, then A¹ is of course C—R⁴. If there is more than one radical R⁴, these can be the same or different.

Preferably, A¹ is CH.

In a preferred embodiment, the ring comprising the group A¹ as ring member carries 0, 1 or 2, preferably 0 or 1 and in particular 1 substituent R⁴. In other words, p is preferably 0, 1 or 2, more preferably 0 or 1 and in particular 1. In case A¹ is CH and p is 1, the substituent R⁴ is preferably bound on the position of A¹. In other words, A¹ is in this case preferably C—R⁴.

In case p is 2, two substituents R⁴ bound on adjacent carbon atoms preferably form together a group selected from —CH₂CH₂CH₂CH₂— and —CH═CH—CH═CH— and more preferably —CH═CH—CH═CH—, thus yielding a fused phenyl ring.

Preferably, B¹ is CH.

q is preferably 0, 1, 2 or 3, more preferably 1, 2 or 3, even more preferably 2 or 3 and in particular 2. If q is 3 and B¹ is CH, then the three substituents R⁵ are preferably bound in the positions 3, 4 and 5 (relative to the 1-position of the attachment point of this ring to the remainder of the molecule), B¹ thus being C—R⁵. If q is 2 and B¹ is CH, then the two substituents R⁵ are preferably bound in the positions 3 and 5, B¹ thus being C—R⁵.

X is preferably selected from the group consisting of C₁-C₄-alkyl, C₁-C₄-haloalkyl, C₁-C₄-haloalkoxy-C₁-C₄-alkyl, C₃-C₆-cycloalkyl and C₃-C₆-halocycloalkyl. More preferably, X is selected from the group consisting of C₁-C₄-alkyl, C₃-C₆-cycloalkyl and C₃-C₆-halocycloalkyl. Even more preferably, X is selected from the group consisting of C₁-C₄-alkyl and C₁-C₄-haloalkyl. In particular, X is C₁-C₄-haloalkyl, specifically C₁-C₂-haloalkyl and more specifically halomethyl, in particular fluoromethyl, such as fluoromethyl, difluoromethyl and trifluoromethyl, or fluorochloromethyl, such as chlorodifluoromethyl or dichlorofluoromethyl. Specifically, X is selected from CF₃, CHF₂ and CF₂Cl and is very specifically trifluoromethyl.

Y is preferably a chemical bond, O or NR³. R³ has one of the meanings given above or preferably one of the preferred meanings given below. More preferably, Y is O or NR³. In particular, Y is NR³.

Preferably, R¹ is selected from the group consisting of hydrogen; cyano; C₁-C₁₀-alkyl which may be partially or fully halogenated and/or may be substituted by one or more, e.g. 1, 2, 3 or 4, preferably 1 or 2, more preferably 1, radicals R⁶; C₁-C₁₀-alkoxy; C₁-C₁₀-haloalkoxy; C₃-C₈-cycloalkyl which may be partially or fully halogenated and/or may be substituted by one or more, e.g. 1, 2, 3 or 4, preferably 1 or 2, more preferably 1, radicats R⁶; C₂-C₁₀-alkenyl which may be partially or fully halogenated and/or may be substituted by one or more, e.g. 1, 2, 3 or 4, preferably 1 or 2, more preferably 1, radicals R⁶; C₂-C₁₀-alkynyl which may be partially or fully halogenated and/or may be substituted by one or more, e.g. 1, 2, 3 or 4, preferably 1 or 2, more preferably 1, radicals R⁶; —C(═O)R⁶; —C(═O)OR⁷; —C(═O)N(R⁸)R⁹; —C(═S)R⁶; —C(═S)OR⁷; —C(═S)N(R⁸)R⁹; phenyl which may be substituted by 1, 2, 3, 4 or 5, preferably 1 or 2, more preferably 1, radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰;

where R⁷, R⁸, R⁹ and R¹⁰ have one of the meanings given above or in particular one of the preferred meanings given below.

More preferably, R¹ is selected from the group consisting of hydrogen; cyano; C₁-C₁₀-alkyl which may be partially or fully halogenated and/or may be substituted by one or more, e.g. 1, 2, 3 or 4, preferably 1 or 2, more preferably 1, radicals R⁶; C₁-C₁₀-alkoxy; C₁-C₁₀-haloalkoxy; C₃-C₈-cycloalkyl which may be partially or fully halogenated and/or may be substituted by one or more, e.g. 1, 2, 3 or 4, preferably 1 or 2, more preferably 1, radicals R⁶; C₂-C₁₀-alkenyl which may be partially or fully halogenated and/or may be substituted by one or more, e.g. 1, 2, 3 or 4, preferably 1 or 2, more preferably 1, radicals R⁶; C₂-C₁₀-alkynyl which may be partially or fully halogenated and/or may be substituted by one or more, e.g. 1, 2, 3 or 4, preferably 1 or 2, more preferably 1, radicals R⁶; —C(═O)R⁶; phenyl which may be substituted by 1, 2, 3, 4 or 5, preferably 1 or 2, more preferably 1, radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰,

where R⁶ and R¹⁰ have one of the meanings given above or in particular one of the preferred meanings given below.

Even more preferably, R¹ is selected from the group consisting of hydrogen; cyano; C₁-C₁₀-alkyl which may be partially or fully halogenated and/or may be substituted by one or more, e.g. 1, 2, 3 or 4, preferably 1 or 2, more preferably 1, radicals R⁶; C₁-C₁₀-alkoxy; C₁-C₁₀-haloalkoxy; C₃-C₈-cycloalkyl which may be partially or fully halogenated and/or may be substituted by one or more, e.g. 1, 2, 3 or 4, preferably 1 or 2, more preferably 1, radicals R⁶, and —C(═O)R⁶; where R⁶ has one of the meanings given above or in particular one of the preferred meanings given below.

In particular, R¹ is selected from the group consisting of hydrogen, cyano, C₁-C₁₀-alkyl, preferably C₁-C₆-alkyl, more preferably C₁-C₄-alkyl, which may be partially or fully halogenated and/or may be substituted by one or more, e.g. 1, 2, 3 or 4, preferably 1 or 2, more preferably 1, radicals R⁶; cyclopropyl; C₁-C₄-alkoxy; C₁-C₄-haloalkoxy, and —C(═O)R⁶; where R⁶ has one of the meanings given above or in particular one of the preferred meanings given below.

Specifically, R¹ is selected from the group consisting of hydrogen; cyano; C₁-C₆-alkyl; C₁-C₄-haloalkyl, specifically C₁-C₄-fluoroalkyl; C₁-C₄-alkoxy; C₁-C₄-haloalkoxy, specifically C₁-C₄-fluoroalkoxy; and —C(═O)R⁶; where R⁶ has one of the meanings given above or preferably one of the preferred meanings given below. More specifically, R¹ is hydrogen.

In case R¹ is selected from C₁-C₁₀-alkyl, preferably C₁-C₆-alkyl, more preferably C₁-C₄-alkyl, which is substituted by one or more, e.g. 1, 2, 3 or 4, preferably 1 or 2, more preferably 1, radicals R⁶, R⁶ is preferably selected from C₃-C₆-cycloalkyl, C₃-C₆-halocycloalkyl, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰, more preferably from a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰, even more preferably from a 5- or 6-membered saturated, partially unsaturated or aromatic heterocyclic ring containing 1, 2 or 3 heteroatoms selected from N, O and S, as ring members, where the heterocyclic ring may be substituted by one or more, e.g. 1, 2 or 3, preferably 1 or 2, more preferably 1, radicals R¹⁰, in particular from a 5- or 6-membered heteroaromatic ring containing 1 heteroatom selected from N, O and S and optionally 1 or two further N atoms, as ring members, where the heteroaromatic ring may be substituted by one or more, e.g. 1, 2 or 3, preferably 1 or 2, more preferably 1, radicals R¹⁰, and is specifically 6-membered heteroaromatic ring selected from pyridinyl, pyridazinyl, pyrimidinyl, pyrazinyl and 1,3,5-triazinyl, preferably from pyridyl and pyrimidinyl, where the heteroaromatic ring may be substituted by one or more, e.g. 1, 2 or 3, preferably 1 or 2, more preferably 1, radicals R¹⁰,

where R¹⁰ has one of the meanings given above or in particular one of the preferred meanings given below.

R² is preferably selected from the group consisting of hydrogen; C₁-C₁₀-alkyl which may be partially or fully halogenated and/or may be substituted by one or more, e.g. 1, 2, 3 or 4, preferably 1 or 2, more preferably 1, radicals R⁶; C₃-C₈-cycloalkyl which may be partially or fully halogenated and/or may be substituted by one or more, e.g. 1, 2, 3 or 4, preferably 1 or 2, more preferably 1, radicals R⁶; C₂-C₁₀-alkenyl which may be partially or fully halogenated and/or may be substituted by one or more, e.g. 1, 2, 3 or 4, preferably 1 or 2, more preferably 1, radicals R⁶; C₂-C₁₀-alkynyl which may be partially or fully halogenated and/or may be substituted by one or more, e.g. 1, 2, 3 or 4, preferably 1 or 2, more preferably 1, radicals R⁶; —C(═O)R⁶; —C(═O)OR⁷; —C(═O)N(R⁸)R⁹; —C(═S)R⁶; —C(═S)OR⁷, —C(═S)N(R⁸)R⁹; —C(═NR⁸)R⁶, phenyl which may be substituted by 1, 2, 3, 4 or 5, preferably 1 or 2, more preferably 1, radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰,

or R² and R³ together form a group ═CR¹¹R¹²; ═S(O)ₘR⁷; ═S(O)ₘN(R⁸)R⁹; ═NR⁸; or ═NOR⁷;
or R² and R³ together form a C₂-C₇ alkylene chain, thus forming, together with the nitrogen atom to which they are bound, a 3-, 4-, 5-, 6-, 7- or 8-membered ring, where the alkylene chain may be interrupted by 1 or 2 O, S and/or NR¹⁸ and/or 1 or 2 of the CH₂ groups of the alkylene chain may be replaced by a group C═O, C═S and/or C═NR¹⁸; and/or the alkylene chain may be substituted by one or more, e.g. 1, 2, 3 or 4, preferably 1 or 2, more preferably 1, radicals selected from the group consisting of halogen, C₁-C₆-haloalkyl, C₁-C₆-alkoxy, C₁-C₆-haloalkoxy, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5, preferably 1 or 2, more preferably 1, radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰,
where R⁶, R⁷, R⁸, R⁹, R¹⁰, R¹¹, R¹² and R¹⁸ have one of the meanings given above or in particular one of the preferred meanings given below.

More preferably, R² is selected from the group consisting of hydrogen; C₁-C₁₀-alkyl which may be partially or fully halogenated and/or may be substituted by one or more, e.g. 1, 2, 3 or 4, preferably 1 or 2, more preferably 1, radicals R⁶; C₃-C₈-cycloalkyl which may be partially or fully halogenated and/or may be substituted by one or more, e.g. 1, 2, 3 or 4, preferably 1 or 2, more preferably 1, radicals R⁶; C₂-C₁₀-alkenyl which may be partially or fully halogenated and/or may be substituted by one or more, e.g. 1, 2, 3 or 4, preferably 1 or 2, more preferably 1, radicals R⁶; C₂-C₁₀-alkynyl which may be partially or fully halogenated and/or may be substituted by one or more, e.g. 1, 2, 3 or 4, preferably 1 or 2, more preferably 1, radicals R⁶; —C(═O)R⁶; —C(═O)OR⁷; —C(═O)N(R⁸)R⁹; —C(═S)R⁶; —C(═S)OR⁷, —C(═S)N(R⁸)R⁹; —C(═NR⁸)R⁶, phenyl which may be substituted by 1, 2, 3, 4 or 5, preferably 1 or 2, more preferably 1, radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰,

where R⁶, R⁷, R⁸, R⁹ and R¹⁰ have one of the meanings given above or in particular one of the preferred meanings given below.

In case Y is a chemical bond, R² is more preferably selected from a substituent bound via a heteroatom, such as —N(R⁸)R⁹; —N(R⁸)C(═O)R⁶; —OR⁷; —SR⁷; —S(O)ₘR⁷; —S(O)ₙN(R⁸)R⁹ and an N-bound 3-, 4-, 5-, 6- or 7-membered saturated, partially unsaturated or aromatic heterocyclic ring containing 1 N atom as ring member and optionally 1 or 2 further heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰, where R⁶, R⁷, R⁸, R⁹ and R¹⁰ have one of the meanings given above or in particular one of the preferred meanings given below.

In case Y is a chemical bond, R² is even more preferably selected from —N(R⁸)R⁹; —N(R⁸)C(═O)R⁶; —OR⁷; —SR⁷; —S(O)ₘR⁷ and S(O)ₙN(R⁸)R⁹, in particular from —N(R⁸)R⁹; —N(R⁸)C(═O)R⁶; —OR⁷ and —SR⁷, and specifically from —N(R⁸)R⁹; —N(R⁸)C(═O)R⁶ and —OR⁷, where R⁶, R⁷, R⁸ and R⁹ have one of the meanings given above or in particular one of the preferred meanings given below.

In case Y is not a chemical bond, R² is preferably selected from the group consisting of hydrogen; C₁-C₁₀-alkyl which may be partially or fully halogenated and/or may be substituted by one or more, e.g. 1, 2, 3 or 4, preferably 1 or 2, more preferably 1, radicals R⁶; C₃-C₈-cycloalkyl which may be partially or fully halogenated and/or may be substituted by one or more, e.g. 1, 2, 3 or 4, preferably 1 or 2, more preferably 1, radicals R⁶; C₂-C₁₀-alkenyl which may be partially or fully halogenated and/or may be substituted by one or more, e.g. 1, 2, 3 or 4, preferably 1 or 2, more preferably 1, radicals R⁶; C₂-C₁₀-alkynyl which may be partially or fully halogenated and/or may be substituted by one or more, e.g. 1, 2, 3 or 4, preferably 1 or 2, more preferably 1, radicals R⁶; —C(═O)R⁶; —C(═O)OR⁷; —C(═O)N(R⁸)R⁹; —C(═S)R⁶; —C(═S)OR⁷, —C(═S)N(R⁸)R⁹; —C(═NR⁸)R⁶, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰,

where R⁶, R⁷, R⁸, R⁹ and R¹⁰ have one of the meanings given above or in particular one of the preferred meanings given below.

In case Y is not a chemical bond, R² is more preferably selected from the group consisting of hydrogen; C₁-C₁₀-alkyl which may be partially or fully halogenated and/or may be substituted by one or more, e.g. 1, 2, 3 or 4, preferably 1 or 2, more preferably 1, radicals R⁶; C₃-C₈-cycloalkyl which may be partially or fully halogenated and/or may be substituted by one or more, e.g. 1, 2, 3 or 4, preferably 1 or 2, more preferably 1, radicals R⁶; —C(═O)R⁶; —C(═O)OR⁷; —C(═O)N(R⁸)R⁹; —C(═S)R⁶; —C(═S)OR⁷, —C(═S)N(R⁸)R⁹; —C(═NR⁸)R⁶, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰,

where R⁶, R⁷, R⁸, R⁹ and R¹⁰ have one of the meanings given above or in particular one of the preferred meanings given below.

Independently from the meaning of Y, R² is even more preferably selected from the group consisting of C₁-C₄-alkyl; C₁-C₄-haloalkyl; a methyl group substituted by a radical R⁶; —C(═O)R⁶; —C(═O)N(R⁸)R⁹; —C(═O)OR⁷; —C(═S)R⁶; —C(═S)N(R⁸)R⁹; —C(═S)OR⁷; and —C(═NR⁸)R⁶; and specifically from —C(═O)N(R⁸)R⁹ and —C(═S)N(R⁸)R⁹; where R⁶, R⁷, R⁸ and R⁹ have one of the meanings given above or in particular one of the preferred meanings given below. It is however preferred that R² has these meanings if Y is not a chemical bond and is preferably O or NR³, specifically NR³.

Independently from the meaning of Y, R² is particularly preferably selected from the group consisting of C₁-C₄-alkyl; C₁-C₄-haloalkyl; a methyl group substituted by a radical R^{6a}; —C(═O)R^{6c}; —C(═O)N(R⁸)R⁹; —C(═O)OR⁷; —C(═S)R^{6c}; —C(═S)N(R⁸)R⁹; —C(═S)OR⁷; and —C(═NR⁸)R^{6d}; and specifically from —C(═O)N(R⁸)R⁹ and —C(═S)N(R⁸)R⁹;

where
R^{6a} is selected from CN, phenyl which may carry 1, 2 or 3, preferably 1 or 2, more preferably 1, substituents R¹⁰, —C(═O)R^{6b}; —C(═O)N(R⁸)R⁹ and —C(═O)OR⁷;
R^{6b} and R^{6c} are independently selected from C₁-C₄-alkyl, C₁-C₄-haloalkyl, phenyl, benzyl and a 5- or 6-membered saturated, partially unsaturated or aromatic heterocyclic ring containing 1, 2 or 3 heteroatoms or heteroatom groups selected from N, O, S, NO, SO and SO₂, as ring members, where the phenyl or heterocyclyl rings in the three last-mentioned radicals may carry 1, 2 or 3, preferably 1 or 2, more preferably 1, substituents selected from halogen, CN, C₁-C₄-alkyl, C₁-C₄-haloalkyl, C₁-C₄-alkoxy and C₁-C₄-haloalkoxy;
R^{6d} is selected from N(R⁸)R⁹;
R⁷ is selected from hydrogen, C₁-C₄-alkyl, C₁-C₄-haloalkyl, phenyl, benzyl and a 5- or 6-membered saturated, partially unsaturated or aromatic heterocyclic ring containing 1, 2 or 3 heteroatoms or heteroatom groups selected from N, O, S, NO, SO and SO₂, as ring members, where the phenyl or heterocyclyl rings in the three last-mentioned radicals may carry 1, 2 or 3, preferably 1 or 2, more preferably 1, substituents selected from halogen, CN, C₁-C₄-haloalkyl, C₁-C₄-alkoxy and C₁-C₄-haloalkoxy;
each R⁸ is independently selected from hydrogen, cyano, C₁-C₆-alkyl which may be partially or fully halogenated and/or may be substituted by one or more radicals R¹⁹, C₂-C₄-alkenyl which may be partially or fully halogenated and/or may be substituted by one or more radicals R¹⁹, C₃-C₆-cycloalkyl, C₃-C₆-halocycloalkyl, C₃-C₆-cycloalkyl-C₁-C₄-alkyl, C₃-C₆-halocycloalkyl-C₁-C₄-alkyl, —S(O)ₘR²⁰, —S(O)ₙN(R²¹)R²², phenyl, benzyl and a 5- or 6-membered saturated, partially unsaturated or aromatic heterocyclic ring containing 1, 2 or 3 heteroatoms or heteroatom groups selected from N, O, S, NO, SO and SO₂, as ring members, where the phenyl or heterocyclyl rings in the three last-mentioned radicals may carry 1, 2 or 3, preferably 1 or 2, more preferably 1, substituents selected from halogen, CN, C₁-C₄-alkyl, C₁-C₄-haloalkyl, C₁-C₄-alkoxy and C₁-C₄-haloalkoxy;
each R⁹ is independently selected from hydrogen, cyano, C₁-C₆-alkyl which may be partially or fully halogenated and/or may be substituted by one or more radicals R¹⁹, C₂-C₄-alkenyl which may be partially or fully halogenated and/or may be substituted by one or more radicals R¹⁹, C₃-C₆-cycloalkyl, C₃-C₆-halocycloalkyl, C₃-C₆-cycloalkyl-C₁-C₄-alkyl, C₃-C₆-halocycloalkyl-C₁-C₄-alkyl, —S(O)ₘR²⁰, —S(O)ₙN(R²¹)R²², phenyl, benzyl and a 5- or 6-membered saturated, partially unsaturated or aromatic heterocyclic ring containing 1, 2 or 3 heteroatoms or heteroatom groups selected from N, O, S, NO, SO and SO₂, as ring members, where the phenyl or heterocyclyl rings in the three last-mentioned radicals may carry 1, 2 or 3, preferably 1 or 2, more preferably 1, substituents selected from halogen, CN, C₁-C₄-alkyl, C₁-C₄-haloalkyl, C₁-C₄-alkoxy and C₁-C₄-haloalkoxy; and
R¹⁰ is selected from halogen, CN, C₁-C₄-alkyl, C₁-C₄-haloalkyl, C₁-C₄-alkoxy and C₁-C₄-haloalkoxy;
   where R¹⁹ has one of the meanings given above or in particular one of the preferred meanings given below; or
R⁸ and R⁹ together form a group ═CR¹¹R¹²; or
R⁸ and R⁹, together with the nitrogen atom to which they are bound, form a 3-, 4-, 5-, 6- or 7-membered saturated, partially unsaturated or aromatic heterocyclic ring which may additionally containing 1 or 2 further heteroatoms or heteroatom groups selected from N, O, S, NO, SO and SO₂, as ring members, where the heterocyclic ring may be substituted by one or more radicals R¹⁰.

It is however preferred that R² has these meanings if Y is not a chemical bond and is preferably O or NR³, specifically NR³.

In particular, R² is selected from —C(═O)N(R⁸)R⁹ and —C(═S)N(R⁸)R⁹, where R⁸ and R⁹ have one of the meanings given above or in particular one of the preferred meanings given below. More preferably, R² is selected from —C(═O)N(R⁸)R⁹ and —C(═S)N(R⁸)R⁹, where
R⁸ is selected from hydrogen and C₁-C₆-alkyl which may be partially or fully halogenated and/or may be substituted by one or more radicals R¹⁹;
R⁹ is selected from hydrogen, C₁-C₆-alkyl which may be partially or fully halogenated and/or may be substituted by one or more radicals R¹⁹, C₃-C₆-cycloalkyl, C₃-C₆-halocycloalkyl, C₃-C₆-cycloalkyl-C₁-C₄-alkyl and C₃-C₆-halocycloalkyl-C₁-C₄-alkyl; and
R¹⁹ has one of the meanings given above or in particular one of the preferred meanings given below.

It is preferred that R² has these meanings if Y is not a chemical bond and is preferably O or NR³, specifically NR³.

Specifically, R² is selected from —C(═O)N(R⁸)R⁹ and —C(═S)N(R⁸)R⁹, where
R⁸ is hydrogen; and
R⁹ is selected from hydrogen, C₁-C₆-alkyl; C₁-C₆-haloalkyl, C₃-C₆-cycloalkyl, C₃-C₆-halocycloalkyl, C₃-C₆-cycloalkyl-C₁-C₄-alkyl and C₃-C₆-halocycloalkyl-C₁-C₄-alkyl.

It is preferred that R² has these meanings if Y is not a chemical bond and is preferably O or NR³, specifically NR³.

R³ is preferably selected from the group consisting of hydrogen; cyano; C₁-C₁₀-alkyl which may be partially or fully halogenated and/or may be substituted by one or more, e.g. 1, 2, 3 or 4, preferably 1 or 2, more preferably 1, radicals R⁶; C₃-C₈-cycloalkyl which may be partially or fully halogenated and/or may be substituted by one or more, e.g. 1, 2, 3 or 4, preferably 1 or 2, more preferably 1, radicals R⁶; C₂-C₁₀-alkenyl which may be partially or fully halogenated and/or may be substituted by one or more, e.g. 1, 2, 3 or 4, preferably 1 or 2, more preferably 1, radicals R⁶; C₂-C₁₀-alkynyl which may be partially or fully halogenated and/or may be substituted by one or more, e.g. 1, 2, 3 or 4, preferably 1 or 2, more preferably 1, radicals R⁶; —N(R⁸)R⁹; —Si(R¹⁴)₂R¹³; —OR⁷; —SR⁷; —S(O)ₘR⁷; —S(O)ₙN(R⁸)R⁹; —C(═O)R⁶; —C(═O)OR⁷; —C(═O)N(R⁸)R⁹; —C(═S)R⁶; —C(═S)OR⁷; —C(═S)N(R⁸)R⁹; —C(═NR⁸)R⁶; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;

or R² and R³ together form a group ═CR¹¹R¹²; ═S(O)ₘR⁷; ═S(O)ₘN(R⁸)R⁹; ═NR⁸; or ═NOR⁷;
or R² and R³ together form a C₂-C₇ alkylene chain, thus forming, together with the nitrogen atom to which they are bound, a 3-, 4-, 5-, 6-, 7- or 8-membered ring, where the alkylene chain may be interrupted by 1 or 2 O, S and/or NR¹⁸ and/or 1 or 2 of the CH₂ groups of the alkylene chain may be replaced by a group C═O, C═S and/or C═NR¹⁸; and/or the alkylene chain may be substituted by one or more, e.g. 1, 2, 3 or 4, preferably 1 or 2, more preferably 1, radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5, preferably 1 or 2, more preferably 1, radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰,
where R⁶, R⁷, R⁸, R⁹, R¹⁰, R¹¹, R¹², R¹³, R¹⁴ and R¹⁸ have one of the meanings given above or in particular one of the preferred meanings given below.

More preferably, R³ is selected from the group consisting of hydrogen; C₁-C₁₀-alkyl which may be partially or fully halogenated and/or may be substituted by one or more, e.g. 1, 2, 3 or 4, preferably 1 or 2, more preferably 1, radicals R⁶; C₃-C₈-cycloalkyl which may be partially or fully halogenated and/or may be substituted by one or more, e.g. 1, 2, 3 or 4, preferably 1 or 2, more preferably 1, radicals R⁶; C₂-C₁₀-alkenyl which may be partially or fully halogenated and/or may be substituted by one or more, e.g. 1, 2, 3 or 4, preferably 1 or 2, more preferably 1, radicals R⁶; C₂-C₁₀-alkynyl which may be partially or fully halogenated and/or may be substituted by one or more, e.g. 1, 2, 3 or 4, preferably 1 or 2, more preferably 1, radicals R⁶; —C(═O)R⁶; —C(═O)OR⁷; —C(═O)N(R⁸)R⁹; —C(═S)R⁶; —C(═S)OR⁷; —C(═S)N(R⁸)R⁹; —C(═NR⁸)R⁶; phenyl which may be substituted by 1, 2, 3, 4 or 5, preferably 1 or 2, more preferably 1, radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰,

where R⁶, R⁷, R⁸, R⁹ and R¹⁰ have one of the meanings given above or in particular one of the preferred meanings given below.

Even more preferably, R³ is selected from the group consisting of hydrogen; C₁-C₁₀-alkyl which may be partially or fully halogenated and/or may be substituted by one or more, e.g. 1, 2, 3 or 4, preferably 1 or 2, more preferably 1, radicals R⁶; —C(═O)R⁶; —C(═O)OR⁷; —C(═O)N(R⁸)R⁹; —C(═S)R⁶; —C(═S)OR⁷; —C(═S)N(R⁸)R⁹ and —C(═NR⁸)R⁶; where R⁶, R⁷, R⁸ and R⁹ have one of the meanings given above and in particular one of the preferred meanings given below. Preferably, in this case, R⁶ as a C₁-C₆-alkyl substituent, is selected from CN, C₃-C₆-cycloalkyl, C₃-C₆-halocycloalkyl, C₁-C₆-alkoxy, C₁-C₆-haloalkoxy, C₁-C₆-alkylthio, C₁-C₆-haloalkylthio and a 5- or 6-membered hetaryl ring containing 1, 2 or 3 heteroatoms selected from N, O and S as ring members and being optionally substituted by 1, 2 or 3 radicals R¹⁰. In this case, R⁶ as a CO substituent, is preferably selected from C₁-C₆-alkyl, C₁-C₆-haloalkyl, C₁-C₆-alkoxy and C₁-C₆-haloalkoxy. In this case, R⁸ and R⁹ are preferably selected from hydrogen and C₁-C₆-alkyl.

In particular, R³ is selected from the group consisting of hydrogen; C₁-C₆-alkyl and C₁-C₄-haloalkyl and is specifically hydrogen.

Preferably, each R⁴ is independently selected from halogen; cyano; nitro; —SCN; SF₅; C₁-C₆-alkyl which may be partially or fully halogenated and/or may be substituted by one or more, e.g. 1, 2, 3 or 4, preferably 1 or 2, more preferably 1, radicals R⁶; C₃-C₈-cycloalkyl which may be partially or fully halogenated and/or may be substituted by one or more, e.g. 1, 2, 3 or 4, preferably 1 or 2, more preferably 1, radicals R⁶; C₂-C₆-alkenyl which may be partially or fully halogenated and/or may be substituted by one or more, e.g. 1, 2, 3 or 4, preferably 1 or 2, more preferably 1, radicals R⁶; C₂-C₆-alkynyl which may be partially or fully halogenated and/or may be substituted by one or more, e.g. 1, 2, 3 or 4, preferably 1 or 2, more preferably 1, radicals R⁶; —Si(R¹⁴)₂R¹³; —OR⁷; —OS(O)ₙR⁷; —SR⁷; —S(O)ₘR⁷; —S(O)ₙN(R⁸)R⁹; —N(R⁸)R⁹; —N(R⁸)C(═O)R⁶; C(═O)R⁶; —C(═O)OR⁷; —C(═NR⁸)H; —C(═NR⁸)R⁶; —C(═O)N(R⁸)R⁹; C(═S)N(R⁸)R⁹; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰;

or two radicals R⁴ bound on adjacent carbon atoms may be together a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR⁸—, —CH₂CH═N—, —CH═CH—NR⁸—, —OCH═N—, and —SCH═N—, thus forming, together with the carbon atoms to which they are bound, a 5- or 6-membered ring, where the hydrogen atoms of the above groups may be replaced by one or more, e.g. 1, 2, 3 or 4, preferably 1 or 2, more preferably 1, substituents selected from halogen, methyl, halomethyl, hydroxyl, methoxy and halomethoxy or one or more, e.g. 1 or 2, CH₂ groups of the above groups may be replaced by a C═O group,
where R⁶, R⁷, R⁸, R⁹, R¹⁰, R¹³ and R¹⁴ have one of the meanings given above or in particular one of the preferred meanings given below.

More preferably, each R⁴ is independently selected from halogen; cyano; nitro; —SCN; C₁-C₆-alkyl which may be partially or fully halogenated and/or may be substituted by one or more, e.g. 1, 2, 3 or 4, preferably 1 or 2, more preferably 1, radicals R⁶; C₃-C₈-cycloalkyl which may be partially or fully halogenated and/or may be substituted by one or more, e.g. 1, 2, 3 or 4, preferably 1 or 2, more preferably 1, radicals R⁶; —OR⁷; —OS(O)ₙR⁷; —SR⁷; —S(O)ₙR⁷; —S(O)ₙN(R⁸)R⁹; —N(R⁸)R⁹; —N(R⁸)C(═O)R⁶; C(═O)R⁶; —C(═O)OR⁷; —C(═NR⁸)R⁶; —C(═O)N(R⁸)R⁹; —C(═S)N(R⁸)R⁹; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰;

or two radicals R⁴ bound on adjacent carbon atoms may be together a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —CH₂CH₂S—, —CH═CHS—, —CH₂SCH₂—, —CH₂CH₂NR⁸—, —CH₂CH═N—, —OCH═N—, and —SCH═N—, thus forming, together with the carbon atoms to which they are bound, a 5- or 6-membered ring, where the hydrogen atoms of the above groups may be replaced by one or more, e.g. 1, 2, 3 or 4, preferably 1 or 2, more preferably 1, substituents selected from halogen, methyl, halomethyl, hydroxyl, methoxy and halomethoxy or one or more, e.g. 1 or 2, CH₂ groups of the above groups may be replaced by a C═O group,
where R⁶, R⁷, R⁸, R⁹ and R¹⁰ have one of the meanings given above or in particular one of the preferred meanings given below.

Even more preferably, each R⁴ is independently selected from halogen; cyano; nitro; —SCN; C₁-C₆-alkyl which may be partially or fully halogenated and/or may be substituted by one or more, e.g. 1, 2, 3 or 4, preferably 1 or 2, more preferably 1, radicals R⁶; C₃-C₈-cycloalkyl which may be partially or fully halogenated and/or may be substituted by one or more, e.g. 1, 2, 3 or 4, preferably 1 or 2, more preferably 1, radicals R⁶; —OR⁷; —OS(O)ₙR⁷; —SR⁷; —S(O)ₘR⁷; —S(O)ₙN(R⁸)R⁹; —N(R⁸)R⁹; C(═O)R⁶; —C(═O)OR⁷; —C(═NR⁸)R⁶; —C(═O)N(R⁸)R⁹; —C(═S)N(R⁸)R⁹ and phenyl which may be substituted by 1, 2, 3, 4 or 5 radicals R¹⁰

where R⁶, R⁷, R⁸, R⁹ and R¹⁰ have one of the meanings given above or in particular one of the preferred meanings given below.

In particular, each R⁴ is independently selected from halogen; cyano; nitro; C₁-C₆-alkyl; C₁-C₆-haloalkyl; C₁-C₆-alkoxy; C₁-C₆-haloalkoxy; and phenyl which may be substituted by 1, 2, 3, 4 or 5 radicals R¹⁰;

or two radicals R⁴ bound on adjacent carbon atoms may be together a group selected from —CH₂CH₂CH₂CH₂— and —CH═CH—CH═CH— and preferably —CH═CH—CH═CH—,
where R¹⁰ has one of the meanings given above or in particular one of the preferred meanings given below.

More particularly, each R⁴ is independently selected from halogen, cyano, C₁-C₆-alkyl, preferably C₁-C₄-alkyl, more preferably methyl, and C₁-C₄-haloalkyl, preferably C₁-C₂-haloalkyl. Specifically, each R⁴ is independently selected from halogen and C₁-C₄-alkyl and is very specifically chlorine or methyl.

Preferably, each R⁵ is independently selected from the group consisting of halogen, cyano, nitro, —SCN, SF₅, C₁-C₆-alkyl, C₁-C₆-alkyl which may be partially or fully halogenated and/or may be substituted by one or more, e.g. 1, 2, 3 or 4, preferably 1 or 2, more preferably 1, radicals R⁶, C₃-C₈-cycloalkyl which may be partially or fully halogenated and/or may be substituted by one or more, e.g. 1, 2, 3 or 4, preferably 1 or 2, more preferably 1, radicals R⁶, C₂-C₆-alkenyl which may be partially or fully halogenated and/or may be substituted by one or more, e.g. 1, 2, 3 or 4, preferably 1 or 2, more preferably 1, radicals R⁶, C₂-C₆-alkynyl which may be partially or fully halogenated and/or may be substituted by one or more, e.g. 1, 2, 3 or 4, preferably 1 or 2, more preferably 1, radicals R⁶, Si(R¹⁴)₂R¹³, OR⁷, OS(O)ₙR⁷, S(O)ₘR⁷, NR⁸R⁹, N(R⁸)C(═O)R⁶, C(═O)R⁶, C(═O)OR⁷, C(═NR⁸)R⁶, C(═S)NR⁶, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰,

where R⁶, R⁷, R⁸, R⁹, R¹⁰, R¹³ and R¹⁴ have one of the meanings given above or in particular one of the preferred meanings given below.

More preferably, each R⁵ is independently selected from the group consisting of halogen, cyano, nitro, C₁-C₆-alkyl, C₁-C₆-alkyl which may be partially or fully halogenated and/or may be substituted by one or more, e.g. 1, 2, 3 or 4, preferably 1 or 2, more preferably 1, radicals R⁶, OR⁷, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰,

where R⁶, R⁷ and R¹⁰ have one of the meanings given above or in particular one of the preferred meanings given below.

Even more preferably, each R⁵ is independently selected from the group consisting of halogen, C₁-C₆-alkyl, C₁-C₆-haloalkyl, C₁-C₆-alkoxy and C₁-C₆-haloalkoxy, in particular from halogen, C₁-C₄-alkyl and C₁-C₂-haloalkyl and is specifically halogen, more specifically chlorine, or C₁-C₂-haloalkyl, specifically CF₃.

In case R⁶ is a substituent on an alkyl, alkenyl or alkynyl group, it is preferably selected from the group consisting of cyano, azido, nitro, —SCN, SF₅, C₃-C₈-cycloalkyl, C₃-C₈-halocycloalkyl, —Si(R¹⁴)₂R¹³, —OR⁷, —OSO₂R⁷, —S(O)ₘR⁷, —S(O)ₙN(R⁸)R⁹, —N(R⁸)R⁹, —C(═O)N(R⁸)R⁹, —C(═S)N(R⁸)R⁹, —C(═O)OR⁷, —C(═O)R¹⁹, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰; or two geminally bound radicals R⁶ together form a group selected from ═CR¹¹R¹², ═S(O)ₘR⁷, ═S(O)ₘN(R⁸)R⁹, ═NR⁸, ═NOR⁷ and ═NNR⁸; or two radicals R⁶, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members,

where R⁷, R⁸, R⁹, R¹⁰, R¹¹, R¹², R¹³, R¹⁴ and R¹⁹ have one of the meanings given above or in particular one of the preferred meanings given below.

In case R⁶ is a substituent on an alkyl, alkenyl or alkynyl group, it is more preferably selected from the group consisting of cyano, C₃-C₈-cycloalkyl, C₃-C₈-halocycloalkyl, —OR⁷, —SR⁷, —C(═O)N(R⁸)R⁹, —C(═S)N(R⁸)R⁹, —C(═O)OR⁷, —C(═O)R¹⁹, —C(═NR⁸)R¹⁹, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;

where R⁷, R⁸, R⁹ and R¹⁰ have one of the meanings given above or in particular one of the preferred meanings given below.

In case R⁶ is a substituent on an alkyl, alkenyl or alkynyl group, it is even more preferably selected from the group consisting of cyano, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, —C(═O)N(R⁸)R⁹, —C(═S)N(R⁸)R⁹, —C(═O)OR⁷, —C(═O)R¹⁹, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;

where R¹⁰ has one of the meanings given above or in particular one of the preferred meanings given below.

In case R⁶ is a substituent on an alkyl, alkenyl or alkynyl group, it is in particular selected from the group consisting of cyano, C₃-C₆-cycloalkyl, C₃-C₆-halocycloalkyl, —C(═O)N(R⁸)R⁹, —C(═S)N(R⁸)R⁹, —C(═O)OR⁷, —C(═O)R¹⁹, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;

where R¹⁰ has one of the meanings given above or in particular one of the preferred meanings given below.

In case R⁶ is a substituent on a cycloalkyl group, it is preferably selected from the group consisting of cyano, azido, nitro, —SCN, SF₅, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁴)₂R¹³, —OSO₂R⁷, —S(O)ₘR⁷, —S(O)ₙN(R⁸)R⁹, —N(R⁸)R⁹, —C(═O)N(R⁸)R⁹, —C(═S)N(R⁸)R⁹, —C(═O)OR⁷, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;

or two geminally bound radicals R⁶ together form a group selected from ═CR¹¹R¹², ═S(O)ₘR⁷, ═S(O)ₘN(R⁸)R⁹, ═NR⁸, ═NOR⁷ and ═NNR⁸;
or two radicals R⁶, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members,
where R⁷, R⁸, R⁹, R¹⁰, R¹¹, R¹², R¹³ and R¹⁴ have one of the meanings given above or in particular one of the preferred meanings given below.

In case R⁶ is a substituent on a cycloalkyl group, it is more preferably selected from the group consisting of halogen, cyano, C₁-C₆-alkyl, C₁-C₆-haloalkyl, C₁-C₆-alkoxy-C₁-C₆-alkyl, —OR⁷, —OSO₂R⁷, —S(O)ₘR⁷, —S(O)ₙN(R⁸)R⁹, —N(R⁸)R⁹, —C(═O)N(R⁸)R⁹, —C(═S)N(R⁸)R⁹, —C(═O)OR⁷, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;

where R⁷, R⁸, R⁹ and R¹⁰ have one of the meanings given above or in particular one of the preferred meanings given below.

In case R⁶ is a substituent on a cycloalkyl group, it is even more preferably selected from the group consisting of halogen, C₁-C₄-alkyl, C₁-C₃-haloalkyl, C₁-C₄-alkoxy and C₁-C₃-haloalkoxy. In particular, R⁶ as a substituent on a cycloalkyl group is selected from halogen, C₁-C₄-alkyl and C₁-C₃-haloalkyl.

In case R⁶ is a substituent on C(═O), C(═S) or C(═NR⁸), it is preferably selected from the group consisting of hydrogen, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, —OR⁷, —SR⁷, —N(R⁸)R⁹, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;

where R⁷, R⁸, R⁹ and R¹⁰ have one of the meanings given above or in particular one of the preferred meanings given below.

In case R⁶ is a substituent on C(═O), C(═S) or C(═NR⁸), it is more preferably selected from the group consisting of C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;

where R¹⁰ has one of the meanings given above or in particular one of the preferred meanings given below.

In case R⁶ is a substituent on C(═O), C(═S) or C(═NR⁸), it is more preferably selected from the group consisting of C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;

where R¹⁰ has one of the meanings given above or in particular one of the preferred meanings given below.

In case R⁶ is a substituent on C(═O), C(═S) or C(═NR⁸), it is even more preferably selected from the group consisting of C₁-C₄-alkyl, C₁-C₃-haloalkyl, C₃-C₆-cycloalkyl, C₃-C₆-halocycloalkyl, C₁-C₄-alkoxy, C₁-C₃-haloalkoxy, phenyl which may be substituted by 1, 2, 3, 4 or 5 radicals R¹⁰, and a 5- or 6-membered heteroaromatic ring containing 1, 2 or 3 heteroatoms selected from N, O and S, as ring members, where the heteroaromatic ring may be substituted by one or more radicals R¹⁰;

where R¹⁰ has one of the meanings given above or in particular one of the preferred meanings given below.

Preferably, each R⁷ is independently selected from the group consisting of hydrogen, C₁-C₆-alkyl, C₁-C₆-haloalkyl, C₃-C₈-cycloalkyl, C₃-C₈-halocycloalkyl, C₃-C₈-cycloalkylC₁-C₄-alkyl, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰, where R¹⁰ has one of the meanings given above or in particular one of the preferred meanings given below.

More preferably, each R⁷ is independently selected from the group consisting of hydrogen, C₁-C₆-alkyl, C₁-C₆-haloalkyl, phenyl which may be substituted by 1, 2, 3, 4 or 5 radicals R¹⁰; and a 5- or 6-membered heteroaromatic ring containing 1, 2 or 3 heteroatoms selected from N, O and S, as ring members, where the heteroaromatic ring may be substituted by one or more radicals R¹⁰; where R¹⁰ has one of the meanings given above or in particular one of the preferred meanings given below.

R⁸ and R⁹ are independently of each other and independently of each occurrence preferably selected from the group consisting of hydrogen, cyano, C₁-C₆-alkyl which may be partially or fully halogenated and/or may be substituted by one or more radicals R¹⁹, C₂-C₆-alkenyl which may be partially or fully halogenated and/or may be substituted by one or more radicals R¹⁹, C₂-C₆-alkynyl which may be partially or fully halogenated and/or may be substituted by one or more radicals R¹⁹, C₃-C₈-cycloalkyl, C₃-C₈-halocycloalkyl, C₃-C₈-cycloalkyl-C₁-C₆-alkyl, S(O)ₘR²⁰, S(O)ₙNR²¹R²², phenyl which may be substituted by 1, 2, 3, 4 or 5 radicals R¹⁰, benzyl wherein the phenyl moiety may be substituted by 1, 2, 3, 4 or 5 radicals R¹⁰, and a 5- or 6-membered heterocyclic ring containing 1, 2 or 3 heteroatoms or heteroatom groups selected from N, O, S, NO, SO and SO₂, as ring members, where the heterocyclic ring may be substituted by one or more radicals R¹⁰; where R¹⁰ has one of the meanings given above or in particular one of the preferred meanings given below; or

R⁸ and R⁹ together form a group ═CR¹¹R¹²; or
R⁸ and R⁹, together with the nitrogen atom to which they are bound, form a 3-, 4-, 5-, 6- or 7-membered saturated, partially unsaturated or aromatic, preferably a saturated, heterocyclic ring which may additionally containing 1 or 2 further heteroatoms or heteroatom groups selected from N, O, S, NO, SO and SO₂, as ring members, where the heterocyclic ring may be substituted by one or more radicals R¹⁰.

In the above preferred embodiment of R⁸ and R⁹, R¹¹ is preferably hydrogen or methyl and R¹² is preferably C₁-C₆-alkoxy, C₁-C₆-haloalkoxy, —C(═O)R¹⁹, —C(═O)OR²⁰, or —C(═O)N(R²¹)R²².

In the above preferred embodiment of R⁸ and R⁹, R⁹, if it does not form together with R⁸ a group ═CR¹¹R¹² or together with R⁸ and the N atom to which they are bound a heterocyclic ring, is preferably selected from hydrogen, cyano, C₁-C₄-alkyl, C₁-C₄-haloalkyl, cyclopropyl, C₁-C₄-alkylcarbonyl, C₁-C₄-haloalkylcarbonyl, C₁-C₄-alkoxycarbonyl and C₁-C₄-haloalkoxycarbonyl and is more preferably hydrogen or C₁-C₄-alkyl.

In the above preferred embodiment of R⁸ and R⁹, R⁸, if it does not form together with R⁹ a group ═CR¹¹R¹² or together with R⁹ and the N atom to which they are bound a heterocyclic ring, is preferably selected from CN, C₁-C₆-alkyl; C₁-C₆-haloalkyl; C₁-C₄-alkyl which carries one radical R¹⁹; C₂-C₆-alkenyl; C₂-C₆-haloalkenyl; C₂-C₄-alkenyl which is substituted by one radical R¹⁹; C₃-C₆-cycloalkyl; C₃-C₆-halocycloalkyl; C₃-C₆-cycloalkylC₁-C₄-alkyl; C₃-C₆-halocycloalkyl-C₁-C₄-alkyl; —S(O)ₘR²⁰; —S(O)ₙN(R²¹)R²²; phenyl; benzyl and a 5- or 6-membered saturated, partially unsaturated or aromatic heterocyclic ring containing 1, 2 or 3 heteroatoms or heteroatom groups selected from N, O, S, NO, SO and SO₂, as ring members, where the phenyl or heterocyclyl rings in the three lastmentioned radicals may carry 1, 2 or 3 substituents selected from halogen, CN, alkyl, C₁-C₄-haloalkyl, C₁-C₄-alkoxy and C₁-C₄-haloalkoxy.

If R⁸ and R⁹, together with the nitrogen atom to which they are bound, form a 3-, 4-, 5-, 6- or 7-membered saturated, partially unsaturated or aromatic heterocyclic ring which may additionally contain 1 or 2 further heteroatoms or heteroatom groups selected from N, O, S, NO, SO and SO₂, as ring members, this is preferably a 3, 5 or 6-membered saturated heterocyclic ring which may additionally contain 1 further heteroatom or heteroatom group selected from N, O, S, NO, SO and SO₂, as ring member.

Specifically, R⁸ and R⁹ are independently of each other and independently of each occurrence selected from the group consisting of hydrogen, C₁-C₆-alkyl, C₁-C₆-haloalkyl, C₂-C₆-alkynyl, C₃-C₈-cycloalkyl-C₁-C₆-alkyl, benzyl wherein the phenyl moiety may be substituted by 1, 2, 3, 4 or 5 radicals R¹⁰, and a 5- or 6-membered heterocyclic ring containing 1, 2 or 3 heteroatoms or heteroatom groups selected from N, O, S, NO, SO and SO₂, as ring members, where the heterocyclic ring may be substituted by one or more radicals R¹⁰. More specifically, R⁹ is hydrogen or C₁-C₄-alkyl and R⁸ has one of the meanings specified above.

Preferably, each R^{8a} is independently selected from the group consisting of hydrogen, cyano, C₁-C₆-alkyl which may be partially or fully halogenated and/or may be substituted by one or more radicals R¹⁹, C₂-C₆-alkenyl which may be partially or fully halogenated and/or may be substituted by one or more radicals R¹⁹, C₂-C₆-alkynyl which may be partially or fully halogenated and/or may be substituted by one or more radicals R¹⁹, C₃-C₈-cycloalkyl, C₃-C₈-halocycloalkyl, C₃-C₈-cycloalkyl-C₁-C₆-alkyl, S(O)ₘR²⁰, S(O)ₙNR²¹R²², phenyl which may be substituted by 1, 2, 3, 4 or 5 radicals R¹⁰, benzyl wherein the phenyl moiety may be substituted by 1, 2, 3, 4 or 5 radicals R¹⁰, and a 5- or 6-membered heterocyclic ring containing 1, 2 or 3 heteroatoms or heteroatom groups selected from N, O, S, NO, SO and SO₂, as ring members, where the heterocyclic ring may be substituted by one or more radicals R¹⁰; where R¹⁰ has one of the meanings given above or in particular one of the preferred meanings given below; or

R⁸ and R⁹ together form a group ═CR¹¹R¹². More preferably, each R^{8a} is independently selected from hydrogen, C₁-C₄-alkyl and C₁-C₄-haloalkyl.

Preferably, each R¹⁰ is independently selected from the group consisting of halogen, cyano, C₁-C₁₀-alkyl which may be partially or fully halogenated and/or may be substituted by one or more radicals R¹⁹, C₃-C₈-cycloalkyl which may be partially or fully halogenated and/or may be substituted by one or more radicals R¹⁰, —OR²⁰, —OS(O)ₙR²⁰, —SR²⁰, —S(O)ₘR²⁰, —S(O)ₙN(R²¹)R²², —N(R²¹)R²², C(═O)R¹⁹, —C(═O)OR²⁰, —C(═O)N(R²¹)R²², phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more radicals independently selected from halogen, cyano, nitro, C₁-C₆-alkyl, C₁-C₆-haloalkyl, C₁-C₆-alkoxy and C₁-C₆-haloalkoxy;

or two radicals R¹⁰ bound on adjacent atoms together form a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, and —O(CH₂)O—, thus forming, together with the atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group,
where R¹⁹, R²⁰, R²¹ and R²² have one of the general or in particular one of the preferred meanings given above.

More preferably, each R¹⁰ is independently selected from the group consisting of halogen, cyano, C₁-C₁₀-alkyl which may be partially or fully halogenated and/or may be substituted by one or more radicals R¹⁹, —OR²⁰, —N(R²¹)R²², C(═O)R¹⁹, —C(═O)OR²⁰, —C(═O)N(R²¹)R²², phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more radicals independently selected from halogen, cyano, nitro, C₁-C₆-alkyl, C₁-C₆-haloalkyl, C₁-C₆-alkoxy and C₁-C₆-haloalkoxy;

where R¹⁹, R²⁰, R²¹ and R²² have one of the general or in particular one of the preferred meanings given above.

Even more preferably, each R¹⁰ is independently selected from the group consisting of halogen, C₁-C₄-alkyl, C₁-C₄-haloalkyl, C₁-C₄-alkoxy and C₁-C₄-haloalkoxy. In particular, each R¹⁰ is independently selected from the group consisting of halogen, C₁-C₄-alkyl and C₁-C₄-haloalkyl and is specifically halogen, more specifically chlorine.

Preferably, R¹¹ and R¹² are, independently of each other and independently of each occurrence, selected from the group consisting of hydrogen, halogen, C₁-C₆-alkyl and C₁-C₆-haloalkyl. More preferably, R¹¹ and R¹² are, independently of each other and independently of each occurrence, selected from the group consisting of hydrogen, halogen and C₁-C₆-alkyl and in particular from the group consisting of hydrogen and halogen. Specifically, they are hydrogen.

Preferably, R¹³ and R¹⁴ are, independently of each other and independently of each occurrence, selected from C₁-C₄-alkyl and are in particular methyl.

Preferably, R¹⁵ and R¹⁶ are, independently of each other and independently of each occurrence, selected from the group consisting of C₁-C₆-alkyl, C₁-C₆-haloalkyl and phenyl which may be substituted by 1, 2, 3, 4, or 5 radicals R¹⁰; where R¹⁰ has one of the general or in particular one of the preferred meanings given above.

Preferably, each R¹⁷ is independently selected from the group consisting of C₁-C₆-alkyl, C₁-C₆-haloalkyl, C₃-C₈-cycloalkyl, C₃-C₈-halocycloalkyl, phenyl and benzyl. More preferably, each R¹⁷ is independently selected from the group consisting of C₁-C₆-alkyl, C₁-C₆-haloalkyl and phenyl and is in particular C₁-C₄-alkyl or C₁-C₃-haloalkyl.

Preferably, each R¹⁸ is independently selected from the group consisting of hydrogen; C₁-C₁₀-alkyl which may be partially or fully halogenated and/or may be substituted by one or more, e.g. 1, 2, 3 or 4, preferably 1 or 2, more preferably 1, radicals R⁶; —C(═O)R⁶; —C(═O)OR⁷; —C(═O)N(R⁸)R⁹; —C(═S)R⁶; —C(═S)OR⁷; —C(═S)N(R⁸)R⁹ and —C(═NR⁸)R⁶; where R⁶, R⁷, R⁸ and R⁹ have one of the general or in particular one of the preferred meanings given above.

More preferably, each R¹⁸ is selected from the group consisting of hydrogen; C₁-C₆-alkyl which may be partially or fully halogenated and/or may be substituted by one or more, e.g. 1, 2, 3 or 4, preferably 1 or 2, more preferably 1, radicals R⁶; —C(═O)R⁶ and —C(═O)N(R⁸)R⁹; where R⁶, R⁸ and R⁹ have one of the general or in particular one of the preferred meanings given above. Preferably, in this case, R⁶ as a C₁-C₆-alkyl substituent, is selected from CN, C₃-C₆-cycloalkyl, C₃-C₆-halocycloalkyl, C₁-C₆-alkoxy, C₁-C₆-haloalkoxy, C₁-C₆-alkylthio, C₁-C₆-haloalkylthio and a 5- or 6-membered hetaryl ring containing 1, 2 or 3 heteroatoms selected from N, O and S as ring members and being optionally substituted by 1, 2 or 3 radicals R¹⁰. In this case, R⁶ as a CO substituent, is preferably selected from C₁-C₆-alkyl, C₁-C₆-haloalkyl, C₁-C₆-alkoxy and C₁-C₆-haloalkoxy. In this case, R⁸ and R⁹ are preferably selected from hydrogen and C₁-C₆-alkyl.

In particular, each R¹⁸ is selected from the group consisting of hydrogen, C₁-C₄-alkyl, C₁-C₄-haloalkyl and —C(═O)R⁶, and is specifically selected from the group consisting of hydrogen, C₁-C₄-alkyl and —C(═O)R⁶, where R⁶ has one of the general or in particular one of the preferred meanings given above and is specifically C₁-C₄-alkyl.

In case R¹⁹ is a substituent on an alkyl, alkenyl or alkynyl group, it is preferably selected from the group consisting of cyano, C₃-C₆-cycloalkyl, C₃-C₆-halocycloalkyl, —OR²⁰, —C(═O)N(R²¹)R²², —C(═S)N(R²¹)R²², —C(═O)OR²⁰, —C(═O)R²⁰, phenyl which may be substituted by 1, 2, 3, 4 or 5 radicals R¹⁰, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R¹⁰;

where
R¹⁰ is selected from halogen, cyano, C₁-C₄-alkyl, C₁-C₄-haloalkyl, C₁-C₄-alkoxy and C₁-C₄-haloalkoxy;
R²⁰ is selected from hydrogen, C₁-C₄-alkyl, C₁-C₄-haloalkyl, phenyl, benzyl, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R¹⁰; and
R²¹ and R²², independently of each other and independently of each occurrence, are selected from hydrogen, C₁-C₄-alkyl, C₁-C₄-haloalkyl, phenyl, benzyl, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R¹⁰.

In case R¹⁹ is a substituent on a cycloalkyl group, it is preferably selected from the group consisting of cyano, C₁-C₄-alkyl, C₁-C₄-haloalkyl, C₃-C₆-cycloalkyl, C₃-C₆-halocycloalkyl, —C(═O)N(R²¹)R²², —C(═S)N(R²¹)R²², —C(═O)OR²⁰, —C(═O)R²⁰), phenyl which may be substituted by 1, 2, 3, 4 or 5 radicals R¹⁰, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R¹⁰;

where
R¹⁰ is selected from halogen, cyano, C₁-C₄-alkyl, C₁-C₄-haloalkyl, C₁-C₄-alkoxy and C₁-C₄-haloalkoxy;
R²⁰ is selected from hydrogen, C₁-C₄-alkyl, C₁-C₄-haloalkyl, phenyl, benzyl, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R¹⁰; and
R²¹ and R²², independently of each other and independently of each occurrence, are selected from hydrogen, C₁-C₄-alkyl, C₁-C₄-haloalkyl, phenyl, benzyl, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R¹⁰.

In case R¹⁹ is a substituent on a C(═O) group, it is preferably selected from the group consisting of hydrogen, C₁-C₄-alkyl, C₁-C₄-haloalkyl, C₃-C₆-cycloalkyl, C₃-C₆-halocycloalkyl, C₃-C₆-cycloalkyl-C₁-C₄-alkyl, phenyl which may be substituted by 1, 2, 3, 4 or 5 radicals R¹⁰, benzyl which may be substituted by 1, 2, 3, 4 or 5 radicals R¹⁰, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R¹⁰; where R¹⁰ is selected from halogen, cyano, C₁-C₄-alkyl, C₁-C₄-haloalkyl, C₁-C₄-alkoxy and C₁-C₄-haloalkoxy.

R²⁰ is preferably selected from the group consisting of hydrogen, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₃-C₆-cycloalkyl-C₁-C₄-alkyl, phenyl which may be substituted by 1, 2, 3, 4 or 5 radicals R¹⁰, benzyl which may be substituted by 1, 2, 3, 4 or 5 radicals R¹⁰, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R¹⁰; where R¹⁰ is selected from halogen, cyano, C₁-C₄-alkyl, C₁-C₄-haloalkyl, C₁-C₄-alkoxy and C₁-C₄-haloalkoxy.

R²¹ and R²², independently of each other and independently of each occurrence, are preferably selected from the group consisting of hydrogen, C₁-C₆-alkyl, C₁-C₆-haloalkyl, C₃-C₈-cycloalkyl, C₃-C₈-halocycloalkyl, C₃-C₈-cycloalkyl-C₁-C₄-alkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁰, benzyl which may be substituted by 1, 2, 3, 4 or 5 radicals R¹⁰, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R¹⁰; where R¹⁰ is selected from halogen, cyano, C₁-C₄-alkyl, C₁-C₄-haloalkyl, C₁-C₄-alkoxy and C₁-C₄-haloalkoxy;

or R²¹ and R²², together with the nitrogen atom to which they are bound, may form a 5- or 6-membered saturated, partially unsaturated or aromatic heterocyclic ring which may additionally containing 1 or 2 further heteroatoms or heteroatom groups selected from N, O, S, NO, SO and SO₂, as ring members, where the heterocyclic ring may be substituted by one or more radicals selected from halogen, C₁-C₆-alkyl, C₁-C₆-haloalkyl, C₁-C₆-alkoxy and C₁-C₆-haloalkoxy.

G is preferably selected from rings G-3, G-4, G-13, G-14, G-16, G-17, G-18, G-19, G-21, G-26, G-27 and G-28. More preferably, G is selected from rings G-13, G-14 and G-16 and is preferably ring G-14 or ring G-16.

Preferably, G is bound via the attachment point “*” to the phenyl or pyridyl group comprising B¹ as ring member and via the attachment point “#” to the phenyl or pyridyl group comprising A¹ as ring member.

One particularly preferred embodiment of the invention refers to compounds of the formula I-1

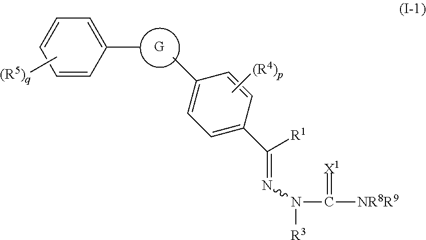

where X¹ is O or S and G, X, R¹, R³, R⁴, R⁵, R⁸, R⁹, R^{8a}, p and q have one of the above-given general or, in particular, one of the above-given preferred meanings.

In particular, the invention relates to compounds I-1, where
G is a bivalent heterocyclic ring selected from G-13, G-14 and G-16 and is preferably G-14 or G-16;
X¹ is O or S;
p is 0, 1 or 2;
q is 0, 1, 2 or 3; and
   X, R¹, R³, R⁴, R⁵, R⁸, R⁹ and R^{8a} have one of the above-given general or, in particular, one of the above-given preferred meanings.

In compounds of the formula I-1, R¹ is preferably hydrogen.

In compounds of the formula I-1, R³ is preferably hydrogen.

In compounds of the formula I-1, R⁴ is preferably halogen, cyano, C₁-C₄-alkyl or C₁-C₄-haloalkyl, more preferably halogen or C₁-C₄-alkyl and specifically chlorine or methyl.

In compounds of the formula I-1, R⁵ is preferably halogen or C₁-C₄-haloalkyl, more preferably chlorine or CF₃ and specifically chlorine.

In compounds of the formula I-1, R⁸ is preferably selected from hydrogen and C₁-C₆-alkyl which may be partially or fully halogenated and/or may be substituted by one or more radicals R¹⁹, where R¹⁹ has one of the above-given general or, in particular, one of the above-given preferred meanings; R⁸ is specifically hydrogen.

In compounds of the formula I-1, R⁹ is preferably is selected from hydrogen, C₁-C₆-alkyl which may be partially or fully halogenated and/or may be substituted by one or more radicals R¹⁹, C₃-C₆-cycloalkyl, C₃-C₆-halocycloalkyl, C₃-C₆-cycloalkyl-C₁-C₄-alkyl and C₃-C₆-halocycloalkyl-C₁-C₄-alkyl, and more preferably from hydrogen, C₁-C₆-alkyl, C₁-C₄-haloalkyl, C₃-C₆-cycloalkyl and C₃-C₆-cycloalkyl-methyl.

In compounds of the formula I-1, R^{8a} in G-13 is preferably selected from hydrogen, methyl, ethyl and 2,2,2-trifluoroethyl.

In compounds of the formula I-1, X in G-13, G-14 and G-16 is preferably selected from CF₃, CHF₂ and CF₂Cl and is more preferably CF₃.

Particularly preferably, in compounds 1-1
G is a bivalent heterocyclic ring selected from G-13, G-14 and G-16, where in G-13 R^{8a} is selected from hydrogen, methyl, ethyl and 2,2,2-trifluoroethyl and in G-13, G-14 and G-16 X is CF₃, and is preferably G-14 or G-16 wherein X is CF₃;
X¹ is O or S;
R¹ is hydrogen;
R³ is hydrogen;
R⁴ is halogen or C₁-C₄-alkyl, preferably chlorine or methyl;
R⁵ is chlorine or CF₃, preferably chlorine;
R⁸ is hydrogen;
R⁹ is selected from hydrogen, C₁-C₆-alkyl which may be partially or fully halogenated and/or may be substituted by one or more radicals R¹⁹, C₃-C₆-cycloalkyl, C₃-C₆-halocycloalkyl, C₃-C₆-cycloalkyl-C₁-C₄-alkyl and C₃-C₆-halocycloalkyl-C₁-C₄-alkyl, preferably from hydrogen, C₁-C₆-alkyl, C₁-C₄-haloalkyl, C₃-C₆-cycloalkyl and C₃-C₆-cycloalkyl-methyl;
p is 0, 1 or 2, preferably 1; and
q is 0, 1, 2 or 3, preferably 2.

Examples of preferred compounds are compounds of the following formulae I.1 to I.66, where the variables have one of the general or preferred meanings given above. Examples of preferred compounds which are represented by the formulae I.1 to I.66 are the individual compounds compiled in the tables 1 to 24420 below, where the variables Y and R² have the meanings given in one row of table A. Moreover, the meanings mentioned for the individual variables in the tables are per se, independently of the combination in which they are mentioned, a particularly preferred embodiment of the substituents in question.

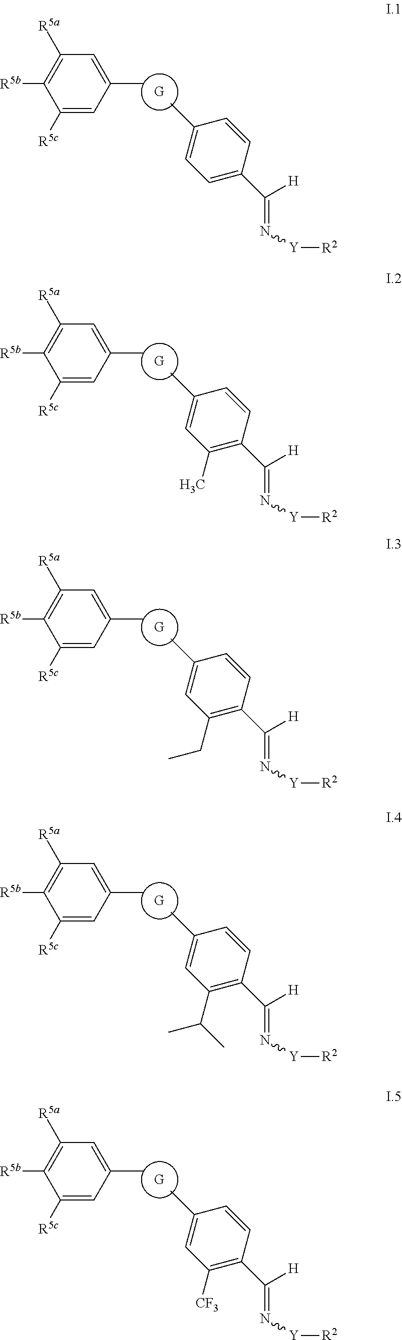

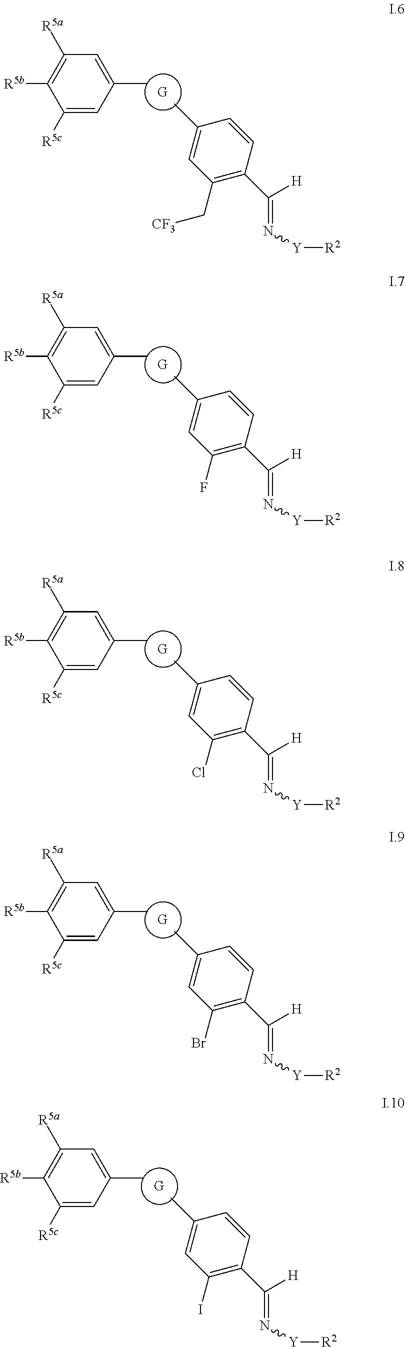

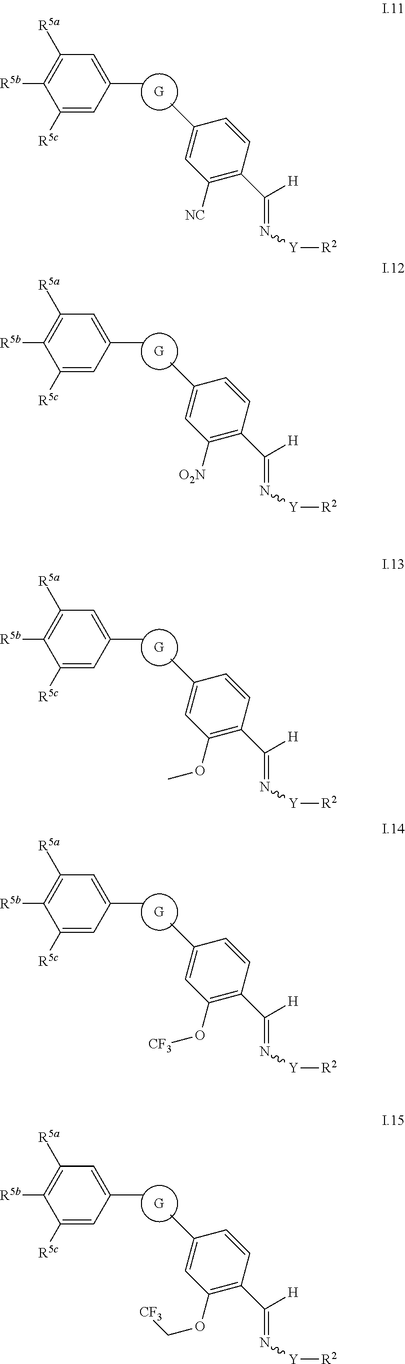

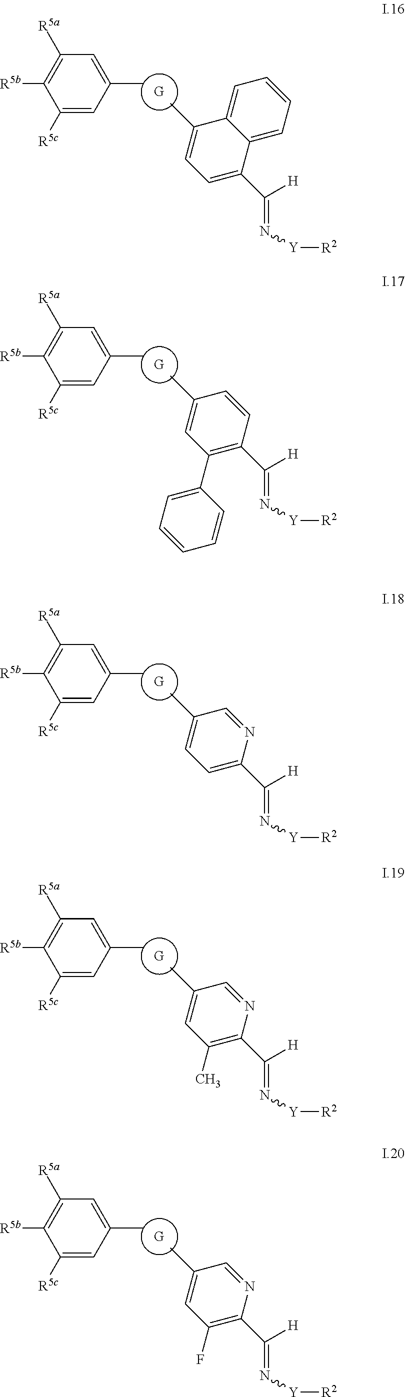

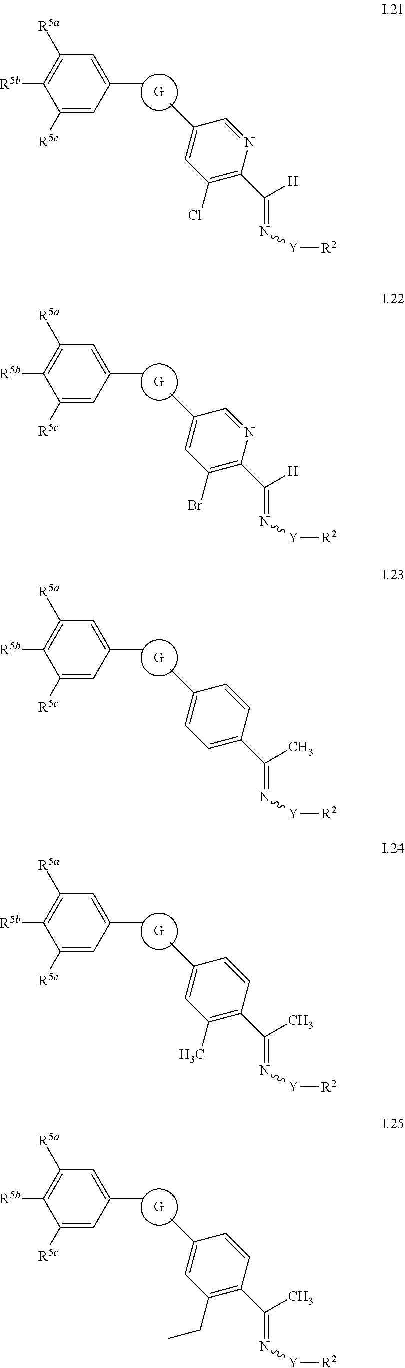

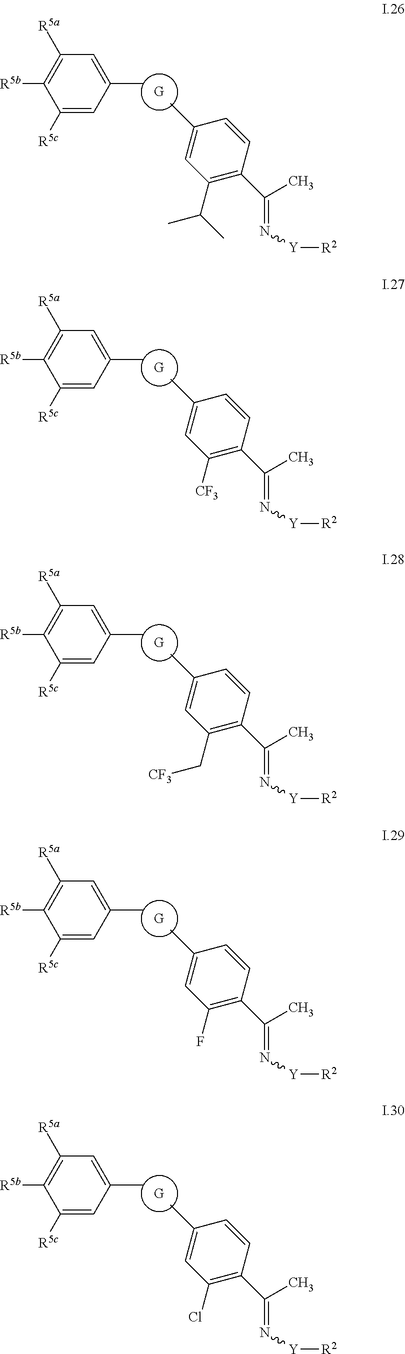

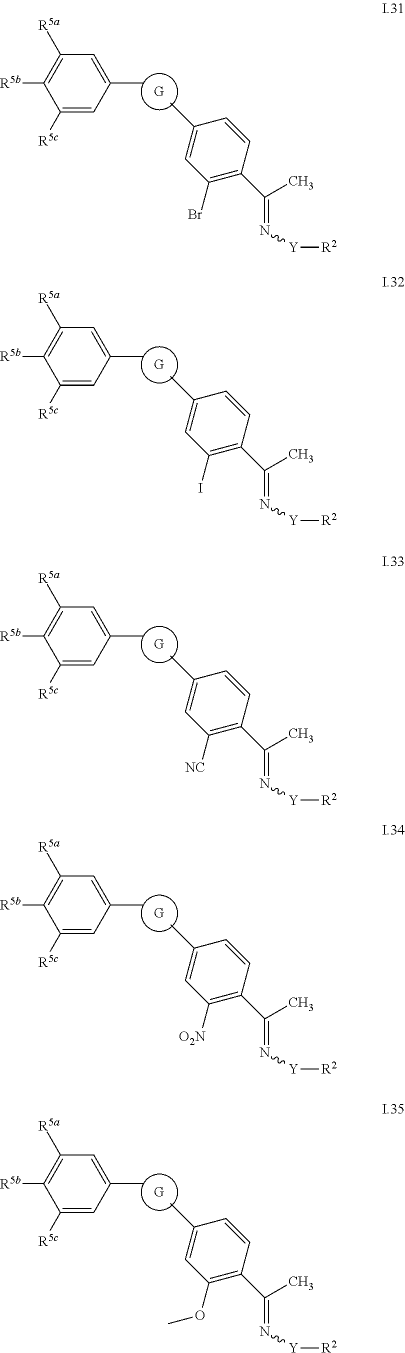

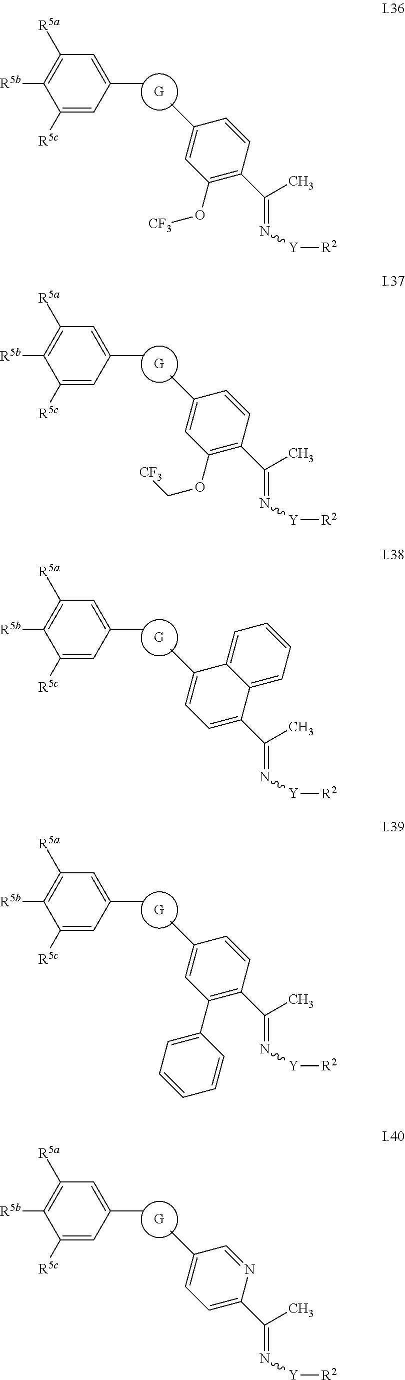

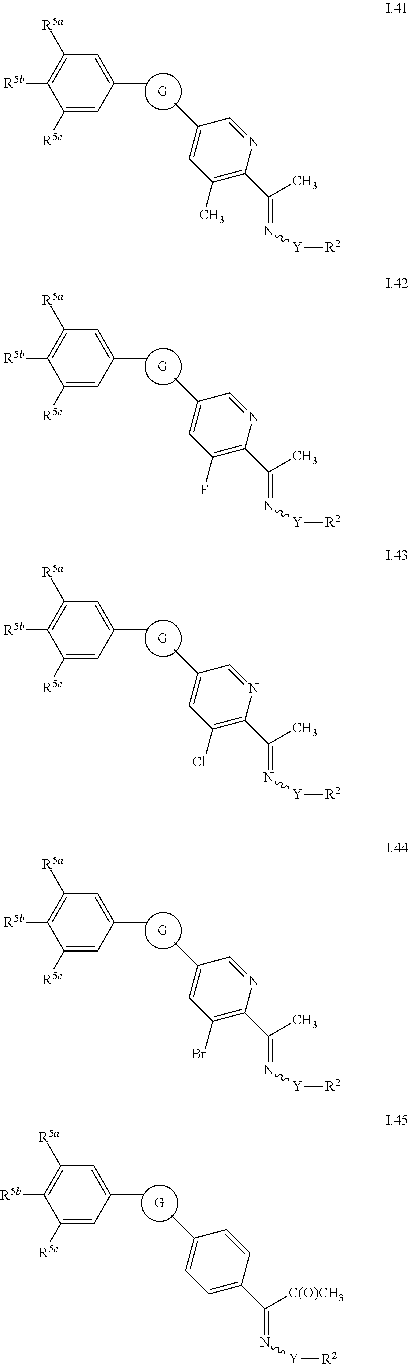

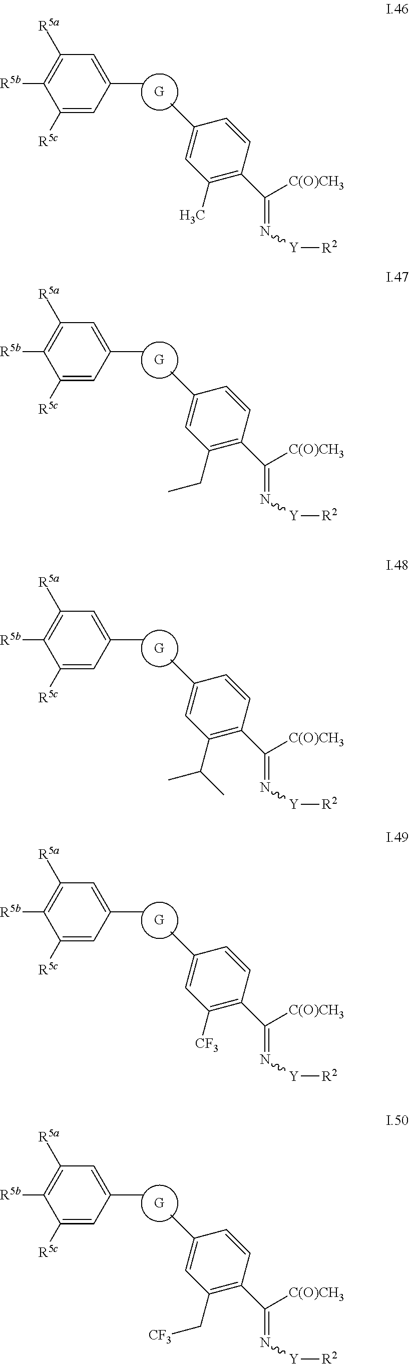

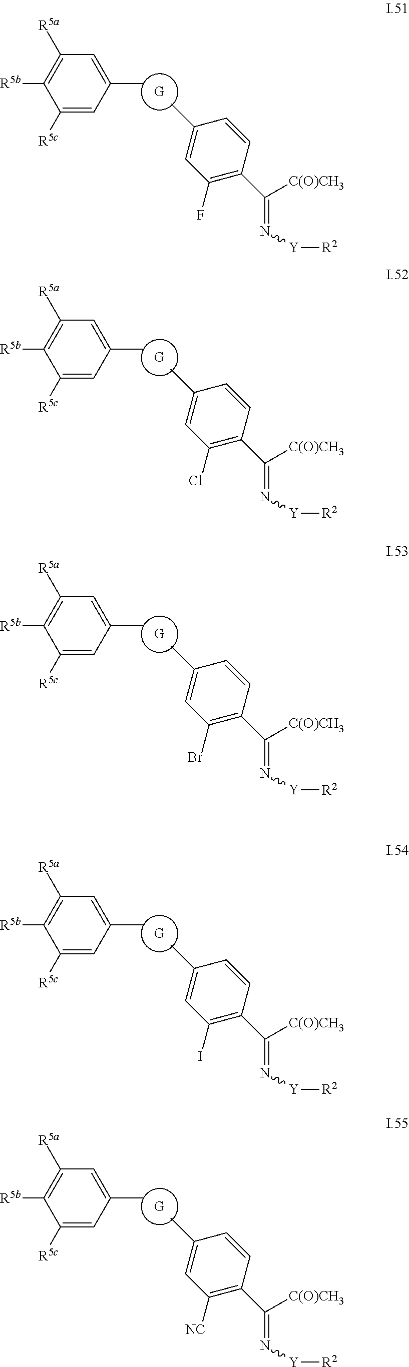

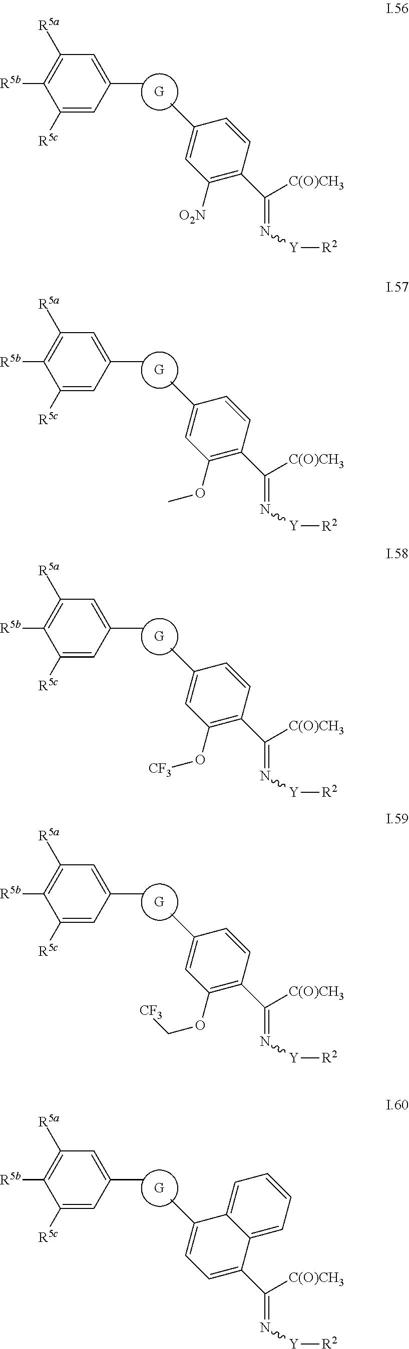

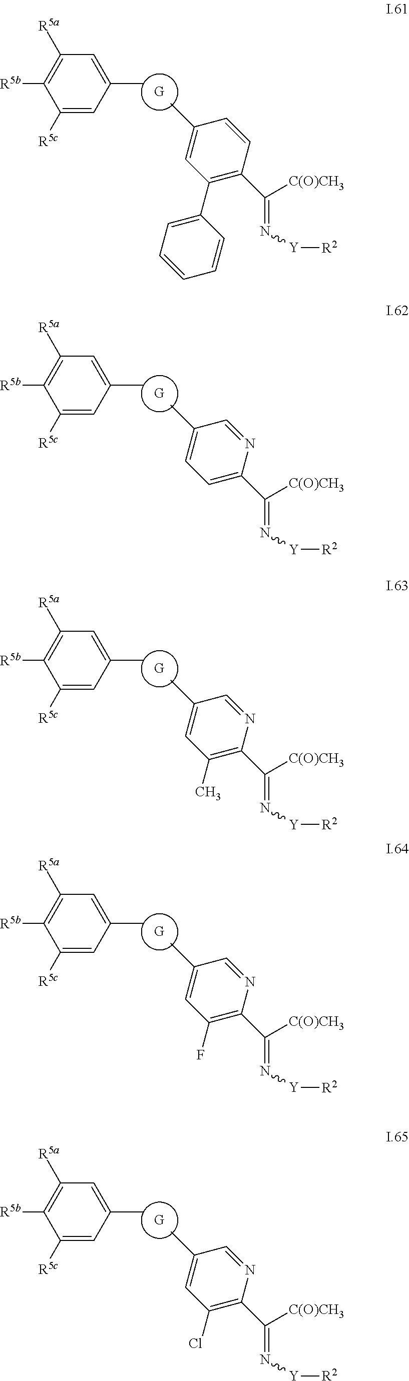

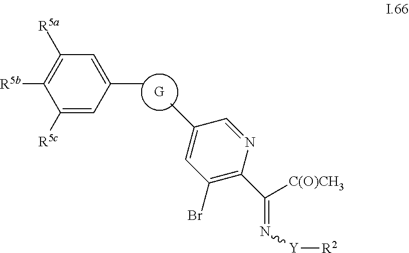

### Table 1

Compounds of the formula I.1 in which R^{5a} and R^{5c} are chlorine, R^{5b} is H, G is G-1.1 and the combination of Y and R² for a compound corresponds in each case to one row of Table A.

### Table 2

Compounds of the formula I.1 in which R^{5a} and R^{5c} are bromine, R^{5b} is H, G is G-1.1 and the combination of Y and R² for a compound corresponds in each case to one row of Table A.

### Table 3

Compounds of the formula I.1 in which R^{5a} and R^{5c} are fluorine, R^{5b} is H, G is G-1.1 and the combination of Y and R² for a compound corresponds in each case to one row of Table A.

### Table 4

Compounds of the formula I.1 in which R^{5a} and R^{5c} are methyl, R^{5b} is H, G is G-1.1 and the combination of Y and R² for a compound corresponds in each case to one row of Table A.

### Table 5

Compounds of the formula I.1 in which R^{5a} and R^{5c} are CF₃, R^{5b} is H, G is G-1.1 and the combination of Y and R² for a compound corresponds in each case to one row of Table A.

### Table 6

Compounds of the formula I.1 in which R^{5a} is chlorine and R^{5b} and R^{5c} are H, G is G-1.1 and the combination of Y and R² for a compound corresponds in each case to one row of Table A.

### Table 7

Compounds of the formula I.1 in which R^{5a} is CF₃ and R^{5b} and R^{5c} are H, G is G-1.1 and the combination of Y and R² for a compound corresponds in each case to one row of Table A.

### Table 8

Compounds of the formula I.1 in which R^{5a}, R^{5b} and R^{5c} are chlorine, G is G-1.1 and the combination of Y and R² for a compound corresponds in each case to one row of Table A.

### Table 9

Compounds of the formula I.1 in which R^{5a}, R^{5b} and R^{5c} are fluorine, G is G-1.1 and the combination of Y and R² for a compound corresponds in each case to one row of Table A.

### Table 10

Compounds of the formula I.1 in which R^{5a} and R^{5c} are chlorine and R^{5b} is fluorine, G is G-1.1 and the combination of Y and R² for a compound corresponds in each case to one row of Table A.

### Tables 11 to 20

Compounds of the formula I.1 in which the combination of R^{5a}, R^{5b} and R^{5c} is as defined in Tables 1 to 10, G is G-2.1 and the combination of Y and R² for a compound corresponds in each case to one row of Table A.

### Tables 21 to 30

Compounds of the formula I.1 in which the combination of R^{5a}, R^{5b} and R^{5c} is as defined in Tables 1 to 10, G is G-3.1 and the combination of Y and R² for a compound corresponds in each case to one row of Table A.

### Tables 31 to 40

Compounds of the formula I.1 in which the combination of R^{5a}, R^{5b} and R^{5c} is as defined in Tables 1 to 10, G is G-4.1 and the combination of Y and R² for a compound corresponds in each case to one row of Table A.

### Tables 41 to 50

Compounds of the formula I.1 in which the combination of R^{5a}, R^{5b} and R^{5c} is as defined in Tables 1 to 10, G is G-5.1 and the combination of Y and R² for a compound corresponds in each case to one row of Table A.

### Tables 51 to 60

Compounds of the formula I.1 in which the combination of R^{5a}, R^{5b} and R^{5c} is as defined in Tables 1 to 10, G is G-6.1 and the combination of Y and R² for a compound corresponds in each case to one row of Table A.

### Tables 61 to 70

Compounds of the formula I.1 in which the combination of R^{5a}, R^{5b} and R^{5c} is as defined in Tables 1 to 10, G is G-7.1 and the combination of Y and R² for a compound corresponds in each case to one row of Table A.

### Tables 71 to 80

Compounds of the formula I.1 in which the combination of R^{5a}, R^{5b} and R^{5c} is as defined in Tables 1 to 10, G is G-8.1 and the combination of Y and R² for a compound corresponds in each case to one row of Table A.

### Tables 81 to 90

Compounds of the formula I.1 in which the combination of R^{5a}, R^{5b} and R^{5c} is as defined in Tables 1 to 10, G is G-9.1 and the combination of Y and R² for a compound corresponds in each case to one row of Table A.

### Tables 91 to 100

Compounds of the formula I.1 in which the combination of R^{5a}, R^{5b} and R^{5c} is as defined in Tables 1 to 10, G is G-10.1 and the combination of Y and R² for a compound corresponds in each case to one row of Table A.

### Tables 101 to 110

Compounds of the formula I.1 in which the combination of R^{5a}, R^{5b} and R^{5c} is as defined in Tables 1 to 10, G is G-11.1 and the combination of Y and R² for a compound corresponds in each case to one row of Table A.

### Tables 111 to 120

Compounds of the formula I.1 in which the combination of R^{5a}, R^{5b} and R^{5c} is as defined in Tables 1 to 10, G is G-12.1 and the combination of Y and R² for a compound corresponds in each case to one row of Table A.

### Tables 121 to 130

Compounds of the formula I.1 in which the combination of R^{5a}, R^{5b} and R^{5c} is as defined in Tables 1 to 10, G is G-13.1 and the combination of Y and R² for a compound corresponds in each case to one row of Table A.

### Tables 131 to 140

Compounds of the formula I.1 in which the combination of R^{5a}, R^{5b} and R^{5c} is as defined in Tables 1 to 10, G is G-13.2 and the combination of Y and R² for a compound corresponds in each case to one row of Table A.

### Tables 141 to 150

Compounds of the formula I.1 in which the combination of R^{5a}, R^{5b} and R^{5c} is as defined in Tables 1 to 10, G is G-13.3 and the combination of Y and R² for a compound corresponds in each case to one row of Table A.

### Tables 151 to 160

Compounds of the formula I.1 in which the combination of R^{5a}, R^{5b} and R^{5c} is as defined in Tables 1 to 10, G is G-13.4 and the combination of Y and R² for a compound corresponds in each case to one row of Table A.

### Tables 161 to 170

Compounds of the formula I.1 in which the combination of R^{5a}, R^{5b} and R^{5c} is as defined in Tables 1 to 10, G is G-14.1 and the combination of Y and R² for a compound corresponds in each case to one row of Table A.

### Tables 171 to 180

Compounds of the formula I.1 in which the combination of R^{5a}, R^{5b} and R^{5c} is as defined in Tables 1 to 10, G is G-15.1 and the combination of Y and R² for a compound corresponds in each case to one row of Table A.

### Tables 181 to 190

Compounds of the formula I.1 in which the combination of R^{5a}, R^{5b} and R^{5c} is as defined in Tables 1 to 10, G is G-16.1 and the combination of Y and R² for a compound corresponds in each case to one row of Table A.

### Tables 191 to 200

Compounds of the formula I.1 in which the combination of R^{5a}, R^{5b} and R^{5c} is as defined in Tables 1 to 10, G is G-17.1 and the combination of Y and R² for a compound corresponds in each case to one row of Table A.

### Tables 201 to 210

Compounds of the formula I.1 in which the combination of R^{5a}, R^{5b} and R^{5c} is as defined in Tables 1 to 10, G is G-17.2 and the combination of Y and R² for a compound corresponds in each case to one row of Table A.

### Tables 211 to 220

Compounds of the formula I.1 in which the combination of R^{5a}, R^{5b} and R^{5c} is as defined in Tables 1 to 10, G is G-17.3 and the combination of Y and R² for a compound corresponds in each case to one row of Table A.

### Tables 221 to 230

Compounds of the formula I.1 in which the combination of R^{5a}, R^{5b} and R^{5c} is as defined in Tables 1 to 10, G is G-17.4 and the combination of Y and R² for a compound corresponds in each case to one row of Table A.

### Tables 231 to 240

Compounds of the formula I.1 in which the combination of R^{5a}, R^{5b} and R^{5c} is as defined in Tables 1 to 10, G is G-18.1 and the combination of Y and R² for a compound corresponds in each case to one row of Table A.

### Tables 241 to 250

Compounds of the formula I.1 in which the combination of R^{5a}, R^{5b} and R^{5c} is as defined in Tables 1 to 10, G is G-19.1 and the combination of Y and R² for a compound corresponds in each case to one row of Table A.

### Tables 251 to 260

Compounds of the formula I.1 in which the combination of R^{5a}, R^{5b} and R^{5c} is as defined in Tables 1 to 10, G is G-20.1 and the combination of Y and R² for a compound corresponds in each case to one row of Table A.

### Tables 261 to 270

Compounds of the formula I.1 in which the combination of R^{5a}, R^{5b} and R^{5c} is as defined in Tables 1 to 10, G is G-20.2 and the combination of Y and R² for a compound corresponds in each case to one row of Table A.

### Tables 271 to 280

Compounds of the formula I.1 in which the combination of R^{5a}, R^{5b} and R^{5c} is as defined in Tables 1 to 10, G is G-20.3 and the combination of Y and R² for a compound corresponds in each case to one row of Table A.

### Tables 281 to 290

Compounds of the formula I.1 in which the combination of R^{5a}, R^{5b} and R^{5c} is as defined in Tables 1 to 10, G is G-20.4 and the combination of Y and R² for a compound corresponds in each case to one row of Table A.

### Tables 291 to 300

Compounds of the formula I.1 in which the combination of R^{5a}, R^{5b} and R^{5c} is as defined in Tables 1 to 10, G is G-21.1 and the combination of Y and R² for a compound corresponds in each case to one row of Table A.

### Tables 301 to 310

Compounds of the formula I.1 in which the combination of R^{5a}, R^{5b} and R^{5c} is as defined in Tables 1 to 10, G is G-22.1 and the combination of Y and R² for a compound corresponds in each case to one row of Table A.

### Tables 311 to 320

Compounds of the formula I.1 in which the combination of R^{5a}, R^{5b} and R^{5c} is as defined in Tables 1 to 10, G is G-23.1 and the combination of Y and R² for a compound corresponds in each case to one row of Table A.

### Tables 321 to 330

Compounds of the formula I.1 in which the combination of R^{5a}, R^{5b} and R^{5c} is as defined in Tables 1 to 10, G is G-24.1 and the combination of Y and R² for a compound corresponds in each case to one row of Table A.

### Tables 331 to 340

Compounds of the formula I.1 in which the combination of R^{5a}, R^{5b} and R^{5c} is as defined in Tables 1 to 10, G is G-25.1 and the combination of Y and R² for a compound corresponds in each case to one row of Table A.

### Tables 341 to 350

Compounds of the formula I.1 in which the combination of R^{5a}, R^{5b} and R^{5c} is as defined in Tables 1 to 10, G is G-26.1 and the combination of Y and R² for a compound corresponds in each case to one row of Table A.

### Tables 351 to 360

Compounds of the formula I.1 in which the combination of R^{5a}, R^{5b} and R^{5c} is as defined in Tables 1 to 10, G is G-27.1 and the combination of Y and R² for a compound corresponds in each case to one row of Table A.

### Tables 361 to 370

Compounds of the formula I.1 in which the combination of R^{5a}, R^{5b} and R^{5c} is as defined in Tables 1 to 10, G is G-28.1 and the combination of Y and R² for a compound corresponds in each case to one row of Table A.

### Tables 371 to 740

Compounds of the formula I.2 in which the combination of R^{5a}, R^{5b}, R^{5c} and G is as defined in Tables 1 to 370 and the combination of Y and R² for a compound corresponds in each case to one row of Table A.

### Tables 741 to 1110

Compounds of the formula I.3 in which the combination of R^{5a}, R^{5b}, R^{5c} and G is as defined in Tables 1 to 370 and the combination of Y and R² for a compound corresponds in each case to one row of Table A.

### Tables 1111 to 1480

Compounds of the formula I.4 in which the combination of R^{5a}, R^{5b}, R^{5c} and G is as defined in Tables 1 to 370 and the combination of Y and R² for a compound corresponds in each case to one row of Table A.

### Tables 1481 to 1850

Compounds of the formula I.5 in which the combination of R^{5a}, R^{5b}, R^{5c} and G is as defined in Tables 1 to 370 and the combination of Y and R² for a compound corresponds in each case to one row of Table A.

### Tables 1851 to 2220

Compounds of the formula I.6 in which the combination of R^{5a}, R^{5b}, R^{5c} and G is as defined in Tables 1 to 370 and the combination of Y and R² for a compound corresponds in each case to one row of Table A.

### Tables 2221 to 2590

Compounds of the formula I.7 in which the combination of R^{5a}, R^{5b}, R^{5c} and G is as defined in Tables 1 to 370 and the combination of Y and R² for a compound corresponds in each case to one row of Table A.

### Tables 2591 to 2960

Compounds of the formula I.8 in which the combination of R^{5a}, R^{5b}, R^{5c} and G is as defined in Tables 1 to 370 and the combination of Y and R² for a compound corresponds in each case to one row of Table A.

### Tables 2961 to 3330

Compounds of the formula I.9 in which the combination of R^{5a}, R^{5b}, R^{5c} and G is as defined in Tables 1 to 370 and the combination of Y and R² for a compound corresponds in each case to one row of Table A.

### Tables 3331 to 3700

Compounds of the formula I.10 in which the combination of R^{5a}, R^{5b}, R^{5c} and G is as defined in Tables 1 to 370 and the combination of Y and R² for a compound corresponds in each case to one row of Table A.

### Tables 3701 to 4070

Compounds of the formula I.11 in which the combination of R^{5a}, R^{5b}, R^{5c} and G is as defined in Tables 1 to 370 and the combination of Y and R² for a compound corresponds in each case to one row of Table A.

### Tables 4071 to 4440

Compounds of the formula I.12 in which the combination of R^{5a}, R^{5b}, R^{5c} and G is as defined in Tables 1 to 370 and the combination of Y and R² for a compound corresponds in each case to one row of Table A.

### Tables 4441 to 4810

Compounds of the formula I.13 in which the combination of R^{5a}, R^{5b}, R^{5c} and G is as defined in Tables 1 to 370 and the combination of Y and R² for a compound corresponds in each case to one row of Table A.

### Tables 4811 to 5180

Compounds of the formula I.14 in which the combination of R^{5a}, R^{5b}, R^{5c} and G is as defined in Tables 1 to 370 and the combination of Y and R² for a compound corresponds in each case to one row of Table A.

### Tables 5181 to 5550

Compounds of the formula I.15 in which the combination of R^{5a}, R^{5b}, R^{5c} and G is as defined in Tables 1 to 370 and the combination of Y and R² for a compound corresponds in each case to one row of Table A.

### Tables 5551 to 5920

Compounds of the formula I.16 in which the combination of R^{5a}, R^{5b}, R^{5c} and G is as defined in Tables 1 to 370 and the combination of Y and R² for a compound corresponds in each case to one row of Table A.

### Tables 5921 to 6290

Compounds of the formula I.17 in which the combination of R^{5a}, R^{5b}, R^{5c} and G is as defined in Tables 1 to 370 and the combination of Y and R² for a compound corresponds in each case to one row of Table A.

### Tables 6291 to 6660

Compounds of the formula I.18 in which the combination of R^{5a}, R^{5b}, R^{5c} and G is as defined in Tables 1 to 370 and the combination of Y and R² for a compound corresponds in each case to one row of Table A.

### Tables 6661 to 7030

Compounds of the formula I.19 in which the combination of R^{5a}, R^{5b}, R^{5c} and G is as defined in Tables 1 to 370 and the combination of Y and R² for a compound corresponds in each case to one row of Table A.

### Tables 7031 to 7400

Compounds of the formula I.20 in which the combination of R^{5a}, R^{5b}, R^{5c} and G is as defined in Tables 1 to 370 and the combination of Y and R² for a compound corresponds in each case to one row of Table A.

### Tables 7401 to 7770

Compounds of the formula I.21 in which the combination of R^{5a}, R^{5b}, R^{5c} and G is as defined in Tables 1 to 370 and the combination of Y and R² for a compound corresponds in each case to one row of Table A.

### Tables 7771 to 8140

Compounds of the formula I.22 in which the combination of R^{5a}, R^{5b}, R^{5c} and G is as defined in Tables 1 to 370 and the combination of Y and R² for a compound corresponds in each case to one row of Table A.

### Tables 8141 to 8510

Compounds of the formula I.23 in which the combination of R^{5a}, R^{5b}, R^{5c} and G is as defined in Tables 1 to 370 and the combination of Y and R² for a compound corresponds in each case to one row of Table A.

Tables 8511 to 8880

Compounds of the formula I.24 in which the combination of R^{5a}, R^{5b}, R^{5c} and G is as defined in Tables 1 to 370 and the combination of Y and R² for a compound corresponds in each case to one row of Table A.

### Tables 8881 to 9250

Compounds of the formula I.25 in which the combination of R^{5a}, R^{5b}, R^{5c} and G is as defined in Tables 1 to 370 and the combination of Y and R² for a compound corresponds in each case to one row of Table A.

### Tables 9251 to 9620

Compounds of the formula I.26 in which the combination of R^{5a}, R^{5b}, R^{5c} and G is as defined in Tables 1 to 370 and the combination of Y and R² for a compound corresponds in each case to one row of Table A.

### Tables 9621 to 9990

Compounds of the formula I.27 in which the combination of R^{5a}, R^{5b}, R^{5c} and G is as defined in Tables 1 to 370 and the combination of Y and R² for a compound corresponds in each case to one row of Table A.

### Tables 9991 to 10360

Compounds of the formula I.28 in which the combination of R^{5a}, R^{5b}, R^{5c} and G is as defined in Tables 1 to 370 and the combination of Y and R² for a compound corresponds in each case to one row of Table A.

### Tables 10361 to 10730

Compounds of the formula I.29 in which the combination of R^{5a}, R^{5b}, R^{5c} and G is as defined in Tables 1 to 370 and the combination of Y and R² for a compound corresponds in each case to one row of Table A.

### Tables 10731 to 11100

Compounds of the formula I.30 in which the combination of R^{5a}, R^{5b}, R^{5c} and G is as defined in Tables 1 to 370 and the combination of Y and R² for a compound corresponds in each case to one row of Table A.

### Tables 11101 to 11470

Compounds of the formula I.31 in which the combination of R^{5a}, R^{5b}, R^{5c} and G is as defined in Tables 1 to 370 and the combination of Y and R² for a compound corresponds in each case to one row of Table A.

Tables 11471 to 11840

Compounds of the formula I.32 in which the combination of R^{5a}, R^{5b}, R^{5c} and G is as defined in Tables 1 to 370 and the combination of Y and R² for a compound corresponds in each case to one row of Table A.

### Tables 11841 to 12210

Compounds of the formula I.33 in which the combination of R^{5a}, R^{5b}, R^{5c} and G is as defined in Tables 1 to 370 and the combination of Y and R² for a compound corresponds in each case to one row of Table A.

### Tables 12211 to 12580

Compounds of the formula I.34 in which the combination of R^{5a}, R^{5b}, R^{5c} and G is as defined in Tables 1 to 370 and the combination of Y and R² for a compound corresponds in each case to one row of Table A.

### Tables 12581 to 12950

Compounds of the formula I.35 in which the combination of R^{5a}, R^{5b}, R^{5c} and G is as defined in Tables 1 to 370 and the combination of Y and R² for a compound corresponds in each case to one row of Table A.

### Tables 12951 to 13320

Compounds of the formula I.36 in which the combination of R^{5a}, R^{5b}, R^{5c} and G is as defined in Tables 1 to 370 and the combination of Y and R² for a compound corresponds in each case to one row of Table A.

Tables 13321 to 13690

Compounds of the formula I.37 in which the combination of R^{5a}, R^{5b}, R^{5c} and G is as defined in Tables 1 to 370 and the combination of Y and R² for a compound corresponds in each case to one row of Table A.

### Tables 13691 to 14060

Compounds of the formula I.38 in which the combination of R^{5a}, R^{5b}, R^{5c} and G is as defined in Tables 1 to 370 and the combination of Y and R² for a compound corresponds in each case to one row of Table A.

### Tables 14061 to 14430

Compounds of the formula I.39 in which the combination of R^{5a}, R^{5b}, R^{5c} and G is as defined in Tables 1 to 370 and the combination of Y and R² for a compound corresponds in each case to one row of Table A.

### Tables 14431 to 14800

Compounds of the formula I.40 in which the combination of R^{5a}, R^{5b}, R^{5c} and G is as defined in Tables 1 to 370 and the combination of Y and R² for a compound corresponds in each case to one row of Table A.

### Tables 14801 to 15170

Compounds of the formula I.41 in which the combination of R^{5a}, R^{5b}, R^{5c} and G is as defined in Tables 1 to 370 and the combination of Y and R² for a compound corresponds in each case to one row of Table A.

### Tables 15171 to 15540

Compounds of the formula I.42 in which the combination of R^{5a}, R^{5b}, R^{5c} and G is as defined in Tables 1 to 370 and the combination of Y and R² for a compound corresponds in each case to one row of Table A.

Tables 15541 to 15910

Compounds of the formula I.43 in which the combination of R^{5a}, R^{5b}, R^{5c} and G is as defined in Tables 1 to 370 and the combination of Y and R² for a compound corresponds in each case to one row of Table A.

### Tables 15911 to 16280

Compounds of the formula I.44 in which the combination of R^{5a}, R^{5b}, R^{5c} and G is as defined in Tables 1 to 370 and the combination of Y and R² for a compound corresponds in each case to one row of Table A.

### Tables 16281 to 16650

Compounds of the formula I.45 in which the combination of R^{5a}, R^{5b}, R^{5c} and G is as defined in Tables 1 to 370 and the combination of Y and R² for a compound corresponds in each case to one row of Table A.

### Tables 16651 to 17020

Compounds of the formula I.46 in which the combination of R^{5a}, R^{5b}, R^{5c} and G is as defined in Tables 1 to 370 and the combination of Y and R² for a compound corresponds in each case to one row of Table A.

### Tables 17021 to 17390

Compounds of the formula I.47 in which the combination of R^{5a}, R^{5b}, R^{5c} and G is as defined in Tables 1 to 370 and the combination of Y and R² for a compound corresponds in each case to one row of Table A.

### Tables 17391 to 17760

Compounds of the formula I.48 in which the combination of R^{5a}, R^{5b}, R^{5c} and G is as defined in Tables 1 to 370 and the combination of Y and R² for a compound corresponds in each case to one row of Table A.

### Tables 17761 to 18130

Compounds of the formula I.49 in which the combination of R^{5a}, R^{5b}, R^{5c} and G is as defined in Tables 1 to 370 and the combination of Y and R² for a compound corresponds in each case to one row of Table A.

### Tables 18131 to 18500

Compounds of the formula I.50 in which the combination of R^{5a}, R^{5b}, R^{5c} and G is as defined in Tables 1 to 370 and the combination of Y and R² for a compound corresponds in each case to one row of Table A.

### Tables 18501 to 18870

Compounds of the formula I.51 in which the combination of R^{5a}, R^{5b}, R^{5c} and G is as defined in Tables 1 to 370 and the combination of Y and R² for a compound corresponds in each case to one row of Table A.

### Tables 18871 to 19240

Compounds of the formula I.52 in which the combination of R^{5a}, R^{5b}, R^{5c} and G is as defined in Tables 1 to 370 and the combination of Y and R² for a compound corresponds in each case to one row of Table A.

### Tables 19241 to 19610

Compounds of the formula I.53 in which the combination of R^{5a}, R^{5b}, R^{5c} and G is as defined in Tables 1 to 370 and the combination of Y and R² for a compound corresponds in each case to one row of Table A.

### Tables 19611 to 19980

Compounds of the formula I.54 in which the combination of R^{5a}, R^{5b}, R^{5c} and G is as defined in Tables 1 to 370 and the combination of Y and R² for a compound corresponds in each case to one row of Table A.

### Tables 19981 to 20350

Compounds of the formula I.55 in which the combination of R^{5a}, R^{5b}, R^{5c} and G is as defined in Tables 1 to 370 and the combination of Y and R² for a compound corresponds in each case to one row of Table A.

### Tables 20351 to 20720

Compounds of the formula I.56 in which the combination of R^{5a}, R^{5b}, R^{5c} and G is as defined in Tables 1 to 370 and the combination of Y and R² for a compound corresponds in each case to one row of Table A.

### Tables 20721 to 21090

Compounds of the formula I.57 in which the combination of R^{5a}, R^{5b}, R^{5c} and G is as defined in Tables 1 to 370 and the combination of Y and R² for a compound corresponds in each case to one row of Table A.

### Tables 21091 to 21460

Compounds of the formula I.58 in which the combination of R^{5a}, R^{5b}, R^{5c} and G is as defined in Tables 1 to 370 and the combination of Y and R² for a compound corresponds in each case to one row of Table A.

### Tables 21461 to 21830

Compounds of the formula I.59 in which the combination of R^{5a}, R^{5b}, R^{5c} and G is as defined in Tables 1 to 370 and the combination of Y and R² for a compound corresponds in each case to one row of Table A.

### Tables 21831 to 22200

Compounds of the formula I.60 in which the combination of R^{5a}, R^{5b}, R^{5c} and G is as defined in Tables 1 to 370 and the combination of Y and R² for a compound corresponds in each case to one row of Table A.

### Tables 22201 to 22570

Compounds of the formula I.61 in which the combination of R^{5a}, R^{5b}, R^{5c} and G is as defined in Tables 1 to 370 and the combination of Y and R² for a compound corresponds in each case to one row of Table A.

### Tables 22571 to 22940

Compounds of the formula I.62 in which the combination of R^{5a}, R^{5b}, R^{5c} and G is as defined in Tables 1 to 370 and the combination of Y and R² for a compound corresponds in each case to one row of Table A.

### Tables 22941 to 23310

Compounds of the formula I.63 in which the combination of R^{5a}, R^{5b}, R^{5c} and G is as defined in Tables 1 to 370 and the combination of Y and R² for a compound corresponds in each case to one row of Table A.

### Tables 23311 to 23680

Compounds of the formula I.64 in which the combination of R^{5a}, R^{5b}, R^{5c} and G is as defined in Tables 1 to 370 and the combination of Y and R² for a compound corresponds in each case to one row of Table A.

### Tables 23681 to 24050

Compounds of the formula I.65 in which the combination of R^{5a}, R^{5b}, R^{5c} and G is as defined in Tables 1 to 370 and the combination of Y and R² for a compound corresponds in each case to one row of Table A.

### Tables 24051 to 24420

Compounds of the formula I.66 in which the combination of R^{5a}, R^{5b}, R^{5c} and G is as defined in Tables 1 to 370 and the combination of Y and R² for a compound corresponds in each case to one row of Table A.

### Rings G

In these rings, “*” is the attachment point to the “left” phenyl ring carrying the substituents R^{5a}, R^{5b} and R^{5c} and “#” is the attachment point to the “right” phenyl or pyridyl ring carrying the imino group —C(R¹)═N—Y—R².

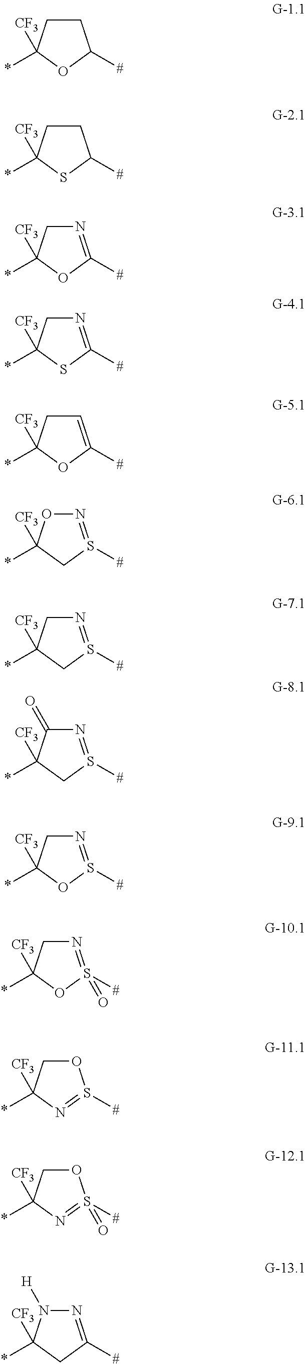

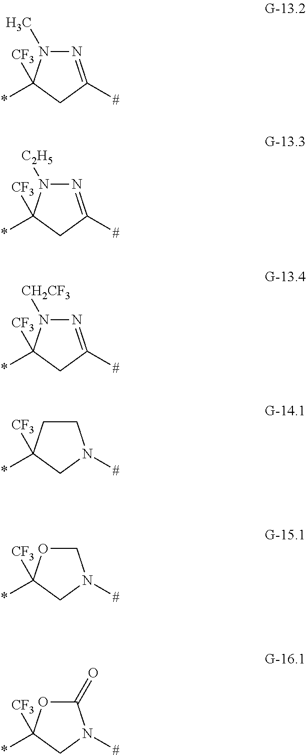

TABLE A

| No. | Y | R² |
|---|---|---|
| 1. | O | H |
| 2. | O | CH₃ |
| 3. | O | CF₃ |
| 4. | O | CH₂CH₃ |
| 5. | O | CH₂CF₃ |
| 6. | O | CH₂CH₂CH₃ |
| 7. | O | CH₂CH₂CF₃ |
| 8. | O | CH₂CF₂CF₃ |
| 9. | O | CH(CH₃)₂ |
| 10. | O | (CH₂)₃CH₃ |
| 11. | O | (CH₂)₃CF₃ |
| 12. | O | CH₂-^{c}propyl |
| 13. | O | CH(CH₃)-^{c}propyl |
| 14. | O | CH₂CN |
| 15. | O | CH₂C(═O)OH |
| 16. | O | CH₂C(═O)OCH₃ |
| 17. | O | CH₂C(═O)OCH₂CH₃ |
| 18. | O | CH₂C(═O)OCH₂CH₂CH₃ |
| 19. | O | CH₂C(═O)OCH(CH₃)₂ |
| 20. | O | CH₂C(═O)O(CH₂)₃CH₃ |
| 21. | O | CH₂C(═O)OCH₂CH(CH₃)₂ |
| 22. | O | CH₂C(═O)OC(CH₃)₃ |
| 23. | O | CH₂C(═O)NH₂ |
| 24. | O | CH₂C(═O)NHCH₃ |
| 25. | O | CH₂C(═O)N(CH₃)₂ |
| 26. | O | CH₂C(═O)NHCF₃ |
| 27. | O | CH₂C(═O)N(CF₃)₂ |
| 28. | O | CH₂C(═O)NHCH₂CH₃ |
| 29. | O | CH₂C(═O)N(CH₂CH₃)₂ |
| 30. | O | CH₂C(═O)NHCH₂CF₃ |
| 31. | O | CH₂C(═O)N(CH₂CF₃)₂ |
| 32. | O | CH₂C(═O)NHCH₂CH₂CH₃ |
| 33. | O | CH₂C(═O)NHCH(CH₃)₂ |
| 34. | O | CH₂C(═O)NH(CH₂)₃CH₃ |
| 35. | O | CH₂C(═O)NHCH₂-^{c}propyl |
| 36. | O | CH₂C(═O)NHCH₂—C₆H₅ |
| 37. | O | CH₂C(═O)NH-propargyl |
| 38. | O | CH₂C(═O)NHCH₂-4-Cl—C₆H₄ |
| 39. | O | CH₂C(═O)-morpholin-4-yl |
| 40. | O | C₆H₅ |
| 41. | O | 2-F—C₆H₄ |
| 42. | O | 3-F—C₆H₄ |
| 43. | O | 4-F—C₆H₄ |
| 44. | O | 2-Cl—C₆H₄ |
| 45. | O | 3-Cl—C₆H₄ |
| 46. | O | 4-Cl—C₆H₄ |
| 47. | O | 2-Br—C₆H₄ |
| 48. | O | 3-Br—C₆H₄ |
| 49. | O | 4-Br—C₆H₄ |
| 50. | O | C(═O)CH₃ |
| 51. | O | C(═O)CH₂CH₃ |
| 52. | O | C(═O)CH₂OCH₃ |
| 53. | O | C(═O)(CH₂)₂CH₃ |
| 54. | O | C(═O)CH(CH₃)₂ |
| 55. | O | C(═O)C₆H₅ |
| 56. | O | C(═O)-2-F—C₆H₄ |
| 57. | O | C(═O)-3-F—C₆H₄ |
| 58. | O | C(═O)-4-F—C₆H₄ |
| 59. | O | C(═O)-2-Cl—C₆H₄ |
| 60. | O | C(═O)-3-Cl—C₆H₄ |
| 61. | O | C(═O)-4-Cl—C₆H₄ |
| 62. | O | C(═O)-2-Br—C₆H₄ |
| 63. | O | C(═O)-3-Br—C₆H₄ |
| 64. | O | C(═O)-4-Br—C₆H₄ |
| 65. | O | C(═O)-2-pyridyl |
| 66. | O | C(═O)CF₃ |
| 67. | O | C(═O)CH₂CF₃ |
| 68. | O | C(═O)CH₂CCl₃ |
| 69. | O | C(═O)OCH₂Cl₃ |
| 70. | O | C(═O)OH |
| 71. | O | C(═O)OCH₃ |
| 72. | O | C(═O)OCH₂CH₃ |
| 73. | O | C(═O)OCH₂CH₂CH₃ |
| 74. | O | C(═O)OCH(CH₃)₂ |
| 75. | O | C(═O)O(CH₂)₃CH₃ |
| 76. | O | C(═O)OCH₂CH(CH₃)₂ |
| 77. | O | C(═O)OC(CH₃)₃ |
| 78. | O | C(═O)NH₂ |
| 79. | O | C(═O)NHCH₃ |
| 80. | O | C(═O)N(CH₃)₂ |
| 81. | O | C(═O)NHCF₃ |
| 82. | O | C(═O)N(CF₃)₂ |
| 83. | O | C(═O)NHCH₂CH₃ |
| 84. | O | C(═O)N(CH₂CH₃)₂ |
| 85. | O | C(═O)N(CH₃)CH₂CH₃ |
| 86. | O | C(═O)NHCH₂CF₃ |
| 87. | O | C(═O)N(CH₂CF₃)₂ |
| 88. | O | C(═O)N(CH₃)CH₂CF₃ |
| 89. | O | C(═O)NHCH₂CH₂CH₃ |
| 90. | O | C(═O)N(CH₃)CH₂CH₂CH₃ |
| 91. | O | C(═O)NHCH(CH₃)₂ |
| 92. | O | C(═O)NH(CH₂)₃CH₃ |
| 93. | O | C(═O)N(CH₃)—(CH₂)₃CH₃ |
| 94. | O | C(═O)N[(CH₂)₃CH₃]₂ |
| 95. | O | C(═O)N(CH₃)—CH₂—C₆H₅ |
| 96. | O | C(═O)NH-propargyl |
| 97. | O | C(═O)N(CH₃)-propargyl |
| 98. | O | C(═O)NH—CH₂-4-Cl—C₆H₄ |
| 99. | O | C(═O)N(CH₃)—CH₂-4-Cl—C₆H₄ |
| 100. | O | C(═O)morpholin-4-yl |
| 101. | O | C(═O)NH-3-thiolyl-1,1-dioxid |
| 102. | O | C(═O)N(CH₃)-3-thiolyl-1,1-dioxid |
| 103. | O | C(═O)-azirid-1-yl |
| 104. | O | C(═O)-pyrrolidin-1-yl |
| 105. | O | C(═O)-piperidin-1-yl |
| 106. | O | C(═O)-thiomorpholin-4-yl |
| 107. | O | C(═O)NH—CH₂CHF₂ |
| 108. | O | C(═O)NH—CH₂CH₂CHF₂ |
| 109. | O | C(═O)NH—CH₂CH₂CF₃ |
| 110. | O | C(═O)NH-cyclopropyl |
| 111. | O | C(═O)NH-cyclobutyl |
| 112. | O | C(═O)NH-cyclopentyl |
| 113. | O | C(═O)NH-cyclohexyl |
| 114. | O | C(═O)NH—CH₂-cyclopropyl |
| 115. | O | C(═O)NH—CH₂-cyclobutyl |
| 116. | O | C(═O)NH—CH₂-cyclopentyl |
| 117. | O | C(═O)NH—CH₂-cyclohexyl |
| 118. | O | C(═O)NH—CN |
| 119. | O | C(═O)NH—CH₂—CN |
| 120. | O | C(═O)NH—CH₂—CH═CH₂ |
| 121. | O | C(═O)NH—CH₂—CH═C(Cl)₂ |
| 122. | O | C(═O)NH—CH₂—CH═CH-phenyl |
| 123. | O | C(═O)NH—CH₂—CH═CH-(4-Cl-phenyl) |
| 124. | O | C(═O)NH—CH₂—SCH₃ |
| 125. | O | C(═O)NH—CH₂—SCF₃ |
| 126. | O | C(═O)NH—CH₂—CH₂—SCH₃ |
| 127. | O | C(═O)NH—CH₂—CH₂—SCF₃ |
| 128. | O | C(═O)NH—CH₂—SO₂—CH₃ |
| 129. | O | C(═O)NH—CH₂—SO₂—CF₃ |
| 130. | O | C(═O)NH—CH₂—CH₂—SO₂—CH₃ |
| 131. | O | C(═O)NH—CH₂—CH₂—SO₂—CF₃ |
| 132. | O | C(═O)NH—CH₂—CO—NH₂ |
| 133. | O | C(═O)NH—CH₂—CO—NHCH₃ |
| 134. | O | C(═O)NH—CH₂—CO—N(CH₃)₂ |
| 135. | O | C(═O)NH—CH₂—CO—NHCF₃ |
| 136. | O | C(═O)NH—CH₂—CO—N(CF₃)₂ |
| 137. | O | C(═O)NH—CH₂—CO—NHCH₂CH₃ |
| 138. | O | C(═O)NH—CH₂—CO—N(CH₂CH₃)₂ |
| 139. | O | C(═O)NH—CH₂—CO—NHCH₂CF₃ |
| 140. | O | C(═O)NH—CH₂—CO—N(CH₂CF₃)₂ |
| 141. | O | C(═O)NH—CH₂—CO—NHCH₂CH₂CH₃ |
| 142. | O | C(═O)NH—CH₂—CO—N(CH₂CH₂CH₃)₂ |
| 143. | O | C(═O)NH—CH₂—CO—NHCH₂CH₂CF₃ |
| 144. | O | C(═O)NH—CH₂—CO—N(CH₂CH₂CF₃)₂ |
| 145. | O | C(═O)NH—CH₂—CO—NHCH(CH₃)₂ |
| 146. | O | C(═O)NH—CH₂—CO—NHCH(CF₃)₂ |
| 147. | O | C(═O)NH—CH₂—CO—NH-cyclopropyl |
| 148. | O | C(═O)NH—CH₂—CO—NH—CH₂-cyclopropyl |
| 149. | O | C(═O)NH—CH₂—CO—OH |
| 150. | O | C(═O)NH—CH₂—CO—OCH₃ |
| 151. | O | C(═O)NH—CH₂—CO—OCF₃ |
| 152. | O | C(═O)NH—CH₂—CO—OCH₂CH₃ |
| 153. | O | C(═O)NH—CH₂—CO—OCH₂CF₃ |
| 154. | O | C(═O)NH—CH₂—CO—OCH₂CH₂CH₃ |
| 155. | O | C(═O)NH—CH₂—CO—OCH(CH₃)₂ |
| 156. | O | C(═O)NH—CH₂—CO—OCH₂CH₂CH₂CH₃ |
| 157. | O | C(═O)NH—CH₂—CO—OCH(CH₃)CH₂CH₃ |
| 158. | O | C(═O)NH—CH₂—CO—OCH₂CH(CH₃)₂ |
| 159. | O | C(═O)NH—CH₂—CO—OC(CH₃)₃ |
| 160. | O | C(═O)NH-A-1 |
| 161. | O | C(═O)NH-A-2 |
| 162. | O | C(═O)NH-A-3 |
| 163. | O | C(═O)NH-A-4 |
| 164. | O | C(═O)NH-A-5 |
| 165. | O | C(═O)NH-A-6 |
| 166. | O | C(═O)NH-A-7 |
| 167. | O | C(═O)NH-A-8 |
| 168. | O | C(═O)NH-A-9 |
| 169. | O | C(═O)NH-A-10 |
| 170. | O | C(═O)NH-A-11 |
| 171. | O | C(═O)NH-A-12 |
| 172. | O | C(═O)NH-A-13 |
| 173. | O | C(═O)NH-A-14 |
| 174. | O | C(═O)NH-A-15 |
| 175. | O | C(═O)NH-A-16 |
| 176. | O | C(═O)NH-A-17 |
| 177. | O | C(═O)NH-A-18 |
| 178. | O | C(═O)NH-A-19 |
| 179. | O | C(═O)NH-A-20 |
| 180. | O | C(═O)NH-A-21 |
| 181. | O | C(═O)NH-A-22 |
| 182. | O | C(═O)NH-A-23 |
| 183. | O | C(═O)NH-A-24 |
| 184. | O | C(═O)NH-A-25 |
| 185. | O | C(═O)NH-A-26 |
| 186. | O | C(═O)NH-A-27 |
| 187. | O | C(═O)NH-A-28 |
| 188. | O | C(═O)NH-A-29 |
| 189. | O | C(═O)NH-A-30 |
| 190. | O | C(═O)NH-A-31 |
| 191. | O | C(═O)NH-A-32 |
| 192. | O | C(═O)NH-A-33 |
| 193. | O | C(═O)NH—CH₂-A-1 |
| 194. | O | C(═O)NH—CH₂-A-2 |
| 195. | O | C(═O)NH—CH₂-A-3 |
| 196. | O | C(═O)NH—CH₂-A-4 |
| 197. | O | C(═O)NH—CH₂-A-5 |
| 198. | O | C(═O)NH—CH₂-A-6 |
| 199. | O | C(═O)NH—CH₂-A-7 |
| 200. | O | C(═O)NH—CH₂-A-8 |
| 201. | O | C(═O)NH—CH₂-A-9 |
| 202. | O | C(═O)NH—CH₂-A-10 |
| 203. | O | C(═O)NH—CH₂-A-11 |
| 204. | O | C(═O)NH—CH₂-A-12 |
| 205. | O | C(═O)NH—CH₂-A-13 |
| 206. | O | C(═O)NH—CH₂-A-14 |
| 207. | O | C(═O)NH—CH₂-A-15 |
| 208. | O | C(═O)NH—CH₂-A-16 |
| 209. | O | C(═O)NH—CH₂-A-17 |
| 210. | O | C(═O)NH—CH₂-A-18 |
| 211. | O | C(═O)NH—CH₂-A-19 |
| 212. | O | C(═O)NH—CH₂-A-20 |
| 213. | O | C(═O)NH—CH₂-A-21 |
| 214. | O | C(═O)NH—CH₂-A-22 |
| 215. | O | C(═O)NH—CH₂-A-23 |
| 216. | O | C(═O)NH—CH₂-A-24 |
| 217. | O | C(═O)NH—CH₂-A-25 |
| 218. | O | C(═O)NH—CH₂-A-26 |
| 219. | O | C(═O)NH—CH₂-A-27 |
| 220. | O | C(═O)NH—CH₂-A-28 |
| 221. | O | C(═O)NH—CH₂-A-29 |
| 222. | O | C(═O)NH—CH₂-A-30 |
| 223. | O | C(═O)NH—CH₂-A-31 |
| 224. | O | C(═O)NH—CH₂-A-32 |
| 225. | O | C(═O)NH—CH₂-A-33 |
| 226. | O | C(═O)NH—SO₂—CH₃ |
| 227. | O | C(═O)NH—SO₂—CF₃ |
| 228. | O | C(═O)NH—SO₂—CH₂CH₃ |
| 229. | O | C(═O)NH—SO₂—CH₂CF₃ |
| 230. | O | C(═O)NH—SO₂—CH₂CH₂CH₃ |
| 231. | O | C(═O)NH—SO₂—CH₂CH₂CF₃ |
| 232. | O | C(═O)NH—SO₂—CH₂CF₂CF₃ |
| 233. | O | C(═O)NH—SO₂—CH(CH₃)₂ |
| 234. | O | C(═O)NH—SO₂—CH(CF₃)₂ |
| 235. | O | C(═O)N(CH₃)—CH₂CHF₂ |
| 236. | O | C(═O)N(CH₃)—CH₂CH₂CHF₂ |
| 237. | O | C(═O)N(CH₃)—CH₂CH₂CF₃ |
| 238. | O | C(═O)N(CH₃)-cyclopropyl |
| 239. | O | C(═O)N(CH₃)-cyclobutyl |
| 240. | O | C(═O)N(CH₃)-cyclopentyl |
| 241. | O | C(═O)N(CH₃)-cyclohexyl |
| 242. | O | C(═O)N(CH₃)—CH₂-cyclopropyl |
| 243. | O | C(═O)N(CH₃)—CH₂-cyclobutyl |
| 244. | O | C(═O)N(CH₃)—CH₂-cyclopentyl |
| 245. | O | C(═O)N(CH₃)—CH₂-cyclohexyl |
| 246. | O | C(═O)N(CH₃)—CN |
| 247. | O | C(═O)N(CH₃)—CH₂—CN |
| 248. | O | C(═O)N(CH₃)—CH₂—CH═CH₂ |
| 249. | O | C(═O)N(CH₃)—CH₂—CH═C(Cl)₂ |
| 250. | O | C(═O)N(CH₃)—CH₂—CH═CH-phenyl |
| 251. | O | C(═O)N(CH₃)—CH₂—CH═CH-(4-Cl-phenyl) |
| 252. | O | C(═O)N(CH₃)—CH₂—SCH₃ |
| 253. | O | C(═O)N(CH₃)—CH₂—SCF₃ |
| 254. | O | C(═O)N(CH₃)—CH₂—CH₂—SCH₃ |
| 255. | O | C(═O)N(CH₃)—CH₂—CH₂—SCF₃ |
| 256. | O | C(═O)N(CH₃)—CH₂—SO₂—CH₃ |
| 257. | O | C(═O)N(CH₃)—CH₂—SO₂—CF₃ |
| 258. | O | C(═O)N(CH₃)—CH₂—CH₂—SO₂—CH₃ |
| 259. | O | C(═O)N(CH₃)—CH₂—CH₂—SO₂—CF₃ |
| 260. | O | C(═O)N(CH₃)—CH₂—CO—NH₂ |
| 261. | O | C(═O)N(CH₃)—CH₂—CO—NHCH₃ |
| 262. | O | C(═O)N(CH₃)—CH₂—CO—N(CH₃)₂ |
| 263. | O | C(═O)N(CH₃)—CH₂—CO—NHCF₃ |
| 264. | O | C(═O)N(CH₃)—CH₂—CO—N(CF₃)₂ |
| 265. | O | C(═O)N(CH₃)—CH₂—CO—NHCH₂CH₃ |
| 266. | O | C(═O)N(CH₃)—CH₂—CO—N(CH₂CH₃)₂ |
| 267. | O | C(═O)N(CH₃)—CH₂—CO—NHCH₂CF₃ |
| 268. | O | C(═O)N(CH₃)—CH₂—CO—N(CH₂CF₃)₂ |
| 269. | O | C(═O)N(CH₃)—CH₂—CO—NHCH₂CH₂CH₃ |
| 270. | O | C(═O)N(CH₃)—CH₂—CO—N(CH₂CH₂CH₃)₂ |
| 271. | O | C(═O)N(CH₃)—CH₂—CO—NHCH₂CH₂CF₃ |
| 272. | O | C(═O)N(CH₃)—CH₂—CO—N(CH₂CH₂CF₃)₂ |
| 273. | O | C(═O)N(CH₃)—CH₂—CO—NHCH(CH₃)₂ |
| 274. | O | C(═O)N(CH₃)—CH₂—CO—NHCH(CF₃)₂ |
| 275. | O | C(═O)N(CH₃)—CH₂—CO—NH-cyclopropyl |
| 276. | O | C(═O)N(CH₃)—CH₂—CO—NH—CH₂- |
| | | cyclopropyl |
| 277. | O | C(═O)N(CH₃)—CH₂—CO—OH |
| 278. | O | C(═O)N(CH₃)—CH₂—CO—OCH₃ |
| 279. | O | C(═O)N(CH₃)—CH₂—CO—OCF₃ |
| 280. | O | C(═O)N(CH₃)—CH₂—CO—OCH₂CH₃ |
| 281. | O | C(═O)N(CH₃)—CH₂—CO—OCH₂CF₃ |
| 282. | O | C(═O)N(CH₃)—CH₂—CO—OCH₂CH₂CH₃ |
| 283. | O | C(═O)N(CH₃)—CH₂—CO—OCH(CH₃)₂ |
| 284. | O | C(═O)N(CH₃)—CH₂—CO—OCH₂CH₂CH₂CH₃ |
| 285. | O | C(═O)N(CH₃)—CH₂—CO—OCH(CH₃)CH₂CH₃ |
| 286. | O | C(═O)N(CH₃)—CH₂—CO—OCH₂CH(CH₃)₂ |
| 287. | O | C(═O)N(CH₃)—CH₂—CO—OC(CH₃)₃ |
| 288. | O | C(═O)N(CH₃)-A-1 |
| 289. | O | C(═O)N(CH₃)-A-2 |
| 290. | O | C(═O)N(CH₃)-A-3 |
| 291. | O | C(═O)N(CH₃)-A-4 |
| 292. | O | C(═O)N(CH₃)-A-5 |
| 293. | O | C(═O)N(CH₃)-A-6 |
| 294. | O | C(═O)N(CH₃)-A-7 |
| 295. | O | C(═O)N(CH₃)-A-8 |
| 296. | O | C(═O)N(CH₃)-A-9 |
| 297. | O | C(═O)N(CH₃)-A-10 |
| 298. | O | C(═O)N(CH₃)-A-11 |
| 299. | O | C(═O)N(CH₃)-A-12 |
| 300. | O | C(═O)N(CH₃)-A-13 |
| 301. | O | C(═O)N(CH₃)-A-14 |
| 302. | O | C(═O)N(CH₃)-A-15 |
| 303. | O | C(═O)N(CH₃)-A-16 |
| 304. | O | C(═O)N(CH₃)-A-17 |
| 305. | O | C(═O)N(CH₃)-A-18 |
| 306. | O | C(═O)N(CH₃)-A-19 |
| 307. | O | C(═O)N(CH₃)-A-20 |
| 308. | O | C(═O)N(CH₃)-A-21 |
| 309. | O | C(═O)N(CH₃)-A-22 |
| 310. | O | C(═O)N(CH₃)-A-23 |
| 311. | O | C(═O)N(CH₃)-A-24 |
| 312. | O | C(═O)N(CH₃)-A-25 |
| 313. | O | C(═O)N(CH₃)-A-26 |
| 314. | O | C(═O)N(CH₃)-A-27 |
| 315. | O | C(═O)N(CH₃)-A-28 |
| 316. | O | C(═O)N(CH₃)-A-29 |
| 317. | O | C(═O)N(CH₃)-A-30 |
| 318. | O | C(═O)N(CH₃)-A-31 |
| 319. | O | C(═O)N(CH₃)-A-32 |
| 320. | O | C(═O)N(CH₃)-A-33 |
| 321. | O | C(═O)N(CH₃)—CH₂-A-1 |
| 322. | O | C(═O)N(CH₃)—CH₂-A-2 |
| 323. | O | C(═O)N(CH₃)—CH₂-A-3 |
| 324. | O | C(═O)N(CH₃)—CH₂-A-4 |
| 325. | O | C(═O)N(CH₃)—CH₂-A-5 |
| 326. | O | C(═O)N(CH₃)—CH₂-A-6 |
| 327. | O | C(═O)N(CH₃)—CH₂-A-7 |
| 328. | O | C(═O)N(CH₃)—CH₂-A-8 |
| 329. | O | C(═O)N(CH₃)—CH₂-A-9 |
| 330. | O | C(═O)N(CH₃)—CH₂-A-10 |
| 331. | O | C(═O)N(CH₃)—CH₂-A-11 |
| 332. | O | C(═O)N(CH₃)—CH₂-A-12 |
| 333. | O | C(═O)N(CH₃)—CH₂-A-13 |
| 334. | O | C(═O)N(CH₃)—CH₂-A-14 |
| 335. | O | C(═O)N(CH₃)—CH₂-A-15 |
| 336. | O | C(═O)N(CH₃)—CH₂-A-16 |
| 337. | O | C(═O)N(CH₃)—CH₂-A-17 |
| 338. | O | C(═O)N(CH₃)—CH₂-A-18 |
| 339. | O | C(═O)N(CH₃)—CH₂-A-19 |
| 340. | O | C(═O)N(CH₃)—CH₂-A-20 |
| 341. | O | C(═O)N(CH₃)—CH₂-A-21 |
| 342. | O | C(═O)N(CH₃)—CH₂-A-22 |
| 343. | O | C(═O)N(CH₃)—CH₂-A-23 |
| 344. | O | C(═O)N(CH₃)—CH₂-A-24 |
| 345. | O | C(═O)N(CH₃)—CH₂-A-25 |
| 346. | O | C(═O)N(CH₃)—CH₂-A-26 |
| 347. | O | C(═O)N(CH₃)—CH₂-A-27 |
| 348. | O | C(═O)N(CH₃)—CH₂-A-28 |
| 349. | O | C(═O)N(CH₃)—CH₂-A-29 |
| 350. | O | C(═O)N(CH₃)—CH₂-A-30 |
| 351. | O | C(═O)N(CH₃)—CH₂-A-31 |
| 352. | O | C(═O)N(CH₃)—CH₂-A-32 |
| 353. | O | C(═O)N(CH₃)—CH₂-A-33 |
| 354. | O | C(═O)N(CH₃)—SO₂—CH₃ |
| 355. | O | C(═O)N(CH₃)—SO₂—CF₃ |
| 356. | O | C(═O)N(CH₃)—SO₂—CH₂CH₃ |
| 357. | O | C(═O)N(CH₃)—SO₂—CH₂CF₃ |
| 358. | O | C(═O)N(CH₃)—SO₂—CH₂CH₂CH₃ |
| 359. | O | C(═O)N(CH₃)—SO₂—CH₂CH₂CF₃ |
| 360. | O | C(═O)N(CH₃)—SO₂—CH₂CF₂CF₃ |
| 361. | O | C(═O)N(CH₃)—SO₂—CH(CH₃)₂ |
| 362. | O | C(═O)N(CH₃)—SO₂—CH(CF₃)₂ |
| 363. | O | C(═O)NH—SO₂—NH₂ |
| 364. | O | C(═O)NH—SO₂—NHCH₃ |
| 365. | O | C(═O)NH—SO₂—N(CH₃)₂ |
| 366. | O | C(═O)NH—SO₂—NHCF₃ |
| 367. | O | C(═O)NH—SO₂—N(CF₃)₂ |
| 368. | O | C(═O)NH—SO₂—NHCH₂CH₃ |
| 369. | O | C(═O)NH—SO₂—N(CH₂CH₃)₂ |
| 370. | O | C(═O)NH—SO₂—NHCH₂CF₃ |
| 371. | O | C(═O)NH—SO₂—N(CH₂CF₃)₂ |
| 372. | O | C(═O)NH—SO₂—N(CH₃)CH₂CH₃ |
| 373. | O | C(═O)NH—SO₂—N(CH₃)CH₂CF₃ |
| 374. | O | C(═O)NH—SO₂—N(CF₃)CH₂CH₃ |
| 375. | O | C(═O)NH—SO₂—NHCH₂CH₂CH₃ |
| 376. | O | C(═O)NH—SO₂—N(CH₂CH₂CH₃)₂ |
| 377. | O | C(═O)NH—SO₂—NHCH₂CH₂CF₃ |
| 378. | O | C(═O)NH—SO₂—N(CH₂CH₂CF₃)₂ |
| 379. | O | C(═O)NH—SO₂—N(CH₃)CH₂CH₂CH₃ |
| 380. | O | C(═O)NH—SO₂—N(CH₃)CH₂CH₂CF₃ |
| 381. | O | C(═O)NH—SO₂—N(CF₃)CH₂CH₂CH₃ |
| 382. | O | C(═O)NH—SO₂—NHCH(CH₃)₂ |
| 383. | O | C(═O)NH—SO₂—NHCH(CF₃)₂ |
| 384. | O | C(═O)NH—SO₂—N(CH₃)CH(CH₃)₂ |
| 385. | O | C(═O)NH—SO₂—N(CH₃)CH(CF₃)₂ |
| 386. | O | C(═O)NH—SO₂—N(CF₃)CH(CH₃)₂ |
| 387. | O | C(═O)NH—SO₂—NHCH₂CH₂CH₂CH₃ |
| 388. | O | C(═O)NH—SO₂—N(CH₂CH₂CH₂CH₃)₂ |
| 389. | O | C(═O)NH—SO₂—N(CH₃)CH₂CH₂CH₂CH₃ |
| 390. | O | C(═O)N(CH₃)—SO₂—NH₂ |
| 391. | O | C(═O)N(CH₃)—SO₂—NHCH₃ |
| 392. | O | C(═O)N(CH₃)—SO₂—N(CH₃)₂ |
| 393. | O | C(═O)N(CH₃)—SO₂—NHCF₃ |
| 394. | O | C(═O)N(CH₃)—SO₂—N(CF₃)₂ |
| 395. | O | C(═O)N(CH₃)—SO₂—NHCH₂CH₃ |
| 396. | O | C(═O)N(CH₃)—SO₂—N(CH₂CH₃)₂ |
| 397. | O | C(═O)N(CH₃)—SO₂—NHCH₂CF₃ |
| 398. | O | C(═O)N(CH₃)—SO₂—N(CH₂CF₃)₂ |
| 399. | O | C(═O)N(CH₃)—SO₂—N(CH₃)CH₂CH₃ |
| 400. | O | C(═O)N(CH₃)—SO₂—N(CH₃)CH₂CF₃ |
| 401. | O | C(═O)N(CH₃)—SO₂—N(CF₃)CH₂CH₃ |
| 402. | O | C(═O)N(CH₃)—SO₂—NHCH₂CH₂CH₃ |
| 403. | O | C(═O)N(CH₃)—SO₂—N(CH₂CH₂CH₃)₂ |
| 404. | O | C(═O)N(CH₃)—SO₂—NHCH₂CH₂CF₃ |
| 405. | O | C(═O)N(CH₃)—SO₂—N(CH₂CH₂CF₃)₂ |
| 406. | O | C(═O)N(CH₃)—SO₂—N(CH₃)CH₂CH₂CH₃ |
| 407. | O | C(═O)N(CH₃)—SO₂—N(CH₃)CH₂CH₂CF₃ |
| 408. | O | C(═O)N(CH₃)—SO₂—N(CF₃)CH₂CH₂CH₃ |
| 409. | O | C(═O)N(CH₃)—SO₂—NHCH(CH₃)₂ |
| 410. | O | C(═O)N(CH₃)—SO₂—NHCH(CF₃)₂ |
| 411. | O | C(═O)N(CH₃)—SO₂—N(CH₃)CH(CH₃)₂ |
| 412. | O | C(═O)N(CH₃)—SO₂—N(CH₃)CH(CF₃)₂ |
| 413. | O | C(═O)N(CH₃)—SO₂—N(CF₃)CH(CH₃)₂ |
| 414. | O | C(═O)N(CH₃)—SO₂—NHCH₂CH₂CH₂CH₃ |
| 415. | O | C(═O)N(CH₃)—SO₂—N(CH₂CH₂CH₂CH₃)₂ |
| 416. | O | C(═O)N(CH₃)—SO₂—N(CH₃)CH₂CH₂CH₂CH₃ |
| 417. | O | C(═O)—N═CHOCH₃ |
| 418. | O | C(═O)—N═CHOCH₂CH₃ |
| 419. | O | C(═O)—N═CHOCH₂CH₂CH₃ |
| 420. | O | C(═O)—N═CHOCH(CH₃)₂ |
| 421. | O | C(═O)—N═CHOCF₃ |
| 422. | O | C(═O)—N═CHOCH₂CF₃ |
| 423. | O | C(═O)—N═CHOCH₂CH₂CF₃ |
| 424. | O | C(═O)—N═CHOCH(CF₃)₂ |
| 425. | O | C(═O)—N═CH—CO—OCH₃ |
| 426. | O | C(═O)—N═CH—CO—OCH₂CH₃ |
| 427. | O | C(═O)—N═CH—CO—OCH₂CH₂CH₃ |
| 428. | O | C(═O)—N═CH—CO—OCH(CH₃)₂ |
| 429. | O | C(═O)—N═CH—CO—OCF₃ |
| 430. | O | C(═O)—N═CH—CO—OCH₂CF₃ |
| 431. | O | C(═O)—N═CH—CO—OCH₂CH₂CF₃ |
| 432. | O | C(═O)—N═CH—CO—OCH(CF₃)₂ |
| 433. | O | C(═O)—N═CH—CO—NHCH₃ |
| 434. | O | C(═O)—N═CH—CO—N(CH₃)₂ |
| 435. | O | C(═O)—N═CH—CO—NHCH₂CH₃ |
| 436. | O | C(═O)—N═CH—CO—N(CH₂CH₃)₂ |
| 437. | O | C(═O)—N═CH—CO—N(CH₃)CH₂CH₃ |
| 438. | O | C(═O)—N═CH—CO—NHCH₂CH₂CH₃ |
| 439. | O | C(═O)—N═CH—CO—N(CH₂CH₂CH₃)₂ |
| 440. | O | C(═O)—N═CH—CO—N(CH₃)CH₂CH₂CH₃ |
| 441. | O | C(═O)—N═CH—CO—NHCH(CH₃)₂ |
| 442. | O | C(═O)—N═CH—CO—N(CH₃)CH(CH₃)₂ |
| 443. | O | C(═O)—N═CH—CO—NHCF₃ |
| 444. | O | C(═O)—N═CH—CO—N(CF₃)₂ |
| 445. | O | C(═O)—N═CH—CO—NHCH₂CF₃ |
| 446. | O | C(═O)—N═CH—CO—N(CH₂CF₃)₂ |
| 447. | O | C(═O)—N═CH—CO—N(CH₃)CH₂CF₃ |
| 448. | O | C(═O)—N═CH—CO—N(CF₃)CH₂CF₃ |
| 449. | O | C(═O)—N═CH—CO—NHCH₂CH₂CF₃ |
| 450. | O | C(═O)—N═CH—CO—N(CH₂CH₂CF₃)₂ |
| 451. | O | C(═O)—N═CH—CO—N(CH₃)CH₂CH₂CF₃ |
| 452. | O | C(═O)—N═CH—CO—N(CF₃)CH₂CH₂CH₃ |
| 453. | O | C(═O)—N═CH—CO—NHCH(CF₃)₂ |
| 454. | O | C(═O)—N═CH—CO—N(CH₃)CH(CF₃)₂ |
| 455. | O | C(═O)—N═CH—CO—N(CF₃)CH(CH₃)₂ |
| 456. | O | C(═NH)NH₂ |
| 457. | O | C(═NH)NHCH₃ |
| 458. | O | C(═NH)N(CH₃)₂ |
| 459. | O | C(═NH)NHCF₃ |
| 460. | O | C(═NH)N(CF₃)₂ |
| 461. | O | C(═NH)NHCH₂CH₃ |
| 462. | O | C(═NH)N(CH₂CH₃)₂ |
| 463. | O | C(═NH)NHCH₂CF₃ |
| 464. | O | C(═NH)N(CH₂CF₃)₂ |
| 465. | O | C(═NH)NHCH₂CH₂CH₃ |
| 466. | O | C(═NH)NHCH(CH₃)₂ |
| 467. | O | C(═NH)NH(CH₂)₃CH₃ |
| 468. | O | C(═NH)NH(CH₂)₄CH₃ |
| 469. | O | C(═NH)NH(CH₂)₅CH₃ |
| 470. | O | C(═NH)NHCH₂-^{c}propyl |
| 471. | O | C(═NH)NHCH₂—C₆H₅ |
| 472. | O | C(═NCH₃)NH₂ |
| 473. | O | C(═NCH₃)NHCH₃ |
| 474. | O | C(═NCH₃)N(CH₃)₂ |
| 475. | O | C(═NCH₃)NHCF₃ |
| 476. | O | C(═NCH₃)N(CF₃)₂ |
| 477. | O | C(═NCH₃)NHCH₂CH₃ |
| 478. | O | C(═NCH₃)N(CH₂CH₃)₂ |
| 479. | O | C(═NCH₃)NHCH₂CF₃ |
| 480. | O | C(═NCH₃)N(CH₂CF₃)₂ |
| 481. | O | C(═NCH₃)NHCH₂CH₂CH₃ |
| 482. | O | C(═NCH₃)NHCH(CH₃)₂ |
| 483. | O | C(═NCH₃)NH(CH₂)₃CH₃ |
| 484. | O | C(═NCH₃)NH(CH₂)₄CH₃ |
| 485. | O | C(═NCH₃)NH(CH₂)₅CH₃ |
| 486. | O | C(═NCH₃)NHCH₂-^{c}propyl |
| 487. | O | C(═NCH₃)NHCH₂—C₆H₅ |
| 488. | O | CH₂C₆H₅ |
| 489. | O | CH₂CH₂C₆H₅ |
| 490. | O | CH₂-2-F—C₆H₄ |
| 491. | O | CH₂-3-F—C₆H₄ |
| 492. | O | CH₂-4-F—C₆H₄ |
| 493. | O | CH₂-2-Cl—C₆H₄ |
| 494. | O | CH₂-3-Cl—C₆H₄ |
| 495. | O | CH₂-4-Cl—C₆H₄ |
| 496. | O | CH₂-2-Br—C₆H₄ |
| 497. | O | CH₂-3-Br—C₆H₄ |
| 498. | O | CH₂-4-Br—C₆H₄ |
| 499. | O | CH₂-2-MeO—C₆H₄ |
| 500. | O | CH₂-3-MeO—C₆H₄ |
| 501. | O | CH₂-4-MeO—C₆H₄ |
| 502. | O | CH₂-2-F—C₆H₄ |
| 503. | O | CH₂-3-F—C₆H₄ |
| 504. | O | CH₂-4-F—C₆H₄ |
| 505. | O | A-1 |
| 506. | O | A-2 |
| 507. | O | A-3 |
| 508. | O | A-4 |
| 509. | O | A-5 |
| 510. | O | A-6 |
| 511. | O | A-7 |
| 512. | O | A-8 |
| 513. | O | A-9 |
| 514. | O | A-10 |
| 515. | O | A-11 |
| 516. | O | A-12 |
| 517. | O | A-13 |
| 518. | O | A-14 |
| 519. | O | A-15 |
| 520. | O | A-16 |
| 521. | O | A-17 |
| 522. | O | A-18 |
| 523. | O | A-19 |
| 524. | O | A-20 |
| 525. | O | A-21 |
| 526. | O | A-22 |
| 527. | O | A-23 |
| 528. | O | A-24 |
| 529. | O | A-25 |
| 530. | O | A-26 |
| 531. | O | A-27 |
| 532. | O | A-28 |
| 533. | O | A-29 |
| 534. | O | A-30 |
| 535. | O | A-31 |
| 536. | O | A-32 |
| 537. | O | A-33 |
| 538. | O | CH₂-A-1 |
| 539. | O | CH₂-A-2 |
| 540. | O | CH₂-A-3 |
| 541. | O | CH₂-A-4 |
| 542. | O | CH₂-A-5 |
| 543. | O | CH₂-A-6 |
| 544. | O | CH₂-A-7 |
| 545. | O | CH₂-A-8 |
| 546. | O | CH₂-A-9 |
| 547. | O | CH₂-A-10 |
| 548. | O | CH₂-A-11 |
| 549. | O | CH₂-A-12 |
| 550. | O | CH₂-A-13 |
| 551. | O | CH₂-A-14 |
| 552. | O | CH₂-A-15 |
| 553. | O | CH₂-A-16 |
| 554. | O | CH₂-A-17 |
| 555. | O | CH₂-A-18 |
| 556. | O | CH₂-A-19 |
| 557. | O | CH₂-A-20 |
| 558. | O | CH₂-A-21 |
| 559. | O | CH₂-A-22 |
| 560. | O | CH₂-A-23 |
| 561. | O | CH₂-A-24 |
| 562. | O | CH₂-A-25 |
| 563. | O | CH₂-A-26 |
| 564. | O | CH₂-A-27 |
| 565. | O | CH₂-A-28 |
| 566. | O | CH₂-A-29 |
| 567. | O | CH₂-A-30 |
| 568. | O | CH₂-A-31 |
| 569. | O | CH₂-A-32 |
| 570. | O | CH₂-A-33 |
| 571. | O | C(═O)-A-1 |
| 572. | O | C(═O)-A-2 |
| 573. | O | C(═O)-A-3 |
| 574. | O | C(═O)-A-4 |
| 575. | O | C(═O)-A-5 |
| 576. | O | C(═O)-A-6 |
| 577. | O | C(═O)-A-7 |
| 578. | O | C(═O)-A-8 |
| 579. | O | C(═O)-A-9 |
| 580. | O | C(═O)-A-10 |
| 581. | O | C(═O)-A-11 |
| 582. | O | C(═O)-A-12 |
| 583. | O | C(═O)-A-13 |
| 584. | O | C(═O)-A-14 |
| 585. | O | C(═O)-A-15 |
| 586. | O | C(═O)-A-16 |
| 587. | O | C(═O)-A-17 |
| 588. | O | C(═O)-A-18 |
| 589. | O | C(═O)-A-19 |
| 590. | O | C(═O)-A-20 |
| 591. | O | C(═O)-A-21 |
| 592. | O | C(═O)-A-22 |
| 593. | O | C(═O)-A-23 |
| 594. | O | C(═O)-A-24 |
| 595. | O | C(═O)-A-25 |
| 596. | O | C(═O)-A-26 |
| 597. | O | C(═O)-A-27 |
| 598. | O | C(═O)-A-28 |
| 599. | O | C(═O)-A-29 |
| 600. | O | C(═O)-A-30 |
| 601. | O | C(═O)-A-31 |
| 602. | O | C(═O)-A-32 |
| 603. | O | C(═O)-A-33 |
| 604. | NH | C(═O)NH₂ |
| 605. | NH | C(═O)NHCH₃ |
| 606. | NH | C(═O)N(CH₃)₂ |
| 607. | NH | C(═O)NHCF₃ |
| 608. | NH | C(═O)N(CF₃)₂ |
| 609. | NH | C(═O)NHCH₂CH₃ |
| 610. | NH | C(═O)N(CH₂CH₃)₂ |
| 611. | NH | C(═O)N(CH₃)CH₂CH₃ |
| 612. | NH | C(═O)NHCH₂CF₃ |
| 613. | NH | C(═O)N(CH₂CF₃)₂ |
| 614. | NH | C(═O)N(CH₃)CH₂CF₃ |
| 615. | NH | C(═O)NHCH₂CH₂CH₃ |
| 616. | NH | C(═O)N(CH₃)CH₂CH₂CH₃ |
| 617. | NH | C(═O)NHCH(CH₃)₂ |
| 618. | NH | C(═O)NH(CH₂)₃CH₃ |
| 619. | NH | C(═O)N(CH₃)—(CH₂)₃CH₃ |
| 620. | NH | C(═O)N[(CH₂)₃CH₃]₂ |
| 621. | NH | C(═O)N(CH₃)—CH₂—C₆H₅ |
| 622. | NH | C(═O)NH-propargyl |
| 623. | NH | C(═O)N(CH₃)-propargyl |
| 624. | NH | C(═O)NH—CH₂-4-Cl—C₆H₄ |
| 625. | NH | C(═O)N(CH₃)—CH₂-4-Cl—C₆H₄ |
| 626. | NH | C(═O)morpholin-4-yl |
| 627. | NH | C(═O)NH-3-thiolyl-1,1-dioxid |
| 628. | NH | C(═O)N(CH₃)-3-thiolyl-1,1-dioxid |
| 629. | NH | C(═O)-azirid-1-yl |
| 630. | NH | C(═O)-pyrrolidin-1-yl |
| 631. | NH | C(═O)-piperidin-1-yl |
| 632. | NH | C(═O)-thiomorpholin-4-yl |
| 633. | NH | C(═O)NH—CH₂CHF₂ |
| 634. | NH | C(═O)NH—CH₂CH₂CHF₂ |
| 635. | NH | C(═O)NH—CH₂CH₂CF₃ |
| 636. | NH | C(═O)NH-cyclopropyl |
| 637. | NH | C(═O)NH-cyclobutyl |
| 638. | NH | C(═O)NH-cyclopentyl |
| 639. | NH | C(═O)NH-cyclohexyl |
| 640. | NH | C(═O)NH—CH₂-cyclopropyl |
| 641. | NH | C(═O)NH—CH₂-cyclobutyl |
| 642. | NH | C(═O)NH—CH₂-cyclopentyl |
| 643. | NH | C(═O)NH—CH₂-cyclohexyl |
| 644. | NH | C(═O)NH—CN |
| 645. | NH | C(═O)NH—CH₂—CN |
| 646. | NH | C(═O)NH—CH₂—CH═CH₂ |
| 647. | NH | C(═O)NH—CH₂—CH═C(Cl)₂ |
| 648. | NH | C(═O)NH—CH₂—CH═CH-phenyl |
| 649. | NH | C(═O)NH—CH₂—CH═CH-(4-Cl-phenyl) |
| 650. | NH | C(═O)NH—CH₂—SCH₃ |
| 651. | NH | C(═O)NH—CH₂—SCF₃ |
| 652. | NH | C(═O)NH—CH₂—CH₂—SCH₃ |
| 653. | NH | C(═O)NH—CH₂—CH₂—SCF₃ |
| 654. | NH | C(═O)NH—CH₂—SO₂—CH₃ |
| 655. | NH | C(═O)NH—CH₂—SO₂—CF₃ |
| 656. | NH | C(═O)NH—CH₂—CH₂—SO₂—CH₃ |
| 657. | NH | C(═O)NH—CH₂—CH₂—SO₂—CF₃ |
| 658. | NH | C(═O)NH—CH₂—CO—NH₂ |
| 659. | NH | C(═O)NH—CH₂—CO—NHCH₃ |
| 660. | NH | C(═O)NH—CH₂—CO—N(CH₃)₂ |
| 661. | NH | C(═O)NH—CH₂—CO—NHCF₃ |
| 662. | NH | C(═O)NH—CH₂—CO—N(CF₃)₂ |
| 663. | NH | C(═O)NH—CH₂—CO—NHCH₂CH₃ |
| 664. | NH | C(═O)NH—CH₂—CO—N(CH₂CH₃)₂ |
| 665. | NH | C(═O)NH—CH₂—CO—NHCH₂CF₃ |
| 666. | NH | C(═O)NH—CH₂—CO—N(CH₂CF₃)₂ |
| 667. | NH | C(═O)NH—CH₂—CO—NHCH₂CH₂CH₃ |
| 668. | NH | C(═O)NH—CH₂—CO—N(CH₂CH₂CH₃)₂ |
| 669. | NH | C(═O)NH—CH₂—CO—NHCH₂CH₂CF₃ |
| 670. | NH | C(═O)NH—CH₂—CO—N(CH₂CH₂CF₃)₂ |
| 671. | NH | C(═O)NH—CH₂—CO—NHCH(CH₃)₂ |
| 672. | NH | C(═O)NH—CH₂—CO—NHCH(CF₃)₂ |
| 673. | NH | C(═O)NH—CH₂—CO—NH-cyclopropyl |
| 674. | NH | C(═O)NH—CH₂—CO—NH—CH₂-cyclopropyl |
| 675. | NH | C(═O)NH—CH₂—CO—OH |
| 676. | NH | C(═O)NH—CH₂—CO—OCH₃ |
| 677. | NH | C(═O)NH—CH₂—CO—OCF₃ |
| 678. | NH | C(═O)NH—CH₂—CO—OCH₂CH₃ |
| 679. | NH | C(═O)NH—CH₂—CO—OCH₂CF₃ |
| 680. | NH | C(═O)NH—CH₂—CO—OCH₂CH₂CH₃ |
| 681. | NH | C(═O)NH—CH₂—CO—OCH(CH₃)₂ |
| 682. | NH | C(═O)NH—CH₂—CO—OCH₂CH₂CH₂CH₃ |
| 683. | NH | C(═O)NH—CH₂—CO—OCH(CH₃)CH₂CH₃ |
| 684. | NH | C(═O)NH—CH₂—CO—OCH₂CH(CH₃)₂ |
| 685. | NH | C(═O)NH—CH₂—CO—OC(CH₃)₃ |
| 686. | NH | C(═O)NH-A-1 |
| 687. | NH | C(═O)NH-A-2 |
| 688. | NH | C(═O)NH-A-3 |
| 689. | NH | C(═O)NH-A-4 |
| 690. | NH | C(═O)NH-A-5 |
| 691. | NH | C(═O)NH-A-6 |
| 692. | NH | C(═O)NH-A-7 |
| 693. | NH | C(═O)NH-A-8 |
| 694. | NH | C(═O)NH-A-9 |
| 695. | NH | C(═O)NH-A-10 |
| 696. | NH | C(═O)NH-A-11 |
| 697. | NH | C(═O)NH-A-12 |
| 698. | NH | C(═O)NH-A-13 |
| 699. | NH | C(═O)NH-A-14 |
| 700. | NH | C(═O)NH-A-15 |
| 701. | NH | C(═O)NH-A-16 |
| 702. | NH | C(═O)NH-A-17 |
| 703. | NH | C(═O)NH-A-18 |
| 704. | NH | C(═O)NH-A-19 |
| 705. | NH | C(═O)NH-A-20 |
| 706. | NH | C(═O)NH-A-21 |
| 707. | NH | C(═O)NH-A-22 |
| 708. | NH | C(═O)NH-A-23 |
| 709. | NH | C(═O)NH-A-24 |
| 710. | NH | C(═O)NH-A-25 |
| 711. | NH | C(═O)NH-A-26 |
| 712. | NH | C(═O)NH-A-27 |
| 713. | NH | C(═O)NH-A-28 |
| 714. | NH | C(═O)NH-A-29 |
| 715. | NH | C(═O)NH-A-30 |
| 716. | NH | C(═O)NH-A-31 |
| 717. | NH | C(═O)NH-A-32 |
| 718. | NH | C(═O)NH-A-33 |
| 719. | NH | C(═O)NH—CH₂-A-1 |
| 720. | NH | C(═O)NH—CH₂-A-2 |
| 721. | NH | C(═O)NH—CH₂-A-3 |
| 722. | NH | C(═O)NH—CH₂-A-4 |
| 723. | NH | C(═O)NH—CH₂-A-5 |
| 724. | NH | C(═O)NH—CH₂-A-6 |
| 725. | NH | C(═O)NH—CH₂-A-7 |
| 726. | NH | C(═O)NH—CH₂-A-8 |
| 727. | NH | C(═O)NH—CH₂-A-9 |
| 728. | NH | C(═O)NH—CH₂-A-10 |
| 729. | NH | C(═O)NH—CH₂-A-11 |
| 730. | NH | C(═O)NH—CH₂-A-12 |
| 731. | NH | C(═O)NH—CH₂-A-13 |
| 732. | NH | C(═O)NH—CH₂-A-14 |
| 733. | NH | C(═O)NH—CH₂-A-15 |
| 734. | NH | C(═O)NH—CH₂-A-16 |
| 735. | NH | C(═O)NH—CH₂-A-17 |
| 736. | NH | C(═O)NH—CH₂-A-18 |
| 737. | NH | C(═O)NH—CH₂-A-19 |
| 738. | NH | C(═O)NH—CH₂-A-20 |
| 739. | NH | C(═O)NH—CH₂-A-21 |
| 740. | NH | C(═O)NH—CH₂-A-22 |
| 741. | NH | C(═O)NH—CH₂-A-23 |
| 742. | NH | C(═O)NH—CH₂-A-24 |
| 743. | NH | C(═O)NH—CH₂-A-25 |
| 744. | NH | C(═O)NH—CH₂-A-26 |
| 745. | NH | C(═O)NH—CH₂-A-27 |
| 746. | NH | C(═O)NH—CH₂-A-28 |
| 747. | NH | C(═O)NH—CH₂-A-29 |
| 748. | NH | C(═O)NH—CH₂-A-30 |
| 749. | NH | C(═O)NH—CH₂-A-31 |
| 750. | NH | C(═O)NH—CH₂-A-32 |
| 751. | NH | C(═O)NH—CH₂-A-33 |
| 752. | NH | C(═O)NH—SO₂—CH₃ |
| 753. | NH | C(═O)NH—SO₂—CF₃ |
| 754. | NH | C(═O)NH—SO₂—CH₂CH₃ |
| 755. | NH | C(═O)NH—SO₂—CH₂CF₃ |
| 756. | NH | C(═O)NH—SO₂—CH₂CH₂CH₃ |
| 757. | NH | C(═O)NH—SO₂—CH₂CH₂CF₃ |
| 758. | NH | C(═O)NH—SO₂—CH₂CF₂CF₃ |
| 759. | NH | C(═O)NH—SO₂—CH(CH₃)₂ |
| 760. | NH | C(═O)NH—SO₂—CH(CF₃)₂ |
| 761. | NH | C(═O)N(CH₃)—CH₂CHF₂ |
| 762. | NH | C(═O)N(CH₃)—CH₂CH₂CHF₂ |
| 763. | NH | C(═O)N(CH₃)—CH₂CH₂CF₃ |
| 764. | NH | C(═O)N(CH₃)-cyclopropyl |
| 765. | NH | C(═O)N(CH₃)-cyclobutyl |
| 766. | NH | C(═O)N(CH₃)-cyclopentyl |
| 767. | NH | C(═O)N(CH₃)-cyclohexyl |
| 768. | NH | C(═O)N(CH₃)—CH₂-cyclopropyl |
| 769. | NH | C(═O)N(CH₃)—CH₂-cyclobutyl |
| 770. | NH | C(═O)N(CH₃)—CH₂-cyclopentyl |
| 771. | NH | C(═O)N(CH₃)—CH₂-cyclohexyl |
| 772. | NH | C(═O)N(CH₃)—CN |
| 773. | NH | C(═O)N(CH₃)—CH₂—CN |
| 774. | NH | C(═O)N(CH₃)—CH₂—CH═CH₂ |
| 775. | NH | C(═O)N(CH₃)—CH₂—CH═C(Cl)₂ |
| 776. | NH | C(═O)N(CH₃)—CH₂—CH═CH-phenyl |
| 777. | NH | C(═O)N(CH₃)—CH₂—CH═CH-(4-Cl-phenyl) |
| 778. | NH | C(═O)N(CH₃)—CH₂—SCH₃ |
| 779. | NH | C(═O)N(CH₃)—CH₂—SCF₃ |
| 780. | NH | C(═O)N(CH₃)—CH₂—CH₂—SCH₃ |
| 781. | NH | C(═O)N(CH₃)—CH₂—CH₂—SCF₃ |
| 782. | NH | C(═O)N(CH₃)—CH₂—SO₂—CH₃ |
| 783. | NH | C(═O)N(CH₃)—CH₂—SO₂—CF₃ |
| 784. | NH | C(═O)N(CH₃)—CH₂—CH₂—SO₂—CH₃ |
| 785. | NH | C(═O)N(CH₃)—CH₂—CH₂—SO₂—CF₃ |
| 786. | NH | C(═O)N(CH₃)—CH₂—CO—NH₂ |
| 787. | NH | C(═O)N(CH₃)—CH₂—CO—NHCH₃ |
| 788. | NH | C(═O)N(CH₃)—CH₂—CO—N(CH₃)₂ |
| 789. | NH | C(═O)N(CH₃)—CH₂—CO—NHCF₃ |
| 790. | NH | C(═O)N(CH₃)—CH₂—CO—N(CF₃)₂ |
| 791. | NH | C(═O)N(CH₃)—CH₂—CO—NHCH₂CH₃ |
| 792. | NH | C(═O)N(CH₃)—CH₂—CO—N(CH₂CH₃)₂ |
| 793. | NH | C(═O)N(CH₃)—CH₂—CO—NHCH₂CF₃ |
| 794. | NH | C(═O)N(CH₃)—CH₂—CO—N(CH₂CF₃)₂ |
| 795. | NH | C(═O)N(CH₃)—CH₂—CO—NHCH₂CH₂CH₃ |
| 796. | NH | C(═O)N(CH₃)—CH₂—CO—N(CH₂CH₂CH₃)₂ |
| 797. | NH | C(═O)N(CH₃)—CH₂—CO—NHCH₂CH₂CF₃ |
| 798. | NH | C(═O)N(CH₃)—CH₂—CO—N(CH₂CH₂CF₃)₂ |
| 799. | NH | C(═O)N(CH₃)—CH₂—CO—NHCH(CH₃)₂ |
| 800. | NH | C(═O)N(CH₃)—CH₂—CO—NHCH(CF₃)₂ |
| 801. | NH | C(═O)N(CH₃)—CH₂—CO—NH-cyclopropyl |
| 802. | NH | C(═O)N(CH₃)—CH₂—CO—NH—CH₂- |
| | | cyclopropyl |
| 803. | NH | C(═O)N(CH₃)—CH₂—CO—OH |
| 804. | NH | C(═O)N(CH₃)—CH₂—CO—OCH₃ |
| 805. | NH | C(═O)N(CH₃)—CH₂—CO—OCF₃ |
| 806. | NH | C(═O)N(CH₃)—CH₂—CO—OCH₂CH₃ |
| 807. | NH | C(═O)N(CH₃)—CH₂—CO—OCH₂CF₃ |
| 808. | NH | C(═O)N(CH₃)—CH₂—CO—OCH₂CH₂CH₃ |
| 809. | NH | C(═O)N(CH₃)—CH₂—CO—OCH(CH₃)₂ |
| 810. | NH | C(═O)N(CH₃)—CH₂—CO—OCH₂CH₂CH₂CH₃ |
| 811. | NH | C(═O)N(CH₃)—CH₂—CO—OCH(CH₃)CH₂CH₃ |
| 812. | NH | C(═O)N(CH₃)—CH₂—CO—OCH₂CH(CH₃)₂ |
| 813. | NH | C(═O)N(CH₃)—CH₂—CO—OC(CH₃)₃ |
| 814. | NH | C(═O)N(CH₃)-A-1 |
| 815. | NH | C(═O)N(CH₃)-A-2 |
| 816. | NH | C(═O)N(CH₃)-A-3 |
| 817. | NH | C(═O)N(CH₃)-A-4 |
| 818. | NH | C(═O)N(CH₃)-A-5 |
| 819. | NH | C(═O)N(CH₃)-A-6 |
| 820. | NH | C(═O)N(CH₃)-A-7 |
| 821. | NH | C(═O)N(CH₃)-A-8 |
| 822. | NH | C(═O)N(CH₃)-A-9 |
| 823. | NH | C(═O)N(CH₃)-A-10 |
| 824. | NH | C(═O)N(CH₃)-A-11 |
| 825. | NH | C(═O)N(CH₃)-A-12 |
| 826. | NH | C(═O)N(CH₃)-A-13 |
| 827. | NH | C(═O)N(CH₃)-A-14 |
| 828. | NH | C(═O)N(CH₃)-A-15 |
| 829. | NH | C(═O)N(CH₃)-A-16 |
| 830. | NH | C(═O)N(CH₃)-A-17 |
| 831. | NH | C(═O)N(CH₃)-A-18 |
| 832. | NH | C(═O)N(CH₃)-A-19 |
| 833. | NH | C(═O)N(CH₃)-A-20 |
| 834. | NH | C(═O)N(CH₃)-A-21 |
| 835. | NH | C(═O)N(CH₃)-A-22 |
| 836. | NH | C(═O)N(CH₃)-A-23 |
| 837. | NH | C(═O)N(CH₃)-A-24 |
| 838. | NH | C(═O)N(CH₃)-A-25 |
| 839. | NH | C(═O)N(CH₃)-A-26 |
| 840. | NH | C(═O)N(CH₃)-A-27 |
| 841. | NH | C(═O)N(CH₃)-A-28 |
| 842. | NH | C(═O)N(CH₃)-A-29 |
| 843. | NH | C(═O)N(CH₃)-A-30 |
| 844. | NH | C(═O)N(CH₃)-A-31 |
| 845. | NH | C(═O)N(CH₃)-A-32 |
| 846. | NH | C(═O)N(CH₃)-A-33 |
| 847. | NH | C(═O)N(CH₃)—CH₂-A-1 |
| 848. | NH | C(═O)N(CH₃)—CH₂-A-2 |
| 849. | NH | C(═O)N(CH₃)—CH₂-A-3 |
| 850. | NH | C(═O)N(CH₃)—CH₂-A-4 |
| 851. | NH | C(═O)N(CH₃)—CH₂-A-5 |
| 852. | NH | C(═O)N(CH₃)—CH₂-A-6 |
| 853. | NH | C(═O)N(CH₃)—CH₂-A-7 |
| 854. | NH | C(═O)N(CH₃)—CH₂-A-8 |
| 855. | NH | C(═O)N(CH₃)—CH₂-A-9 |
| 856. | NH | C(═O)N(CH₃)—CH₂-A-10 |
| 857. | NH | C(═O)N(CH₃)—CH₂-A-11 |
| 858. | NH | C(═O)N(CH₃)—CH₂-A-12 |
| 859. | NH | C(═O)N(CH₃)—CH₂-A-13 |
| 860. | NH | C(═O)N(CH₃)—CH₂-A-14 |
| 861. | NH | C(═O)N(CH₃)—CH₂-A-15 |
| 862. | NH | C(═O)N(CH₃)—CH₂-A-16 |
| 863. | NH | C(═O)N(CH₃)—CH₂-A-17 |
| 864. | NH | C(═O)N(CH₃)—CH₂-A-18 |
| 865. | NH | C(═O)N(CH₃)—CH₂-A-19 |
| 866. | NH | C(═O)N(CH₃)—CH₂-A-20 |
| 867. | NH | C(═O)N(CH₃)—CH₂-A-21 |
| 868. | NH | C(═O)N(CH₃)—CH₂-A-22 |
| 869. | NH | C(═O)N(CH₃)—CH₂-A-23 |
| 870. | NH | C(═O)N(CH₃)—CH₂-A-24 |
| 871. | NH | C(═O)N(CH₃)—CH₂-A-25 |
| 872. | NH | C(═O)N(CH₃)—CH₂-A-26 |
| 873. | NH | C(═O)N(CH₃)—CH₂-A-27 |
| 874. | NH | C(═O)N(CH₃)—CH₂-A-28 |
| 875. | NH | C(═O)N(CH₃)—CH₂-A-29 |
| 876. | NH | C(═O)N(CH₃)—CH₂-A-30 |
| 877. | NH | C(═O)N(CH₃)—CH₂-A-31 |
| 878. | NH | C(═O)N(CH₃)—CH₂-A-32 |
| 879. | NH | C(═O)N(CH₃)—CH₂-A-33 |
| 880. | NH | C(═O)N(CH₃)—SO₂—CH₃ |
| 881. | NH | C(═O)N(CH₃)—SO₂—CF₃ |
| 882. | NH | C(═O)N(CH₃)—SO₂—CH₂CH₃ |
| 883. | NH | C(═O)N(CH₃)—SO₂—CH₂CF₃ |
| 884. | NH | C(═O)N(CH₃)—SO₂—CH₂CH₂CH₃ |
| 885. | NH | C(═O)N(CH₃)—SO₂—CH₂CH₂CF₃ |
| 886. | NH | C(═O)N(CH₃)—SO₂—CH₂CF₂CF₃ |
| 887. | NH | C(═O)N(CH₃)—SO₂—CH(CH₃)₂ |
| 888. | NH | C(═O)N(CH₃)—SO₂—CH(CF₃)₂ |
| 889. | NH | C(═O)NH—SO₂—NH₂ |
| 890. | NH | C(═O)NH—SO₂—NHCH₃ |
| 891. | NH | C(═O)NH—SO₂—N(CH₃)₂ |
| 892. | NH | C(═O)NH—SO₂—NHCF₃ |
| 893. | NH | C(═O)NH—SO₂—N(CF₃)₂ |
| 894. | NH | C(═O)NH—SO₂—NHCH₂CH₃ |
| 895. | NH | C(═O)NH—SO₂—N(CH₂CH₃)₂ |
| 896. | NH | C(═O)NH—SO₂—NHCH₂CF₃ |
| 897. | NH | C(═O)NH—SO₂—N(CH₂CF₃)₂ |
| 898. | NH | C(═O)NH—SO₂—N(CH₃)CH₂CH₃ |
| 899. | NH | C(═O)NH—SO₂—N(CH₃)CH₂CF₃ |
| 900. | NH | C(═O)NH—SO₂—N(CF₃)CH₂CH₃ |
| 901. | NH | C(═O)NH—SO₂—NHCH₂CH₂CH₃ |
| 902. | NH | C(═O)NH—SO₂—N(CH₂CH₂CH₃)₂ |
| 903. | NH | C(═O)NH—SO₂—NHCH₂CH₂CF₃ |
| 904. | NH | C(═O)NH—SO₂—N(CH₂CH₂CF₃)₂ |
| 905. | NH | C(═O)NH—SO₂—N(CH₃)CH₂CH₂CH₃ |
| 906. | NH | C(═O)NH—SO₂—N(CH₃)CH₂CH₂CF₃ |
| 907. | NH | C(═O)NH—SO₂—N(CF₃)CH₂CH₂CH₃ |
| 908. | NH | C(═O)NH—SO₂—NHCH(CH₃)₂ |
| 909. | NH | C(═O)NH—SO₂—NHCH(CF₃)₂ |
| 910. | NH | C(═O)NH—SO₂—N(CH₃)CH(CH₃)₂ |
| 911. | NH | C(═O)NH—SO₂—N(CH₃)CH(CF₃)₂ |
| 912. | NH | C(═O)NH—SO₂—N(CF₃)CH(CH₃)₂ |
| 913. | NH | C(═O)NH—SO₂—NHCH₂CH₂CH₂CH₃ |
| 914. | NH | C(═O)NH—SO₂—N(CH₂CH₂CH₂CH₃)₂ |
| 915. | NH | C(═O)NH—SO₂—N(CH₃)CH₂CH₂CH₂CH₃ |
| 916. | NH | C(═O)N(CH₃)—SO₂—NH₂ |
| 917. | NH | C(═O)N(CH₃)—SO₂—NHCH₃ |
| 918. | NH | C(═O)N(CH₃)—SO₂—N(CH₃)₂ |
| 919. | NH | C(═O)N(CH₃)—SO₂—NHCF₃ |
| 920. | NH | C(═O)N(CH₃)—SO₂—N(CF₃)₂ |
| 921. | NH | C(═O)N(CH₃)—SO₂—NHCH₂CH₃ |
| 922. | NH | C(═O)N(CH₃)—SO₂—N(CH₂CH₃)₂ |
| 923. | NH | C(═O)N(CH₃)—SO₂—NHCH₂CF₃ |
| 924. | NH | C(═O)N(CH₃)—SO₂—N(CH₂CF₃)₂ |
| 925. | NH | C(═O)N(CH₃)—SO₂—N(CH₃)CH₂CH₃ |
| 926. | NH | C(═O)N(CH₃)—SO₂—N(CH₃)CH₂CF₃ |
| 927. | NH | C(═O)N(CH₃)—SO₂—N(CF₃)CH₂CH₃ |
| 928. | NH | C(═O)N(CH₃)—SO₂—NHCH₂CH₂CH₃ |
| 929. | NH | C(═O)N(CH₃)—SO₂—N(CH₂CH₂CH₃)₂ |
| 930. | NH | C(═O)N(CH₃)—SO₂—NHCH₂CH₂CF₃ |
| 931. | NH | C(═O)N(CH₃)—SO₂—N(CH₂CH₂CF₃)₂ |
| 932. | NH | C(═O)N(CH₃)—SO₂—N(CH₃)CH₂CH₂CH₃ |
| 933. | NH | C(═O)N(CH₃)—SO₂—N(CH₃)CH₂CH₂CF₃ |
| 934. | NH | C(═O)N(CH₃)—SO₂—N(CF₃)CH₂CH₂CH₃ |
| 935. | NH | C(═O)N(CH₃)—SO₂—NHCH(CH₃)₂ |
| 936. | NH | C(═O)N(CH₃)—SO₂—NHCH(CF₃)₂ |
| 937. | NH | C(═O)N(CH₃)—SO₂—N(CH₃)CH(CH₃)₂ |
| 938. | NH | C(═O)N(CH₃)—SO₂—N(CH₃)CH(CF₃)₂ |
| 939. | NH | C(═O)N(CH₃)—SO₂—N(CF₃)CH(CH₃)₂ |
| 940. | NH | C(═O)N(CH₃)—SO₂—NHCH₂CH₂CH₂CH₃ |
| 941. | NH | C(═O)N(CH₃)—SO₂—N(CH₂CH₂CH₂CH₃)₂ |
| 942. | NH | C(═O)N(CH₃)—SO₂—N(CH₃)CH₂CH₂CH₂CH₃ |
| 943. | NH | C(═O)—N═CHOCH₃ |
| 944. | NH | C(═O)—N═CHOCH₂CH₃ |
| 945. | NH | C(═O)—N═CHOCH₂CH₂CH₃ |
| 946. | NH | C(═O)—N═CHOCH(CH₃)₂ |
| 947. | NH | C(═O)—N═CHOCF₃ |
| 948. | NH | C(═O)—N═CHOCH₂CF₃ |
| 949. | NH | C(═O)—N═CHOCH₂CH₂CF₃ |
| 950. | NH | C(═O)—N═CHOCH(CF₃)₂ |
| 951. | NH | C(═O)—N═CH—CO—OCH₃ |
| 952. | NH | C(═O)—N═CH—CO—OCH₂CH₃ |
| 953. | NH | C(═O)—N═CH—CO—OCH₂CH₂CH₃ |
| 954. | NH | C(═O)—N═CH—CO—OCH(CH₃)₂ |
| 955. | NH | C(═O)—N═CH—CO—OCF₃ |
| 956. | NH | C(═O)—N═CH—CO—OCH₂CF₃ |
| 957. | NH | C(═O)—N═CH—CO—OCH₂CH₂CF₃ |
| 958. | NH | C(═O)—N═CH—CO—OCH(CF₃)₂ |
| 959. | NH | C(═O)—N═CH—CO—NHCH₃ |
| 960. | NH | C(═O)—N═CH—CO—N(CH₃)₂ |
| 961. | NH | C(═O)—N═CH—CO—NHCH₂CH₃ |
| 962. | NH | C(═O)—N═CH—CO—N(CH₂CH₃)₂ |
| 963. | NH | C(═O)—N═CH—CO—N(CH₃)CH₂CH₃ |
| 964. | NH | C(═O)—N═CH—CO—NHCH₂CH₂CH₃ |
| 965. | NH | C(═O)—N═CH—CO—N(CH₂CH₂CH₃)₂ |
| 966. | NH | C(═O)—N═CH—CO—N(CH₃)CH₂CH₂CH₃ |
| 967. | NH | C(═O)—N═CH—CO—NHCH(CH₃)₂ |
| 968. | NH | C(═O)—N═CH—CO—N(CH₃)CH(CH₃)₂ |
| 969. | NH | C(═O)—N═CH—CO—NHCF₃ |
| 970. | NH | C(═O)—N═CH—CO—N(CF₃)₂ |
| 971. | NH | C(═O)—N═CH—CO—NHCH₂CF₃ |
| 972. | NH | C(═O)—N═CH—CO—N(CH₂CF₃)₂ |
| 973. | NH | C(═O)—N═CH—CO—N(CH₃)CH₂CF₃ |
| 974. | NH | C(═O)—N═CH—CO—N(CF₃)CH₂CF₃ |
| 975. | NH | C(═O)—N═CH—CO—NHCH₂CH₂CF₃ |
| 976. | NH | C(═O)—N═CH—CO—N(CH₂CH₂CF₃)₂ |
| 977. | NH | C(═O)—N═CH—CO—N(CH₃)CH₂CH₂CF₃ |
| 978. | NH | C(═O)—N═CH—CO—N(CF₃)CH₂CH₂CH₃ |
| 979. | NH | C(═O)—N═CH—CO—NHCH(CF₃)₂ |
| 980. | NH | C(═O)—N═CH—CO—N(CH₃)CH(CF₃)₂ |
| 981. | NH | C(═O)—N═CH—CO—N(CF₃)CH(CH₃)₂ |
| 982. | NH | C(═S)NH₂ |
| 983. | NH | C(═S)NHCH₃ |
| 984. | NH | C(═S)N(CH₃)₂ |
| 985. | NH | C(═S)NHCF₃ |
| 986. | NH | C(═S)N(CF₃)₂ |
| 987. | NH | C(═S)NHCH₂CH₃ |
| 988. | NH | C(═S)N(CH₂CH₃)₂ |
| 989. | NH | C(═S)N(CH₃)CH₂CH₃ |
| 990. | NH | C(═S)NHCH₂CF₃ |
| 991. | NH | C(═S)N(CH₂CF₃)₂ |
| 992. | NH | C(═S)N(CH₃)CH₂CF₃ |
| 993. | NH | C(═S)NHCH₂CH₂CH₃ |
| 994. | NH | C(═S)N(CH₃)CH₂CH₂CH₃ |
| 995. | NH | C(═S)NHCH(CH₃)₂ |
| 996. | NH | C(═S)NH(CH₂)₃CH₃ |
| 997. | NH | C(═S)N(CH₃)—(CH₂)₃CH₃ |
| 998. | NH | C(═S)N[(CH₂)₃CH₃]₂ |
| 999. | NH | C(═S)N(CH₃)—CH₂—C₆H₅ |
| 1000. | NH | C(═S)NH-propargyl |
| 1001. | NH | C(═S)N(CH₃)-propargyl |
| 1002. | NH | C(═S)NH—CH₂-4-Cl—C₆H₄ |
| 1003. | NH | C(═S)N(CH₃)—CH₂-4-Cl—C₆H₄ |
| 1004. | NH | C(═S)morpholin-4-yl |
| 1005. | NH | C(═S)NH-3-thiolyl-1,1-dioxid |
| 1006. | NH | C(═S)N(CH₃)-3-thiolyl-1,1-dioxid |
| 1007. | NH | C(═S)-azirid-1-yl |
| 1008. | NH | C(═S)-pyrrolidin-1-yl |
| 1009. | NH | C(═S)-piperidin-1-yl |
| 1010. | NH | C(═S)-thiomorpholin-4-yl |
| 1011. | NH | C(═S)NH—CH₂CHF₂ |
| 1012. | NH | C(═S)NH—CH₂CH₂CHF₂ |
| 1013. | NH | C(═S)NH—CH₂CH₂CF₃ |
| 1014. | NH | C(═S)NH-cyclopropyl |
| 1015. | NH | C(═S)NH-cyclobutyl |
| 1016. | NH | C(═S)NH-cyclopentyl |
| 1017. | NH | C(═S)NH-cyclohexyl |
| 1018. | NH | C(═S)NH—CH₂-cyclopropyl |
| 1019. | NH | C(═S)NH—CH₂-cyclobutyl |
| 1020. | NH | C(═S)NH—CH₂-cyclopentyl |
| 1021. | NH | C(═S)NH—CH₂-cyclohexyl |
| 1022. | NH | C(═S)NH—CN |
| 1023. | NH | C(═S)NH—CH₂—CN |
| 1024. | NH | C(═S)NH—CH₂—CH═CH₂ |
| 1025. | NH | C(═S)NH—CH₂—CH═C(Cl)₂ |
| 1026. | NH | C(═S)NH—CH₂—CH═CH-phenyl |
| 1027. | NH | C(═S)NH—CH₂—CH═CH-(4-Cl-phenyl) |
| 1028. | NH | C(═S)NH—CH₂—SCH₃ |
| 1029. | NH | C(═S)NH—CH₂—SCF₃ |
| 1030. | NH | C(═S)NH—CH₂—CH₂—SCH₃ |
| 1031. | NH | C(═S)NH—CH₂—CH₂—SCF₃ |
| 1032. | NH | C(═S)NH—CH₂—SO₂—CH₃ |
| 1033. | NH | C(═S)NH—CH₂—SO₂—CF₃ |
| 1034. | NH | C(═S)NH—CH₂—CH₂—SO₂—CH₃ |
| 1035. | NH | C(═S)NH—CH₂—CH₂—SO₂—CF₃ |
| 1036. | NH | C(═S)NH—CH₂—CO—NH₂ |
| 1037. | NH | C(═S)NH—CH₂—CO—NHCH₃ |
| 1038. | NH | C(═S)NH—CH₂—CO—N(CH₃)₂ |
| 1039. | NH | C(═S)NH—CH₂—CO—NHCF₃ |
| 1040. | NH | C(═S)NH—CH₂—CO—N(CF₃)₂ |
| 1041. | NH | C(═S)NH—CH₂—CO—NHCH₂CH₃ |
| 1042. | NH | C(═S)NH—CH₂—CO—N(CH₂CH₃)₂ |
| 1043. | NH | C(═S)NH—CH₂—CO—NHCH₂CF₃ |
| 1044. | NH | C(═S)NH—CH₂—CO—N(CH₂CF₃)₂ |
| 1045. | NH | C(═S)NH—CH₂—CO—NHCH₂CH₂CH₃ |
| 1046. | NH | C(═S)NH—CH₂—CO—N(CH₂CH₂CH₃)₂ |
| 1047. | NH | C(═S)NH—CH₂—CO—NHCH₂CH₂CF₃ |
| 1048. | NH | C(═S)NH—CH₂—CO—N(CH₂CH₂CF₃)₂ |
| 1049. | NH | C(═S)NH—CH₂—CO—NHCH(CH₃)₂ |
| 1050. | NH | C(═S)NH—CH₂—CO—NHCH(CF₃)₂ |
| 1051. | NH | C(═S)NH—CH₂—CO—NH-cyclopropyl |
| 1052. | NH | C(═S)NH—CH₂—CO—NH—CH₂-cyclopropyl |
| 1053. | NH | C(═S)NH—CH₂—CO—OH |
| 1054. | NH | C(═S)NH—CH₂—CO—OCH₃ |
| 1055. | NH | C(═S)NH—CH₂—CO—OCF₃ |
| 1056. | NH | C(═S)NH—CH₂—CO—OCH₂CH₃ |
| 1057. | NH | C(═S)NH—CH₂—CO—OCH₂CF₃ |
| 1058. | NH | C(═S)NH—CH₂—CO—OCH₂CH₂CH₃ |
| 1059. | NH | C(═S)NH—CH₂—CO—OCH(CH₃)₂ |
| 1060. | NH | C(═S)NH—CH₂—CO—OCH₂CH₂CH₂CH₃ |
| 1061. | NH | C(═S)NH—CH₂—CO—OCH(CH₃)CH₂CH₃ |
| 1062. | NH | C(═S)NH—CH₂—CO—OCH₂CH(CH₃)₂ |
| 1063. | NH | C(═S)NH—CH₂—CO—OC(CH₃)₃ |
| 1064. | NH | C(═S)NH-A-1 |
| 1065. | NH | C(═S)NH-A-2 |
| 1066. | NH | C(═S)NH-A-3 |
| 1067. | NH | C(═S)NH-A-4 |
| 1068. | NH | C(═S)NH-A-5 |
| 1069. | NH | C(═S)NH-A-6 |
| 1070. | NH | C(═S)NH-A-7 |
| 1071. | NH | C(═S)NH-A-8 |
| 1072. | NH | C(═S)NH-A-9 |
| 1073. | NH | C(═S)NH-A-10 |
| 1074. | NH | C(═S)NH-A-11 |
| 1075. | NH | C(═S)NH-A-12 |
| 1076. | NH | C(═S)NH-A-13 |
| 1077. | NH | C(═S)NH-A-14 |
| 1078. | NH | C(═S)NH-A-15 |
| 1079. | NH | C(═S)NH-A-16 |
| 1080. | NH | C(═S)NH-A-17 |
| 1081. | NH | C(═S)NH-A-18 |
| 1082. | NH | C(═S)NH-A-19 |
| 1083. | NH | C(═S)NH-A-20 |
| 1084. | NH | C(═S)NH-A-21 |
| 1085. | NH | C(═S)NH-A-22 |
| 1086. | NH | C(═S)NH-A-23 |
| 1087. | NH | C(═S)NH-A-24 |
| 1088. | NH | C(═S)NH-A-25 |
| 1089. | NH | C(═S)NH-A-26 |
| 1090. | NH | C(═S)NH-A-27 |
| 1091. | NH | C(═S)NH-A-28 |
| 1092. | NH | C(═S)NH-A-29 |
| 1093. | NH | C(═S)NH-A-30 |
| 1094. | NH | C(═S)NH-A-31 |
| 1095. | NH | C(═S)NH-A-32 |
| 1096. | NH | C(═S)NH-A-33 |
| 1097. | NH | C(═S)NH—CH₂-A-1 |
| 1098. | NH | C(═S)NH—CH₂-A-2 |
| 1099. | NH | C(═S)NH—CH₂-A-3 |
| 1100. | NH | C(═S)NH—CH₂-A-4 |
| 1101. | NH | C(═S)NH—CH₂-A-5 |
| 1102. | NH | C(═S)NH—CH₂-A-6 |
| 1103. | NH | C(═S)NH—CH₂-A-7 |
| 1104. | NH | C(═S)NH—CH₂-A-8 |
| 1105. | NH | C(═S)NH—CH₂-A-9 |
| 1106. | NH | C(═S)NH—CH₂-A-10 |
| 1107. | NH | C(═S)NH—CH₂-A-11 |
| 1108. | NH | C(═S)NH—CH₂-A-12 |
| 1109. | NH | C(═S)NH—CH₂-A-13 |
| 1110. | NH | C(═S)NH—CH₂-A-14 |
| 1111. | NH | C(═S)NH—CH₂-A-15 |
| 1112. | NH | C(═S)NH—CH₂-A-16 |
| 1113. | NH | C(═S)NH—CH₂-A-17 |
| 1114. | NH | C(═S)NH—CH₂-A-18 |
| 1115. | NH | C(═S)NH—CH₂-A-19 |
| 1116. | NH | C(═S)NH—CH₂-A-20 |
| 1117. | NH | C(═S)NH—CH₂-A-21 |
| 1118. | NH | C(═S)NH—CH₂-A-22 |
| 1119. | NH | C(═S)NH—CH₂-A-23 |
| 1120. | NH | C(═S)NH—CH₂-A-24 |
| 1121. | NH | C(═S)NH—CH₂-A-25 |
| 1122. | NH | C(═S)NH—CH₂-A-26 |
| 1123. | NH | C(═S)NH—CH₂-A-27 |
| 1124. | NH | C(═S)NH—CH₂-A-28 |
| 1125. | NH | C(═S)NH—CH₂-A-29 |
| 1126. | NH | C(═S)NH—CH₂-A-30 |
| 1127. | NH | C(═S)NH—CH₂-A-31 |
| 1128. | NH | C(═S)NH—CH₂-A-32 |
| 1129. | NH | C(═S)NH—CH₂-A-33 |
| 1130. | NH | C(═S)NH—SO₂—CH₃ |
| 1131. | NH | C(═S)NH—SO₂—CF₃ |
| 1132. | NH | C(═S)NH—SO₂—CH₂CH₃ |
| 1133. | NH | C(═S)NH—SO₂—CH₂CF₃ |
| 1134. | NH | C(═S)NH—SO₂—CH₂CH₂CH₃ |
| 1135. | NH | C(═S)NH—SO₂—CH₂CH₂CF₃ |
| 1136. | NH | C(═S)NH—SO₂—CH₂CF₂CF₃ |
| 1137. | NH | C(═S)NH—SO₂—CH(CH₃)₂ |
| 1138. | NH | C(═S)NH—SO₂—CH(CF₃)₂ |
| 1139. | NH | C(═S)N(CH₃)—CH₂CHF₂ |
| 1140. | NH | C(═S)N(CH₃)—CH₂CH₂CHF₂ |
| 1141. | NH | C(═S)N(CH₃)—CH₂CH₂CF₃ |
| 1142. | NH | C(═S)N(CH₃)-cyclopropyl |
| 1143. | NH | C(═S)N(CH₃)-cyclobutyl |
| 1144. | NH | C(═S)N(CH₃)-cyclopentyl |
| 1145. | NH | C(═S)N(CH₃)-cyclohexyl |
| 1146. | NH | C(═S)N(CH₃)—CH₂-cyclopropyl |
| 1147. | NH | C(═S)N(CH₃)—CH₂-cyclobutyl |
| 1148. | NH | C(═S)N(CH₃)—CH₂-cyclopentyl |
| 1149. | NH | C(═S)N(CH₃)—CH₂-cyclohexyl |
| 1150. | NH | C(═S)N(CH₃)—CN |
| 1151. | NH | C(═S)N(CH₃)—CH₂—CN |
| 1152. | NH | C(═S)N(CH₃)—CH₂—CH═CH₂ |
| 1153. | NH | C(═S)N(CH₃)—CH₂—CH═C(Cl)₂ |
| 1154. | NH | C(═S)N(CH₃)—CH₂—CH═CH-phenyl |
| 1155. | NH | C(═S)N(CH₃)—CH₂—CH═CH-(4-Cl-phenyl) |
| 1156. | NH | C(═S)N(CH₃)—CH₂—SCH₃ |
| 1157. | NH | C(═S)N(CH₃)—CH₂—SCF₃ |
| 1158. | NH | C(═S)N(CH₃)—CH₂—CH₂—SCH₃ |
| 1159. | NH | C(═S)N(CH₃)—CH₂—CH₂—SCF₃ |
| 1160. | NH | C(═S)N(CH₃)—CH₂—SO₂—CH₃ |
| 1161. | NH | C(═S)N(CH₃)—CH₂—SO₂—CF₃ |
| 1162. | NH | C(═S)N(CH₃)—CH₂—CH₂—SO₂—CH₃ |
| 1163. | NH | C(═S)N(CH₃)—CH₂—CH₂—SO₂—CF₃ |
| 1164. | NH | C(═S)N(CH₃)—CH₂—CO—NH₂ |
| 1165. | NH | C(═S)N(CH₃)—CH₂—CO—NHCH₃ |
| 1166. | NH | C(═S)N(CH₃)—CH₂—CO—N(CH₃)₂ |
| 1167. | NH | C(═S)N(CH₃)—CH₂—CO—NHCF₃ |
| 1168. | NH | C(═S)N(CH₃)—CH₂—CO—N(CF₃)₂ |
| 1169. | NH | C(═S)N(CH₃)—CH₂—CO—NHCH₂CH₃ |
| 1170. | NH | C(═S)N(CH₃)—CH₂—CO—N(CH₂CH₃)₂ |
| 1171. | NH | C(═S)N(CH₃)—CH₂—CO—NHCH₂CF₃ |
| 1172. | NH | C(═S)N(CH₃)—CH₂—CO—N(CH₂CF₃)₂ |
| 1173. | NH | C(═S)N(CH₃)—CH₂—CO—NHCH₂CH₂CH₃ |
| 1174. | NH | C(═S)N(CH₃)—CH₂—CO—N(CH₂CH₂CH₃)₂ |
| 1175. | NH | C(═S)N(CH₃)—CH₂—CO—NHCH₂CH₂CF₃ |
| 1176. | NH | C(═S)N(CH₃)—CH₂—CO—N(CH₂CH₂CF₃)₂ |
| 1177. | NH | C(═S)N(CH₃)—CH₂—CO—NHCH(CH₃)₂ |
| 1178. | NH | C(═S)N(CH₃)—CH₂—CO—NHCH(CF₃)₂ |
| 1179. | NH | C(═S)N(CH₃)—CH₂—CO—NH-cyclopropyl |
| 1180. | NH | C(═S)N(CH₃)—CH₂—CO—NH—CH₂- |
| | | cyclopropyl |
| 1181. | NH | C(═S)N(CH₃)—CH₂—CO—OH |
| 1182. | NH | C(═S)N(CH₃)—CH₂—CO—OCH₃ |
| 1183. | NH | C(═S)N(CH₃)—CH₂—CO—OCF₃ |
| 1184. | NH | C(═S)N(CH₃)—CH₂—CO—OCH₂CH₃ |
| 1185. | NH | C(═S)N(CH₃)—CH₂—CO—OCH₂CF₃ |
| 1186. | NH | C(═S)N(CH₃)—CH₂—CO—OCH₂CH₂CH₃ |
| 1187. | NH | C(═S)N(CH₃)—CH₂—CO—OCH(CH₃)₂ |
| 1188. | NH | C(═S)N(CH₃)—CH₂—CO—OCH₂CH₂CH₂CH₃ |
| 1189. | NH | C(═S)N(CH₃)—CH₂—CO—OCH(CH₃)CH₂CH₃ |
| 1190. | NH | C(═S)N(CH₃)—CH₂—CO—OCH₂CH(CH₃)₂ |
| 1191. | NH | C(═S)N(CH₃)—CH₂—CO—OC(CH₃)₃ |
| 1192. | NH | C(═S)N(CH₃)-A-1 |
| 1193. | NH | C(═S)N(CH₃)-A-2 |
| 1194. | NH | C(═S)N(CH₃)-A-3 |
| 1195. | NH | C(═S)N(CH₃)-A-4 |
| 1196. | NH | C(═S)N(CH₃)-A-5 |
| 1197. | NH | C(═S)N(CH₃)-A-6 |
| 1198. | NH | C(═S)N(CH₃)-A-7 |
| 1199. | NH | C(═S)N(CH₃)-A-8 |
| 1200. | NH | C(═S)N(CH₃)-A-9 |
| 1201. | NH | C(═S)N(CH₃)-A-10 |
| 1202. | NH | C(═S)N(CH₃)-A-11 |
| 1203. | NH | C(═S)N(CH₃)-A-12 |
| 1204. | NH | C(═S)N(CH₃)-A-13 |
| 1205. | NH | C(═S)N(CH₃)-A-14 |
| 1206. | NH | C(═S)N(CH₃)-A-15 |
| 1207. | NH | C(═S)N(CH₃)-A-16 |
| 1208. | NH | C(═S)N(CH₃)-A-17 |
| 1209. | NH | C(═S)N(CH₃)-A-18 |
| 1210. | NH | C(═S)N(CH₃)-A-19 |
| 1211. | NH | C(═S)N(CH₃)-A-20 |
| 1212. | NH | C(═S)N(CH₃)-A-21 |
| 1213. | NH | C(═S)N(CH₃)-A-22 |
| 1214. | NH | C(═S)N(CH₃)-A-23 |
| 1215. | NH | C(═S)N(CH₃)-A-24 |
| 1216. | NH | C(═S)N(CH₃)-A-25 |
| 1217. | NH | C(═S)N(CH₃)-A-26 |
| 1218. | NH | C(═S)N(CH₃)-A-27 |
| 1219. | NH | C(═S)N(CH₃)-A-28 |
| 1220. | NH | C(═S)N(CH₃)-A-29 |
| 1221. | NH | C(═S)N(CH₃)-A-30 |
| 1222. | NH | C(═S)N(CH₃)-A-31 |
| 1223. | NH | C(═S)N(CH₃)-A-32 |
| 1224. | NH | C(═S)N(CH₃)-A-33 |
| 1225. | NH | C(═S)N(CH₃)—CH₂-A-1 |
| 1226. | NH | C(═S)N(CH₃)—CH₂-A-2 |
| 1227. | NH | C(═S)N(CH₃)—CH₂-A-3 |
| 1228. | NH | C(═S)N(CH₃)—CH₂-A-4 |
| 1229. | NH | C(═S)N(CH₃)—CH₂-A-5 |
| 1230. | NH | C(═S)N(CH₃)—CH₂-A-6 |
| 1231. | NH | C(═S)N(CH₃)—CH₂-A-7 |
| 1232. | NH | C(═S)N(CH₃)—CH₂-A-8 |
| 1233. | NH | C(═S)N(CH₃)—CH₂-A-9 |
| 1234. | NH | C(═S)N(CH₃)—CH₂-A-10 |
| 1235. | NH | C(═S)N(CH₃)—CH₂-A-11 |
| 1236. | NH | C(═S)N(CH₃)—CH₂-A-12 |
| 1237. | NH | C(═S)N(CH₃)—CH₂-A-13 |
| 1238. | NH | C(═S)N(CH₃)—CH₂-A-14 |
| 1239. | NH | C(═S)N(CH₃)—CH₂-A-15 |
| 1240. | NH | C(═S)N(CH₃)—CH₂-A-16 |
| 1241. | NH | C(═S)N(CH₃)—CH₂-A-17 |
| 1242. | NH | C(═S)N(CH₃)—CH₂-A-18 |
| 1243. | NH | C(═S)N(CH₃)—CH₂-A-19 |
| 1244. | NH | C(═S)N(CH₃)—CH₂-A-20 |
| 1245. | NH | C(═S)N(CH₃)—CH₂-A-21 |
| 1246. | NH | C(═S)N(CH₃)—CH₂-A-22 |
| 1247. | NH | C(═S)N(CH₃)—CH₂-A-23 |
| 1248. | NH | C(═S)N(CH₃)—CH₂-A-24 |
| 1249. | NH | C(═S)N(CH₃)—CH₂-A-25 |
| 1250. | NH | C(═S)N(CH₃)—CH₂-A-26 |
| 1251. | NH | C(═S)N(CH₃)—CH₂-A-27 |
| 1252. | NH | C(═S)N(CH₃)—CH₂-A-28 |
| 1253. | NH | C(═S)N(CH₃)—CH₂-A-29 |
| 1254. | NH | C(═S)N(CH₃)—CH₂-A-30 |
| 1255. | NH | C(═S)N(CH₃)—CH₂-A-31 |
| 1256. | NH | C(═S)N(CH₃)—CH₂-A-32 |
| 1257. | NH | C(═S)N(CH₃)—CH₂-A-33 |
| 1258. | NH | C(═S)N(CH₃)—SO₂—CH₃ |
| 1259. | NH | C(═S)N(CH₃)—SO₂—CF₃ |
| 1260. | NH | C(═S)N(CH₃)—SO₂—CH₂CH₃ |
| 1261. | NH | C(═S)N(CH₃)—SO₂—CH₂CF₃ |
| 1262. | NH | C(═S)N(CH₃)—SO₂—CH₂CH₂CH₃ |
| 1263. | NH | C(═S)N(CH₃)—SO₂—CH₂CH₂CF₃ |
| 1264. | NH | C(═S)N(CH₃)—SO₂—CH₂CF₂CF₃ |
| 1265. | NH | C(═S)N(CH₃)—SO₂—CH(CH₃)₂ |
| 1266. | NH | C(═S)N(CH₃)—SO₂—CH(CF₃)₂ |
| 1267. | NH | C(═S)NH—SO₂—NH₂ |
| 1268. | NH | C(═S)NH—SO₂—NHCH₃ |
| 1269. | NH | C(═S)NH—SO₂—N(CH₃)₂ |
| 1270. | NH | C(═S)NH—SO₂—NHCF₃ |
| 1271. | NH | C(═S)NH—SO₂—N(CF₃)₂ |
| 1272. | NH | C(═S)NH—SO₂—NHCH₂CH₃ |
| 1273. | NH | C(═S)NH—SO₂—N(CH₂CH₃)₂ |
| 1274. | NH | C(═S)NH—SO₂—NHCH₂CF₃ |
| 1275. | NH | C(═S)NH—SO₂—N(CH₂CF₃)₂ |
| 1276. | NH | C(═S)NH—SO₂—N(CH₃)CH₂CH₃ |
| 1277. | NH | C(═S)NH—SO₂—N(CH₃)CH₂CF₃ |
| 1278. | NH | C(═S)NH—SO₂—N(CF₃)CH₂CH₃ |
| 1279. | NH | C(═S)NH—SO₂—NHCH₂CH₂CH₃ |
| 1280. | NH | C(═S)NH—SO₂—N(CH₂CH₂CH₃)₂ |
| 1281. | NH | C(═S)NH—SO₂—NHCH₂CH₂CF₃ |
| 1282. | NH | C(═S)NH—SO₂—N(CH₂CH₂CF₃)₂ |
| 1283. | NH | C(═S)NH—SO₂—N(CH₃)CH₂CH₂CH₃ |
| 1284. | NH | C(═S)NH—SO₂—N(CH₃)CH₂CH₂CF₃ |
| 1285. | NH | C(═S)NH—SO₂—N(CF₃)CH₂CH₂CH₃ |
| 1286. | NH | C(═S)NH—SO₂—NHCH(CH₃)₂ |
| 1287. | NH | C(═S)NH—SO₂—NHCH(CF₃)₂ |
| 1288. | NH | C(═S)NH—SO₂—N(CH₃)CH(CH₃)₂ |
| 1289. | NH | C(═S)NH—SO₂—N(CH₃)CH(CF₃)₂ |
| 1290. | NH | C(═S)NH—SO₂—N(CF₃)CH(CH₃)₂ |
| 1291. | NH | C(═S)NH—SO₂—NHCH₂CH₂CH₂CH₃ |
| 1292. | NH | C(═S)NH—SO₂—N(CH₂CH₂CH₂CH₃)₂ |
| 1293. | NH | C(═S)NH—SO₂—N(CH₃)CH₂CH₂CH₂CH₃ |
| 1294. | NH | C(═S)N(CH₃)—SO₂—NH₂ |
| 1295. | NH | C(═S)N(CH₃)—SO₂—NHCH₃ |
| 1296. | NH | C(═S)N(CH₃)—SO₂—N(CH₃)₂ |
| 1297. | NH | C(═S)N(CH₃)—SO₂—NHCF₃ |
| 1298. | NH | C(═S)N(CH₃)—SO₂—N(CF₃)₂ |
| 1299. | NH | C(═S)N(CH₃)—SO₂—NHCH₂CH₃ |
| 1300. | NH | C(═S)N(CH₃)—SO₂—N(CH₂CH₃)₂ |
| 1301. | NH | C(═S)N(CH₃)—SO₂—NHCH₂CF₃ |
| 1302. | NH | C(═S)N(CH₃)—SO₂—N(CH₂CF₃)₂ |
| 1303. | NH | C(═S)N(CH₃)—SO₂—N(CH₃)CH₂CH₃ |
| 1304. | NH | C(═S)N(CH₃)—SO₂—N(CH₃)CH₂CF₃ |
| 1305. | NH | C(═S)N(CH₃)—SO₂—N(CF₃)CH₂CH₃ |
| 1306. | NH | C(═S)N(CH₃)—SO₂—NHCH₂CH₂CH₃ |
| 1307. | NH | C(═S)N(CH₃)—SO₂—N(CH₂CH₂CH₃)₂ |
| 1308. | NH | C(═S)N(CH₃)—SO₂—NHCH₂CH₂CF₃ |
| 1309. | NH | C(═S)N(CH₃)—SO₂—N(CH₂CH₂CF₃)₂ |
| 1310. | NH | C(═S)N(CH₃)—SO₂—N(CH₃)CH₂CH₂CH₃ |
| 1311. | NH | C(═S)N(CH₃)—SO₂—N(CH₃)CH₂CH₂CF₃ |
| 1312. | NH | C(═S)N(CH₃)—SO₂—N(CF₃)CH₂CH₂CH₃ |
| 1313. | NH | C(═S)N(CH₃)—SO₂—NHCH(CH₃)₂ |
| 1314. | NH | C(═S)N(CH₃)—SO₂—NHCH(CF₃)₂ |
| 1315. | NH | C(═S)N(CH₃)—SO₂—N(CH₃)CH(CH₃)₂ |
| 1316. | NH | C(═S)N(CH₃)—SO₂—N(CH₃)CH(CF₃)₂ |
| 1317. | NH | C(═S)N(CH₃)—SO₂—N(CF₃)CH(CH₃)₂ |
| 1318. | NH | C(═S)N(CH₃)—SO₂—NHCH₂CH₂CH₂CH₃ |
| 1319. | NH | C(═S)N(CH₃)—SO₂—N(CH₂CH₂CH₂CH₃)₂ |
| 1320. | NH | C(═S)N(CH₃)—SO₂—N(CH₃)CH₂CH₂CH₂CH₃ |
| 1321. | NH | C(═S)—N═CHOCH₃ |
| 1322. | NH | C(═S)—N═CHOCH₂CH₃ |
| 1323. | NH | C(═S)—N═CHOCH₂CH₂CH₃ |
| 1324. | NH | C(═S)—N═CHOCH(CH₃)₂ |
| 1325. | NH | C(═S)—N═CHOCF₃ |
| 1326. | NH | C(═S)—N═CHOCH₂CF₃ |
| 1327. | NH | C(═S)—N═CHOCH₂CH₂CF₃ |
| 1328. | NH | C(═S)—N═CHOCH(CF₃)₂ |
| 1329. | NH | C(═S)—N═CH—CO—OCH₃ |
| 1330. | NH | C(═S)—N═CH—CO—OCH₂CH₃ |
| 1331. | NH | C(═S)—N═CH—CO—OCH₂CH₂CH₃ |
| 1332. | NH | C(═S)—N═CH—CO—OCH(CH₃)₂ |
| 1333. | NH | C(═S)—N═CH—CO—OCF₃ |
| 1334. | NH | C(═S)—N═CH—CO—OCH₂CF₃ |
| 1335. | NH | C(═S)—N═CH—CO—OCH₂CH₂CF₃ |
| 1336. | NH | C(═S)—N═CH—CO—OCH(CF₃)₂ |
| 1337. | NH | C(═S)—N═CH—CO—NHCH₃ |
| 1338. | NH | C(═S)—N═CH—CO—N(CH₃)₂ |
| 1339. | NH | C(═S)—N═CH—CO—NHCH₂CH₃ |
| 1340. | NH | C(═S)—N═CH—CO—N(CH₂CH₃)₂ |
| 1341. | NH | C(═S)—N═CH—CO—N(CH₃)CH₂CH₃ |
| 1342. | NH | C(═S)—N═CH—CO—NHCH₂CH₂CH₃ |
| 1343. | NH | C(═S)—N═CH—CO—N(CH₂CH₂CH₃)₂ |
| 1344. | NH | C(═S)—N═CH—CO—N(CH₃)CH₂CH₂CH₃ |
| 1345. | NH | C(═S)—N═CH—CO—NHCH(CH₃)₂ |
| 1346. | NH | C(═S)—N═CH—CO—N(CH₃)CH(CH₃)₂ |
| 1347. | NH | C(═S)—N═CH—CO—NHCF₃ |
| 1348. | NH | C(═S)—N═CH—CO—N(CF₃)₂ |
| 1349. | NH | C(═S)—N═CH—CO—NHCH₂CF₃ |
| 1350. | NH | C(═S)—N═CH—CO—N(CH₂CF₃)₂ |
| 1351. | NH | C(═S)—N═CH—CO—N(CH₃)CH₂CF₃ |
| 1352. | NH | C(═S)—N═CH—CO—N(CF₃)CH₂CF₃ |
| 1353. | NH | C(═S)—N═CH—CO—NHCH₂CH₂CF₃ |
| 1354. | NH | C(═S)—N═CH—CO—N(CH₂CH₂CF₃)₂ |
| 1355. | NH | C(═S)—N═CH—CO—N(CH₃)CH₂CH₂CF₃ |
| 1356. | NH | C(═S)—N═CH—CO—N(CF₃)CH₂CH₂CH₃ |
| 1357. | NH | C(═S)—N═CH—CO—NHCH(CF₃)₂ |
| 1358. | NH | C(═S)—N═CH—CO—N(CH₃)CH(CF₃)₂ |
| 1359. | NH | C(═S)—N═CH—CO—N(CF₃)CH(CH₃)₂ |
| 1360. | NCH₃ | C(═O)NH₂ |
| 1361. | NCH₃ | C(═O)NHCH₃ |
| 1362. | NCH₃ | C(═O)N(CH₃)₂ |
| 1363. | NCH₃ | C(═O)NHCF₃ |
| 1364. | NCH₃ | C(═O)N(CF₃)₂ |
| 1365. | NCH₃ | C(═O)NHCH₂CH₃ |
| 1366. | NCH₃ | C(═O)N(CH₂CH₃)₂ |
| 1367. | NCH₃ | C(═O)N(CH₃)CH₂CH₃ |
| 1368. | NCH₃ | C(═O)NHCH₂CF₃ |
| 1369. | NCH₃ | C(═O)N(CH₂CF₃)₂ |
| 1370. | NCH₃ | C(═O)N(CH₃)CH₂CF₃ |
| 1371. | NCH₃ | C(═O)NHCH₂CH₂CH₃ |
| 1372. | NCH₃ | C(═O)N(CH₃)CH₂CH₂CH₃ |
| 1373. | NCH₃ | C(═O)NHCH(CH₃)₂ |
| 1374. | NCH₃ | C(═O)NH(CH₂)₃CH₃ |
| 1375. | NCH₃ | C(═O)N(CH₃)—(CH₂)₃CH₃ |
| 1376. | NCH₃ | C(═O)N[(CH₂)₃CH₃]₂ |
| 1377. | NCH₃ | C(═O)N(CH₃)—CH₂—C₆H₅ |
| 1378. | NCH₃ | C(═O)NH-propargyl |
| 1379. | NCH₃ | C(═O)N(CH₃)-propargyl |
| 1380. | NCH₃ | C(═O)NH—CH₂-4-Cl-C₆H₄ |
| 1381. | NCH₃ | C(═O)N(CH₃)-CH₂-4-Cl-C₆H₄ |
| 1382. | NCH₃ | C(═O)morpholin-4-yl |
| 1383. | NCH₃ | C(═O)NH-3-thiolyl-1,1-dioxid |
| 1384. | NCH₃ | C(═O)N(CH₃)-3-thiolyl-1,1-dioxid |
| 1385. | NCH₃ | C(═O)-azirid-1-yl |
| 1386. | NCH₃ | C(═O)-pyrrolidin-1-yl |
| 1387. | NCH₃ | C(═O)-piperidin-1-yl |
| 1388. | NCH₃ | C(═O)-thiomorpholin-4-yl |
| 1389. | NCH₃ | C(═O)NH—CH₂CHF₂ |
| 1390. | NCH₃ | C(═O)NH—CH₂CH₂CHF₂ |
| 1391. | NCH₃ | C(═O)NH—CH₂CH₂CF₃ |
| 1392. | NCH₃ | C(═O)NH-cyclopropyl |
| 1393. | NCH₃ | C(═O)NH-cyclobutyl |
| 1394. | NCH₃ | C(═O)NH-cyclopentyl |
| 1395. | NCH₃ | C(═O)NH-cyclohexyl |
| 1396. | NCH₃ | C(═O)NH—CH₂-cyclopropyl |
| 1397. | NCH₃ | C(═O)NH—CH₂-cyclobutyl |
| 1398. | NCH₃ | C(═O)NH—CH₂-cyclopentyl |
| 1399. | NCH₃ | C(═O)NH—CH₂-cyclohexyl |
| 1400. | NCH₃ | C(═O)NH—CN |
| 1401. | NCH₃ | C(═O)NH—CH₂—CN |
| 1402. | NCH₃ | C(═O)NH—CH₂—CH═CH₂ |
| 1403. | NCH₃ | C(═O)NH—CH₂—CH═C(Cl)₂ |
| 1404. | NCH₃ | C(═O)NH—CH₂—CH═CH-phenyl |
| 1405. | NCH₃ | C(═O)NH—CH₂—CH═CH-(4-Cl-phenyl) |
| 1406. | NCH₃ | C(═O)NH—CH₂—SCH₃ |
| 1407. | NCH₃ | C(═O)NH—CH₂—SCF₃ |
| 1408. | NCH₃ | C(═O)NH—CH₂—CH₂—SCH₃ |
| 1409. | NCH₃ | C(═O)NH—CH₂—CH₂—SCF₃ |
| 1410. | NCH₃ | C(═O)NH—CH₂—SO₂—CH₃ |
| 1411. | NCH₃ | C(═O)NH—CH₂—SO₂—CF₃ |
| 1412. | NCH₃ | C(═O)NH—CH₂—CH₂—SO₂—CH₃ |
| 1413. | NCH₃ | C(═O)NH—CH₂—CH₂—SO₂—CF₃ |
| 1414. | NCH₃ | C(═O)NH—CH₂—CO—NH₂ |
| 1415. | NCH₃ | C(═O)NH—CH₂—CO—NHCH₃ |
| 1416. | NCH₃ | C(═O)NH—CH₂—CO—N(CH₃)₂ |
| 1417. | NCH₃ | C(═O)NH—CH₂—CO—NHCF₃ |
| 1418. | NCH₃ | C(═O)NH—CH₂—CO—N(CF₃)₂ |
| 1419. | NCH₃ | C(═O)NH—CH₂—CO—NHCH₂CH₃ |
| 1420. | NCH₃ | C(═O)NH—CH₂—CO—N(CH₂CH₃)₂ |
| 1421. | NCH₃ | C(═O)NH—CH₂—CO—NHCH₂CF₃ |
| 1422. | NCH₃ | C(═O)NH—CH₂—CO—N(CH₂CF₃)₂ |
| 1423. | NCH₃ | C(═O)NH—CH₂—CO—NHCH₂CH₂CH₃ |
| 1424. | NCH₃ | C(═O)NH—CH₂—CO—N(CH₂CH₂CH₃)₂ |
| 1425. | NCH₃ | C(═O)NH—CH₂—CO—NHCH₂CH₂CF₃ |
| 1426. | NCH₃ | C(═O)NH—CH₂—CO—N(CH₂CH₂CF₃)₂ |
| 1427. | NCH₃ | C(═O)NH—CH₂—CO—NHCH(CH₃)₂ |
| 1428. | NCH₃ | C(═O)NH—CH₂—CO—NHCH(CF₃)₂ |
| 1429. | NCH₃ | C(═O)NH—CH₂—CO—NH-cyclopropyl |
| 1430. | NCH₃ | C(═O)NH—CH₂—CO—NH—CH₂-cyclopropyl |
| 1431. | NCH₃ | C(═O)NH—CH₂—CO—OH |
| 1432. | NCH₃ | C(═O)NH—CH₂—CO—OCH₃ |
| 1433. | NCH₃ | C(═O)NH—CH₂—CO—OCF₃ |
| 1434. | NCH₃ | C(═O)NH—CH₂—CO—OCH₂CH₃ |
| 1435. | NCH₃ | C(═O)NH—CH₂—CO—OCH₂CF₃ |
| 1436. | NCH₃ | C(═O)NH—CH₂—CO—OCH₂CH₂CH₃ |
| 1437. | NCH₃ | C(═O)NH—CH₂—CO—OCH(CH₃)₂ |
| 1438. | NCH₃ | C(═O)NH—CH₂—CO—OCH₂CH₂CH₂CH₃ |
| 1439. | NCH₃ | C(═O)NH—CH₂—CO—OCH(CH₃)CH₂CH₃ |
| 1440. | NCH₃ | C(═O)NH—CH₂—CO—OCH₂CH(CH₃)₂ |
| 1441. | NCH₃ | C(═O)NH—CH₂—CO—OC(CH₃)₃ |
| 1442. | NCH₃ | C(═O)NH-A-1 |
| 1443. | NCH₃ | C(═O)NH-A-2 |
| 1444. | NCH₃ | C(═O)NH-A-3 |
| 1445. | NCH₃ | C(═O)NH-A-4 |
| 1446. | NCH₃ | C(═O)NH-A-5 |
| 1447. | NCH₃ | C(═O)NH-A-6 |
| 1448. | NCH₃ | C(═O)NH-A-7 |
| 1449. | NCH₃ | C(═O)NH-A-8 |
| 1450. | NCH₃ | C(═O)NH-A-9 |
| 1451. | NCH₃ | C(═O)NH-A-10 |
| 1452. | NCH₃ | C(═O)NH-A-11 |
| 1453. | NCH₃ | C(═O)NH-A-12 |
| 1454. | NCH₃ | C(═O)NH-A-13 |
| 1455. | NCH₃ | C(═O)NH-A-14 |
| 1456. | NCH₃ | C(═O)NH-A-15 |
| 1457. | NCH₃ | C(═O)NH-A-16 |
| 1458. | NCH₃ | C(═O)NH-A-17 |
| 1459. | NCH₃ | C(═O)NH-A-18 |
| 1460. | NCH₃ | C(═O)NH-A-19 |
| 1461. | NCH₃ | C(═O)NH-A-20 |
| 1462. | NCH₃ | C(═O)NH-A-21 |
| 1463. | NCH₃ | C(═O)NH-A-22 |
| 1464. | NCH₃ | C(═O)NH-A-23 |
| 1465. | NCH₃ | C(═O)NH-A-24 |
| 1466. | NCH₃ | C(═O)NH-A-25 |
| 1467. | NCH₃ | C(═O)NH-A-26 |
| 1468. | NCH₃ | C(═O)NH-A-27 |
| 1469. | NCH₃ | C(═O)NH-A-28 |
| 1470. | NCH₃ | C(═O)NH-A-29 |
| 1471. | NCH₃ | C(═O)NH-A-30 |
| 1472. | NCH₃ | C(═O)NH-A-31 |
| 1473. | NCH₃ | C(═O)NH-A-32 |
| 1474. | NCH₃ | C(═O)NH-A-33 |
| 1475. | NCH₃ | C(═O)NH—CH₂-A-1 |
| 1476. | NCH₃ | C(═O)NH—CH₂-A-2 |
| 1477. | NCH₃ | C(═O)NH—CH₂-A-3 |
| 1478. | NCH₃ | C(═O)NH—CH₂-A-4 |
| 1479. | NCH₃ | C(═O)NH—CH₂-A-5 |
| 1480. | NCH₃ | C(═O)NH—CH₂-A-6 |
| 1481. | NCH₃ | C(═O)NH—CH₂-A-7 |
| 1482. | NCH₃ | C(═O)NH—CH₂-A-8 |
| 1483. | NCH₃ | C(═O)NH—CH₂-A-9 |
| 1484. | NCH₃ | C(═O)NH—CH₂-A-10 |
| 1485. | NCH₃ | C(═O)NH—CH₂-A-11 |
| 1486. | NCH₃ | C(═O)NH—CH₂-A-12 |
| 1487. | NCH₃ | C(═O)NH—CH₂-A-13 |
| 1488. | NCH₃ | C(═O)NH—CH₂-A-14 |
| 1489. | NCH₃ | C(═O)NH—CH₂-A-15 |
| 1490. | NCH₃ | C(═O)NH—CH₂-A-16 |
| 1491. | NCH₃ | C(═O)NH—CH₂-A-17 |
| 1492. | NCH₃ | C(═O)NH—CH₂-A-18 |
| 1493. | NCH₃ | C(═O)NH—CH₂-A-19 |
| 1494. | NCH₃ | C(═O)NH—CH₂-A-20 |
| 1495. | NCH₃ | C(═O)NH—CH₂-A-21 |
| 1496. | NCH₃ | C(═O)NH—CH₂-A-22 |
| 1497. | NCH₃ | C(═O)NH—CH₂-A-23 |
| 1498. | NCH₃ | C(═O)NH—CH₂-A-24 |
| 1499. | NCH₃ | C(═O)NH—CH₂-A-25 |
| 1500. | NCH₃ | C(═O)NH—CH₂-A-26 |
| 1501. | NCH₃ | C(═O)NH—CH₂-A-27 |
| 1502. | NCH₃ | C(═O)NH—CH₂-A-28 |
| 1503. | NCH₃ | C(═O)NH—CH₂-A-29 |
| 1504. | NCH₃ | C(═O)NH—CH₂-A-30 |
| 1505. | NCH₃ | C(═O)NH—CH₂-A-31 |
| 1506. | NCH₃ | C(═O)NH—CH₂-A-32 |
| 1507. | NCH₃ | C(═O)NH—CH₂-A-33 |
| 1508. | NCH₃ | C(═O)NH—SO₂—CH₃ |
| 1509. | NCH₃ | C(═O)NH—SO₂—CF₃ |
| 1510. | NCH₃ | C(═O)NH—SO₂—CH₂CH₃ |
| 1511. | NCH₃ | C(═O)NH—SO₂—CH₂CF₃ |
| 1512. | NCH₃ | C(═O)NH—SO₂—CH₂CH₂CH₃ |
| 1513. | NCH₃ | C(═O)NH—SO₂—CH₂CH₂CF₃ |
| 1514. | NCH₃ | C(═O)NH—SO₂—CH₂CF₂CF₃ |
| 1515. | NCH₃ | C(═O)NH—SO₂—CH(CH₃)₂ |
| 1516. | NCH₃ | C(═O)NH—SO₂—CH(CF₃)₂ |
| 1517. | NCH₃ | C(═O)N(CH₃)—CH₂CHF₂ |
| 1518. | NCH₃ | C(═O)N(CH₃)—CH₂CH₂CHF₂ |
| 1519. | NCH₃ | C(═O)N(CH₃)—CH₂CH₂CF₃ |
| 1520. | NCH₃ | C(═O)N(CH₃)-cyclopropyl |
| 1521. | NCH₃ | C(═O)N(CH₃)-cyclobutyl |
| 1522. | NCH₃ | C(═O)N(CH₃)-cyclopentyl |
| 1523. | NCH₃ | C(═O)N(CH₃)-cyclohexyl |
| 1524. | NCH₃ | C(═O)N(CH₃)—CH₂-cyclopropyl |
| 1525. | NCH₃ | C(═O)N(CH₃)—CH₂-cyclobutyl |
| 1526. | NCH₃ | C(═O)N(CH₃)—CH₂-cyclopentyl |
| 1527. | NCH₃ | C(═O)N(CH₃)—CH₂-cyclohexyl |
| 1528. | NCH₃ | C(═O)N(CH₃)—CN |
| 1529. | NCH₃ | C(═O)N(CH₃)—CH₂—CN |
| 1530. | NCH₃ | C(═O)N(CH₃)—CH₂—CH═CH₂ |
| 1531. | NCH₃ | C(═O)N(CH₃)—CH₂—CH═C(Cl)₂ |
| 1532. | NCH₃ | C(═O)N(CH₃)—CH₂—CH═CH-phenyl |
| 1533. | NCH₃ | C(═O)N(CH₃)—CH₂—CH═CH-(4-Cl-phenyl) |
| 1534. | NCH₃ | C(═O)N(CH₃)—CH₂—SCH₃ |
| 1535. | NCH₃ | C(═O)N(CH₃)—CH₂—SCF₃ |
| 1536. | NCH₃ | C(═O)N(CH₃)—CH₂—CH₂—SCH₃ |
| 1537. | NCH₃ | C(═O)N(CH₃)—CH₂—CH₂—SCF₃ |
| 1538. | NCH₃ | C(═O)N(CH₃)—CH₂—SO₂—CH₃ |
| 1539. | NCH₃ | C(═O)N(CH₃)—CH₂—SO₂—CF₃ |
| 1540. | NCH₃ | C(═O)N(CH₃)—CH₂—CH₂—SO₂—CH₃ |
| 1541. | NCH₃ | C(═O)N(CH₃)—CH₂—CH₂—SO₂—CF₃ |
| 1542. | NCH₃ | C(═O)N(CH₃)—CH₂—CO—NH₂ |
| 1543. | NCH₃ | C(═O)N(CH₃)—CH₂—CO—NHCH₃ |
| 1544. | NCH₃ | C(═O)N(CH₃)—CH₂—CO—N(CH₃)₂ |
| 1545. | NCH₃ | C(═O)N(CH₃)—CH₂—CO—NHCF₃ |
| 1546. | NCH₃ | C(═O)N(CH₃)—CH₂—CO—N(CF₃)₂ |
| 1547. | NCH₃ | C(═O)N(CH₃)—CH₂—CO—NHCH₂CH₃ |
| 1548. | NCH₃ | C(═O)N(CH₃)—CH₂—CO—N(CH₂CH₃)₂ |
| 1549. | NCH₃ | C(═O)N(CH₃)—CH₂—CO—NHCH₂CF₃ |
| 1550. | NCH₃ | C(═O)N(CH₃)—CH₂—CO—N(CH₂CF₃)₂ |
| 1551. | NCH₃ | C(═O)N(CH₃)—CH₂—CO—NHCH₂CH₂CH₃ |
| 1552. | NCH₃ | C(═O)N(CH₃)—CH₂—CO—N(CH₂CH₂CH₃)₂ |
| 1553. | NCH₃ | C(═O)N(CH₃)—CH₂—CO—NHCH₂CH₂CF₃ |
| 1554. | NCH₃ | C(═O)N(CH₃)—CH₂—CO—N(CH₂CH₂CF₃)₂ |
| 1555. | NCH₃ | C(═O)N(CH₃)—CH₂—CO—NHCH(CH₃)₂ |
| 1556. | NCH₃ | C(═O)N(CH₃)—CH₂—CO—NHCH(CF₃)₂ |
| 1557. | NCH₃ | C(═O)N(CH₃)—CH₂—CO—NH-cyclopropyl |
| 1558. | NCH₃ | C(═O)N(CH₃)—CH₂—CO—NH—CH₂- |
| | | cyclopropyl |
| 1559. | NCH₃ | C(═O)N(CH₃)—CH₂—CO—OH |
| 1560. | NCH₃ | C(═O)N(CH₃)—CH₂—CO—OCH₃ |
| 1561. | NCH₃ | C(═O)N(CH₃)—CH₂—CO—OCF₃ |
| 1562. | NCH₃ | C(═O)N(CH₃)—CH₂—CO—OCH₂CH₃ |
| 1563. | NCH₃ | C(═O)N(CH₃)—CH₂—CO—OCH₂CF₃ |
| 1564. | NCH₃ | C(═O)N(CH₃)—CH₂—CO—OCH₂CH₂CH₃ |
| 1565. | NCH₃ | C(═O)N(CH₃)—CH₂—CO—OCH(CH₃)₂ |
| 1566. | NCH₃ | C(═O)N(CH₃)—CH₂—CO—OCH₂CH₂CH₂CH₃ |
| 1567. | NCH₃ | C(═O)N(CH₃)—CH₂—CO—OCH(CH₃)CH₂CH₃ |
| 1568. | NCH₃ | C(═O)N(CH₃)—CH₂—CO—OCH₂CH(CH₃)₂ |
| 1569. | NCH₃ | C(═O)N(CH₃)—CH₂—CO—OC(CH₃)₃ |
| 1570. | NCH₃ | C(═O)N(CH₃)-A-1 |
| 1571. | NCH₃ | C(═O)N(CH₃)-A-2 |
| 1572. | NCH₃ | C(═O)N(CH₃)-A-3 |
| 1573. | NCH₃ | C(═O)N(CH₃)-A-4 |
| 1574. | NCH₃ | C(═O)N(CH₃)-A-5 |
| 1575. | NCH₃ | C(═O)N(CH₃)-A-6 |
| 1576. | NCH₃ | C(═O)N(CH₃)-A-7 |
| 1577. | NCH₃ | C(═O)N(CH₃)-A-8 |
| 1578. | NCH₃ | C(═O)N(CH₃)-A-9 |
| 1579. | NCH₃ | C(═O)N(CH₃)-A-10 |
| 1580. | NCH₃ | C(═O)N(CH₃)-A-11 |
| 1581. | NCH₃ | C(═O)N(CH₃)-A-12 |
| 1582. | NCH₃ | C(═O)N(CH₃)-A-13 |
| 1583. | NCH₃ | C(═O)N(CH₃)-A-14 |
| 1584. | NCH₃ | C(═O)N(CH₃)-A-15 |
| 1585. | NCH₃ | C(═O)N(CH₃)-A-16 |
| 1586. | NCH₃ | C(═O)N(CH₃)-A-17 |
| 1587. | NCH₃ | C(═O)N(CH₃)-A-18 |
| 1588. | NCH₃ | C(═O)N(CH₃)-A-19 |
| 1589. | NCH₃ | C(═O)N(CH₃)-A-20 |
| 1590. | NCH₃ | C(═O)N(CH₃)-A-21 |
| 1591. | NCH₃ | C(═O)N(CH₃)-A-22 |
| 1592. | NCH₃ | C(═O)N(CH₃)-A-23 |
| 1593. | NCH₃ | C(═O)N(CH₃)-A-24 |
| 1594. | NCH₃ | C(═O)N(CH₃)-A-25 |
| 1595. | NCH₃ | C(═O)N(CH₃)-A-26 |
| 1596. | NCH₃ | C(═O)N(CH₃)-A-27 |
| 1597. | NCH₃ | C(═O)N(CH₃)-A-28 |
| 1598. | NCH₃ | C(═O)N(CH₃)-A-29 |
| 1599. | NCH₃ | C(═O)N(CH₃)-A-30 |
| 1600. | NCH₃ | C(═O)N(CH₃)-A-31 |
| 1601. | NCH₃ | C(═O)N(CH₃)-A-32 |
| 1602. | NCH₃ | C(═O)N(CH₃)-A-33 |
| 1603. | NCH₃ | C(═O)N(CH₃)—CH₂-A-1 |
| 1604. | NCH₃ | C(═O)N(CH₃)—CH₂-A-2 |
| 1605. | NCH₃ | C(═O)N(CH₃)—CH₂-A-3 |
| 1606. | NCH₃ | C(═O)N(CH₃)—CH₂-A-4 |
| 1607. | NCH₃ | C(═O)N(CH₃)—CH₂-A-5 |
| 1608. | NCH₃ | C(═O)N(CH₃)—CH₂-A-6 |
| 1609. | NCH₃ | C(═O)N(CH₃)—CH₂-A-7 |
| 1610. | NCH₃ | C(═O)N(CH₃)—CH₂-A-8 |
| 1611. | NCH₃ | C(═O)N(CH₃)—CH₂-A-9 |
| 1612. | NCH₃ | C(═O)N(CH₃)—CH₂-A-10 |
| 1613. | NCH₃ | C(═O)N(CH₃)—CH₂-A-11 |
| 1614. | NCH₃ | C(═O)N(CH₃)—CH₂-A-12 |
| 1615. | NCH₃ | C(═O)N(CH₃)—CH₂-A-13 |
| 1616. | NCH₃ | C(═O)N(CH₃)—CH₂-A-14 |
| 1617. | NCH₃ | C(═O)N(CH₃)—CH₂-A-15 |
| 1618. | NCH₃ | C(═O)N(CH₃)—CH₂-A-16 |
| 1619. | NCH₃ | C(═O)N(CH₃)—CH₂-A-17 |
| 1620. | NCH₃ | C(═O)N(CH₃)—CH₂-A-18 |
| 1621. | NCH₃ | C(═O)N(CH₃)—CH₂-A-19 |
| 1622. | NCH₃ | C(═O)N(CH₃)—CH₂-A-20 |
| 1623. | NCH₃ | C(═O)N(CH₃)—CH₂-A-21 |
| 1624. | NCH₃ | C(═O)N(CH₃)—CH₂-A-22 |
| 1625. | NCH₃ | C(═O)N(CH₃)—CH₂-A-23 |
| 1626. | NCH₃ | C(═O)N(CH₃)—CH₂-A-24 |
| 1627. | NCH₃ | C(═O)N(CH₃)—CH₂-A-25 |
| 1628. | NCH₃ | C(═O)N(CH₃)—CH₂-A-26 |
| 1629. | NCH₃ | C(═O)N(CH₃)—CH₂-A-27 |
| 1630. | NCH₃ | C(═O)N(CH₃)—CH₂-A-28 |
| 1631. | NCH₃ | C(═O)N(CH₃)—CH₂-A-29 |
| 1632. | NCH₃ | C(═O)N(CH₃)—CH₂-A-30 |
| 1633. | NCH₃ | C(═O)N(CH₃)—CH₂-A-31 |
| 1634. | NCH₃ | C(═O)N(CH₃)—CH₂-A-32 |
| 1635. | NCH₃ | C(═O)N(CH₃)—CH₂-A-33 |
| 1636. | NCH₃ | C(═O)N(CH₃)—SO₂—CH₃ |
| 1637. | NCH₃ | C(═O)N(CH₃)—SO₂—CF₃ |
| 1638. | NCH₃ | C(═O)N(CH₃)—SO₂—CH₂CH₃ |
| 1639. | NCH₃ | C(═O)N(CH₃)—SO₂—CH₂CF₃ |
| 1640. | NCH₃ | C(═O)N(CH₃)—SO₂—CH₂CH₂CH₃ |
| 1641. | NCH₃ | C(═O)N(CH₃)—SO₂—CH₂CH₂CF₃ |
| 1642. | NCH₃ | C(═O)N(CH₃)—SO₂—CH₂CF₂CF₃ |
| 1643. | NCH₃ | C(═O)N(CH₃)—SO₂—CH(CH₃)₂ |
| 1644. | NCH₃ | C(═O)N(CH₃)—SO₂—CH(CF₃)₂ |
| 1645. | NCH₃ | C(═O)NH—SO₂—NH₂ |
| 1646. | NCH₃ | C(═O)NH—SO₂—NHCH₃ |
| 1647. | NCH₃ | C(═O)NH—SO₂—N(CH₃)₂ |
| 1648. | NCH₃ | C(═O)NH—SO₂—NHCF₃ |
| 1649. | NCH₃ | C(═O)NH—SO₂—N(CF₃)₂ |
| 1650. | NCH₃ | C(═O)NH—SO₂—NHCH₂CH₃ |
| 1651. | NCH₃ | C(═O)NH—SO₂—N(CH₂CH₃)₂ |
| 1652. | NCH₃ | C(═O)NH—SO₂—NHCH₂CF₃ |
| 1653. | NCH₃ | C(═O)NH—SO₂—N(CH₂CF₃)₂ |
| 1654. | NCH₃ | C(═O)NH—SO₂—N(CH₃)CH₂CH₃ |
| 1655. | NCH₃ | C(═O)NH—SO₂—N(CH₃)CH₂CF₃ |
| 1656. | NCH₃ | C(═O)NH—SO₂—N(CF₃)CH₂CH₃ |
| 1657. | NCH₃ | C(═O)NH—SO₂—NHCH₂CH₂CH₃ |
| 1658. | NCH₃ | C(═O)NH—SO₂—N(CH₂CH₂CH₃)₂ |
| 1659. | NCH₃ | C(═O)NH—SO₂—NHCH₂CH₂CF₃ |
| 1660. | NCH₃ | C(═O)NH—SO₂—N(CH₂CH₂CF₃)₂ |
| 1661. | NCH₃ | C(═O)NH—SO₂—N(CH₃)CH₂CH₂CH₃ |
| 1662. | NCH₃ | C(═O)NH—SO₂—N(CH₃)CH₂CH₂CF₃ |
| 1663. | NCH₃ | C(═O)NH—SO₂—N(CF₃)CH₂CH₂CH₃ |
| 1664. | NCH₃ | C(═O)NH—SO₂—NHCH(CH₃)₂ |
| 1665. | NCH₃ | C(═O)NH—SO₂—NHCH(CF₃)₂ |
| 1666. | NCH₃ | C(═O)NH—SO₂—N(CH₃)CH(CH₃)₂ |
| 1667. | NCH₃ | C(═O)NH—SO₂—N(CH₃)CH(CF₃)₂ |
| 1668. | NCH₃ | C(═O)NH—SO₂—N(CF₃)CH(CH₃)₂ |
| 1669. | NCH₃ | C(═O)NH—SO₂—NHCH₂CH₂CH₂CH₃ |
| 1670. | NCH₃ | C(═O)NH—SO₂—N(CH₂CH₂CH₂CH₃)₂ |
| 1671. | NCH₃ | C(═O)NH—SO₂—N(CH₃)CH₂CH₂CH₂CH₃ |
| 1672. | NCH₃ | C(═O)N(CH₃)—SO₂—NH₂ |
| 1673. | NCH₃ | C(═O)N(CH₃)—SO₂—NHCH₃ |
| 1674. | NCH₃ | C(═O)N(CH₃)—SO₂—N(CH₃)₂ |
| 1675. | NCH₃ | C(═O)N(CH₃)—SO₂—NHCF₃ |
| 1676. | NCH₃ | C(═O)N(CH₃)—SO₂—N(CF₃)₂ |
| 1677. | NCH₃ | C(═O)N(CH₃)—SO₂—NHCH₂CH₃ |
| 1678. | NCH₃ | C(═O)N(CH₃)—SO₂—N(CH₂CH₃)₂ |
| 1679. | NCH₃ | C(═O)N(CH₃)—SO₂—NHCH₂CF₃ |
| 1680. | NCH₃ | C(═O)N(CH₃)—SO₂—N(CH₂CF₃)₂ |
| 1681. | NCH₃ | C(═O)N(CH₃)—SO₂—N(CH₃)CH₂CH₃ |
| 1682. | NCH₃ | C(═O)N(CH₃)—SO₂—N(CH₃)CH₂CF₃ |
| 1683. | NCH₃ | C(═O)N(CH₃)—SO₂—N(CF₃)CH₂CH₃ |
| 1684. | NCH₃ | C(═O)N(CH₃)—SO₂—NHCH₂CH₂CH₃ |
| 1685. | NCH₃ | C(═O)N(CH₃)—SO₂—N(CH₂CH₂CH₃)₂ |
| 1686. | NCH₃ | C(═O)N(CH₃)—SO₂—NHCH₂CH₂CF₃ |
| 1687. | NCH₃ | C(═O)N(CH₃)—SO₂—N(CH₂CH₂CF₃)₂ |
| 1688. | NCH₃ | C(═O)N(CH₃)—SO₂—N(CH₃)CH₂CH₂CH₃ |
| 1689. | NCH₃ | C(═O)N(CH₃)—SO₂—N(CH₃)CH₂CH₂CF₃ |
| 1690. | NCH₃ | C(═O)N(CH₃)—SO₂—N(CF₃)CH₂CH₂CH₃ |
| 1691. | NCH₃ | C(═O)N(CH₃)—SO₂—NHCH(CH₃)₂ |
| 1692. | NCH₃ | C(═O)N(CH₃)—SO₂—NHCH(CF₃)₂ |
| 1693. | NCH₃ | C(═O)N(CH₃)—SO₂—N(CH₃)CH(CH₃)₂ |
| 1694. | NCH₃ | C(═O)N(CH₃)—SO₂—N(CH₃)CH(CF₃)₂ |
| 1695. | NCH₃ | C(═O)N(CH₃)—SO₂—N(CF₃)CH(CH₃)₂ |
| 1696. | NCH₃ | C(═O)N(CH₃)—SO₂—NHCH₂CH₂CH₂CH₃ |
| 1697. | NCH₃ | C(═O)N(CH₃)—SO₂—N(CH₂CH₂CH₂CH₃)₂ |
| 1698. | NCH₃ | C(═O)N(CH₃)—SO₂—N(CH₃)CH₂CH₂CH₂CH₃ |
| 1699. | NCH₃ | C(═O)—N═CHOCH₃ |
| 1700. | NCH₃ | C(═O)—N═CHOCH₂CH₃ |
| 1701. | NCH₃ | C(═O)—N═CHOCH₂CH₂CH₃ |
| 1702. | NCH₃ | C(═O)—N═CHOCH(CH₃)₂ |
| 1703. | NCH₃ | C(═O)—N═CHOCF₃ |
| 1704. | NCH₃ | C(═O)—N═CHOCH₂CF₃ |
| 1705. | NCH₃ | C(═O)—N═CHOCH₂CH₂CF₃ |
| 1706. | NCH₃ | C(═O)—N═CHOCH(CF₃)₂ |
| 1707. | NCH₃ | C(═O)—N═CH—CO—OCH₃ |
| 1708. | NCH₃ | C(═O)—N═CH—CO—OCH₂CH₃ |
| 1709. | NCH₃ | C(═O)—N═CH—CO—OCH₂CH₂CH₃ |
| 1710. | NCH₃ | C(═O)—N═CH—CO—OCH(CH₃)₂ |
| 1711. | NCH₃ | C(═O)—N═CH—CO—OCF₃ |
| 1712. | NCH₃ | C(═O)—N═CH—CO—OCH₂CF₃ |
| 1713. | NCH₃ | C(═O)—N═CH—CO—OCH₂CH₂CF₃ |
| 1714. | NCH₃ | C(═O)—N═CH—CO—OCH(CF₃)₂ |
| 1715. | NCH₃ | C(═O)—N═CH—CO—NHCH₃ |
| 1716. | NCH₃ | C(═O)—N═CH—CO—N(CH₃)₂ |
| 1717. | NCH₃ | C(═O)—N═CH—CO—NHCH₂CH₃ |
| 1718. | NCH₃ | C(═O)—N═CH—CO—N(CH₂CH₃)₂ |
| 1719. | NCH₃ | C(═O)—N═CH—CO—N(CH₃)CH₂CH₃ |
| 1720. | NCH₃ | C(═O)—N═CH—CO—NHCH₂CH₂CH₃ |
| 1721. | NCH₃ | C(═O)—N═CH—CO—N(CH₂CH₂CH₃)₂ |
| 1722. | NCH₃ | C(═O)—N═CH—CO—N(CH₃)CH₂CH₂CH₃ |
| 1723. | NCH₃ | C(═O)—N═CH—CO—NHCH(CH₃)₂ |
| 1724. | NCH₃ | C(═O)—N═CH—CO—N(CH₃)CH(CH₃)₂ |
| 1725. | NCH₃ | C(═O)—N═CH—CO—NHCF₃ |
| 1726. | NCH₃ | C(═O)—N═CH—CO—N(CF₃)₂ |
| 1727. | NCH₃ | C(═O)—N═CH—CO—NHCH₂CF₃ |
| 1728. | NCH₃ | C(═O)—N═CH—CO—N(CH₂CF₃)₂ |
| 1729. | NCH₃ | C(═O)—N═CH—CO—N(CH₃)CH₂CF₃ |
| 1730. | NCH₃ | C(═O)—N═CH—CO—N(CF₃)CH₂CF₃ |
| 1731. | NCH₃ | C(═O)—N═CH—CO—NHCH₂CH₂CF₃ |
| 1732. | NCH₃ | C(═O)—N═CH—CO—N(CH₂CH₂CF₃)₂ |
| 1733. | NCH₃ | C(═O)—N═CH—CO—N(CH₃)CH₂CH₂CF₃ |
| 1734. | NCH₃ | C(═O)—N═CH—CO—N(CF₃)CH₂CH₂CH₃ |
| 1735. | NCH₃ | C(═O)—N═CH—CO—NHCH(CF₃)₂ |
| 1736. | NCH₃ | C(═O)—N═CH—CO—N(CH₃)CH(CF₃)₂ |
| 1737. | NCH₃ | C(═O)—N═CH—CO—N(CF₃)CH(CH₃)₂ |
| 1738. | NCH₃ | C(═S)NH₂ |
| 1739. | NCH₃ | C(═S)NHCH₃ |
| 1740. | NCH₃ | C(═S)N(CH₃)₂ |
| 1741. | NCH₃ | C(═S)NHCF₃ |
| 1742. | NCH₃ | C(═S)N(CF₃)₂ |
| 1743. | NCH₃ | C(═S)NHCH₂CH₃ |
| 1744. | NCH₃ | C(═S)N(CH₂CH₃)₂ |
| 1745. | NCH₃ | C(═S)N(CH₃)CH₂CH₃ |
| 1746. | NCH₃ | C(═S)NHCH₂CF₃ |
| 1747. | NCH₃ | C(═S)N(CH₂CF₃)₂ |
| 1748. | NCH₃ | C(═S)N(CH₃)CH₂CF₃ |
| 1749. | NCH₃ | C(═S)NHCH₂CH₂CH₃ |
| 1750. | NCH₃ | C(═S)N(CH₃)CH₂CH₂CH₃ |
| 1751. | NCH₃ | C(═S)NHCH(CH₃)₂ |
| 1752. | NCH₃ | C(═S)NH(CH₂)₃CH₃ |
| 1753. | NCH₃ | C(═S)N(CH₃)—(CH₂)₃CH₃ |
| 1754. | NCH₃ | C(═S)N[(CH₂)₃CH₃]₂ |
| 1755. | NCH₃ | C(═S)N(CH₃)—CH₂—C₆H₅ |
| 1756. | NCH₃ | C(═S)NH-propargyl |
| 1757. | NCH₃ | C(═S)N(CH₃)-propargyl |
| 1758. | NCH₃ | C(═S)NH—CH₂-4-Cl—C₆H₄ |
| 1759. | NCH₃ | C(═S)N(CH₃)—CH₂-4-Cl—C₆H₄ |
| 1760. | NCH₃ | C(═S)morpholin-4-yl |
| 1761. | NCH₃ | C(═S)NH-3-thiolyl-1,1-dioxid |
| 1762. | NCH₃ | C(═S)N(CH₃)-3-thiolyl-1,1-dioxid |
| 1763. | NCH₃ | C(═S)-azirid-1-yl |
| 1764. | NCH₃ | C(═S)-pyrrolidin-1-yl |
| 1765. | NCH₃ | C(═S)-piperidin-1-yl |
| 1766. | NCH₃ | C(═S)-thiomorpholin-4-yl |
| 1767. | NCH₃ | C(═S)NH—CH₂CHF₂ |
| 1768. | NCH₃ | C(═S)NH—CH₂CH₂CHF₂ |
| 1769. | NCH₃ | C(═S)NH—CH₂CH₂CF₃ |
| 1770. | NCH₃ | C(═S)NH-cyclopropyl |
| 1771. | NCH₃ | C(═S)NH-cyclobutyl |
| 1772. | NCH₃ | C(═S)NH-cyclopentyl |
| 1773. | NCH₃ | C(═S)NH-cyclohexyl |
| 1774. | NCH₃ | C(═S)NH—CH₂-cyclopropyl |
| 1775. | NCH₃ | C(═S)NH—CH₂-cyclobutyl |
| 1776. | NCH₃ | C(═S)NH—CH₂-cyclopentyl |
| 1777. | NCH₃ | C(═S)NH—CH₂-cyclohexyl |
| 1778. | NCH₃ | C(═S)NH—CN |
| 1779. | NCH₃ | C(═S)NH—CH₂—CN |
| 1780. | NCH₃ | C(═S)NH—CH₂—CH═CH₂ |
| 1781. | NCH₃ | C(═S)NH—CH₂—CH═C(Cl)₂ |
| 1782. | NCH₃ | C(═S)NH—CH₂—CH═CH-phenyl |
| 1783. | NCH₃ | C(═S)NH—CH₂—CH═CH-(4-Cl-phenyl) |
| 1784. | NCH₃ | C(═S)NH—CH₂—SCH₃ |
| 1785. | NCH₃ | C(═S)NH—CH₂—SCF₃ |
| 1786. | NCH₃ | C(═S)NH—CH₂—CH₂—SCH₃ |
| 1787. | NCH₃ | C(═S)NH—CH₂—CH₂—SCF₃ |
| 1788. | NCH₃ | C(═S)NH—CH₂—SO₂—CH₃ |
| 1789. | NCH₃ | C(═S)NH—CH₂—SO₂—CF₃ |
| 1790. | NCH₃ | C(═S)NH—CH₂—CH₂—SO₂—CH₃ |
| 1791. | NCH₃ | C(═S)NH—CH₂—CH₂—SO₂—CF₃ |
| 1792. | NCH₃ | C(═S)NH—CH₂—CO—NH₂ |
| 1793. | NCH₃ | C(═S)NH—CH₂—CO—NHCH₃ |
| 1794. | NCH₃ | C(═S)NH—CH₂—CO—N(CH₃)₂ |
| 1795. | NCH₃ | C(═S)NH—CH₂—CO—NHCF₃ |
| 1796. | NCH₃ | C(═S)NH—CH₂—CO—N(CF₃)₂ |
| 1797. | NCH₃ | C(═S)NH—CH₂—CO—NHCH₂CH₃ |
| 1798. | NCH₃ | C(═S)NH—CH₂—CO—N(CH₂CH₃)₂ |
| 1799. | NCH₃ | C(═S)NH—CH₂—CO—NHCH₂CF₃ |
| 1800. | NCH₃ | C(═S)NH—CH₂—CO—N(CH₂CF₃)₂ |
| 1801. | NCH₃ | C(═S)NH—CH₂—CO—NHCH₂CH₂CH₃ |
| 1802. | NCH₃ | C(═S)NH—CH₂—CO—N(CH₂CH₂CH₃)₂ |
| 1803. | NCH₃ | C(═S)NH—CH₂—CO—NHCH₂CH₂CF₃ |
| 1804. | NCH₃ | C(═S)NH—CH₂—CO—N(CH₂CH₂CF₃)₂ |
| 1805. | NCH₃ | C(═S)NH—CH₂—CO—NHCH(CH₃)₂ |
| 1806. | NCH₃ | C(═S)NH—CH₂—CO—NHCH(CF₃)₂ |
| 1807. | NCH₃ | C(═S)NH—CH₂—CO—NH-cyclopropyl |
| 1808. | NCH₃ | C(═S)NH—CH₂—CO—NH—CH₂-cyclopropyl |
| 1809. | NCH₃ | C(═S)NH—CH₂—CO—OH |
| 1810. | NCH₃ | C(═S)NH—CH₂—CO—OCH₃ |
| 1811. | NCH₃ | C(═S)NH—CH₂—CO—OCF₃ |
| 1812. | NCH₃ | C(═S)NH—CH₂—CO—OCH₂CH₃ |
| 1813. | NCH₃ | C(═S)NH—CH₂—CO—OCH₂CF₃ |
| 1814. | NCH₃ | C(═S)NH—CH₂—CO—OCH₂CH₂CH₃ |
| 1815. | NCH₃ | C(═S)NH—CH₂—CO—OCH(CH₃)₂ |
| 1816. | NCH₃ | C(═S)NH—CH₂—CO—OCH₂CH₂CH₂CH₃ |
| 1817. | NCH₃ | C(═S)NH—CH₂—CO—OCH(CH₃)CH₂CH₃ |
| 1818. | NCH₃ | C(═S)NH—CH₂—CO—OCH₂CH(CH₃)₂ |
| 1819. | NCH₃ | C(═S)NH—CH₂—CO—OC(CH₃)₃ |
| 1820. | NCH₃ | C(═S)NH-A-1 |
| 1821. | NCH₃ | C(═S)NH-A-2 |
| 1822. | NCH₃ | C(═S)NH-A-3 |
| 1823. | NCH₃ | C(═S)NH-A-4 |
| 1824. | NCH₃ | C(═S)NH-A-5 |
| 1825. | NCH₃ | C(═S)NH-A-6 |
| 1826. | NCH₃ | C(═S)NH-A-7 |
| 1827. | NCH₃ | C(═S)NH-A-8 |
| 1828. | NCH₃ | C(═S)NH-A-9 |
| 1829. | NCH₃ | C(═S)NH-A-10 |
| 1830. | NCH₃ | C(═S)NH-A-11 |
| 1831. | NCH₃ | C(═S)NH-A-12 |
| 1832. | NCH₃ | C(═S)NH-A-13 |
| 1833. | NCH₃ | C(═S)NH-A-14 |
| 1834. | NCH₃ | C(═S)NH-A-15 |
| 1835. | NCH₃ | C(═S)NH-A-16 |
| 1836. | NCH₃ | C(═S)NH-A-17 |
| 1837. | NCH₃ | C(═S)NH-A-18 |
| 1838. | NCH₃ | C(═S)NH-A-19 |
| 1839. | NCH₃ | C(═S)NH-A-20 |
| 1840. | NCH₃ | C(═S)NH-A-21 |
| 1841. | NCH₃ | C(═S)NH-A-22 |
| 1842. | NCH₃ | C(═S)NH-A-23 |
| 1843. | NCH₃ | C(═S)NH-A-24 |
| 1844. | NCH₃ | C(═S)NH-A-25 |
| 1845. | NCH₃ | C(═S)NH-A-26 |
| 1846. | NCH₃ | C(═S)NH-A-27 |
| 1847. | NCH₃ | C(═S)NH-A-28 |
| 1848. | NCH₃ | C(═S)NH-A-29 |
| 1849. | NCH₃ | C(═S)NH-A-30 |
| 1850. | NCH₃ | C(═S)NH-A-31 |
| 1851. | NCH₃ | C(═S)NH-A-32 |
| 1852. | NCH₃ | C(═S)NH-A-33 |
| 1853. | NCH₃ | C(═S)NH—CH₂-A-1 |
| 1854. | NCH₃ | C(═S)NH—CH₂-A-2 |
| 1855. | NCH₃ | C(═S)NH—CH₂-A-3 |
| 1856. | NCH₃ | C(═S)NH—CH₂-A-4 |
| 1857. | NCH₃ | C(═S)NH—CH₂-A-5 |
| 1858. | NCH₃ | C(═S)NH—CH₂-A-6 |
| 1859. | NCH₃ | C(═S)NH—CH₂-A-7 |
| 1860. | NCH₃ | C(═S)NH—CH₂-A-8 |
| 1861. | NCH₃ | C(═S)NH—CH₂-A-9 |
| 1862. | NCH₃ | C(═S)NH—CH₂-A-10 |
| 1863. | NCH₃ | C(═S)NH—CH₂-A-11 |
| 1864. | NCH₃ | C(═S)NH—CH₂-A-12 |
| 1865. | NCH₃ | C(═S)NH—CH₂-A-13 |
| 1866. | NCH₃ | C(═S)NH—CH₂-A-14 |
| 1867. | NCH₃ | C(═S)NH—CH₂-A-15 |
| 1868. | NCH₃ | C(═S)NH—CH₂-A-16 |
| 1869. | NCH₃ | C(═S)NH—CH₂-A-17 |
| 1870. | NCH₃ | C(═S)NH—CH₂-A-18 |
| 1871. | NCH₃ | C(═S)NH—CH₂-A-19 |
| 1872. | NCH₃ | C(═S)NH—CH₂-A-20 |
| 1873. | NCH₃ | C(═S)NH—CH₂-A-21 |
| 1874. | NCH₃ | C(═S)NH—CH₂-A-22 |
| 1875. | NCH₃ | C(═S)NH—CH₂-A-23 |
| 1876. | NCH₃ | C(═S)NH—CH₂-A-24 |
| 1877. | NCH₃ | C(═S)NH—CH₂-A-25 |
| 1878. | NCH₃ | C(═S)NH—CH₂-A-26 |
| 1879. | NCH₃ | C(═S)NH—CH₂-A-27 |
| 1880. | NCH₃ | C(═S)NH—CH₂-A-28 |
| 1881. | NCH₃ | C(═S)NH—CH₂-A-29 |
| 1882. | NCH₃ | C(═S)NH—CH₂-A-30 |
| 1883. | NCH₃ | C(═S)NH—CH₂-A-31 |
| 1884. | NCH₃ | C(═S)NH—CH₂-A-32 |
| 1885. | NCH₃ | C(═S)NH—CH₂-A-33 |
| 1886. | NCH₃ | C(═S)NH—SO₂—CH₃ |
| 1887. | NCH₃ | C(═S)NH—SO₂—CF₃ |
| 1888. | NCH₃ | C(═S)NH—SO₂—CH₂CH₃ |
| 1889. | NCH₃ | C(═S)NH—SO₂—CH₂CF₃ |
| 1890. | NCH₃ | C(═S)NH—SO₂—CH₂CH₂CH₃ |
| 1891. | NCH₃ | C(═S)NH—SO₂—CH₂CH₂CF₃ |
| 1892. | NCH₃ | C(═S)NH—SO₂—CH₂CF₂CF₃ |
| 1893. | NCH₃ | C(═S)NH—SO₂—CH(CH₃)₂ |
| 1894. | NCH₃ | C(═S)NH—SO₂—CH(CF₃)₂ |
| 1895. | NCH₃ | C(═S)N(CH₃)—CH₂CHF₂ |
| 1896. | NCH₃ | C(═S)N(CH₃)—CH₂CH₂CHF₂ |
| 1897. | NCH₃ | C(═S)N(CH₃)—CH₂CH₂CF₃ |
| 1898. | NCH₃ | C(═S)N(CH₃)-cyclopropyl |
| 1899. | NCH₃ | C(═S)N(CH₃)-cyclobutyl |
| 1900. | NCH₃ | C(═S)N(CH₃)-cyclopentyl |
| 1901. | NCH₃ | C(═S)N(CH₃)-cyclohexyl |
| 1902. | NCH₃ | C(═S)N(CH₃)—CH₂-cyclopropyl |
| 1903. | NCH₃ | C(═S)N(CH₃)—CH₂-cyclobutyl |
| 1904. | NCH₃ | C(═S)N(CH₃)—CH₂-cyclopentyl |
| 1905. | NCH₃ | C(═S)N(CH₃)—CH₂-cyclohexyl |
| 1906. | NCH₃ | C(═S)N(CH₃)—CN |
| 1907. | NCH₃ | C(═S)N(CH₃)—CH₂—CN |
| 1908. | NCH₃ | C(═S)N(CH₃)—CH₂—CH═CH₂ |
| 1909. | NCH₃ | C(═S)N(CH₃)—CH₂—CH═C(Cl)₂ |
| 1910. | NCH₃ | C(═S)N(CH₃)—CH₂—CH═CH-phenyl |
| 1911. | NCH₃ | C(═S)N(CH₃)—CH₂—CH═CH—(4-Cl-phenyl) |
| 1912. | NCH₃ | C(═S)N(CH₃)—CH₂—SCH₃ |
| 1913. | NCH₃ | C(═S)N(CH₃)—CH₂—SCF₃ |
| 1914. | NCH₃ | C(═S)N(CH₃)—CH₂—CH₂—SCH₃ |
| 1915. | NCH₃ | C(═S)N(CH₃)—CH₂—CH₂—SCF₃ |
| 1916. | NCH₃ | C(═S)N(CH₃)—CH₂—SO₂—CH₃ |
| 1917. | NCH₃ | C(═S)N(CH₃)—CH₂—SO₂—CF₃ |
| 1918. | NCH₃ | C(═S)N(CH₃)—CH₂—CH₂—SO₂—CH₃ |
| 1919. | NCH₃ | C(═S)N(CH₃)—CH₂—CH₂—SO₂—CF₃ |
| 1920. | NCH₃ | C(═S)N(CH₃)—CH₂—CO—NH₂ |
| 1921. | NCH₃ | C(═S)N(CH₃)—CH₂—CO—NHCH₃ |
| 1922. | NCH₃ | C(═S)N(CH₃)—CH₂—CO—N(CH₃)₂ |
| 1923. | NCH₃ | C(═S)N(CH₃)—CH₂—CO—NHCF₃ |
| 1924. | NCH₃ | C(═S)N(CH₃)—CH₂—CO—N(CF₃)₂ |
| 1925. | NCH₃ | C(═S)N(CH₃)—CH₂—CO—NHCH₂CH₃ |
| 1926. | NCH₃ | C(═S)N(CH₃)—CH₂—CO—N(CH₂CH₃)₂ |
| 1927. | NCH₃ | C(═S)N(CH₃)—CH₂—CO—NHCH₂CF₃ |
| 1928. | NCH₃ | C(═S)N(CH₃)—CH₂—CO—N(CH₂CF₃)₂ |
| 1929. | NCH₃ | C(═S)N(CH₃)—CH₂—CO—NHCH₂CH₂CH₃ |
| 1930. | NCH₃ | C(═S)N(CH₃)—CH₂—CO—N(CH₂CH₂CH₃)₂ |
| 1931. | NCH₃ | C(═S)N(CH₃)—CH₂—CO—NHCH₂CH₂CF₃ |
| 1932. | NCH₃ | C(═S)N(CH₃)—CH₂—CO—N(CH₂CH₂CF₃)₂ |
| 1933. | NCH₃ | C(═S)N(CH₃)—CH₂—CO—NHCH(CH₃)₂ |
| 1934. | NCH₃ | C(═S)N(CH₃)—CH₂—CO—NHCH(CF₃)₂ |
| 1935. | NCH₃ | C(═S)N(CH₃)—CH₂—CO—NH-cyclopropyl |
| 1936. | NCH₃ | C(═S)N(CH₃)—CH₂—CO—NH—CH₂- |
| | | cyclopropyl |
| 1937. | NCH₃ | C(═S)N(CH₃)—CH₂—CO—OH |
| 1938. | NCH₃ | C(═S)N(CH₃)—CH₂—CO—OCH₃ |
| 1939. | NCH₃ | C(═S)N(CH₃)—CH₂—CO—OCF₃ |
| 1940. | NCH₃ | C(═S)N(CH₃)—CH₂—CO—OCH₂CH₃ |
| 1941. | NCH₃ | C(═S)N(CH₃)—CH₂—CO—OCH₂CF₃ |
| 1942. | NCH₃ | C(═S)N(CH₃)—CH₂—CO—OCH₂CH₂CH₃ |
| 1943. | NCH₃ | C(═S)N(CH₃)—CH₂—CO—OCH(CH₃)₂ |
| 1944. | NCH₃ | C(═S)N(CH₃)—CH₂—CO—OCH₂CH₂CH₂CH₃ |
| 1945. | NCH₃ | C(═S)N(CH₃)—CH₂—CO—OCH(CH₃)CH₂CH₃ |
| 1946. | NCH₃ | C(═S)N(CH₃)—CH₂—CO—OCH₂CH(CH₃)₂ |
| 1947. | NCH₃ | C(═S)N(CH₃)—CH₂—CO—OC(CH₃)₃ |
| 1948. | NCH₃ | C(═S)N(CH₃)-A-1 |
| 1949. | NCH₃ | C(═S)N(CH₃)-A-2 |
| 1950. | NCH₃ | C(═S)N(CH₃)-A-3 |
| 1951. | NCH₃ | C(═S)N(CH₃)-A-4 |
| 1952. | NCH₃ | C(═S)N(CH₃)-A-5 |
| 1953. | NCH₃ | C(═S)N(CH₃)-A-6 |
| 1954. | NCH₃ | C(═S)N(CH₃)-A-7 |
| 1955. | NCH₃ | C(═S)N(CH₃)-A-8 |
| 1956. | NCH₃ | C(═S)N(CH₃)-A-9 |
| 1957. | NCH₃ | C(═S)N(CH₃)-A-10 |
| 1958. | NCH₃ | C(═S)N(CH₃)-A-11 |
| 1959. | NCH₃ | C(═S)N(CH₃)-A-12 |
| 1960. | NCH₃ | C(═S)N(CH₃)-A-13 |
| 1961. | NCH₃ | C(═S)N(CH₃)-A-14 |
| 1962. | NCH₃ | C(═S)N(CH₃)-A-15 |
| 1963. | NCH₃ | C(═S)N(CH₃)-A-16 |
| 1964. | NCH₃ | C(═S)N(CH₃)-A-17 |
| 1965. | NCH₃ | C(═S)N(CH₃)-A-18 |
| 1966. | NCH₃ | C(═S)N(CH₃)-A-19 |
| 1967. | NCH₃ | C(═S)N(CH₃)-A-20 |
| 1968. | NCH₃ | C(═S)N(CH₃)-A-21 |
| 1969. | NCH₃ | C(═S)N(CH₃)-A-22 |
| 1970. | NCH₃ | C(═S)N(CH₃)-A-23 |
| 1971. | NCH₃ | C(═S)N(CH₃)-A-24 |
| 1972. | NCH₃ | C(═S)N(CH₃)-A-25 |
| 1973. | NCH₃ | C(═S)N(CH₃)-A-26 |
| 1974. | NCH₃ | C(═S)N(CH₃)-A-27 |
| 1975. | NCH₃ | C(═S)N(CH₃)-A-28 |
| 1976. | NCH₃ | C(═S)N(CH₃)-A-29 |
| 1977. | NCH₃ | C(═S)N(CH₃)-A-30 |
| 1978. | NCH₃ | C(═S)N(CH₃)-A-31 |
| 1979. | NCH₃ | C(═S)N(CH₃)-A-32 |
| 1980. | NCH₃ | C(═S)N(CH₃)-A-33 |
| 1981. | NCH₃ | C(═S)N(CH₃)—CH₂-A-1 |
| 1982. | NCH₃ | C(═S)N(CH₃)—CH₂-A-2 |
| 1983. | NCH₃ | C(═S)N(CH₃)—CH₂-A-3 |
| 1984. | NCH₃ | C(═S)N(CH₃)—CH₂-A-4 |
| 1985. | NCH₃ | C(═S)N(CH₃)—CH₂-A-5 |
| 1986. | NCH₃ | C(═S)N(CH₃)—CH₂-A-6 |
| 1987. | NCH₃ | C(═S)N(CH₃)—CH₂-A-7 |
| 1988. | NCH₃ | C(═S)N(CH₃)—CH₂-A-8 |
| 1989. | NCH₃ | C(═S)N(CH₃)—CH₂-A-9 |
| 1990. | NCH₃ | C(═S)N(CH₃)—CH₂-A-10 |
| 1991. | NCH₃ | C(═S)N(CH₃)—CH₂-A-11 |
| 1992. | NCH₃ | C(═S)N(CH₃)—CH₂-A-12 |
| 1993. | NCH₃ | C(═S)N(CH₃)—CH₂-A-13 |
| 1994. | NCH₃ | C(═S)N(CH₃)—CH₂-A-14 |
| 1995. | NCH₃ | C(═S)N(CH₃)—CH₂-A-15 |
| 1996. | NCH₃ | C(═S)N(CH₃)—CH₂-A-16 |
| 1997. | NCH₃ | C(═S)N(CH₃)—CH₂-A-17 |
| 1998. | NCH₃ | C(═S)N(CH₃)—CH₂-A-18 |
| 1999. | NCH₃ | C(═S)N(CH₃)—CH₂-A-19 |
| 2000. | NCH₃ | C(═S)N(CH₃)—CH₂-A-20 |
| 2001. | NCH₃ | C(═S)N(CH₃)—CH₂-A-21 |
| 2002. | NCH₃ | C(═S)N(CH₃)—CH₂-A-22 |
| 2003. | NCH₃ | C(═S)N(CH₃)—CH₂-A-23 |
| 2004. | NCH₃ | C(═S)N(CH₃)—CH₂-A-24 |
| 2005. | NCH₃ | C(═S)N(CH₃)—CH₂-A-25 |
| 2006. | NCH₃ | C(═S)N(CH₃)—CH₂-A-26 |
| 2007. | NCH₃ | C(═S)N(CH₃)—CH₂-A-27 |
| 2008. | NCH₃ | C(═S)N(CH₃)—CH₂-A-28 |
| 2009. | NCH₃ | C(═S)N(CH₃)—CH₂-A-29 |
| 2010. | NCH₃ | C(═S)N(CH₃)—CH₂-A-30 |
| 2011. | NCH₃ | C(═S)N(CH₃)—CH₂-A-31 |
| 2012. | NCH₃ | C(═S)N(CH₃)—CH₂-A-32 |
| 2013. | NCH₃ | C(═S)N(CH₃)—CH₂-A-33 |
| 2014. | NCH₃ | C(═S)N(CH₃)—SO₂—CH₃ |
| 2015. | NCH₃ | C(═S)N(CH₃)—SO₂—CF₃ |
| 2016. | NCH₃ | C(═S)N(CH₃)—SO₂—CH₂CH₃ |
| 2017. | NCH₃ | C(═S)N(CH₃)—SO₂—CH₂CF₃ |
| 2018. | NCH₃ | C(═S)N(CH₃)—SO₂—CH₂CH₂CH₃ |
| 2019. | NCH₃ | C(═S)N(CH₃)—SO₂—CH₂CH₂CF₃ |
| 2020. | NCH₃ | C(═S)N(CH₃)—SO₂—CH₂CF₂CF₃ |
| 2021. | NCH₃ | C(═S)N(CH₃)—SO₂—CH(CH₃)₂ |
| 2022. | NCH₃ | C(═S)N(CH₃)—SO₂—CH(CF₃)₂ |
| 2023. | NCH₃ | C(═S)NH—SO₂—NH₂ |
| 2024. | NCH₃ | C(═S)NH—SO₂—NHCH₃ |
| 2025. | NCH₃ | C(═S)NH—SO₂—N(CH₃)₂ |
| 2026. | NCH₃ | C(═S)NH—SO₂—NHCF₃ |
| 2027. | NCH₃ | C(═S)NH—SO₂—N(CF₃)₂ |
| 2028. | NCH₃ | C(═S)NH—SO₂—NHCH₂CH₃ |
| 2029. | NCH₃ | C(═S)NH—SO₂—N(CH₂CH₃)₂ |
| 2030. | NCH₃ | C(═S)NH—SO₂—NHCH₂CF₃ |
| 2031. | NCH₃ | C(═S)NH—SO₂—N(CH₂CF₃)₂ |
| 2032. | NCH₃ | C(═S)NH—SO₂—N(CH₃)CH₂CH₃ |
| 2033. | NCH₃ | C(═S)NH—SO₂—N(CH₃)CH₂CF₃ |
| 2034. | NCH₃ | C(═S)NH—SO₂—N(CF₃)CH₂CH₃ |
| 2035. | NCH₃ | C(═S)NH—SO₂—NHCH₂CH₂CH₃ |
| 2036. | NCH₃ | C(═S)NH—SO₂—N(CH₂CH₂CH₃)₂ |
| 2037. | NCH₃ | C(═S)NH—SO₂—NHCH₂CH₂CF₃ |
| 2038. | NCH₃ | C(═S)NH—SO₂—N(CH₂CH₂CF₃)₂ |
| 2039. | NCH₃ | C(═S)NH—SO₂—N(CH₃)CH₂CH₂CH₃ |
| 2040. | NCH₃ | C(═S)NH—SO₂—N(CH₃)CH₂CH₂CF₃ |
| 2041. | NCH₃ | C(═S)NH—SO₂—N(CF₃)CH₂CH₂CH₃ |
| 2042. | NCH₃ | C(═S)NH—SO₂—NHCH(CH₃)₂ |
| 2043. | NCH₃ | C(═S)NH—SO₂—NHCH(CF₃)₂ |
| 2044. | NCH₃ | C(═S)NH—SO₂—N(CH₃)CH(CH₃)₂ |
| 2045. | NCH₃ | C(═S)NH—SO₂—N(CH₃)CH(CF₃)₂ |
| 2046. | NCH₃ | C(═S)NH—SO₂—N(CF₃)CH(CH₃)₂ |
| 2047. | NCH₃ | C(═S)NH—SO₂—NHCH₂CH₂CH₂CH₃ |
| 2048. | NCH₃ | C(═S)NH—SO₂—N(CH₂CH₂CH₂CH₃)₂ |
| 2049. | NCH₃ | C(═S)NH—SO₂—N(CH₃)CH₂CH₂CH₂CH₃ |
| 2050. | NCH₃ | C(═S)N(CH₃)—SO₂—NH₂ |
| 2051. | NCH₃ | C(═S)N(CH₃)—SO₂—NHCH₃ |
| 2052. | NCH₃ | C(═S)N(CH₃)—SO₂—N(CH₃)₂ |
| 2053. | NCH₃ | C(═S)N(CH₃)—SO₂—NHCF₃ |
| 2054. | NCH₃ | C(═S)N(CH₃)—SO₂—N(CF₃)₂ |
| 2055. | NCH₃ | C(═S)N(CH₃)—SO₂—NHCH₂CH₃ |
| 2056. | NCH₃ | C(═S)N(CH₃)—SO₂—N(CH₂CH₃)₂ |
| 2057. | NCH₃ | C(═S)N(CH₃)—SO₂—NHCH₂CF₃ |
| 2058. | NCH₃ | C(═S)N(CH₃)—SO₂—N(CH₂CF₃)₂ |
| 2059. | NCH₃ | C(═S)N(CH₃)—SO₂—N(CH₃)CH₂CH₃ |
| 2060. | NCH₃ | C(═S)N(CH₃)—SO₂—N(CH₃)CH₂CF₃ |
| 2061. | NCH₃ | C(═S)N(CH₃)—SO₂—N(CF₃)CH₂CH₃ |
| 2062. | NCH₃ | C(═S)N(CH₃)—SO₂—NHCH₂CH₂CH₃ |
| 2063. | NCH₃ | C(═S)N(CH₃)—SO₂—N(CH₂CH₂CH₃)₂ |
| 2064. | NCH₃ | C(═S)N(CH₃)—SO₂—NHCH₂CH₂CF₃ |
| 2065. | NCH₃ | C(═S)N(CH₃)—SO₂—N(CH₂CH₂CF₃)₂ |
| 2066. | NCH₃ | C(═S)N(CH₃)—SO₂—N(CH₃)CH₂CH₂CH₃ |
| 2067. | NCH₃ | C(═S)N(CH₃)—SO₂—N(CH₃)CH₂CH₂CF₃ |
| 2068. | NCH₃ | C(═S)N(CH₃)—SO₂—N(CF₃)CH₂CH₂CH₃ |
| 2069. | NCH₃ | C(═S)N(CH₃)—SO₂—NHCH(CH₃)₂ |
| 2070. | NCH₃ | C(═S)N(CH₃)—SO₂—NHCH(CF₃)₂ |
| 2071. | NCH₃ | C(═S)N(CH₃)—SO₂—N(CH₃)CH(CH₃)₂ |
| 2072. | NCH₃ | C(═S)N(CH₃)—SO₂—N(CH₃)CH(CF₃)₂ |
| 2073. | NCH₃ | C(═S)N(CH₃)—SO₂—N(CF₃)CH(CH₃)₂ |
| 2074. | NCH₃ | C(═S)N(CH₃)—SO₂—NHCH₂CH₂CH₂CH₃ |
| 2075. | NCH₃ | C(═S)N(CH₃)—SO₂—N(CH₂CH₂CH₂CH₃)₂ |
| 2076. | NCH₃ | C(═S)N(CH₃)—SO₂—N(CH₃)CH₂CH₂CH₂CH₃ |
| 2077. | NCH₃ | C(═S)—N═CHOCH₃ |
| 2078. | NCH₃ | C(═S)—N═CHOCH₂CH₃ |
| 2079. | NCH₃ | C(═S)—N═CHOCH₂CH₂CH₃ |
| 2080. | NCH₃ | C(═S)—N═CHOCH(CH₃)₂ |
| 2081. | NCH₃ | C(═S)—N═CHOCF₃ |
| 2082. | NCH₃ | C(═S)—N═CHOCH₂CF₃ |
| 2083. | NCH₃ | C(═S)—N═CHOCH₂CH₂CF₃ |
| 2084. | NCH₃ | C(═S)—N═CHOCH(CF₃)₂ |
| 2085. | NCH₃ | C(═S)—N═CH—CO—OCH₃ |
| 2086. | NCH₃ | C(═S)—N═CH—CO—OCH₂CH₃ |
| 2087. | NCH₃ | C(═S)—N═CH—CO—OCH₂CH₂CH₃ |
| 2088. | NCH₃ | C(═S)—N═CH—CO—OCH(CH₃)₂ |
| 2089. | NCH₃ | C(═S)—N═CH—CO—OCF₃ |
| 2090. | NCH₃ | C(═S)—N═CH—CO—OCH₂CF₃ |
| 2091. | NCH₃ | C(═S)—N═CH—CO—OCH₂CH₂CF₃ |
| 2092. | NCH₃ | C(═S)—N═CH—CO—OCH(CF₃)₂ |
| 2093. | NCH₃ | C(═S)—N═CH—CO—NHCH₃ |
| 2094. | NCH₃ | C(═S)—N═CH—CO—N(CH₃)₂ |
| 2095. | NCH₃ | C(═S)—N═CH—CO—NHCH₂CH₃ |
| 2096. | NCH₃ | C(═S)—N═CH—CO—N(CH₂CH₃)₂ |
| 2097. | NCH₃ | C(═S)—N═CH—CO—N(CH₃)CH₂CH₃ |
| 2098. | NCH₃ | C(═S)—N═CH—CO—NHCH₂CH₂CH₃ |
| 2099. | NCH₃ | C(═S)—N═CH—CO—N(CH₂CH₂CH₃)₂ |
| 2100. | NCH₃ | C(═S)—N═CH—CO—N(CH₃)CH₂CH₂CH₃ |
| 2101. | NCH₃ | C(═S)—N═CH—CO—NHCH(CH₃)₂ |
| 2102. | NCH₃ | C(═S)—N═CH—CO—N(CH₃)CH(CH₃)₂ |
| 2103. | NCH₃ | C(═S)—N═CH—CO—NHCF₃ |
| 2104. | NCH₃ | C(═S)—N═CH—CO—N(CF₃)₂ |
| 2105. | NCH₃ | C(═S)—N═CH—CO—NHCH₂CF₃ |
| 2106. | NCH₃ | C(═S)—N═CH—CO—N(CH₂CF₃)₂ |
| 2107. | NCH₃ | C(═S)—N═CH—CO—N(CH₃)CH₂CF₃ |
| 2108. | NCH₃ | C(═S)—N═CH—CO—N(CF₃)CH₂CF₃ |
| 2109. | NCH₃ | C(═S)—N═CH—CO—NHCH₂CH₂CF₃ |
| 2110. | NCH₃ | C(═S)—N═CH—CO—N(CH₂CH₂CF₃)₂ |
| 2111. | NCH₃ | C(═S)—N═CH—CO—N(CH₃)CH₂CH₂CF₃ |
| 2112. | NCH₃ | C(═S)—N═CH—CO—N(CF₃)CH₂CH₂CH₃ |
| 2113. | NCH₃ | C(═S)—N═CH—CO—NHCH(CF₃)₂ |
| 2114. | NCH₃ | C(═S)—N═CH—CO—N(CH₃)CH(CF₃)₂ |
| 2115. | NCH₃ | C(═S)—N═CH—CO—N(CF₃)CH(CH₃)₂ |
| 2116. | NC(O)CH₃ | C(═O)NH₂ |
| 2117. | NC(O)CH₃ | C(═O)NHCH₃ |
| 2118. | NC(O)CH₃ | C(═O)N(CH₃)₂ |
| 2119. | NC(O)CH₃ | C(═O)NHCF₃ |
| 2120. | NC(O)CH₃ | C(═O)N(CF₃)₂ |
| 2121. | NC(O)CH₃ | C(═O)NHCH₂CH₃ |
| 2122. | NC(O)CH₃ | C(═O)N(CH₂CH₃)₂ |
| 2123. | NC(O)CH₃ | C(═O)N(CH₃)CH₂CH₃ |
| 2124. | NC(O)CH₃ | C(═O)NHCH₂CF₃ |
| 2125. | NC(O)CH₃ | C(═O)N(CH₂CF₃)₂ |
| 2126. | NC(O)CH₃ | C(═O)N(CH₃)CH₂CF₃ |
| 2127. | NC(O)CH₃ | C(═O)NHCH₂CH₂CH₃ |
| 2128. | NC(O)CH₃ | C(═O)N(CH₃)CH₂CH₂CH₃ |
| 2129. | NC(O)CH₃ | C(═O)NHCH(CH₃)₂ |
| 2130. | NC(O)CH₃ | C(═O)NH(CH₂)₃CH₃ |
| 2131. | NC(O)CH₃ | C(═O)N(CH₃)—(CH₂)₃CH₃ |
| 2132. | NC(O)CH₃ | C(═O)N[(CH₂)₃CH₃]₂ |
| 2133. | NC(O)CH₃ | C(═O)N(CH₃)—CH₂—C₆H₅ |
| 2134. | NC(O)CH₃ | C(═O)NH-propargyl |
| 2135. | NC(O)CH₃ | C(═O)N(CH₃)-propargyl |
| 2136. | NC(O)CH₃ | C(═O)NH—CH₂-4-Cl—C₆H₄ |
| 2137. | NC(O)CH₃ | C(═O)N(CH₃)—CH₂-4-Cl—C₆H₄ |
| 2138. | NC(O)CH₃ | C(═O)morpholin-4-yl |
| 2139. | NC(O)CH₃ | C(═O)NH-3-thiolyl-1,1-dioxid |
| 2140. | NC(O)CH₃ | C(═O)N(CH₃)-3-thiolyl-1,1-dioxid |
| 2141. | NC(O)CH₃ | C(═O)-azirid-1-yl |
| 2142. | NC(O)CH₃ | C(═O)-pyrrolidin-1-yl |
| 2143. | NC(O)CH₃ | C(═O)-piperidin-1-yl |
| 2144. | NC(O)CH₃ | C(═O)-thiomorpholin-4-yl |
| 2145. | NC(O)CH₃ | C(═O)NH—CH₂CHF₂ |
| 2146. | NC(O)CH₃ | C(═O)NH—CH₂CH₂CHF₂ |
| 2147. | NC(O)CH₃ | C(═O)NH—CH₂CH₂CF₃ |
| 2148. | NC(O)CH₃ | C(═O)NH-cyclopropyl |
| 2149. | NC(O)CH₃ | C(═O)NH-cyclobutyl |
| 2150. | NC(O)CH₃ | C(═O)NH-cyclopentyl |
| 2151. | NC(O)CH₃ | C(═O)NH-cyclohexyl |
| 2152. | NC(O)CH₃ | C(═O)NH—CH₂-cyclopropyl |
| 2153. | NC(O)CH₃ | C(═O)NH—CH₂-cyclobutyl |
| 2154. | NC(O)CH₃ | C(═O)NH—CH₂-cyclopentyl |
| 2155. | NC(O)CH₃ | C(═O)NH—CH₂-cyclohexyl |
| 2156. | NC(O)CH₃ | C(═O)NH—CN |
| 2157. | NC(O)CH₃ | C(═O)NH—CH₂—CN |
| 2158. | NC(O)CH₃ | C(═O)NH—CH₂—CH═CH₂ |
| 2159. | NC(O)CH₃ | C(═O)NH—CH₂—CH═C(Cl)₂ |
| 2160. | NC(O)CH₃ | C(═O)NH—CH₂—CH═CH-phenyl |
| 2161. | NC(O)CH₃ | C(═O)NH—CH₂—CH═CH-(4-Cl-phenyl) |
| 2162. | NC(O)CH₃ | C(═O)NH—CH₂—SCH₃ |
| 2163. | NC(O)CH₃ | C(═O)NH—CH₂—SCF₃ |
| 2164. | NC(O)CH₃ | C(═O)NH—CH₂—CH₂—SCH₃ |
| 2165. | NC(O)CH₃ | C(═O)NH—CH₂—CH₂—SCF₃ |
| 2166. | NC(O)CH₃ | C(═O)NH—CH₂—SO₂—CH₃ |
| 2167. | NC(O)CH₃ | C(═O)NH—CH₂—SO₂—CF₃ |
| 2168. | NC(O)CH₃ | C(═O)NH—CH₂—CH₂—SO₂—CH₃ |
| 2169. | NC(O)CH₃ | C(═O)NH—CH₂—CH₂—SO₂—CF₃ |
| 2170. | NC(O)CH₃ | C(═O)NH—CH₂—CO—NH₂ |
| 2171. | NC(O)CH₃ | C(═O)NH—CH₂—CO—NHCH₃ |
| 2172. | NC(O)CH₃ | C(═O)NH—CH₂—CO—N(CH₃)₂ |
| 2173. | NC(O)CH₃ | C(═O)NH—CH₂—CO—NHCF₃ |
| 2174. | NC(O)CH₃ | C(═O)NH—CH₂—CO—N(CF₃)₂ |
| 2175. | NC(O)CH₃ | C(═O)NH—CH₂—CO—NHCH₂CH₃ |
| 2176. | NC(O)CH₃ | C(═O)NH—CH₂—CO—N(CH₂CH₃)₂ |
| 2177. | NC(O)CH₃ | C(═O)NH—CH₂—CO—NHCH₂CF₃ |
| 2178. | NC(O)CH₃ | C(═O)NH—CH₂—CO—N(CH₂CF₃)₂ |
| 2179. | NC(O)CH₃ | C(═O)NH—CH₂—CO—NHCH₂CH₂CH₃ |
| 2180. | NC(O)CH₃ | C(═O)NH—CH₂—CO—N(CH₂CH₂CH₃)₂ |
| 2181. | NC(O)CH₃ | C(═O)NH—CH₂—CO—NHCH₂CH₂CF₃ |
| 2182. | NC(O)CH₃ | C(═O)NH—CH₂—CO—N(CH₂CH₂CF₃)₂ |
| 2183. | NC(O)CH₃ | C(═O)NH—CH₂—CO—NHCH(CH₃)₂ |
| 2184. | NC(O)CH₃ | C(═O)NH—CH₂—CO—NHCH(CF₃)₂ |
| 2185. | NC(O)CH₃ | C(═O)NH—CH₂—CO—NH-cyclopropyl |
| 2186. | NC(O)CH₃ | C(═O)NH—CH₂—CO—NH—CH₂-cyclopropyl |
| 2187. | NC(O)CH₃ | C(═O)NH—CH₂—CO—OH |
| 2188. | NC(O)CH₃ | C(═O)NH—CH₂—CO—OCH₃ |
| 2189. | NC(O)CH₃ | C(═O)NH—CH₂—CO—OCF₃ |
| 2190. | NC(O)CH₃ | C(═O)NH—CH₂—CO—OCH₂CH₃ |
| 2191. | NC(O)CH₃ | C(═O)NH—CH₂—CO—OCH₂CF₃ |
| 2192. | NC(O)CH₃ | C(═O)NH—CH₂—CO—OCH₂CH₂CH₃ |
| 2193. | NC(O)CH₃ | C(═O)NH—CH₂—CO—OCH(CH₃)₂ |
| 2194. | NC(O)CH₃ | C(═O)NH—CH₂—CO—OCH₂CH₂CH₂CH₃ |
| 2195. | NC(O)CH₃ | C(═O)NH—CH₂—CO—OCH(CH₃)CH₂CH₃ |
| 2196. | NC(O)CH₃ | C(═O)NH—CH₂—CO—OCH₂CH(CH₃)₂ |
| 2197. | NC(O)CH₃ | C(═O)NH—CH₂—CO—OC(CH₃)₃ |
| 2198. | NC(O)CH₃ | C(═O)NH-A-1 |
| 2199. | NC(O)CH₃ | C(═O)NH-A-2 |
| 2200. | NC(O)CH₃ | C(═O)NH-A-3 |
| 2201. | NC(O)CH₃ | C(═O)NH-A-4 |
| 2202. | NC(O)CH₃ | C(═O)NH-A-5 |
| 2203. | NC(O)CH₃ | C(═O)NH-A-6 |
| 2204. | NC(O)CH₃ | C(═O)NH-A-7 |
| 2205. | NC(O)CH₃ | C(═O)NH-A-8 |
| 2206. | NC(O)CH₃ | C(═O)NH-A-9 |
| 2207. | NC(O)CH₃ | C(═O)NH-A-10 |
| 2208. | NC(O)CH₃ | C(═O)NH-A-11 |
| 2209. | NC(O)CH₃ | C(═O)NH-A-12 |
| 2210. | NC(O)CH₃ | C(═O)NH-A-13 |
| 2211. | NC(O)CH₃ | C(═O)NH-A-14 |
| 2212. | NC(O)CH₃ | C(═O)NH-A-15 |
| 2213. | NC(O)CH₃ | C(═O)NH-A-16 |
| 2214. | NC(O)CH₃ | C(═O)NH-A-17 |
| 2215. | NC(O)CH₃ | C(═O)NH-A-18 |
| 2216. | NC(O)CH₃ | C(═O)NH-A-19 |
| 2217. | NC(O)CH₃ | C(═O)NH-A-20 |
| 2218. | NC(O)CH₃ | C(═O)NH-A-21 |
| 2219. | NC(O)CH₃ | C(═O)NH-A-22 |
| 2220. | NC(O)CH₃ | C(═O)NH-A-23 |
| 2221. | NC(O)CH₃ | C(═O)NH-A-24 |
| 2222. | NC(O)CH₃ | C(═O)NH-A-25 |
| 2223. | NC(O)CH₃ | C(═O)NH-A-26 |
| 2224. | NC(O)CH₃ | C(═O)NH-A-27 |
| 2225. | NC(O)CH₃ | C(═O)NH-A-28 |
| 2226. | NC(O)CH₃ | C(═O)NH-A-29 |
| 2227. | NC(O)CH₃ | C(═O)NH-A-30 |
| 2228. | NC(O)CH₃ | C(═O)NH-A-31 |
| 2229. | NC(O)CH₃ | C(═O)NH-A-32 |
| 2230. | NC(O)CH₃ | C(═O)NH-A-33 |
| 2231. | NC(O)CH₃ | C(═O)NH—CH₂-A-1 |
| 2232. | NC(O)CH₃ | C(═O)NH—CH₂-A-2 |
| 2233. | NC(O)CH₃ | C(═O)NH—CH₂-A-3 |
| 2234. | NC(O)CH₃ | C(═O)NH—CH₂-A-4 |
| 2235. | NC(O)CH₃ | C(═O)NH—CH₂-A-5 |
| 2236. | NC(O)CH₃ | C(═O)NH—CH₂-A-6 |
| 2237. | NC(O)CH₃ | C(═O)NH—CH₂-A-7 |
| 2238. | NC(O)CH₃ | C(═O)NH—CH₂-A-8 |
| 2239. | NC(O)CH₃ | C(═O)NH—CH₂-A-9 |
| 2240. | NC(O)CH₃ | C(═O)NH—CH₂-A-10 |
| 2241. | NC(O)CH₃ | C(═O)NH—CH₂-A-11 |
| 2242. | NC(O)CH₃ | C(═O)NH—CH₂-A-12 |
| 2243. | NC(O)CH₃ | C(═O)NH—CH₂-A-13 |
| 2244. | NC(O)CH₃ | C(═O)NH—CH₂-A-14 |
| 2245. | NC(O)CH₃ | C(═O)NH—CH₂-A-15 |
| 2246. | NC(O)CH₃ | C(═O)NH—CH₂-A-16 |
| 2247. | NC(O)CH₃ | C(═O)NH—CH₂-A-17 |
| 2248. | NC(O)CH₃ | C(═O)NH—CH₂-A-18 |
| 2249. | NC(O)CH₃ | C(═O)NH—CH₂-A-19 |
| 2250. | NC(O)CH₃ | C(═O)NH—CH₂-A-20 |
| 2251. | NC(O)CH₃ | C(═O)NH—CH₂-A-21 |
| 2252. | NC(O)CH₃ | C(═O)NH—CH₂-A-22 |
| 2253. | NC(O)CH₃ | C(═O)NH—CH₂-A-23 |
| 2254. | NC(O)CH₃ | C(═O)NH—CH₂-A-24 |
| 2255. | NC(O)CH₃ | C(═O)NH—CH₂-A-25 |
| 2256. | NC(O)CH₃ | C(═O)NH—CH₂-A-26 |
| 2257. | NC(O)CH₃ | C(═O)NH—CH₂-A-27 |
| 2258. | NC(O)CH₃ | C(═O)NH—CH₂-A-28 |
| 2259. | NC(O)CH₃ | C(═O)NH—CH₂-A-29 |
| 2260. | NC(O)CH₃ | C(═O)NH—CH₂-A-30 |
| 2261. | NC(O)CH₃ | C(═O)NH—CH₂-A-31 |
| 2262. | NC(O)CH₃ | C(═O)NH—CH₂-A-32 |
| 2263. | NC(O)CH₃ | C(═O)NH—CH₂-A-33 |
| 2264. | NC(O)CH₃ | C(═O)NH—SO₂—CH₃ |
| 2265. | NC(O)CH₃ | C(═O)NH—SO₂—CF₃ |
| 2266. | NC(O)CH₃ | C(═O)NH—SO₂—CH₂CH₃ |
| 2267. | NC(O)CH₃ | C(═O)NH—SO₂—CH₂CF₃ |
| 2268. | NC(O)CH₃ | C(═O)NH—SO₂—CH₂CH₂CH₃ |
| 2269. | NC(O)CH₃ | C(═O)NH—SO₂—CH₂CH₂CF₃ |
| 2270. | NC(O)CH₃ | C(═O)NH—SO₂—CH₂CF₂CF₃ |
| 2271. | NC(O)CH₃ | C(═O)NH—SO₂—CH(CH₃)₂ |
| 2272. | NC(O)CH₃ | C(═O)NH—SO₂—CH(CF₃)₂ |
| 2273. | NC(O)CH₃ | C(═O)N(CH₃)—CH₂CHF₂ |
| 2274. | NC(O)CH₃ | C(═O)N(CH₃)—CH₂CH₂CHF₂ |
| 2275. | NC(O)CH₃ | C(═O)N(CH₃)—CH₂CH₂CF₃ |
| 2276. | NC(O)CH₃ | C(═O)N(CH₃)-cyclopropyl |
| 2277. | NC(O)CH₃ | C(═O)N(CH₃)-cyclobutyl |
| 2278. | NC(O)CH₃ | C(═O)N(CH₃)-cyclopentyl |
| 2279. | NC(O)CH₃ | C(═O)N(CH₃)-cyclohexyl |
| 2280. | NC(O)CH₃ | C(═O)N(CH₃)—CH₂-cyclopropyl |
| 2281. | NC(O)CH₃ | C(═O)N(CH₃)—CH₂-cyclobutyl |
| 2282. | NC(O)CH₃ | C(═O)N(CH₃)—CH₂-cyclopentyl |
| 2283. | NC(O)CH₃ | C(═O)N(CH₃)—CH₂-cyclohexyl |
| 2284. | NC(O)CH₃ | C(═O)N(CH₃)—CN |
| 2285. | NC(O)CH₃ | C(═O)N(CH₃)—CH₂—CN |
| 2286. | NC(O)CH₃ | C(═O)N(CH₃)—CH₂—CH═CH₂ |
| 2287. | NC(O)CH₃ | C(═O)N(CH₃)—CH₂—CH═C(Cl)₂ |
| 2288. | NC(O)CH₃ | C(═O)N(CH₃)—CH₂—CH═CH-phenyl |
| 2289. | NC(O)CH₃ | C(═O)N(CH₃)—CH₂—CH═CH-(4-Cl-phenyl) |
| 2290. | NC(O)CH₃ | C(═O)N(CH₃)—CH₂—SCH₃ |
| 2291. | NC(O)CH₃ | C(═O)N(CH₃)—CH₂—SCF₃ |
| 2292. | NC(O)CH₃ | C(═O)N(CH₃)—CH₂—CH₂—SCH₃ |
| 2293. | NC(O)CH₃ | C(═O)N(CH₃)—CH₂—CH₂—SCF₃ |
| 2294. | NC(O)CH₃ | C(═O)N(CH₃)—CH₂—SO₂—CH₃ |
| 2295. | NC(O)CH₃ | C(═O)N(CH₃)—CH₂—SO₂—CF₃ |
| 2296. | NC(O)CH₃ | C(═O)N(CH₃)—CH₂—CH₂—SO₂—CH₃ |
| 2297. | NC(O)CH₃ | C(═O)N(CH₃)—CH₂—CH₂—SO₂—CF₃ |
| 2298. | NC(O)CH₃ | C(═O)N(CH₃)—CH₂—CO—NH₂ |
| 2299. | NC(O)CH₃ | C(═O)N(CH₃)—CH₂—CO—NHCH₃ |
| 2300. | NC(O)CH₃ | C(═O)N(CH₃)—CH₂—CO—N(CH₃)₂ |
| 2301. | NC(O)CH₃ | C(═O)N(CH₃)—CH₂—CO—NHCF₃ |
| 2302. | NC(O)CH₃ | C(═O)N(CH₃)—CH₂—CO—N(CF₃)₂ |
| 2303. | NC(O)CH₃ | C(═O)N(CH₃)—CH₂—CO—NHCH₂CH₃ |
| 2304. | NC(O)CH₃ | C(═O)N(CH₃)—CH₂—CO—N(CH₂CH₃)₂ |
| 2305. | NC(O)CH₃ | C(═O)N(CH₃)—CH₂—CO—NHCH₂CF₃ |
| 2306. | NC(O)CH₃ | C(═O)N(CH₃)—CH₂—CO—N(CH₂CF₃)₂ |
| 2307. | NC(O)CH₃ | C(═O)N(CH₃)—CH₂—CO—NHCH₂CH₂CH₃ |
| 2308. | NC(O)CH₃ | C(═O)N(CH₃)—CH₂—CO—N(CH₂CH₂CH₃)₂ |
| 2309. | NC(O)CH₃ | C(═O)N(CH₃)—CH₂—CO—NHCH₂CH₂CF₃ |
| 2310. | NC(O)CH₃ | C(═O)N(CH₃)—CH₂—CO—N(CH₂CH₂CF₃)₂ |
| 2311. | NC(O)CH₃ | C(═O)N(CH₃)—CH₂—CO—NHCH(CH₃)₂ |
| 2312. | NC(O)CH₃ | C(═O)N(CH₃)—CH₂—CO—NHCH(CF₃)₂ |
| 2313. | NC(O)CH₃ | C(═O)N(CH₃)—CH₂—CO—NH-cyclopropyl |
| 2314. | NC(O)CH₃ | C(═O)N(CH₃)—CH₂—CO—NH—CH₂- |
| | | cyclopropyl |
| 2315. | NC(O)CH₃ | C(═O)N(CH₃)—CH₂—CO—OH |
| 2316. | NC(O)CH₃ | C(═O)N(CH₃)—CH₂—CO—OCH₃ |
| 2317. | NC(O)CH₃ | C(═O)N(CH₃)—CH₂—CO—OCF₃ |
| 2318. | NC(O)CH₃ | C(═O)N(CH₃)—CH₂—CO—OCH₂CH₃ |
| 2319. | NC(O)CH₃ | C(═O)N(CH₃)—CH₂—CO—OCH₂CF₃ |
| 2320. | NC(O)CH₃ | C(═O)N(CH₃)—CH₂—CO—OCH₂CH₂CH₃ |
| 2321. | NC(O)CH₃ | C(═O)N(CH₃)—CH₂—CO—OCH(CH₃)₂ |
| 2322. | NC(O)CH₃ | C(═O)N(CH₃)—CH₂—CO—OCH₂CH₂CH₂CH₃ |
| 2323. | NC(O)CH₃ | C(═O)N(CH₃)—CH₂—CO—OCH(CH₃)CH₂CH₃ |
| 2324. | NC(O)CH₃ | C(═O)N(CH₃)—CH₂—CO—OCH₂CH(CH₃)₂ |
| 2325. | NC(O)CH₃ | C(═O)N(CH₃)—CH₂—CO—OC(CH₃)₃ |
| 2326. | NC(O)CH₃ | C(═O)N(CH₃)-A-1 |
| 2327. | NC(O)CH₃ | C(═O)N(CH₃)-A-2 |
| 2328. | NC(O)CH₃ | C(═O)N(CH₃)-A-3 |
| 2329. | NC(O)CH₃ | C(═O)N(CH₃)-A-4 |
| 2330. | NC(O)CH₃ | C(═O)N(CH₃)-A-5 |
| 2331. | NC(O)CH₃ | C(═O)N(CH₃)-A-6 |
| 2332. | NC(O)CH₃ | C(═O)N(CH₃)-A-7 |
| 2333. | NC(O)CH₃ | C(═O)N(CH₃)-A-8 |
| 2334. | NC(O)CH₃ | C(═O)N(CH₃)-A-9 |
| 2335. | NC(O)CH₃ | C(═O)N(CH₃)-A-10 |
| 2336. | NC(O)CH₃ | C(═O)N(CH₃)-A-11 |
| 2337. | NC(O)CH₃ | C(═O)N(CH₃)-A-12 |
| 2338. | NC(O)CH₃ | C(═O)N(CH₃)-A-13 |
| 2339. | NC(O)CH₃ | C(═O)N(CH₃)-A-14 |
| 2340. | NC(O)CH₃ | C(═O)N(CH₃)-A-15 |
| 2341. | NC(O)CH₃ | C(═O)N(CH₃)-A-16 |
| 2342. | NC(O)CH₃ | C(═O)N(CH₃)-A-17 |
| 2343. | NC(O)CH₃ | C(═O)N(CH₃)-A-18 |
| 2344. | NC(O)CH₃ | C(═O)N(CH₃)-A-19 |
| 2345. | NC(O)CH₃ | C(═O)N(CH₃)-A-20 |
| 2346. | NC(O)CH₃ | C(═O)N(CH₃)-A-21 |
| 2347. | NC(O)CH₃ | C(═O)N(CH₃)-A-22 |
| 2348. | NC(O)CH₃ | C(═O)N(CH₃)-A-23 |
| 2349. | NC(O)CH₃ | C(═O)N(CH₃)-A-24 |
| 2350. | NC(O)CH₃ | C(═O)N(CH₃)-A-25 |
| 2351. | NC(O)CH₃ | C(═O)N(CH₃)-A-26 |
| 2352. | NC(O)CH₃ | C(═O)N(CH₃)-A-27 |
| 2353. | NC(O)CH₃ | C(═O)N(CH₃)-A-28 |
| 2354. | NC(O)CH₃ | C(═O)N(CH₃)-A-29 |
| 2355. | NC(O)CH₃ | C(═O)N(CH₃)-A-30 |
| 2356. | NC(O)CH₃ | C(═O)N(CH₃)-A-31 |
| 2357. | NC(O)CH₃ | C(═O)N(CH₃)-A-32 |
| 2358. | NC(O)CH₃ | C(═O)N(CH₃)-A-33 |
| 2359. | NC(O)CH₃ | C(═O)N(CH₃)—CH₂-A-1 |
| 2360. | NC(O)CH₃ | C(═O)N(CH₃)—CH₂-A-2 |
| 2361. | NC(O)CH₃ | C(═O)N(CH₃)—CH₂-A-3 |
| 2362. | NC(O)CH₃ | C(═O)N(CH₃)—CH₂-A-4 |
| 2363. | NC(O)CH₃ | C(═O)N(CH₃)—CH₂-A-5 |
| 2364. | NC(O)CH₃ | C(═O)N(CH₃)—CH₂-A-6 |
| 2365. | NC(O)CH₃ | C(═O)N(CH₃)—CH₂-A-7 |
| 2366. | NC(O)CH₃ | C(═O)N(CH₃)—CH₂-A-8 |
| 2367. | NC(O)CH₃ | C(═O)N(CH₃)—CH₂-A-9 |
| 2368. | NC(O)CH₃ | C(═O)N(CH₃)—CH₂-A-10 |
| 2369. | NC(O)CH₃ | C(═O)N(CH₃)—CH₂-A-11 |
| 2370. | NC(O)CH₃ | C(═O)N(CH₃)—CH₂-A-12 |
| 2371. | NC(O)CH₃ | C(═O)N(CH₃)—CH₂-A-13 |
| 2372. | NC(O)CH₃ | C(═O)N(CH₃)—CH₂-A-14 |
| 2373. | NC(O)CH₃ | C(═O)N(CH₃)—CH₂-A-15 |
| 2374. | NC(O)CH₃ | C(═O)N(CH₃)—CH₂-A-16 |
| 2375. | NC(O)CH₃ | C(═O)N(CH₃)—CH₂-A-17 |
| 2376. | NC(O)CH₃ | C(═O)N(CH₃)—CH₂-A-18 |
| 2377. | NC(O)CH₃ | C(═O)N(CH₃)—CH₂-A-19 |
| 2378. | NC(O)CH₃ | C(═O)N(CH₃)—CH₂-A-20 |
| 2379. | NC(O)CH₃ | C(═O)N(CH₃)—CH₂-A-21 |
| 2380. | NC(O)CH₃ | C(═O)N(CH₃)—CH₂-A-22 |
| 2381. | NC(O)CH₃ | C(═O)N(CH₃)—CH₂-A-23 |
| 2382. | NC(O)CH₃ | C(═O)N(CH₃)—CH₂-A-24 |
| 2383. | NC(O)CH₃ | C(═O)N(CH₃)—CH₂-A-25 |
| 2384. | NC(O)CH₃ | C(═O)N(CH₃)—CH₂-A-26 |
| 2385. | NC(O)CH₃ | C(═O)N(CH₃)—CH₂-A-27 |
| 2386. | NC(O)CH₃ | C(═O)N(CH₃)—CH₂-A-28 |
| 2387. | NC(O)CH₃ | C(═O)N(CH₃)—CH₂-A-29 |
| 2388. | NC(O)CH₃ | C(═O)N(CH₃)—CH₂-A-30 |
| 2389. | NC(O)CH₃ | C(═O)N(CH₃)—CH₂-A-31 |
| 2390. | NC(O)CH₃ | C(═O)N(CH₃)—CH₂-A-32 |
| 2391. | NC(O)CH₃ | C(═O)N(CH₃)—CH₂-A-33 |
| 2392. | NC(O)CH₃ | C(═O)N(CH₃)—SO₂—CH₃ |
| 2393. | NC(O)CH₃ | C(═O)N(CH₃)—SO₂—CF₃ |
| 2394. | NC(O)CH₃ | C(═O)N(CH₃)—SO₂—CH₂CH₃ |
| 2395. | NC(O)CH₃ | C(═O)N(CH₃)—SO₂—CH₂CF₃ |
| 2396. | NC(O)CH₃ | C(═O)N(CH₃)—SO₂—CH₂CH₂CH₃ |
| 2397. | NC(O)CH₃ | C(═O)N(CH₃)—SO₂—CH₂CH₂CF₃ |
| 2398. | NC(O)CH₃ | C(═O)N(CH₃)—SO₂—CH₂CF₂CF₃ |
| 2399. | NC(O)CH₃ | C(═O)N(CH₃)—SO₂—CH(CH₃)₂ |
| 2400. | NC(O)CH₃ | C(═O)N(CH₃)—SO₂—CH(CF₃)₂ |
| 2401. | NC(O)CH₃ | C(═O)NH—SO₂—NH₂ |
| 2402. | NC(O)CH₃ | C(═O)NH—SO₂—NHCH₃ |
| 2403. | NC(O)CH₃ | C(═O)NH—SO₂—N(CH₃)₂ |
| 2404. | NC(O)CH₃ | C(═O)NH—SO₂—NHCF₃ |
| 2405. | NC(O)CH₃ | C(═O)NH—SO₂—N(CF₃)₂ |
| 2406. | NC(O)CH₃ | C(═O)NH—SO₂—NHCH₂CH₃ |
| 2407. | NC(O)CH₃ | C(═O)NH—SO₂—N(CH₂CH₃)₂ |
| 2408. | NC(O)CH₃ | C(═O)NH—SO₂—NHCH₂CF₃ |
| 2409. | NC(O)CH₃ | C(═O)NH—SO₂—N(CH₂CF₃)₂ |
| 2410. | NC(O)CH₃ | C(═O)NH—SO₂—N(CH₃)CH₂CH₃ |
| 2411. | NC(O)CH₃ | C(═O)NH—SO₂—N(CH₃)CH₂CF₃ |
| 2412. | NC(O)CH₃ | C(═O)NH—SO₂—N(CF₃)CH₂CH₃ |
| 2413. | NC(O)CH₃ | C(═O)NH—SO₂—NHCH₂CH₂CH₃ |
| 2414. | NC(O)CH₃ | C(═O)NH—SO₂—N(CH₂CH₂CH₃)₂ |
| 2415. | NC(O)CH₃ | C(═O)NH—SO₂—NHCH₂CH₂CF₃ |
| 2416. | NC(O)CH₃ | C(═O)NH—SO₂—N(CH₂CH₂CF₃)₂ |
| 2417. | NC(O)CH₃ | C(═O)NH—SO₂—N(CH₃)CH₂CH₂CH₃ |
| 2418. | NC(O)CH₃ | C(═O)NH—SO₂—N(CH₃)CH₂CH₂CF₃ |
| 2419. | NC(O)CH₃ | C(═O)NH—SO₂—N(CF₃)CH₂CH₂CH₃ |
| 2420. | NC(O)CH₃ | C(═O)NH—SO₂—NHCH(CH₃)₂ |
| 2421. | NC(O)CH₃ | C(═O)NH—SO₂—NHCH(CF₃)₂ |
| 2422. | NC(O)CH₃ | C(═O)NH—SO₂—N(CH₃)CH(CH₃)₂ |
| 2423. | NC(O)CH₃ | C(═O)NH—SO₂—N(CH₃)CH(CF₃)₂ |
| 2424. | NC(O)CH₃ | C(═O)NH—SO₂—N(CF₃)CH(CH₃)₂ |
| 2425. | NC(O)CH₃ | C(═O)NH—SO₂—NHCH₂CH₂CH₂CH₃ |
| 2426. | NC(O)CH₃ | C(═O)NH—SO₂—N(CH₂CH₂CH₂CH₃)₂ |
| 2427. | NC(O)CH₃ | C(═O)NH—SO₂—N(CH₃)CH₂CH₂CH₂CH₃ |
| 2428. | NC(O)CH₃ | C(═O)N(CH₃)—SO₂—NH₂ |
| 2429. | NC(O)CH₃ | C(═O)N(CH₃)—SO₂—NHCH₃ |
| 2430. | NC(O)CH₃ | C(═O)N(CH₃)—SO₂—N(CH₃)₂ |
| 2431. | NC(O)CH₃ | C(═O)N(CH₃)—SO₂—NHCF₃ |
| 2432. | NC(O)CH₃ | C(═O)N(CH₃)—SO₂—N(CF₃)₂ |
| 2433. | NC(O)CH₃ | C(═O)N(CH₃)—SO₂—NHCH₂CH₃ |
| 2434. | NC(O)CH₃ | C(═O)N(CH₃)—SO₂—N(CH₂CH₃)₂ |
| 2435. | NC(O)CH₃ | C(═O)N(CH₃)—SO₂—NHCH₂CF₃ |
| 2436. | NC(O)CH₃ | C(═O)N(CH₃)—SO₂—N(CH₂CF₃)₂ |
| 2437. | NC(O)CH₃ | C(═O)N(CH₃)—SO₂—N(CH₃)CH₂CH₃ |
| 2438. | NC(O)CH₃ | C(═O)N(CH₃)—SO₂—N(CH₃)CH₂CF₃ |
| 2439. | NC(O)CH₃ | C(═O)N(CH₃)—SO₂—N(CF₃)CH₂CH₃ |
| 2440. | NC(O)CH₃ | C(═O)N(CH₃)—SO₂—NHCH₂CH₂CH₃ |
| 2441. | NC(O)CH₃ | C(═O)N(CH₃)—SO₂—N(CH₂CH₂CH₃)₂ |
| 2442. | NC(O)CH₃ | C(═O)N(CH₃)—SO₂—NHCH₂CH₂CF₃ |
| 2443. | NC(O)CH₃ | C(═O)N(CH₃)—SO₂—N(CH₂CH₂CF₃)₂ |
| 2444. | NC(O)CH₃ | C(═O)N(CH₃)—SO₂—N(CH₃)CH₂CH₂CH₃ |
| 2445. | NC(O)CH₃ | C(═O)N(CH₃)—SO₂—N(CH₃)CH₂CH₂CF₃ |
| 2446. | NC(O)CH₃ | C(═O)N(CH₃)—SO₂—N(CF₃)CH₂CH₂CH₃ |
| 2447. | NC(O)CH₃ | C(═O)N(CH₃)—SO₂—NHCH(CH₃)₂ |
| 2448. | NC(O)CH₃ | C(═O)N(CH₃)—SO₂—NHCH(CF₃)₂ |
| 2449. | NC(O)CH₃ | C(═O)N(CH₃)—SO₂—N(CH₃)CH(CH₃)₂ |
| 2450. | NC(O)CH₃ | C(═O)N(CH₃)—SO₂—N(CH₃)CH(CF₃)₂ |
| 2451. | NC(O)CH₃ | C(═O)N(CH₃)—SO₂—N(CF₃)CH(CH₃)₂ |
| 2452. | NC(O)CH₃ | C(═O)N(CH₃)—SO₂—NHCH₂CH₂CH₂CH₃ |
| 2453. | NC(O)CH₃ | C(═O)N(CH₃)—SO₂—N(CH₂CH₂CH₂CH₃)₂ |
| 2454. | NC(O)CH₃ | C(═O)N(CH₃)—SO₂—N(CH₃)CH₂CH₂CH₂CH₃ |
| 2455. | NC(O)CH₃ | C(═O)—N═CHOCH₃ |
| 2456. | NC(O)CH₃ | C(═O)—N═CHOCH₂CH₃ |
| 2457. | NC(O)CH₃ | C(═O)—N═CHOCH₂CH₂CH₃ |
| 2458. | NC(O)CH₃ | C(═O)—N═CHOCH(CH₃)₂ |
| 2459. | NC(O)CH₃ | C(═O)—N═CHOCF₃ |
| 2460. | NC(O)CH₃ | C(═O)—N═CHOCH₂CF₃ |
| 2461. | NC(O)CH₃ | C(═O)—N═CHOCH₂CH₂CF₃ |
| 2462. | NC(O)CH₃ | C(═O)—N═CHOCH(CF₃)₂ |
| 2463. | NC(O)CH₃ | C(═O)—N═CH—CO—OCH₃ |
| 2464. | NC(O)CH₃ | C(═O)—N═CH—CO—OCH₂CH₃ |
| 2465. | NC(O)CH₃ | C(═O)—N═CH—CO—OCH₂CH₂CH₃ |
| 2466. | NC(O)CH₃ | C(═O)—N═CH—CO—OCH(CH₃)₂ |
| 2467. | NC(O)CH₃ | C(═O)—N═CH—CO—OCF₃ |
| 2468. | NC(O)CH₃ | C(═O)—N═CH—CO—OCH₂CF₃ |
| 2469. | NC(O)CH₃ | C(═O)—N═CH—CO—OCH₂CH₂CF₃ |
| 2470. | NC(O)CH₃ | C(═O)—N═CH—CO—OCH(CF₃)₂ |
| 2471. | NC(O)CH₃ | C(═O)—N═CH—CO—NHCH₃ |
| 2472. | NC(O)CH₃ | C(═O)—N═CH—CO—N(CH₃)₂ |
| 2473. | NC(O)CH₃ | C(═O)—N═CH—CO—NHCH₂CH₃ |
| 2474. | NC(O)CH₃ | C(═O)—N═CH—CO—N(CH₂CH₃)₂ |
| 2475. | NC(O)CH₃ | C(═O)—N═CH—CO—N(CH₃)CH₂CH₃ |
| 2476. | NC(O)CH₃ | C(═O)—N═CH—CO—NHCH₂CH₂CH₃ |
| 2477. | NC(O)CH₃ | C(═O)—N═CH—CO—N(CH₂CH₂CH₃)₂ |
| 2478. | NC(O)CH₃ | C(═O)—N═CH—CO—N(CH₃)CH₂CH₂CH₃ |
| 2479. | NC(O)CH₃ | C(═O)—N═CH—CO—NHCH(CH₃)₂ |
| 2480. | NC(O)CH₃ | C(═O)—N═CH—CO—N(CH₃)CH(CH₃)₂ |
| 2481. | NC(O)CH₃ | C(═O)—N═CH—CO—NHCF₃ |
| 2482. | NC(O)CH₃ | C(═O)—N═CH—CO—N(CF₃)₂ |
| 2483. | NC(O)CH₃ | C(═O)—N═CH—CO—NHCH₂CF₃ |
| 2484. | NC(O)CH₃ | C(═O)—N═CH—CO—N(CH₂CF₃)₂ |
| 2485. | NC(O)CH₃ | C(═O)—N═CH—CO—N(CH₃)CH₂CF₃ |
| 2486. | NC(O)CH₃ | C(═O)—N═CH—CO—N(CF₃)CH₂CF₃ |
| 2487. | NC(O)CH₃ | C(═O)—N═CH—CO—NHCH₂CH₂CF₃ |
| 2488. | NC(O)CH₃ | C(═O)—N═CH—CO—N(CH₂CH₂CF₃)₂ |
| 2489. | NC(O)CH₃ | C(═O)—N═CH—CO—N(CH₃)CH₂CH₂CF₃ |
| 2490. | NC(O)CH₃ | C(═O)—N═CH—CO—N(CF₃)CH₂CH₂CH₃ |
| 2491. | NC(O)CH₃ | C(═O)—N═CH—CO—NHCH(CF₃)₂ |
| 2492. | NC(O)CH₃ | C(═O)—N═CH—CO—N(CH₃)CH(CF₃)₂ |
| 2493. | NC(O)CH₃ | C(═O)—N═CH—CO—N(CF₃)CH(CH₃)₂ |
| 2494. | NC(O)CH₃ | C(═S)NH₂ |
| 2495. | NC(O)CH₃ | C(═S)NHCH₃ |
| 2496. | NC(O)CH₃ | C(═S)N(CH₃)₂ |
| 2497. | NC(O)CH₃ | C(═S)NHCF₃ |
| 2498. | NC(O)CH₃ | C(═S)N(CF₃)₂ |
| 2499. | NC(O)CH₃ | C(═S)NHCH₂CH₃ |
| 2500. | NC(O)CH₃ | C(═S)N(CH₂CH₃)₂ |
| 2501. | NC(O)CH₃ | C(═S)N(CH₃)CH₂CH₃ |
| 2502. | NC(O)CH₃ | C(═S)NHCH₂CF₃ |
| 2503. | NC(O)CH₃ | C(═S)N(CH₂CF₃)₂ |
| 2504. | NC(O)CH₃ | C(═S)N(CH₃)CH₂CF₃ |
| 2505. | NC(O)CH₃ | C(═S)NHCH₂CH₂CH₃ |
| 2506. | NC(O)CH₃ | C(═S)N(CH₃)CH₂CH₂CH₃ |
| 2507. | NC(O)CH₃ | C(═S)NHCH(CH₃)₂ |
| 2508. | NC(O)CH₃ | C(═S)NH(CH₂)₃CH₃ |
| 2509. | NC(O)CH₃ | C(═S)N(CH₃)—(CH₂)₃CH₃ |
| 2510. | NC(O)CH₃ | C(═S)N[(CH₂)₃CH₃]₂ |
| 2511. | NC(O)CH₃ | C(═S)N(CH₃)—CH₂—C₆H₅ |
| 2512. | NC(O)CH₃ | C(═S)NH-propargyl |
| 2513. | NC(O)CH₃ | C(═S)N(CH₃)-propargyl |
| 2514. | NC(O)CH₃ | C(═S)NH—CH₂-4-Cl—C₆H₄ |
| 2515. | NC(O)CH₃ | C(═S)N(CH₃)—CH₂-4-Cl—C₆H₄ |
| 2516. | NC(O)CH₃ | C(═S)morpholin-4-yl |
| 2517. | NC(O)CH₃ | C(═S)NH-3-thiolyl-1,1-dioxid |
| 2518. | NC(O)CH₃ | C(═S)N(CH₃)-3-thiolyl-1,1-dioxid |
| 2519. | NC(O)CH₃ | C(═S)-azirid-1-yl |
| 2520. | NC(O)CH₃ | C(═S)-pyrrolidin-1-yl |
| 2521. | NC(O)CH₃ | C(═S)-piperidin-1-yl |
| 2522. | NC(O)CH₃ | C(═S)-thiomorpholin-4-yl |
| 2523. | NC(O)CH₃ | C(═S)NH—CH₂CHF₂ |
| 2524. | NC(O)CH₃ | C(═S)NH—CH₂CH₂CHF₂ |
| 2525. | NC(O)CH₃ | C(═S)NH—CH₂CH₂CF₃ |
| 2526. | NC(O)CH₃ | C(═S)NH-cyclopropyl |
| 2527. | NC(O)CH₃ | C(═S)NH-cyclobutyl |
| 2528. | NC(O)CH₃ | C(═S)NH-cyclopentyl |
| 2529. | NC(O)CH₃ | C(═S)NH-cyclohexyl |
| 2530. | NC(O)CH₃ | C(═S)NH—CH₂-cyclopropyl |
| 2531. | NC(O)CH₃ | C(═S)NH—CH₂-cyclobutyl |
| 2532. | NC(O)CH₃ | C(═S)NH—CH₂-cyclopentyl |
| 2533. | NC(O)CH₃ | C(═S)NH—CH₂-cyclohexyl |
| 2534. | NC(O)CH₃ | C(═S)NH—CN |
| 2535. | NC(O)CH₃ | C(═S)NH—CH₂—CN |
| 2536. | NC(O)CH₃ | C(═S)NH—CH₂—CH═CH₂ |
| 2537. | NC(O)CH₃ | C(═S)NH—CH₂—CH═C(Cl)₂ |
| 2538. | NC(O)CH₃ | C(═S)NH—CH₂—CH═CH-phenyl |
| 2539. | NC(O)CH₃ | C(═S)NH—CH₂—CH═CH-(4-Cl-phenyl) |
| 2540. | NC(O)CH₃ | C(═S)NH—CH₂—SCH₃ |
| 2541. | NC(O)CH₃ | C(═S)NH—CH₂—SCF₃ |
| 2542. | NC(O)CH₃ | C(═S)NH—CH₂—CH₂—SCH₃ |
| 2543. | NC(O)CH₃ | C(═S)NH—CH₂—CH₂—SCF₃ |
| 2544. | NC(O)CH₃ | C(═S)NH—CH₂—SO₂—CH₃ |
| 2545. | NC(O)CH₃ | C(═S)NH—CH₂—SO₂—CF₃ |
| 2546. | NC(O)CH₃ | C(═S)NH—CH₂—CH₂—SO₂—CH₃ |
| 2547. | NC(O)CH₃ | C(═S)NH—CH₂—CH₂—SO₂—CF₃ |
| 2548. | NC(O)CH₃ | C(═S)NH—CH₂—CO—NH₂ |
| 2549. | NC(O)CH₃ | C(═S)NH—CH₂—CO—NHCH₃ |
| 2550. | NC(O)CH₃ | C(═S)NH—CH₂—CO—N(CH₃)₂ |
| 2551. | NC(O)CH₃ | C(═S)NH—CH₂—CO—NHCF₃ |
| 2552. | NC(O)CH₃ | C(═S)NH—CH₂—CO—N(CF₃)₂ |
| 2553. | NC(O)CH₃ | C(═S)NH—CH₂—CO—NHCH₂CH₃ |
| 2554. | NC(O)CH₃ | C(═S)NH—CH₂—CO—N(CH₂CH₃)₂ |
| 2555. | NC(O)CH₃ | C(═S)NH—CH₂—CO—NHCH₂CF₃ |
| 2556. | NC(O)CH₃ | C(═S)NH—CH₂—CO—N(CH₂CF₃)₂ |
| 2557. | NC(O)CH₃ | C(═S)NH—CH₂—CO—NHCH₂CH₂CH₃ |
| 2558. | NC(O)CH₃ | C(═S)NH—CH₂—CO—N(CH₂CH₂CH₃)₂ |
| 2559. | NC(O)CH₃ | C(═S)NH—CH₂—CO—NHCH₂CH₂CF₃ |
| 2560. | NC(O)CH₃ | C(═S)NH—CH₂—CO—N(CH₂CH₂CF₃)₂ |
| 2561. | NC(O)CH₃ | C(═S)NH—CH₂—CO—NHCH(CH₃)₂ |
| 2562. | NC(O)CH₃ | C(═S)NH—CH₂—CO—NHCH(CF₃)₂ |
| 2563. | NC(O)CH₃ | C(═S)NH—CH₂—CO—NH-cyclopropyl |
| 2564. | NC(O)CH₃ | C(═S)NH—CH₂—CO—NH—CH₂-cyclopropyl |
| 2565. | NC(O)CH₃ | C(═S)NH—CH₂—CO—OH |
| 2566. | NC(O)CH₃ | C(═S)NH—CH₂—CO—OCH₃ |
| 2567. | NC(O)CH₃ | C(═S)NH—CH₂—CO—OCF₃ |
| 2568. | NC(O)CH₃ | C(═S)NH—CH₂—CO—OCH₂CH₃ |
| 2569. | NC(O)CH₃ | C(═S)NH—CH₂—CO—OCH₂CF₃ |
| 2570. | NC(O)CH₃ | C(═S)NH—CH₂—CO—OCH₂CH₂CH₃ |
| 2571. | NC(O)CH₃ | C(═S)NH—CH₂—CO—OCH(CH₃)₂ |
| 2572. | NC(O)CH₃ | C(═S)NH—CH₂—CO—OCH₂CH₂CH₂CH₃ |
| 2573. | NC(O)CH₃ | C(═S)NH—CH₂—CO—OCH(CH₃)CH₂CH₃ |
| 2574. | NC(O)CH₃ | C(═S)NH—CH₂—CO—OCH₂CH(CH₃)₂ |
| 2575. | NC(O)CH₃ | C(═S)NH—CH₂—CO—OC(CH₃)₃ |
| 2576. | NC(O)CH₃ | C(═S)NH-A-1 |
| 2577. | NC(O)CH₃ | C(═S)NH-A-2 |
| 2578. | NC(O)CH₃ | C(═S)NH-A-3 |
| 2579. | NC(O)CH₃ | C(═S)NH-A-4 |
| 2580. | NC(O)CH₃ | C(═S)NH-A-5 |
| 2581. | NC(O)CH₃ | C(═S)NH-A-6 |
| 2582. | NC(O)CH₃ | C(═S)NH-A-7 |
| 2583. | NC(O)CH₃ | C(═S)NH-A-8 |
| 2584. | NC(O)CH₃ | C(═S)NH-A-9 |
| 2585. | NC(O)CH₃ | C(═S)NH-A-10 |
| 2586. | NC(O)CH₃ | C(═S)NH-A-11 |
| 2587. | NC(O)CH₃ | C(═S)NH-A-12 |
| 2588. | NC(O)CH₃ | C(═S)NH-A-13 |
| 2589. | NC(O)CH₃ | C(═S)NH-A-14 |
| 2590. | NC(O)CH₃ | C(═S)NH-A-15 |
| 2591. | NC(O)CH₃ | C(═S)NH-A-16 |
| 2592. | NC(O)CH₃ | C(═S)NH-A-17 |
| 2593. | NC(O)CH₃ | C(═S)NH-A-18 |
| 2594. | NC(O)CH₃ | C(═S)NH-A-19 |
| 2595. | NC(O)CH₃ | C(═S)NH-A-20 |
| 2596. | NC(O)CH₃ | C(═S)NH-A-21 |
| 2597. | NC(O)CH₃ | C(═S)NH-A-22 |
| 2598. | NC(O)CH₃ | C(═S)NH-A-23 |
| 2599. | NC(O)CH₃ | C(═S)NH-A-24 |
| 2600. | NC(O)CH₃ | C(═S)NH-A-25 |
| 2601. | NC(O)CH₃ | C(═S)NH-A-26 |
| 2602. | NC(O)CH₃ | C(═S)NH-A-27 |
| 2603. | NC(O)CH₃ | C(═S)NH-A-28 |
| 2604. | NC(O)CH₃ | C(═S)NH-A-29 |
| 2605. | NC(O)CH₃ | C(═S)NH-A-30 |
| 2606. | NC(O)CH₃ | C(═S)NH-A-31 |
| 2607. | NC(O)CH₃ | C(═S)NH-A-32 |
| 2608. | NC(O)CH₃ | C(═S)NH-A-33 |
| 2609. | NC(O)CH₃ | C(═S)NH—CH₂-A-1 |
| 2610. | NC(O)CH₃ | C(═S)NH—CH₂-A-2 |
| 2611. | NC(O)CH₃ | C(═S)NH—CH₂-A-3 |
| 2612. | NC(O)CH₃ | C(═S)NH—CH₂-A-4 |
| 2613. | NC(O)CH₃ | C(═S)NH—CH₂-A-5 |
| 2614. | NC(O)CH₃ | C(═S)NH—CH₂-A-6 |
| 2615. | NC(O)CH₃ | C(═S)NH—CH₂-A-7 |
| 2616. | NC(O)CH₃ | C(═S)NH—CH₂-A-8 |
| 2617. | NC(O)CH₃ | C(═S)NH—CH₂-A-9 |
| 2618. | NC(O)CH₃ | C(═S)NH—CH₂-A-10 |
| 2619. | NC(O)CH₃ | C(═S)NH—CH₂-A-11 |
| 2620. | NC(O)CH₃ | C(═S)NH—CH₂-A-12 |
| 2621. | NC(O)CH₃ | C(═S)NH—CH₂-A-13 |
| 2622. | NC(O)CH₃ | C(═S)NH—CH₂-A-14 |
| 2623. | NC(O)CH₃ | C(═S)NH—CH₂-A-15 |
| 2624. | NC(O)CH₃ | C(═S)NH—CH₂-A-16 |
| 2625. | NC(O)CH₃ | C(═S)NH—CH₂-A-17 |
| 2626. | NC(O)CH₃ | C(═S)NH—CH₂-A-18 |
| 2627. | NC(O)CH₃ | C(═S)NH—CH₂-A-19 |
| 2628. | NC(O)CH₃ | C(═S)NH—CH₂-A-20 |
| 2629. | NC(O)CH₃ | C(═S)NH—CH₂-A-21 |
| 2630. | NC(O)CH₃ | C(═S)NH—CH₂-A-22 |
| 2631. | NC(O)CH₃ | C(═S)NH—CH₂-A-23 |
| 2632. | NC(O)CH₃ | C(═S)NH—CH₂-A-24 |
| 2633. | NC(O)CH₃ | C(═S)NH—CH₂-A-25 |
| 2634. | NC(O)CH₃ | C(═S)NH—CH₂-A-26 |
| 2635. | NC(O)CH₃ | C(═S)NH—CH₂-A-27 |
| 2636. | NC(O)CH₃ | C(═S)NH—CH₂-A-28 |
| 2637. | NC(O)CH₃ | C(═S)NH—CH₂-A-29 |
| 2638. | NC(O)CH₃ | C(═S)NH—CH₂-A-30 |
| 2639. | NC(O)CH₃ | C(═S)NH—CH₂-A-31 |
| 2640. | NC(O)CH₃ | C(═S)NH—CH₂-A-32 |
| 2641. | NC(O)CH₃ | C(═S)NH—CH₂-A-33 |
| 2642. | NC(O)CH₃ | C(═S)NH—SO₂—CH₃ |
| 2643. | NC(O)CH₃ | C(═S)NH—SO₂—CF₃ |
| 2644. | NC(O)CH₃ | C(═S)NH—SO₂—CH₂CH₃ |
| 2645. | NC(O)CH₃ | C(═S)NH—SO₂—CH₂CF₃ |
| 2646. | NC(O)CH₃ | C(═S)NH—SO₂—CH₂CH₂CH₃ |
| 2647. | NC(O)CH₃ | C(═S)NH—SO₂—CH₂CH₂CF₃ |
| 2648. | NC(O)CH₃ | C(═S)NH—SO₂—CH₂CF₂CF₃ |
| 2649. | NC(O)CH₃ | C(═S)NH—SO₂—CH(CH₃)₂ |
| 2650. | NC(O)CH₃ | C(═S)NH—SO₂—CH(CF₃)₂ |
| 2651. | NC(O)CH₃ | C(═S)N(CH₃)—CH₂CHF₂ |
| 2652. | NC(O)CH₃ | C(═S)N(CH₃)—CH₂CH₂CHF₂ |
| 2653. | NC(O)CH₃ | C(═S)N(CH₃)—CH₂CH₂CF₃ |
| 2654. | NC(O)CH₃ | C(═S)N(CH₃)-cyclopropyl |
| 2655. | NC(O)CH₃ | C(═S)N(CH₃)-cyclobutyl |
| 2656. | NC(O)CH₃ | C(═S)N(CH₃)-cyclopentyl |
| 2657. | NC(O)CH₃ | C(═S)N(CH₃)-cyclohexyl |
| 2658. | NC(O)CH₃ | C(═S)N(CH₃)—CH₂-cyclopropyl |
| 2659. | NC(O)CH₃ | C(═S)N(CH₃)—CH₂-cyclobutyl |
| 2660. | NC(O)CH₃ | C(═S)N(CH₃)—CH₂-cyclopentyl |
| 2661. | NC(O)CH₃ | C(═S)N(CH₃)—CH₂-cyclohexyl |
| 2662. | NC(O)CH₃ | C(═S)N(CH₃)—CN |
| 2663. | NC(O)CH₃ | C(═S)N(CH₃)—CH₂—CN |
| 2664. | NC(O)CH₃ | C(═S)N(CH₃)—CH₂—CH═CH₂ |
| 2665. | NC(O)CH₃ | C(═S)N(CH₃)—CH₂—CH═C(Cl)₂ |
| 2666. | NC(O)CH₃ | C(═S)N(CH₃)—CH₂—CH═CH-phenyl |
| 2667. | NC(O)CH₃ | C(═S)N(CH₃)—CH₂—CH═CH-(4-Cl-phenyl) |
| 2668. | NC(O)CH₃ | C(═S)N(CH₃)—CH₂—SCH₃ |
| 2669. | NC(O)CH₃ | C(═S)N(CH₃)—CH₂—SCF₃ |
| 2670. | NC(O)CH₃ | C(═S)N(CH₃)—CH₂—CH₂—SCH₃ |
| 2671. | NC(O)CH₃ | C(═S)N(CH₃)—CH₂—CH₂—SCF₃ |
| 2672. | NC(O)CH₃ | C(═S)N(CH₃)—CH₂—SO₂—CH₃ |
| 2673. | NC(O)CH₃ | C(═S)N(CH₃)—CH₂—SO₂—CF₃ |
| 2674. | NC(O)CH₃ | C(═S)N(CH₃)—CH₂—CH₂—SO₂—CH₃ |
| 2675. | NC(O)CH₃ | C(═S)N(CH₃)—CH₂—CH₂—SO₂—CF₃ |
| 2676. | NC(O)CH₃ | C(═S)N(CH₃)—CH₂—CO—NH₂ |
| 2677. | NC(O)CH₃ | C(═S)N(CH₃)—CH₂—CO—NHCH₃ |
| 2678. | NC(O)CH₃ | C(═S)N(CH₃)—CH₂—CO—N(CH₃)₂ |
| 2679. | NC(O)CH₃ | C(═S)N(CH₃)—CH₂—CO—NHCF₃ |
| 2680. | NC(O)CH₃ | C(═S)N(CH₃)—CH₂—CO—N(CF₃)₂ |
| 2681. | NC(O)CH₃ | C(═S)N(CH₃)—CH₂—CO—NHCH₂CH₃ |
| 2682. | NC(O)CH₃ | C(═S)N(CH₃)—CH₂—CO—N(CH₂CH₃)₂ |
| 2683. | NC(O)CH₃ | C(═S)N(CH₃)—CH₂—CO—NHCH₂CF₃ |
| 2684. | NC(O)CH₃ | C(═S)N(CH₃)—CH₂—CO—N(CH₂CF₃)₂ |
| 2685. | NC(O)CH₃ | C(═S)N(CH₃)—CH₂—CO—NHCH₂CH₂CH₃ |
| 2686. | NC(O)CH₃ | C(═S)N(CH₃)—CH₂—CO—N(CH₂CH₂CH₃)₂ |
| 2687. | NC(O)CH₃ | C(═S)N(CH₃)—CH₂—CO—NHCH₂CH₂CF₃ |
| 2688. | NC(O)CH₃ | C(═S)N(CH₃)—CH₂—CO—N(CH₂CH₂CF₃)₂ |
| 2689. | NC(O)CH₃ | C(═S)N(CH₃)—CH₂—CO—NHCH(CH₃)₂ |
| 2690. | NC(O)CH₃ | C(═S)N(CH₃)—CH₂—CO—NHCH(CF₃)₂ |
| 2691. | NC(O)CH₃ | C(═S)N(CH₃)—CH₂—CO—NH-cyclopropyl |
| 2692. | NC(O)CH₃ | C(═S)N(CH₃)—CH₂—CO—NH—CH₂- |
| | | cyclopropyl |
| 2693. | NC(O)CH₃ | C(═S)N(CH₃)—CH₂—CO—OH |
| 2694. | NC(O)CH₃ | C(═S)N(CH₃)—CH₂—CO—OCH₃ |
| 2695. | NC(O)CH₃ | C(═S)N(CH₃)—CH₂—CO—OCF₃ |
| 2696. | NC(O)CH₃ | C(═S)N(CH₃)—CH₂—CO—OCH₂CH₃ |
| 2697. | NC(O)CH₃ | C(═S)N(CH₃)—CH₂—CO—OCH₂CF₃ |
| 2698. | NC(O)CH₃ | C(═S)N(CH₃)—CH₂—CO—OCH₂CH₂CH₃ |
| 2699. | NC(O)CH₃ | C(═S)N(CH₃)—CH₂—CO—OCH(CH₃)₂ |
| 2700. | NC(O)CH₃ | C(═S)N(CH₃)—CH₂—CO—OCH₂CH₂CH₂CH₃ |
| 2701. | NC(O)CH₃ | C(═S)N(CH₃)—CH₂—CO—OCH(CH₃)CH₂CH₃ |
| 2702. | NC(O)CH₃ | C(═S)N(CH₃)—CH₂—CO—OCH₂CH(CH₃)₂ |
| 2703. | NC(O)CH₃ | C(═S)N(CH₃)—CH₂—CO—OC(CH₃)₃ |
| 2704. | NC(O)CH₃ | C(═S)N(CH₃)-A-1 |
| 2705. | NC(O)CH₃ | C(═S)N(CH₃)-A-2 |
| 2706. | NC(O)CH₃ | C(═S)N(CH₃)-A-3 |
| 2707. | NC(O)CH₃ | C(═S)N(CH₃)-A-4 |
| 2708. | NC(O)CH₃ | C(═S)N(CH₃)-A-5 |
| 2709. | NC(O)CH₃ | C(═S)N(CH₃)-A-6 |
| 2710. | NC(O)CH₃ | C(═S)N(CH₃)-A-7 |
| 2711. | NC(O)CH₃ | C(═S)N(CH₃)-A-8 |
| 2712. | NC(O)CH₃ | C(═S)N(CH₃)-A-9 |
| 2713. | NC(O)CH₃ | C(═S)N(CH₃)-A-10 |
| 2714. | NC(O)CH₃ | C(═S)N(CH₃)-A-11 |
| 2715. | NC(O)CH₃ | C(═S)N(CH₃)-A-12 |
| 2716. | NC(O)CH₃ | C(═S)N(CH₃)-A-13 |
| 2717. | NC(O)CH₃ | C(═S)N(CH₃)-A-14 |
| 2718. | NC(O)CH₃ | C(═S)N(CH₃)-A-15 |
| 2719. | NC(O)CH₃ | C(═S)N(CH₃)-A-16 |
| 2720. | NC(O)CH₃ | C(═S)N(CH₃)-A-17 |
| 2721. | NC(O)CH₃ | C(═S)N(CH₃)-A-18 |
| 2722. | NC(O)CH₃ | C(═S)N(CH₃)-A-19 |
| 2723. | NC(O)CH₃ | C(═S)N(CH₃)-A-20 |
| 2724. | NC(O)CH₃ | C(═S)N(CH₃)-A-21 |
| 2725. | NC(O)CH₃ | C(═S)N(CH₃)-A-22 |
| 2726. | NC(O)CH₃ | C(═S)N(CH₃)-A-23 |
| 2727. | NC(O)CH₃ | C(═S)N(CH₃)-A-24 |
| 2728. | NC(O)CH₃ | C(═S)N(CH₃)-A-25 |
| 2729. | NC(O)CH₃ | C(═S)N(CH₃)-A-26 |
| 2730. | NC(O)CH₃ | C(═S)N(CH₃)-A-27 |
| 2731. | NC(O)CH₃ | C(═S)N(CH₃)-A-28 |
| 2732. | NC(O)CH₃ | C(═S)N(CH₃)-A-29 |
| 2733. | NC(O)CH₃ | C(═S)N(CH₃)-A-30 |
| 2734. | NC(O)CH₃ | C(═S)N(CH₃)-A-31 |
| 2735. | NC(O)CH₃ | C(═S)N(CH₃)-A-32 |
| 2736. | NC(O)CH₃ | C(═S)N(CH₃)-A-33 |
| 2737. | NC(O)CH₃ | C(═S)N(CH₃)—CH₂-A-1 |
| 2738. | NC(O)CH₃ | C(═S)N(CH₃)—CH₂-A-2 |
| 2739. | NC(O)CH₃ | C(═S)N(CH₃)—CH₂-A-3 |
| 2740. | NC(O)CH₃ | C(═S)N(CH₃)—CH₂-A-4 |
| 2741. | NC(O)CH₃ | C(═S)N(CH₃)—CH₂-A-5 |
| 2742. | NC(O)CH₃ | C(═S)N(CH₃)—CH₂-A-6 |
| 2743. | NC(O)CH₃ | C(═S)N(CH₃)—CH₂-A-7 |
| 2744. | NC(O)CH₃ | C(═S)N(CH₃)—CH₂-A-8 |
| 2745. | NC(O)CH₃ | C(═S)N(CH₃)—CH₂-A-9 |
| 2746. | NC(O)CH₃ | C(═S)N(CH₃)—CH₂-A-10 |
| 2747. | NC(O)CH₃ | C(═S)N(CH₃)—CH₂-A-11 |
| 2748. | NC(O)CH₃ | C(═S)N(CH₃)—CH₂-A-12 |
| 2749. | NC(O)CH₃ | C(═S)N(CH₃)—CH₂-A-13 |
| 2750. | NC(O)CH₃ | C(═S)N(CH₃)—CH₂-A-14 |
| 2751. | NC(O)CH₃ | C(═S)N(CH₃)—CH₂-A-15 |
| 2752. | NC(O)CH₃ | C(═S)N(CH₃)—CH₂-A-16 |
| 2753. | NC(O)CH₃ | C(═S)N(CH₃)—CH₂-A-17 |
| 2754. | NC(O)CH₃ | C(═S)N(CH₃)—CH₂-A-18 |
| 2755. | NC(O)CH₃ | C(═S)N(CH₃)—CH₂-A-19 |
| 2756. | NC(O)CH₃ | C(═S)N(CH₃)—CH₂-A-20 |
| 2757. | NC(O)CH₃ | C(═S)N(CH₃)—CH₂-A-21 |
| 2758. | NC(O)CH₃ | C(═S)N(CH₃)—CH₂-A-22 |
| 2759. | NC(O)CH₃ | C(═S)N(CH₃)—CH₂-A-23 |
| 2760. | NC(O)CH₃ | C(═S)N(CH₃)—CH₂-A-24 |
| 2761. | NC(O)CH₃ | C(═S)N(CH₃)—CH₂-A-25 |
| 2762. | NC(O)CH₃ | C(═S)N(CH₃)—CH₂-A-26 |
| 2763. | NC(O)CH₃ | C(═S)N(CH₃)—CH₂-A-27 |
| 2764. | NC(O)CH₃ | C(═S)N(CH₃)—CH₂-A-28 |
| 2765. | NC(O)CH₃ | C(═S)N(CH₃)—CH₂-A-29 |
| 2766. | NC(O)CH₃ | C(═S)N(CH₃)—CH₂-A-30 |
| 2767. | NC(O)CH₃ | C(═S)N(CH₃)—CH₂-A-31 |
| 2768. | NC(O)CH₃ | C(═S)N(CH₃)—CH₂-A-32 |
| 2769. | NC(O)CH₃ | C(═S)N(CH₃)—CH₂-A-33 |
| 2770. | NC(O)CH₃ | C(═S)N(CH₃)—SO₂—CH₃ |
| 2771. | NC(O)CH₃ | C(═S)N(CH₃)—SO₂—CF₃ |
| 2772. | NC(O)CH₃ | C(═S)N(CH₃)—SO₂—CH₂CH₃ |
| 2773. | NC(O)CH₃ | C(═S)N(CH₃)—SO₂—CH₂CF₃ |
| 2774. | NC(O)CH₃ | C(═S)N(CH₃)—SO₂—CH₂CH₂CH₃ |
| 2775. | NC(O)CH₃ | C(═S)N(CH₃)—SO₂—CH₂CH₂CF₃ |
| 2776. | NC(O)CH₃ | C(═S)N(CH₃)—SO₂—CH₂CF₂CF₃ |
| 2777. | NC(O)CH₃ | C(═S)N(CH₃)—SO₂—CH(CH₃)₂ |
| 2778. | NC(O)CH₃ | C(═S)N(CH₃)—SO₂—CH(CF₃)₂ |
| 2779. | NC(O)CH₃ | C(═S)NH—SO₂—NH₂ |
| 2780. | NC(O)CH₃ | C(═S)NH—SO₂—NHCH₃ |
| 2781. | NC(O)CH₃ | C(═S)NH—SO₂—N(CH₃)₂ |
| 2782. | NC(O)CH₃ | C(═S)NH—SO₂—NHCF₃ |
| 2783. | NC(O)CH₃ | C(═S)NH—SO₂—N(CF₃)₂ |
| 2784. | NC(O)CH₃ | C(═S)NH—SO₂—NHCH₂CH₃ |
| 2785. | NC(O)CH₃ | C(═S)NH—SO₂—N(CH₂CH₃)₂ |
| 2786. | NC(O)CH₃ | C(═S)NH—SO₂—NHCH₂CF₃ |
| 2787. | NC(O)CH₃ | C(═S)NH—SO₂—N(CH₂CF₃)₂ |
| 2788. | NC(O)CH₃ | C(═S)NH—SO₂—N(CH₃)CH₂CH₃ |
| 2789. | NC(O)CH₃ | C(═S)NH—SO₂—N(CH₃)CH₂CF₃ |
| 2790. | NC(O)CH₃ | C(═S)NH—SO₂—N(CF₃)CH₂CH₃ |
| 2791. | NC(O)CH₃ | C(═S)NH—SO₂—NHCH₂CH₂CH₃ |
| 2792. | NC(O)CH₃ | C(═S)NH—SO₂—N(CH₂CH₂CH₃)₂ |
| 2793. | NC(O)CH₃ | C(═S)NH—SO₂—NHCH₂CH₂CF₃ |
| 2794. | NC(O)CH₃ | C(═S)NH—SO₂—N(CH₂CH₂CF₃)₂ |
| 2795. | NC(O)CH₃ | C(═S)NH—SO₂—N(CH₃)CH₂CH₂CH₃ |
| 2796. | NC(O)CH₃ | C(═S)NH—SO₂—N(CH₃)CH₂CH₂CF₃ |
| 2797. | NC(O)CH₃ | C(═S)NH—SO₂—N(CF₃)CH₂CH₂CH₃ |
| 2798. | NC(O)CH₃ | C(═S)NH—SO₂—NHCH(CH₃)₂ |
| 2799. | NC(O)CH₃ | C(═S)NH—SO₂—NHCH(CF₃)₂ |
| 2800. | NC(O)CH₃ | C(═S)NH—SO₂—N(CH₃)CH(CH₃)₂ |
| 2801. | NC(O)CH₃ | C(═S)NH—SO₂—N(CH₃)CH(CF₃)₂ |
| 2802. | NC(O)CH₃ | C(═S)NH—SO₂—N(CF₃)CH(CH₃)₂ |
| 2803. | NC(O)CH₃ | C(═S)NH—SO₂—NHCH₂CH₂CH₂CH₃ |
| 2804. | NC(O)CH₃ | C(═S)NH—SO₂—N(CH₂CH₂CH₂CH₃)₂ |
| 2805. | NC(O)CH₃ | C(═S)NH—SO₂—N(CH₃)CH₂CH₂CH₂CH₃ |
| 2806. | NC(O)CH₃ | C(═S)N(CH₃)—SO₂—NH₂ |
| 2807. | NC(O)CH₃ | C(═S)N(CH₃)—SO₂—NHCH₃ |
| 2808. | NC(O)CH₃ | C(═S)N(CH₃)—SO₂—N(CH₃)₂ |
| 2809. | NC(O)CH₃ | C(═S)N(CH₃)—SO₂—NHCF₃ |
| 2810. | NC(O)CH₃ | C(═S)N(CH₃)—SO₂—N(CF₃)₂ |
| 2811. | NC(O)CH₃ | C(═S)N(CH₃)—SO₂—NHCH₂CH₃ |
| 2812. | NC(O)CH₃ | C(═S)N(CH₃)—SO₂—N(CH₂CH₃)₂ |
| 2813. | NC(O)CH₃ | C(═S)N(CH₃)—SO₂—NHCH₂CF₃ |
| 2814. | NC(O)CH₃ | C(═S)N(CH₃)—SO₂—N(CH₂CF₃)₂ |
| 2815. | NC(O)CH₃ | C(═S)N(CH₃)—SO₂—N(CH₃)CH₂CH₃ |
| 2816. | NC(O)CH₃ | C(═S)N(CH₃)—SO₂—N(CH₃)CH₂CF₃ |
| 2817. | NC(O)CH₃ | C(═S)N(CH₃)—SO₂—N(CF₃)CH₂CH₃ |
| 2818. | NC(O)CH₃ | C(═S)N(CH₃)—SO₂—NHCH₂CH₂CH₃ |
| 2819. | NC(O)CH₃ | C(═S)N(CH₃)—SO₂—N(CH₂CH₂CH₃)₂ |
| 2820. | NC(O)CH₃ | C(═S)N(CH₃)—SO₂—NHCH₂CH₂CF₃ |
| 2821. | NC(O)CH₃ | C(═S)N(CH₃)—SO₂—N(CH₂CH₂CF₃)₂ |
| 2822. | NC(O)CH₃ | C(═S)N(CH₃)—SO₂—N(CH₃)CH₂CH₂CH₃ |
| 2823. | NC(O)CH₃ | C(═S)N(CH₃)—SO₂—N(CH₃)CH₂CH₂CF₃ |
| 2824. | NC(O)CH₃ | C(═S)N(CH₃)—SO₂—N(CF₃)CH₂CH₂CH₃ |
| 2825. | NC(O)CH₃ | C(═S)N(CH₃)—SO₂—NHCH(CH₃)₂ |
| 2826. | NC(O)CH₃ | C(═S)N(CH₃)—SO₂—NHCH(CF₃)₂ |
| 2827. | NC(O)CH₃ | C(═S)N(CH₃)—SO₂—N(CH₃)CH(CH₃)₂ |
| 2828. | NC(O)CH₃ | C(═S)N(CH₃)—SO₂—N(CH₃)CH(CF₃)₂ |
| 2829. | NC(O)CH₃ | C(═S)N(CH₃)—SO₂—N(CF₃)CH(CH₃)₂ |
| 2830. | NC(O)CH₃ | C(═S)N(CH₃)—SO₂—NHCH₂CH₂CH₂CH₃ |
| 2831. | NC(O)CH₃ | C(═S)N(CH₃)—SO₂—N(CH₂CH₂CH₂CH₃)₂ |
| 2832. | NC(O)CH₃ | C(═S)N(CH₃)—SO₂—N(CH₃)CH₂CH₂CH₂CH₃ |
| 2833. | NC(O)CH₃ | C(═S)—N═CHOCH₃ |
| 2834. | NC(O)CH₃ | C(═S)—N═CHOCH₂CH₃ |
| 2835. | NC(O)CH₃ | C(═S)—N═CHOCH₂CH₂CH₃ |
| 2836. | NC(O)CH₃ | C(═S)—N═CHOCH(CH₃)₂ |
| 2837. | NC(O)CH₃ | C(═S)—N═CHOCF₃ |
| 2838. | NC(O)CH₃ | C(═S)—N═CHOCH₂CF₃ |
| 2839. | NC(O)CH₃ | C(═S)—N═CHOCH₂CH₂CF₃ |
| 2840. | NC(O)CH₃ | C(═S)—N═CHOCH(CF₃)₂ |
| 2841. | NC(O)CH₃ | C(═S)—N═CH—CO—OCH₃ |
| 2842. | NC(O)CH₃ | C(═S)—N═CH—CO—OCH₂CH₃ |
| 2843. | NC(O)CH₃ | C(═S)—N═CH—CO—OCH₂CH₂CH₃ |
| 2844. | NC(O)CH₃ | C(═S)—N═CH—CO—OCH(CH₃)₂ |
| 2845. | NC(O)CH₃ | C(═S)—N═CH—CO—OCF₃ |
| 2846. | NC(O)CH₃ | C(═S)—N═CH—CO—OCH₂CF₃ |
| 2847. | NC(O)CH₃ | C(═S)—N═CH—CO—OCH₂CH₂CF₃ |
| 2848. | NC(O)CH₃ | C(═S)—N═CH—CO—OCH(CF₃)₂ |
| 2849. | NC(O)CH₃ | C(═S)—N═CH—CO—NHCH₃ |
| 2850. | NC(O)CH₃ | C(═S)—N═CH—CO—N(CH₃)₂ |
| 2851. | NC(O)CH₃ | C(═S)—N═CH—CO—NHCH₂CH₃ |
| 2852. | NC(O)CH₃ | C(═S)—N═CH—CO—N(CH₂CH₃)₂ |
| 2853. | NC(O)CH₃ | C(═S)—N═CH—CO—N(CH₃)CH₂CH₃ |
| 2854. | NC(O)CH₃ | C(═S)—N═CH—CO—NHCH₂CH₂CH₃ |
| 2855. | NC(O)CH₃ | C(═S)—N═CH—CO—N(CH₂CH₂CH₃)₂ |
| 2856. | NC(O)CH₃ | C(═S)—N═CH—CO—N(CH₃)CH₂CH₂CH₃ |
| 2857. | NC(O)CH₃ | C(═S)—N═CH—CO—NHCH(CH₃)₂ |
| 2858. | NC(O)CH₃ | C(═S)—N═CH—CO—N(CH₃)CH(CH₃)₂ |
| 2859. | NC(O)CH₃ | C(═S)—N═CH—CO—NHCF₃ |
| 2860. | NC(O)CH₃ | C(═S)—N═CH—CO—N(CF₃)₂ |
| 2861. | NC(O)CH₃ | C(═S)—N═CH—CO—NHCH₂CF₃ |
| 2862. | NC(O)CH₃ | C(═S)—N═CH—CO—N(CH₂CF₃)₂ |
| 2863. | NC(O)CH₃ | C(═S)—N═CH—CO—N(CH₃)CH₂CF₃ |
| 2864. | NC(O)CH₃ | C(═S)—N═CH—CO—N(CF₃)CH₂CF₃ |
| 2865. | NC(O)CH₃ | C(═S)—N═CH—CO—NHCH₂CH₂CF₃ |
| 2866. | NC(O)CH₃ | C(═S)—N═CH—CO—N(CH₂CH₂CF₃)₂ |
| 2867. | NC(O)CH₃ | C(═S)—N═CH—CO—N(CH₃)CH₂CH₂CF₃ |
| 2868. | NC(O)CH₃ | C(═S)—N═CH—CO—N(CF₃)CH₂CH₂CH₃ |
| 2869. | NC(O)CH₃ | C(═S)—N═CH—CO—NHCH(CF₃)₂ |
| 2870. | NC(O)CH₃ | C(═S)—N═CH—CO—N(CH₃)CH(CF₃)₂ |
| 2871. | NC(O)CH₃ | C(═S)—N═CH—CO—N(CF₃)CH(CH₃)₂ |

^{c}propyl = cyclopropyl

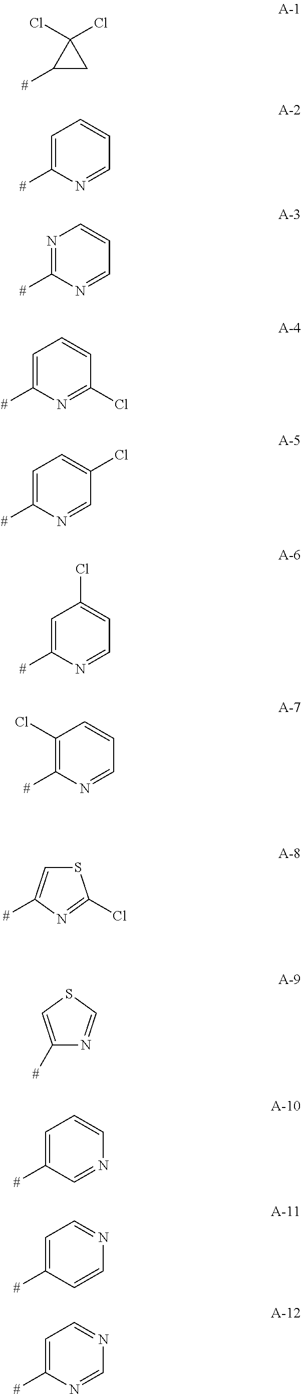

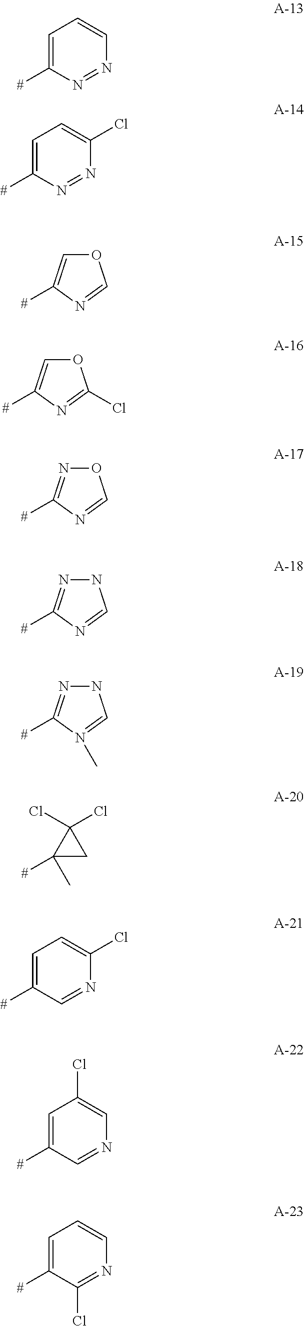

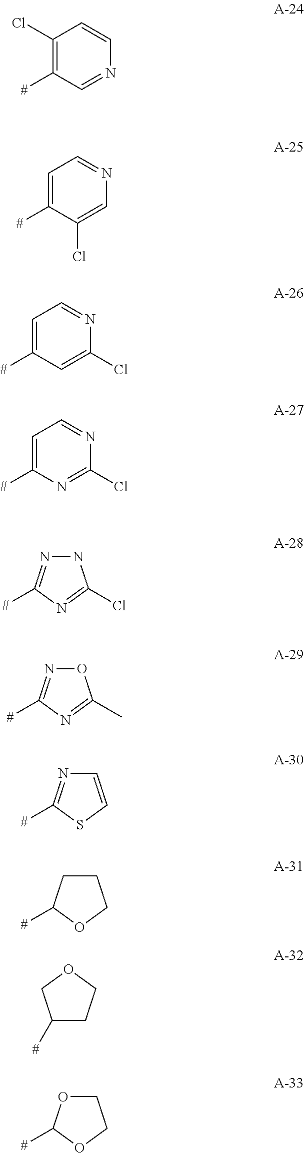

Among the above compounds of formulae I.1 to I.66, preference is given to compounds of formulae I.2 and I.8. Moreover, among the above compounds of formulae I.1 to I.66, preference is given to compounds wherein G is a group G-13.1, G-13.2, G-13.3, G-13.4, G-14.1 or G-16.1 and especially G-14.1. Specific preference is given to compounds of formulae I.2 and 1.8 wherein G is a group G-13.1, G-13.2, G-13.3, G-13.4, G-14.1 or G-16.1 and especially G-14.1.

Compounds of formula I can be prepared by one or more of the following methods and variations as described in schemes 1 to 27. The variables A¹, B¹, X, Y, R¹, R², R⁴, R⁵, p and q are as defined above for formula I.

Compounds of formula I-a (compounds of formula I wherein G=G-3 or G-4) can be prepared by reaction of a aminoalcohol or aminothiol of formula 2 (W=O or S) with an acid halogenide of formula 3 (Z′=halogen, O—C(═O)-alkyl) as outlined in scheme 1. An analogous process has been described, for example, by Clayden et al, Angewandte Chemie, International Edition (2008), 47(27), 5060-5061.

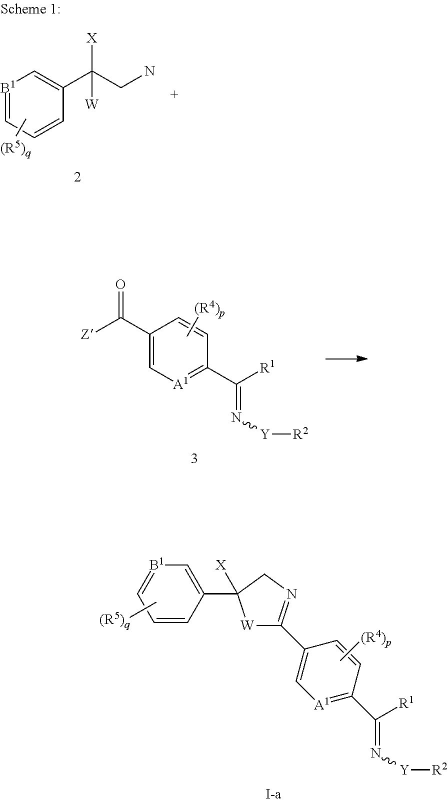

Compounds of formula I-b (compounds of formula I wherein G=G-8) can be prepared as outlined in scheme 2 by a cyclization reaction of carboxylic thioethers of formula 4, as for example described by Mock et al, Synthetic Communications (1988), 18(8), 769-76. In such a reaction process, the carboxylic acid is converted into the corresponding carboxylic amide, followed by oxidation with N-chloro succinimide.

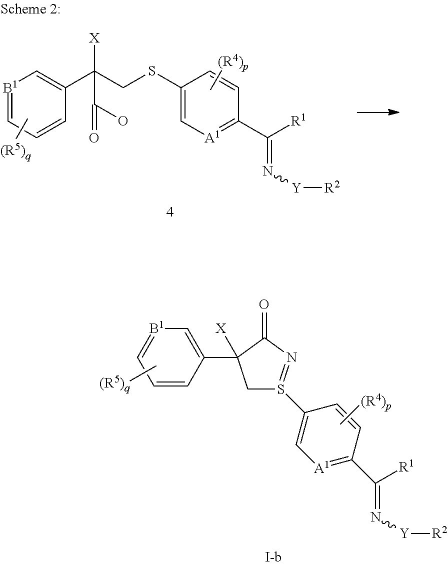

Compounds of formula I-c (compounds of formula I wherein G=G-6 or oxidized form thereof) can be prepared as outlined in scheme 3 by cyclization of compounds of formula 5, as described for example by Wasylenko et al, Journal of the American Chemical Society (2006), 128(40), 13142-13150. Subsequent reduction with diphenyl sulfide, as described for example by Fischer et al., Sulfur Letters (1987), 6(6), 191-196 yields I-c.

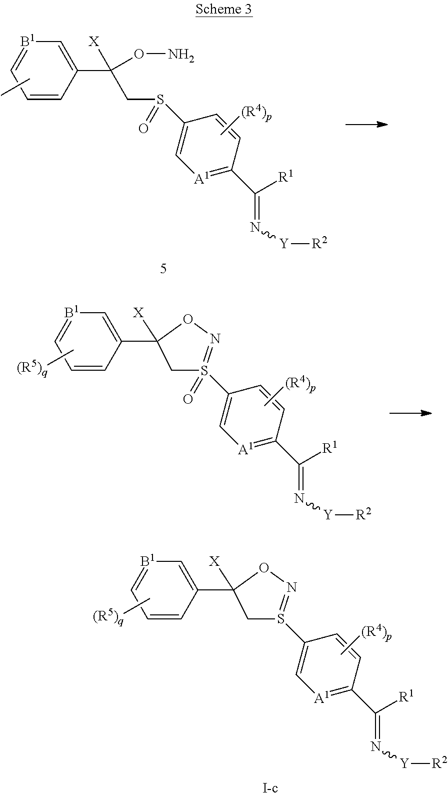

Compounds of formula I can also be prepared as outlined in scheme 4 by condensation of a hydroxamic acid derivative 6 with a Grignard reagent or an organolithium compound as described for example by Reutrakul et al, e-EROS Encyclopedia of Reagents for Organic Synthesis, 2001, Wiley, Chichester, UK for the oximes and by Danko et al, Pest Management Science, 2006, 62, 229-236 for the hydrazones (Z may be a leaving group like halogen, OR″ or SR″). The derived ketoxime 7 is then converted into compounds of formula I by reaction with an alkylating agent as for example described by Huang et al, J. Org. Chem. 2008, 73, 4017-4026.

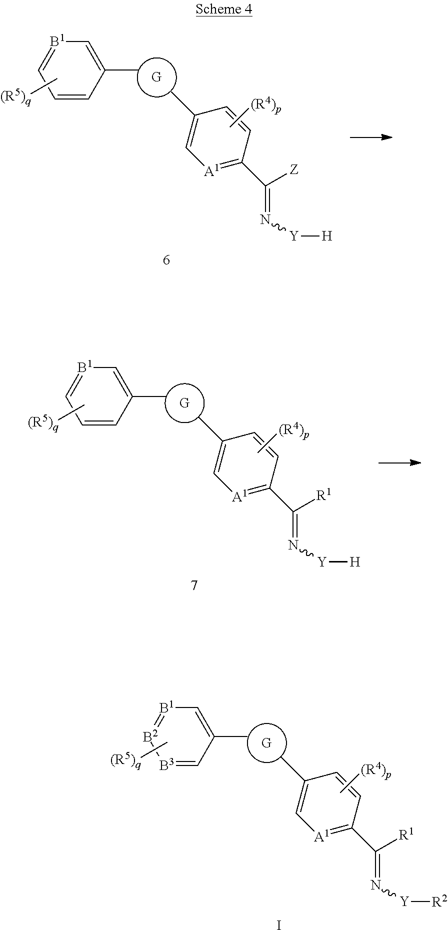

Compounds of formula I can also be prepared as outlined in scheme 5 by reaction of an hydrazone 8 with a formylating agent to yield hydrazone 9, as described for example by Brehme et al, Zeitschrift f. Chemie, 1968, 8, 226-227.

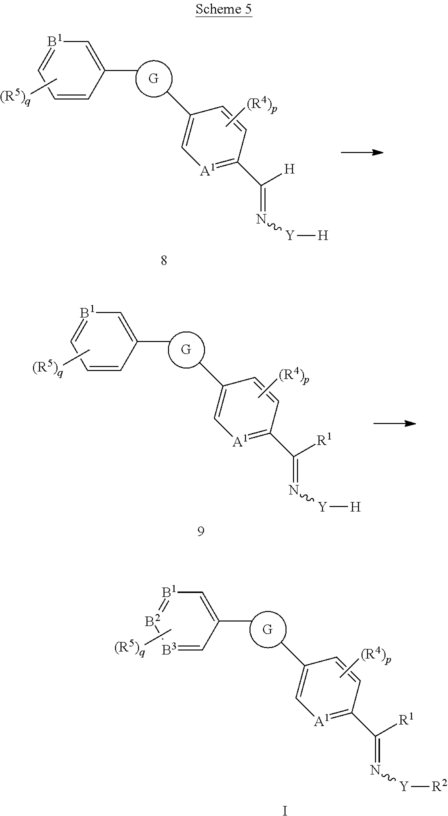

Compounds of formula I can also be prepared as outlined in scheme 6 by reaction of an aldehyde or ketone 10 with a hydroxylamine derivative, as described for example in WO 2006/135763. Alternatively, compounds of formula I can also be prepared by reaction of an aldehyde or ketone 10 with a hydrazine derivative as described for example by Fattorusso et al, J. Med. Chem. 2008, 51, 1333-1343. Compounds of formula 10 can be prepared as described for example by Mihara et al, WO 2008/122375.

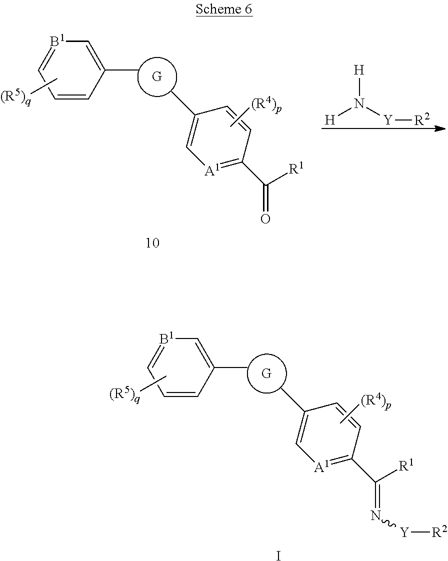

Compounds of formula I can also be prepared as outlined in scheme 7 by reaction of an organolithium reagent or a Grignard reagent 11 with an electrophile as described for example by Fujisawa et al, Chem. Lett. 1983, 51, 1537-1540 for nitro compounds as electrophile or in WO 95/20569 for hydroxamic acid derivatives. Hydrazone compounds of formula I can also be prepared as described for example by Benomar et al, J. Fluorine Chem. 1990, 50, 207-215 (J may be a metal, as for example Li, Na, K or MgX, SnX3; Z may be a leaving group like halogen, OR″ or SR″)

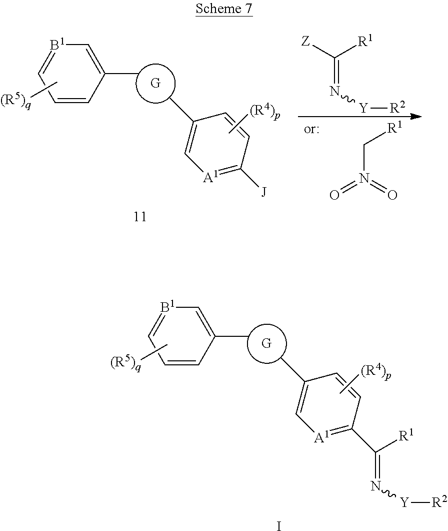

Compounds of formula I can also be prepared as outlined in scheme 8 by reaction of a boronic acid 12 with an electrophile (e.g. a hydroxamic acid chloride) as described for example by Dolliver et al, Can. J. Chem. 2007, 85, 913-922 (M is a boronic acid derivative; Z may be a leaving group like halogen, OR″ or SR″). Compounds of formula 12 can be prepared as described for example in WO 2005/085216.

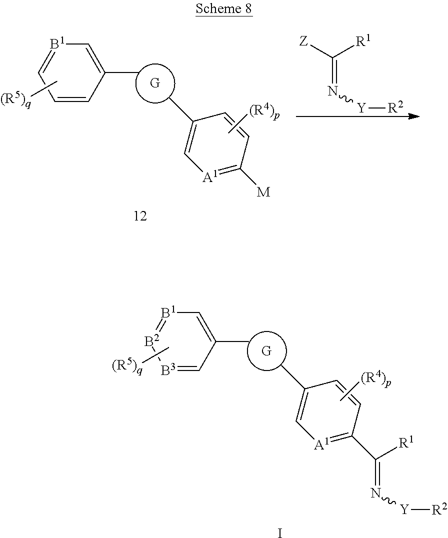

Compounds of formula I can also be prepared as outlined in scheme 9 by reaction of an olefin of formula 13 with a nitrite as described for example by Sugamoto et al, Synlett, 1998, 1270-1272.

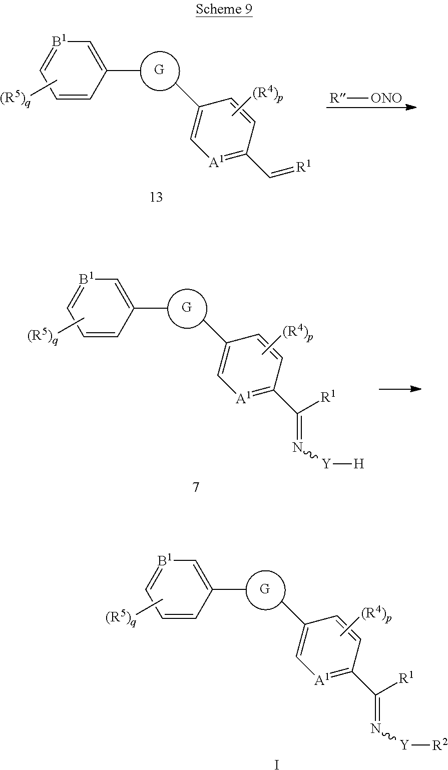

Compounds of formula I-d (compounds of formula I wherein G=G-14) can be prepared as outlined in scheme 10 by reaction of 3-arylpyrrolidine of formula 14 in a Buchwald Hartwig amination with a compound of formula 15, as described for example in WO 2008/128711.

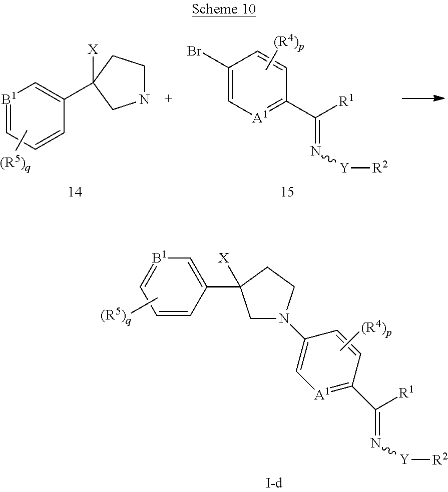

Compounds of formula 10 can be prepared as outlined in scheme 11 by reaction of an organometallic compound of formula 16 with a carboxylic acid derivative (Q may be a metal as for example ZnE, MgE, Li, Na, K, SnE3; with E being a leaving group such as halogen, or OR¹⁶ or S(O)ₙR¹⁶ and wherein n is 0-2); as described e.g. in WO 2008/156721 or by Dieter et al, Tetrahedron (2003), 59(7), 1083-1094. Compounds of formula 10 can also be prepared from secondary alcohols of formula 18 by oxidation, as described for example in US 2007265321. Compounds of formula 18 can be prepared by reaction of compounds of formula 16 with an aldehyde, as described for example by Yamagishi et al, Journal of Organic Chemistry (2009), 74(16), 6350-6353.

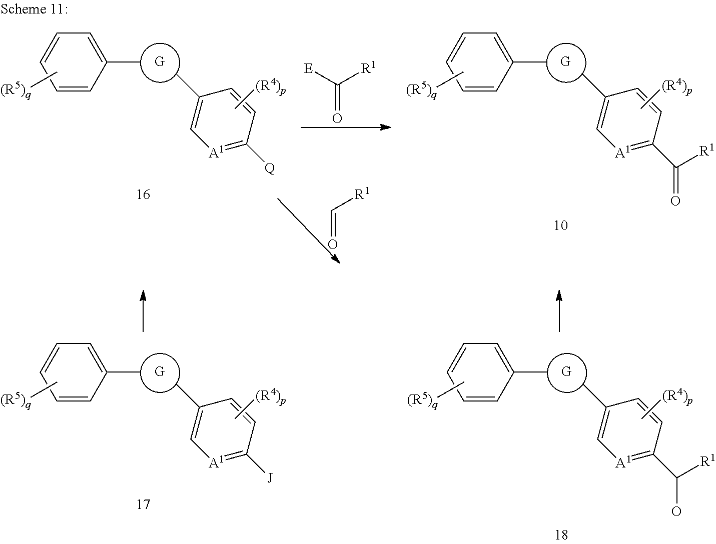

The corresponding metal organyls of formula 16 can be prepared by a halogen-metal exchange reaction of halides of formula 17. The corresponding halides of formula 17 can be prepared as described for example in US 2007066617 or in unpublished PCT/EP2010/055773 (J may be a halogen as for example CI, Br. I):

Compounds of formula 10 can also be prepared as outlined in scheme 12 by acidic hydrolysis of compounds of formula 19 [Z in this case equals R¹], as for example described by Singh et al, European Journal of Organic Chemistry (2008), (32), 5446-5460.

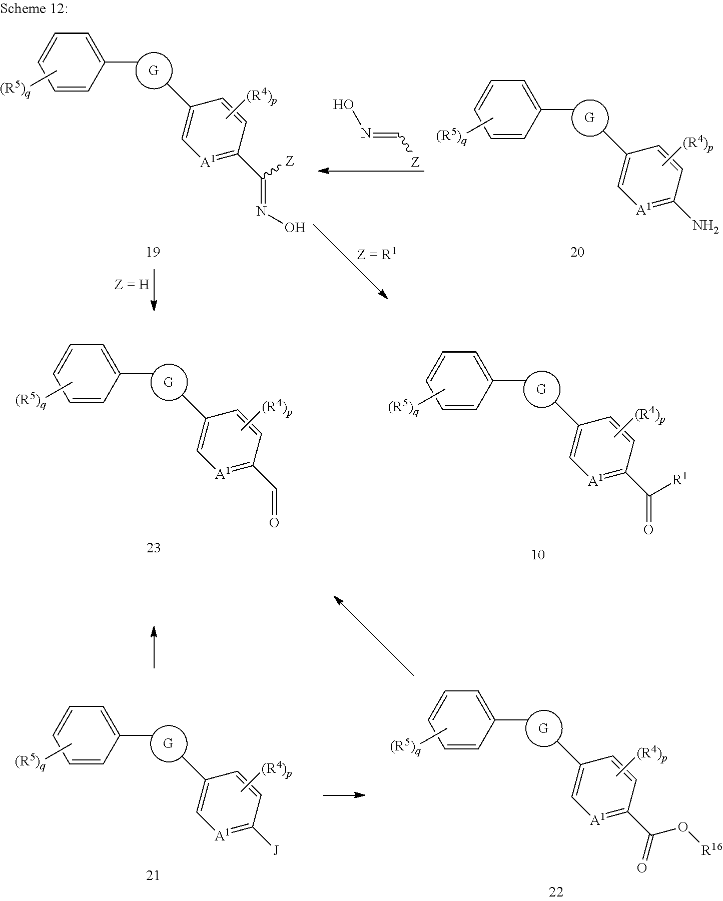

The corresponding aldehydes of formula 23 can be prepared from oximes of formula 19 by acidic hydrolysis (Z in this case equals H), as described for example by Lin et al, Chemistry—A European Journal (2009), 15(10), 2305-2309. Compounds of formula 19 can be prepared by diazotation of an amine of formula 20 and copper-catalyzed reaction with a formoxime or a higher substituted oxime, as described for example by Philipp et al, Justus Liebigs Annalen der Chemie (1936), 523, 285-289 or by Woodward et al, Tetrahedron (1958), 2, 1-57 or in WO 2010/072781 or in WO 2010/072602. The corresponding compounds of formula 20 can be prepared according to WO 2007/125984.

Compounds of formula 23 can also be prepared by palladium catalyzed carbonylation of compounds of formula 21, as described for example by Banard et al, Organic Process Research & Development (2008), 12(4), 566-574. Compounds of formula 21 can also be prepared by reduction or a reduction/oxidation sequence of esters of formula 22, as for example described in WO 2007/017468 (reduction) or in WO 2006/128803 (reduction/oxidation sequence). Compounds of formula 22 can be prepared by carbonylation of compounds of formula 21, as described for example in WO 2005/085216.

Compounds of formula I-e (compounds of formula I wherein G=G-12) can be prepared as outlined in scheme 13, by cyclization of compounds of formula 24 with a hypochlorite, as for example described by Reggelin et al, Tetrahedron Letters (1995), 36(33), 5885-5886. Subsequent reduction with disulfide, as described for example by Fischer et al. in Sulfur Letters (1987), 6(6), 191-196, yield compounds (compounds of formula I wherein G=G-11).

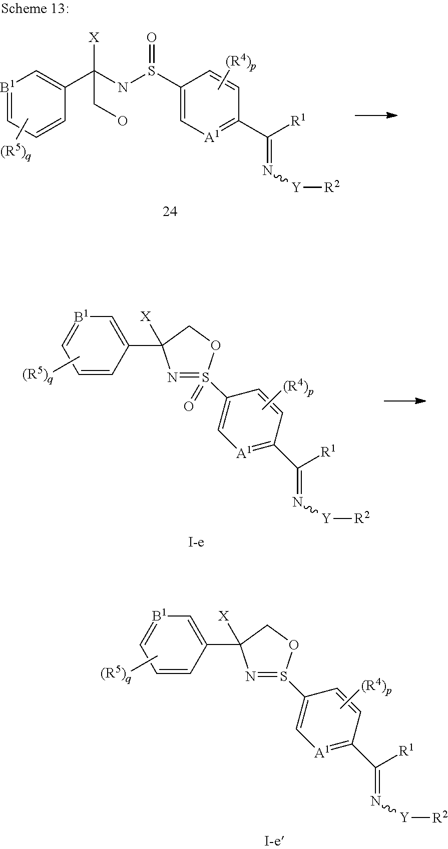

Compounds of formula I-f (compounds of formula I wherein G=G-18) can be prepared as outlined in scheme 14, by reaction of a lactone compound of formula 25 with an aniline of formula 26, as for example described by Kenda et al, Journal of Medicinal Chemistry (2004), 47(3), 530-549. The reaction involved a silyl iodide mediated lactone ring opening, acid activation and reaction with the aniline, followed by ring closure to the corresponding lactam.

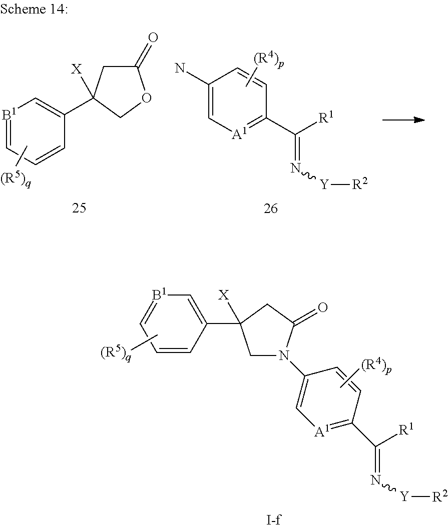

The preparation of lactones of formula 25 is described, for example by Doyle et al, Synthetic Communications (1980), 10(11), 881-888.

Compounds of formula I-g (compounds of formula I wherein G=G-16 or G-17) can be prepared as outlined in scheme 15 by reaction of an aminoalcohol or a diamino compound of formula 27 (Z=O or NR^{8a}) with a carbonyl transfer reagent such as phosgene, diphosgene, triphosgene, carbonyldiimidazole or he like, as for example described in WO 2007/123853.

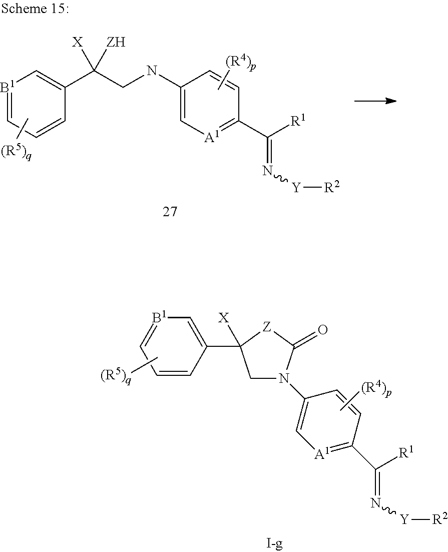

The analogous reaction of 27 with a formaldehyde transfer agent, such as formaldehyde or paraformaldehyde, as described for example in DE 3903993, yields compounds I-v (compounds of formula I wherein G=G-15)

Compounds of formula I-h (compounds of formula I wherein G=G-13) can be prepared as outlined in scheme 16, by reaction of an α,β-unsaturared ketone of formula 28 with a hydrazine compound, as described for example in WO 2007/123855.

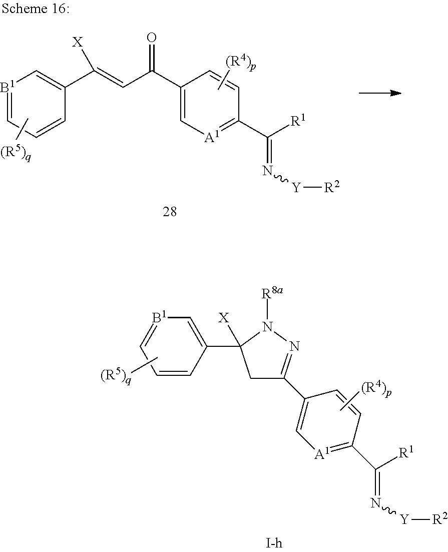

Compounds of formula I-i (compounds of formula I wherein G=G-19) can be prepared as outlined in scheme 17, by reaction of an aryl ketone of formula 29 with an activated hydroxamic acid derivative of formula 30, as described for example in WO 2010/020522.

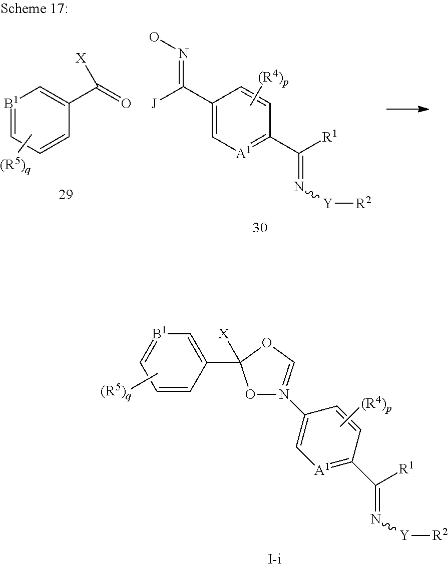

Compounds of formula I-j (compounds of formula I wherein G=G-26) can be prepared as outlined in scheme 18, as described for example in WO 2008/001076 by transition metal catalyzed coupling of an 3-arylpiperidine of formula 31 with an aryl halogenide of formula 32.

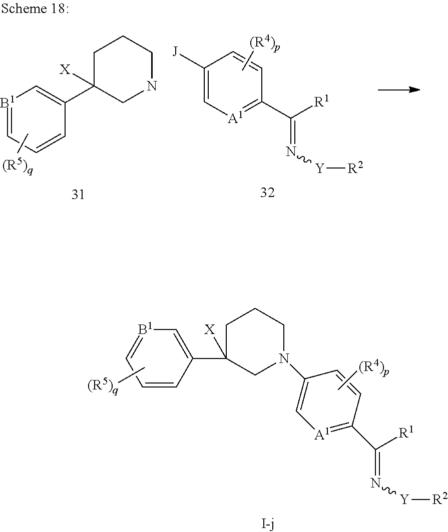

The corresponding 3-aryl piperidines of formula 31 can be prepared according to the method described for example by Cheng et al, Journal of Medicinal Chemistry (1986), 29(4), 531-537.

Compounds of formula I-k (compounds of formula I wherein G=G-1 or G-2; W is O or S) can be prepared as outlined in scheme 19 by chlorination of a compound of formula 35, followed by halogen exchange reaction, as for example described by Chupp et al., Synthesis (1986), (3), 224-226 (chlorination reaction) and U.S. Pat. No. 4,562,286 (halogen exchange reaction). Compounds of formula 35 can be prepared by cyclization of compounds of formula 34, which in turn can be derived by Wittig reaction of compounds of formula 33, as for example described by Hornback et al., Journal of the American Chemical Society (1979), 101(24), 7367-7373.

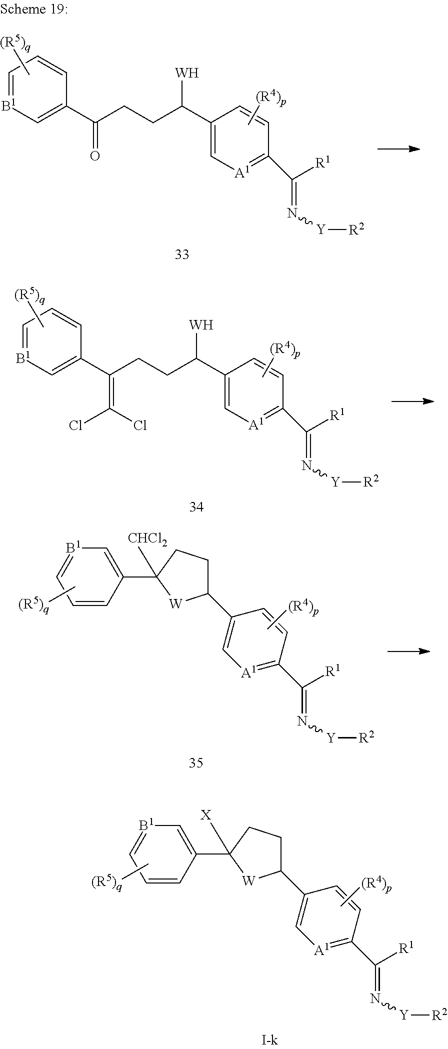

The corresponding ketones of formula 33 can be prepared according to Princival et al., Tetrahedron Letters (2009), 50(46), 6368-6371 or EP 402151.

Compounds of formula I-m (compounds of formula I wherein G=G-10 (n=1) can be prepared as outlined in scheme 20 by cyclization of compounds of formula 36 with a hypochlorite, as for example described by Reggelin et al, Tetrahedron Letters (1995), 36(33), 5885-5886.

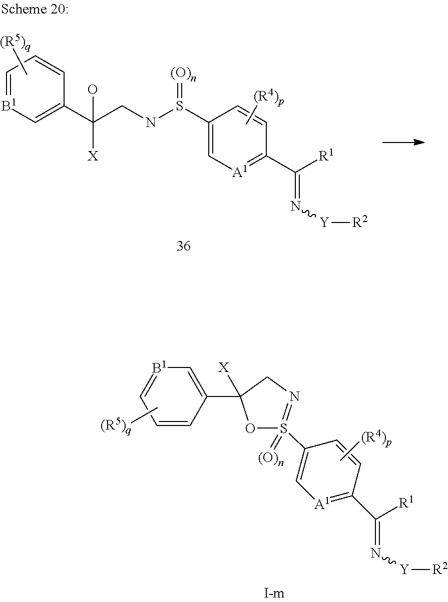

Compounds of formula I-n (compounds of formula I wherein G=G-5) can be prepared as outlined in scheme 21 from compounds of formula 39 by conversion of the nitrile moiety to the carboxylate and subsequent decarboxylation reaction as for example described by Comber et al., Journal of the Chemical Society, Perkin Transactions 1: Organic and Bio-Organic Chemistry (1972-1999) (1991), (11), 2783-2787 (saponification reaction) and Obrien et al., Angewandte Chemie, International Edition (2008), 47(36), 6877-6880 (decarboxylation reaction). Compounds of formula 39 can be prepared by a manganese acetate mediated cyclization reaction of an olefin of formula 37 with a cyano ketone of formula 38 as for example described by Nguyen et al., Synthesis (1997), (8), 899-908.

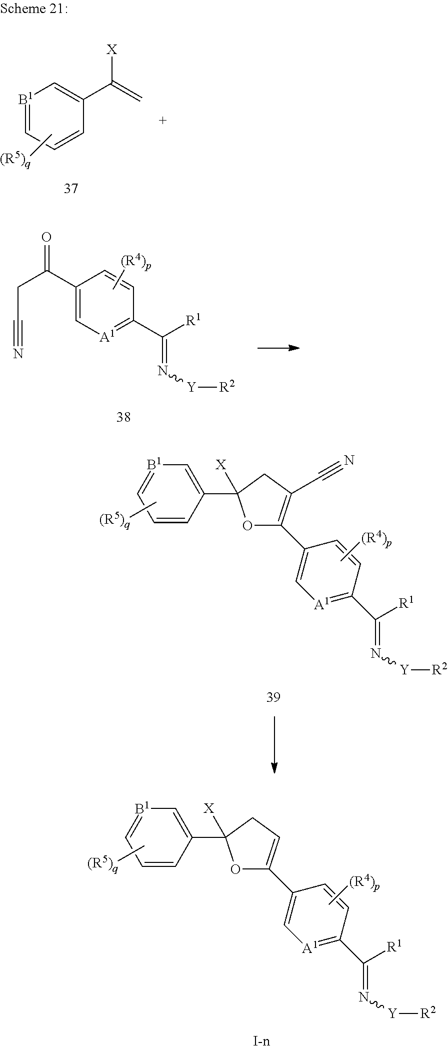

The corresponding styrene compounds of formula 37 can be prepared as described in unpublished PCT/EP2010/055773. The cyano ketones of formula 38 can be prepared according to Kim et al., Bioorganic & Medicinal Chemistry Letters (2010), 20(3), 922-926.

Compounds of formula I-o (compounds of formula I wherein G=G-20) can be prepared as outlined in scheme 22 by cycloaddition of compounds of formula 40 with oxime derivatives of formula 30, as for example described by Lin et al., Synthesis (2003), (10), 1569-1573.

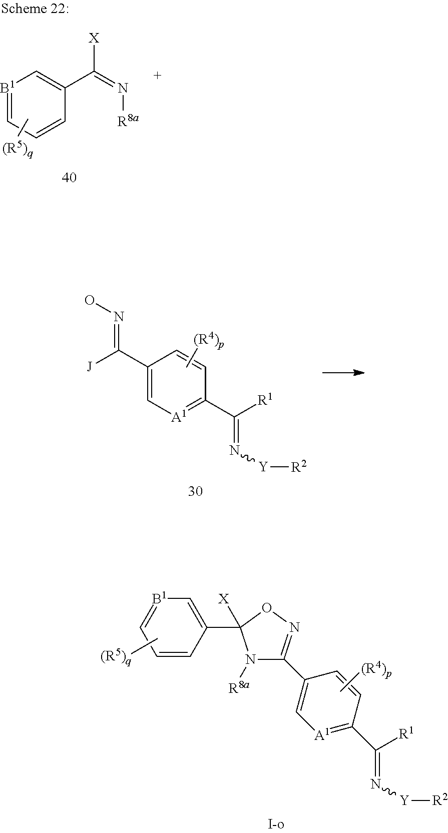

Compounds of formula 30 can be prepared as for example described in WO 2010/072781.

Compounds of formula I-p (compounds of formula I wherein G=G-22) can be prepared as outlined in scheme 23 by reaction of a compound of formula 42 in a Buchwald Hartwig amination with a compound of formula 15, as described for example in WO 2008/128711. Thiopyrrolidinones of formula 42 can be prepared from compounds of formula 41 by ketene addition as for example described by Hyatt et al., Organic Reactions (Hoboken, N.J., United States) (1994), 45. Compounds of formula 41 can be prepared in analogy to the procedures described by Porskamp et al., Journal of Organic Chemistry (1983), 48(24), 4582-4585.

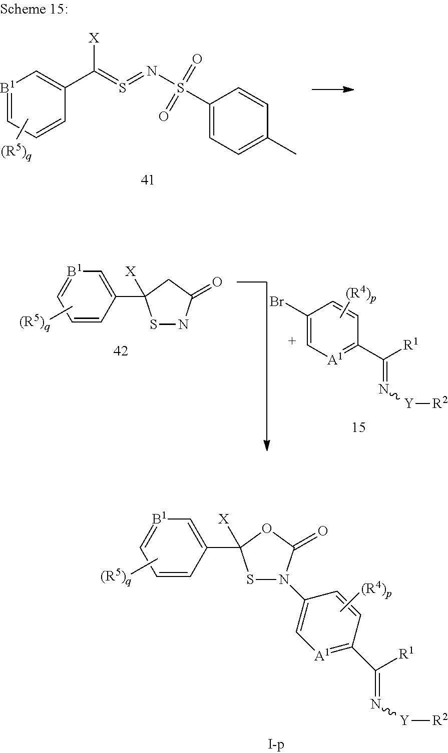

Compounds of formula I-q (compounds of formula I wherein G=G-23) can be prepared as outlined in scheme 24 by ring contraction reaction of a compound of formula 45, as for example described by Burger et al., Angewandte Chemie (1975), 87(22), 816. Compounds of formula 45 can be prepared from an acetophenone of formula 43 and a thioamide of formula 44, as for example described by Burger et al, Chemiker-Zeitung (1986), 110(2), 79-83. The corresponding acetophenones of formula 43 can be prepared according to unpublished PCT/EP2010/055773.

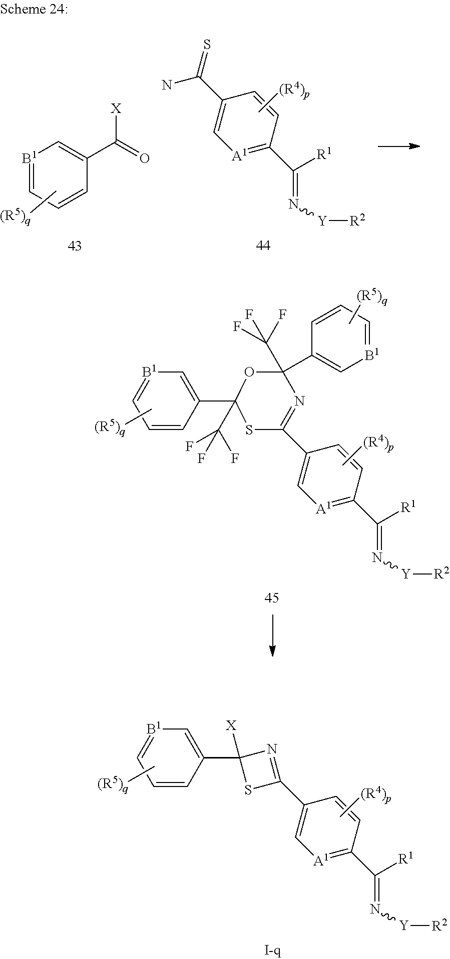

Compounds of formula I-r (compounds of formula I wherein G=G-25) can be prepared as outlined in scheme 25 by amide reduction of a compound of formula 47, as for example described by Stocks et al., Synlett (2007), (16), 2587-2589. Compounds of formula 47 can be prepared by reaction of an aniline of formula 26 with a compound of formula 46, as for example described by Nicolaus et al., Helvetica Chimica Acta (1965), 48(8), 1867-1885.

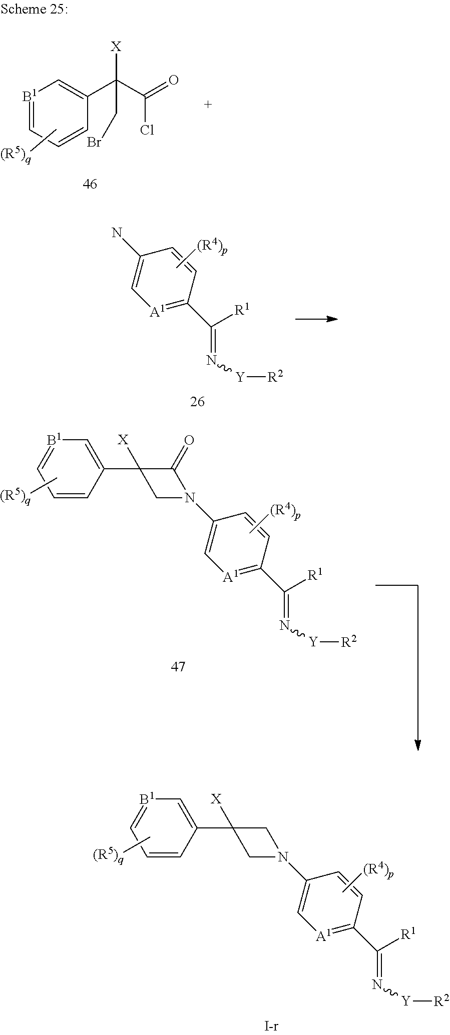

Compounds of formula I-s (compounds of formula I wherein G=G-27) can be prepared as outlined in scheme 26 by Raney nickel reduction of compounds of formula 50, as for example described by Va et al., Journal of the American Chemical Society (2010), 132(24), 8489-8495. Compounds of formula 50 can be prepared by reaction of isocyanates of formula 49 with compounds of formula 48, as for example described by Geffken et al., Chemische Berichte (1979), 112(2), 600-606.

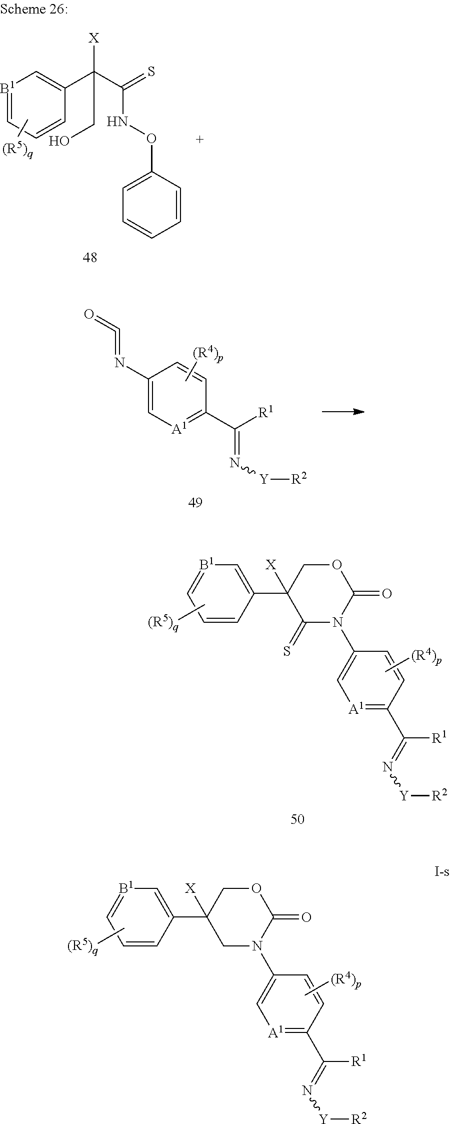

Compounds of formula I-t (compounds of formula I wherein G=G-28) can be prepared as outlined in scheme 27 by thionylation and Raney nickel reduction of compounds of formula 52, as for example described by Va et al., Journal of the American Chemical Society (2010), 132(24), 8489-8495. Subsequent acid decarboxylation should yield compounds of formula I-t. Compounds of formula 52 can be prepared from enones of formula 51 by reaction of malonic ester amide, as for example described by Al-Arab et al., Journal of Heterocyclic Chemistry (1990), 27(3), 523-525.

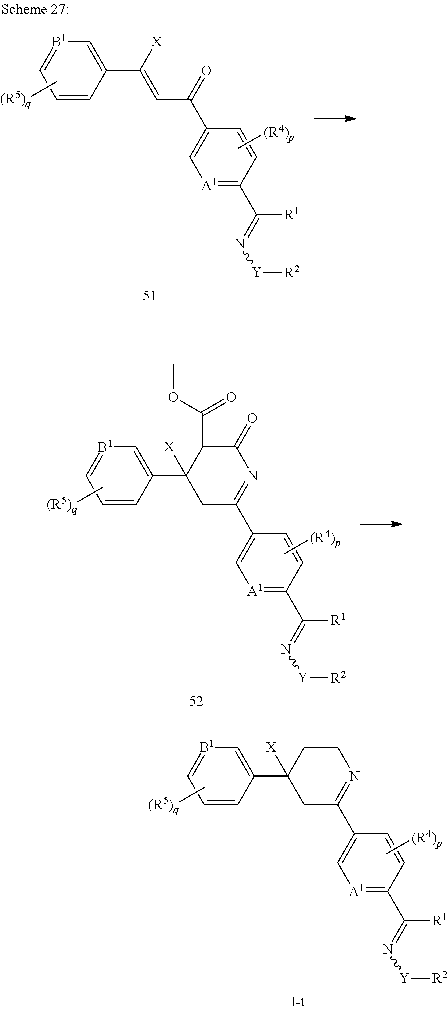

Compounds of formula I-u (compounds of formula I wherein G=G-9) can be prepared from compounds of formula I-m wherein G=G-10 (n=0) after reduction with diphenyl disulfide as for example described by Fischer et al., Sulfur Letters (1987), 6(6), 191-196.

If individual compounds cannot be prepared via the above-described routes, they can be prepared by derivatization of other compounds I or by customary modifications of the synthesis routes described.

The reaction mixtures are worked up in the customary manner, for example by mixing with water, separating the phases, and, if appropriate, purifying the crude products by chromatography, for example on alumina or silica gel. Some of the intermediates and end products may be obtained in the form of colorless or pale brown viscous oils, which are freed or purified from volatile components under reduced pressure and at moderately elevated temperature. If the intermediates and end products are obtained as solids, they may be purified by recrystallization or digestion.

Due to their excellent activity, the compounds of formula I may be used for controlling invertebrate pests.

Accordingly, the present invention also provides an agricultural composition comprising at least one compound of the formula I, as defined above, a stereoisomer thereof and/or at least one agriculturally acceptable salt thereof, and at least one inert liquid and/or solid agriculturally acceptable carrier.

The present invention also provides a veterinary composition comprising at least one compound of the formula I, as defined above, a stereoisomer thereof and/or at least one veterinarily acceptable salt thereof, and at least one inert liquid and/or solid veterinarily acceptable carrier.

Such compositions may contain a single active compound of formula I or a salt thereof or a mixture of several active compounds of formula I or their salts according to the present invention. The composition according to the present invention may comprise an individual isomer or mixtures of isomers as well as individual tautomers or mixtures of tautomers.

The present invention further relates to the use of a compound as defined above, of a stereoisomer and/or of an agriculturally or veterinarily acceptable salt thereof for combating invertebrate pests.

The present invention further relates to the use of a compound as defined above, of a stereoisomer and/or of a veterinarily acceptable salt thereof, for treating or protecting an animal from infestation or infection by invertebrate pests.

Moreover the present invention also provides a method for controlling invertebrate pests which method comprises treating the pests, their food supply, their habitat or their breeding ground or a plant, plant propagation material, soil, area, material or environment in which the pests are growing or may grow, or the materials, plants, plant propagation material, soils, surfaces or spaces to be protected from invertebrate pest attack or infestation with a pesticidally effective amount of at least one imine compound of the formula I as defined above, a stereoisomer thereof and/or at least one agriculturally acceptable salt thereof.

Preferably, the method of the invention serves for protecting plants or plant propagation material (such as seed) and the plant which grows therefrom from animal pest attack or infestation and comprises treating the plants or the plant propagation material (such as seed) with a pesticidally effective amount of a compound of the formula I or an agriculturally acceptable salt thereof as defined above or with a pesticidally effective amount of an agricultural composition as defined above and below. The method of the invention is not limited to the protection of the “substrate” (plant, plant propagation materials, soil material etc.) which has been treated according to the invention, but also has a preventive effect, thus, for example, according protection to a plant which grows from a treated plant propagation materials (such as seed), the plant itself not having been treated.

The invention furthermore relates to plant propagation material (such as seeds), comprising at least one compound of the formula I as defined above, a stereoisomer thereof and/or at least one agriculturally acceptable salt thereof.

The invention also provides a method for treating or protecting an animal from infestation or infection by invertebrate pests which comprises bringing the animal in contact with a pesticidally effective amount of at least one compound of the formula I as defined above, a stereoisomer thereof and/or at least one veterinarily acceptable salt thereof.

The compounds of the formula I and the pestidicidal compositions comprising them are effective agents for controlling arthropod pests and nematodes. Invertebrate pests controlled by the compounds of formula I include for example:

insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lamb-dina fiscellaria, Laphygma exigua, Leucoptera coffeella, Leucoptera scitella, Lithocol-letis blancardella, Lobesia botrana, Loxostege sticticalis, Lymantria dispar, Lymantria monacha, Lyonetia clerkella, Malacosoma neustria, Mamestra brassicae, Orgyia pseu-dotsugata, Ostrinia nubilalis, Panolis flammea, Pectinophora gossypiella, Peridroma saucia, Phalera bucephala, Phthorimaea operculella, Phyllocnistis citrella, Pieris bras-sicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis;
beetles (Coleoptera), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12 punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria;
dipterans (Diptera), for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa;
thrips (Thysanoptera), e.g. Dichromothrips corbetti, Frankliniella fusca, Frankliniella occidentalis, Frankliniella tritici, Scirtothrips citri, Thrips oryzae, Thrips palmi and Thrips tabaci;
hymenopterans (Hymenoptera), e.g. Athalia rosae, Atta cephalotes, Atta sexdens, Atta texana, Hoplocampa minuta, Hoplocampa testudinea, Monomorium pharaonis, Solenopsis geminata and Solenopsis invicta;
heteropterans (Heteroptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor;
homopterans (Homoptera), e.g. Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus persicae, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Sogatella furcifera Trialeurodes vaporariorum, Toxoptera aurantiiand, and Viteus vitifolii;
termites (Isoptera), e.g. Calotermes flavicollis, Leucotermes flavipes, Reticulitermes flavipes, Reticulitermes lucifugus and Termes natalensis;
orthopterans (Orthoptera), e.g. Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus;
Arachnoidea, such as arachnids (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis;
Siphonatera, e.g. Xenopsylla cheopsis, Ceratophyllus spp;

The compositions and compounds of formula I are useful for the control of nematodes, especially plant parasitic nematodes such as root knot nematodes, Meloidogyne hapla, Meloidogyne incognita, Meloidogyne javanica, and other Meloidogyne species;

cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Paratylen-chus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

In a preferred embodiment of the invention the compounds of formula I are used for controlling insects or arachnids, in particular insects of the orders Lepidoptera, Coleoptera, Thysanoptera and Homoptera and arachnids of the order Acarina. The compounds of the formula I according to the present invention are particularly useful for controlling insects of the order Thysanoptera and Homoptera.

The compounds of formula I or the pesticidal compositions comprising them may be used to protect growing plants and crops from attack or infestation by invertebrate pests, especially insects, acaridae or arachnids by contacting the plant/crop with a pesticidally effective amount of compounds of formula I. The term “crop” refers both to growing and harvested crops.

The compounds of formula I can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineers Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, anti-foaming agents, anti-freezing agents, for seed treatment formulation also optionally colorants and/or binders and/or gelling agents.

Examples of suitable solvents are water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (N-methylpyrrolidone [NMP], N-octylpyrrolidone [NOP]), acetates (glycol diacetate), glycols, fatty acid dimethylamides, fatty acids and fatty acid esters. In principle, solvent mixtures may also be used.

Suitable emulsifiers are non-ionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone or water.

Also anti-freezing agents such as glycerin, ethylene glycol, propylene glycol and bactericides such as can be added to the formulation.

Suitable antifoaming agents are for example antifoaming agents based on silicon or magnesium stearate.

A suitable preservative is e.g. dichlorophen.

Seed treatment formulations may additionally comprise binders and optionally colorants.

Binders can be added to improve the adhesion of the active materials on the seeds after treatment. Suitable binders are block copolymers EO/PO surfactants but also polyvinylalcoholsl, polyvinylpyrrolidones, polyacrylates, polymethacrylates, polybute-nes, polyisobutylenes, polystyrene, polyethyleneamines, polyethyleneamides, polyethyleneimines (Lupasol®, Polymin®), polyethers, polyurethans, polyvinylacetate, tylose and copolymers derived from these polymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®).

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers.

Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound(s). In this case, the active compound(s) are employed in a purity of from 90% to 100% by weight, preferably 95% to 100% by weight (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The compounds of formula I can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compound(s) according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1% per weight.

The active compound(s) may also be used successfully in the ultra-low-volume process (ULV), it being possible to apply formulations comprising over 95% by weight of active compound, or even to apply the active compound without additives.

The following are examples of formulations:

1. Products for Dilution with Water for Foliar Applications.

For seed treatment purposes, such products may be applied to the seed diluted or undiluted.

### A) Water-Soluble Concentrates (SL, LS)

10 parts by weight of the active compound(s) are dissolved in 90 parts by weight of water or a water-soluble solvent. As an alternative, wetters or other auxiliaries are added. The active compound(s) dissolves upon dilution with water, whereby a formula-tion with 10% (w/w) of active compound(s) is obtained.

### B) Dispersible Concentrates (DC)

20 parts by weight of the active compound(s) are dissolved in 70 parts by weight of cyclohexanone with addition of 10 parts by weight of a dispersant, for example polyvinylpyrrolidone. Dilution with water gives a dispersion, whereby a formulation with 20% (w/w) of active compound(s) is obtained.

### C) Emulsifiable Concentrates (EC)

15 parts by weight of the active compound(s) are dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compound(s) is obtained.

### D) Emulsions (EW, EO, ES)

25 parts by weight of the active compound(s) are dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound(s) is obtained.

### E) Suspensions (SC, OD, FS)

In an agitated ball mill, 20 parts by weight of the active compound(s) are comminuted with addition of 10 parts by weight of dispersants, wetters and 70 parts by weight of water or of an organic solvent to give a fine active compound(s) suspension. Dilution with water gives a stable suspension of the active compound(s), whereby a formulation with 20% (w/w) of active compound(s) is obtained.

### F) Water-Dispersible Granules and Water-Soluble Granules (WG, SG)

50 parts by weight of the active compound(s) are ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound(s), whereby a formulation with 50% (w/w) of active compound(s) is obtained.

### G) Water-Dispersible Powders and Water-Soluble Powders (WP, SP, SS, WS)

75 parts by weight of the active compound(s) are ground in a rotor-stator mill with addition of 25 parts by weight of dispersants, wetters and silica gel. Dilution with water gives a stable dispersion or solution of the active compound(s), whereby a formulation with 75% (w/w) of active compound(s) is obtained.

### H) Gel-Formulation (GF)

In an agitated ball mill, 20 parts by weight of the active compound(s) are comminuted with addition of 10 parts by weight of dispersants, 1 part by weight of a gelling agent wetters and 70 parts by weight of water or of an organic solvent to give a fine active compound(s) suspension. Dilution with water gives a stable suspension of the active compound(s), whereby a formulation with 20% (w/w) of active compound(s) is obtained.

### 2. Products to be Applied Undiluted for Foliar Applications.

For seed treatment purposes, such products may be applied to the seed diluted or undiluted.

### I) Dustable Powders (DP, DS)

5 parts by weight of the active compound(s) are ground finely and mixed intimately with 95 parts by weight of finely divided kaolin. This gives a dustable product having 5% (w/w) of active compound(s)

### J) Granules (GR, FG, GG, MG)

0.5 parts by weight of the active compound(s) is ground finely and associated with 95.5 parts by weight of carriers, whereby a formulation with 0.5% (w/w) of active compound(s) is obtained. Current methods are extrusion, spray-drying or the fluidized bed. This gives granules to be applied undiluted for foliar use.

### K) ULV Solutions (UL)

10 parts by weight of the active compound(s) are dissolved in 90 parts by weight of an organic solvent, for example xylene. This gives a product having 10% (w/w) of active compound(s), which is applied undiluted for foliar use.

The compounds of formula I are also suitable for the treatment of plant propagation materials (such as seed). Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Other preferred FS formulations of compounds of formula I for seed treatment comprise from 0.5 to 80 wt % of the active ingredient, from 0.05 to 5 wt % of a wetter, from 0.5 to 15 wt % of a dispersing agent, from 0.1 to 5 wt % of a thickener, from 5 to 20 wt % of an anti-freeze agent, from 0.1 to 2 wt % of an anti-foam agent, from 1 to 20 wt % of a pigment and/or a dye, from 0 to 15 wt % of a sticker/adhesion agent, from 0 to 75 wt % of a filler/vehicle, and from 0.01 to 1 wt % of a preservative.

Various types of oils, wetters, adjuvants, herbicides, fungicides, other pesticides, or bactericides may be added to the active ingredients, if appropriate just immediately prior to use (tank mix). These agents usually are admixed with the agents according to the invention in a weight ratio of 1:10 to 10:1.

The compounds of formula I are effective through both contact (via soil, glass, wall, bed net, carpet, plant parts or animal parts), and ingestion (bait, or plant part).

For use against ants, termites, wasps, flies, mosquitos, crickets, or cockroaches, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formula I as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylfomaamide, N methylpyrrolidone, dimethyl sulphoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formula I and their respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula I and their respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and co-polymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and diethylenically unsaturated hydrocarbons, such as styrene, and aliphatic diens, such as butadiene.

The impregnation of curtains and bednets is done in general by dipping the textile material into emulsions or dispersions of the active compounds of formula I or spraying them onto the nets.

Methods which can be employed for treating the seed are, in principle, all suitable seed treatment and especially seed dressing techniques known in the art, such as seed coating (e.g. seed pelleting), seed dusting and seed imbibition (e.g. seed soaking). Here, “seed treatment” refers to all methods that bring seeds and the compounds of formula I into contact with each other, and “seed dressing” to methods of seed treatment which provide the seeds with an amount of the compounds of formula I, i.e. which generate a seed comprising the compound of formula I. In principle, the treatment can be applied to the seed at any time from the harvest of the seed to the sowing of the seed. The seed can be treated immediately before, or during, the planting of the seed, for example using the “planters box” method. However, the treatment may also be carried out several weeks or months, for example up to 12 months, before planting the seed, for example in the form of a seed dressing treatment, without a substantially reduced efficacy being observed.

Expediently, the treatment is applied to unsown seed. As used herein, the term “unsown seed” is meant to include seed at any period from the harvest of the seed to the sowing of the seed in the ground for the purpose of germination and growth of the plant.

Specifically, a procedure is followed in the treatment in which the seed is mixed, in a suitable device, for example a mixing device for solid or solid/liquid mixing partners, with the desired amount of seed treatment formulations, either as such or after previous dilution with water, until the composition is distributed uniformly on the seed. If appropriate, this is followed by a drying step.

The compounds of formula I or veterinarily acceptable salts thereof are in particular also suitable for being used for combating parasites in and on animals.

A further object of the present invention is therefore to provide new methods for controlling parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions containing a parasiticidally effective amount of compounds of formula I or veterinarily acceptable salts thereof and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of formula I or veterinarily acceptable salts thereof or a composition comprising it.

The present invention also provides a non-therapeutic method for treating, controlling, preventing and protecting animals against infestation and infection by parasites, which comprises applying to a locus a parasiticidally effective amount of a compound of formula I or the enantiomers or veterinarily acceptable salts thereof or a composition comprising it.

The invention also provides a process for the preparation of a composition for treating, controlling, preventing or protecting animals against infestation or infection by parasites which comprises including a parasiticidally effective amount of a compound of formula I or the veterinarily acceptable salts thereof or a composition comprising it.

The invention relates further to the use of compounds of formula I for treating, controlling, preventing or protecting animals against infestation or infection by parasites. The invention relates also to the use of a compound of formula I, or a composition comprising it, for the manufacture of a medicament for the therapeutic treatment of animals against infections or infestions by parasites.

Activity of compounds against agricultural pests does not suggest their suitability for control of endo- and ectoparasites in and on animals which requires, for example, low, nonemetic dosages in the case of oral application, metabolic compatibility with the animal, low toxicity, and a safe handling.

Surprisingly, it has been found that compounds of formula I are suitable for combating endo- and ectoparasites in and on animals.

Compounds of formula I or veterinarily acceptable salts thereof and compositions comprising them are preferably used for controlling and preventing infestations and infections animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula I or veterinarily acceptable salts thereof and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula I or veterinarily acceptable salts thereof and compositions comprising them are suitable for systemic and/or non-systemic control of ecto- and/or endoparasites. They are active against all or some stages of development.

The compounds of formula I are especially useful for combating ectoparasites.

The compounds of formula I are especially useful for combating endoparasites.

The compounds of formula I are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus,
cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,

actinedida (Prostigmata) and Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,
bugs (Heteropterida): Cimex lectularius, Cimex hemipterus, Reduvius senilis, Triatoma spp., Rhodnius ssp., Panstrongylus ssp. and Arilus critatus,
Anoplurida, e.g. Haematopinus spp., Linognathus spp., Pediculus spp., Phtirus spp., and Solenopotes spp,
Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

### Roundworms Nematoda:

Wipeworms and Trichinosis (Trichosyringida), e.g. Trichinellidae (Trichinella spp.), (Trichuridae) Trichuris spp., Capillaria spp,
Rhabditida, e.g. Rhabditis spp, Strongyloides spp., Helicephalobus spp,
Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stepha-nurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,
Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,
Camallanida, e.g. Dracunculus medinensis (guinea worm)
Spirurida, e.g. Thelazia spp. Wuchereria spp., Brugia spp., Onchocerca spp., Dirofilari spp. a, Dipetalonema spp., Setaria spp., Elaeophora spp., Spirocerca lupi, and Habronema spp.,
Thorny headed worms (Acanthocephala), e.g. Acanthocephalus spp., Macracanthorhynchus hirudinaceus and Oncicola spp,

### Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp,
Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula I and compositions containing them are particularly useful for the control of pests from the orders Diptera, Siphonaptera and Ixodida.

Moreover, the use of compounds of formula I and compositions containing them for combating mosquitoes is especially preferred.

The use of the compounds of formula I and compositions containing them for combating flies is a further preferred embodiment of the present invention.

Furthermore, the use of the compounds of formula I and compositions containing them for combating fleas is especially preferred.

The use of the compounds of formula I and of the compositions containing them for combating ticks is a further preferred embodiment of the present invention.

The compounds of formula I also are especially useful for combating endoparasites (roundworms nematoda, thorny headed worms and planarians).

The compounds of formula I can be effective through both contact (via soil, glass, wall, bed net, carpet, blankets or animal parts) and ingestion (e.g. baits).

The present invention relates to the therapeutic and the non-therapeutic use of compounds of formula I for controlling and/or combating parasites in and/or on animals.

The compounds of formula I may be used to protect the animals from attack or infestation by parasites by contacting them with a parasitically effective amount of compounds of formula I. As such, “contacting” includes both direct contact (applying the compounds/compositions directly on the parasite, e.g. also at its locus, and optionally also administrating the compounds/composition directly on the animal) and indirect contact (applying the compounds/compositions to the locus of the parasite). The contact of the parasite through application to its locus is an example of a non-therapeutic use of compounds of formula I.

“Locus” as defined above means the habitat, food supply, breeding ground, area, material or environment in which a parasite is growing or may grow outside of the animal. The compounds of the invention can also be applied preventively to places at which occurrence of the pests or parasites is expected.

Administration to the animal can be carried out both prophylactically and therapeutically.

Administration of the active compounds is carried out directly or in the form of suitable preparations, orally, topically/dermally or parenterally.

For oral administration to warm-blooded animals, the compounds of formula I may be formulated as animal feeds, animal feed premixes, animal feed concentrates, pills, solutions, pastes, suspensions, drenches, gels, tablets, boluses and capsules. In addition, the compounds of formulae I may be administered to the animals in their drinking water. For oral administration, the dosage form chosen should provide the animal with 0.01 mg/kg to 100 mg/kg of animal body weight per day of the compounds of formula I, preferably with 0.5 mg/kg to 100 mg/kg of animal body weight per day.

Alternatively, the compounds of formula I may be administered to animals parenterally, for example, by intraruminal, intramuscular, intravenous or subcutaneous injection. The compounds of formula I may be dispersed or dissolved in a physiologically acceptable carrier for subcutaneous injection. Alternatively, the compounds of formula I may be formulated into an implant for subcutaneous administration. In addition the compounds of formula I may be transdermally administered to animals. For parenteral administration, the dosage form chosen should provide the animal with 0.01 mg/kg to 100 mg/kg of animal body weight per day of the compounds of formula I.

The compounds of formula I may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds of formula I. In addition, the compounds of formula I may be formulated as ear tags for animals, particularly quadrupeds such as cattle and sheep.

Suitable preparations are:

Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methylpyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g. dipropylenglycol monomethylether, ketons such as acetone, methylethylketone, aromatic hydrocarbons, vegetable and synthetic oils, dimethylformamide, dimethylacetamide, transcutol, solketal, propylencarbonate, and mixtures thereof.

It may be advantageous to add thickeners during preparation. Suitable thickeners are inorganic thickeners such as bentonites, colloidal silicic acid, aluminium monostearate, organic thickeners such as cellulose derivatives, polyvinyl alcohols and their copolymers, acrylates and methacrylates.

Gels are applied to or spread on the skin or introduced into body cavities. Gels are prepared by treating solutions which have been prepared as described in the case of the injection solutions with sufficient thickener that a clear material having an ointment-like consistency results. The thickeners employed are the thickeners given above.

Pour-on formulations are poured or sprayed onto limited areas of the skin, the active compound penetrating the skin and acting systemically.

Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added.

Suitable solvents are water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, DMF, dimethylacetamide, n-alkylpyrrolidones such as methylpyrrolidone, n-butylpyrrolidone or n-octylpyrrolidone, N methylpyrrolidone, 2-pyrrolidone, 2,2-dimethyl-4-oxy-methylene-1,3-diox-olane and glycerol formal.

Suitable colorants are all colorants permitted for use on animals and which can be dissolved or suspended.

Suitable absorption-promoting substances are, for example, DMSO, spreading oils such as isopropyl myristate, dipropylene glycol pelargonate, silicone oils and copolymers thereof with polyethers, fatty acid esters, triglycerides, fatty alcohols.

Suitable antioxidants are sulfites or metabisulfites such as potassium metabisulfite, ascorbic acid, butylhydroxytoluene, butylhydroxyanisole, tocopherol.

Suitable light stabilizers are, for example, novantisolic acid.

Suitable adhesives are, for example, cellulose derivatives, starch derivatives, polyacrylates, natural polymers such as alginates, gelatin.

Emulsions can be administered orally, dermally or as injections.

Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

Suitable hydrophobic phases (oils) are:

liquid paraffins, silicone oils, natural vegetable oils such as sesame oil, almond oil, castor oil, synthetic triglycerides such as caprylic/capric biglyceride, triglyceride mixture with vegetable fatty acids of the chain length C₈-C₁₂ or other specially selected natural fatty acids, partial glyceride mixtures of saturated or unsaturated fatty acids possibly also containing hydroxyl groups, mono- and diglycerides of the C₈-C₁₀ fatty acids, fatty acid esters such as ethyl stearate, di-n-butyryl adipate, hexyl laurate, dipropylene glycol perlargonate, esters of a branched fatty acid of medium chain length with saturated fatty alcohols of chain length C₁₆-C₁₈, isopropyl myristate, isopropyl palmitate, caprylic/capric acid esters of saturated fatty alcohols of chain length C₁₂-C₁₈, isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter, fatty alcohols such as isotridecyl alcohol, 2-octyl-dodecanol, cetylstearyl alcohol, oleyl alcohol, and fatty acids such as oleic acid and mixtures thereof.

Suitable hydrophilic phases are: water, alcohols such as propylene glycol, glycerol, sorbitol and mixtures thereof.

Suitable emulsifiers are:

non-ionic surfactants, e.g. polyethoxylated castor oil, polyethoxylated sorbitan monooleate, sorbitan monostearate, glycerol monostearate, polyoxyethyl stearate, alkylphenol polyglycol ether;
ampholytic surfactants such as di-sodium N-lauryl-p-iminodipropionate or lecithin;
anionic surfactants, such as sodium lauryl sulfate, fatty alcohol ether sulfates, mono/dialkyl polyglycol ether orthophosphoric acid ester monoethanolamine salt;
cation-active surfactants, such as cetyltrimethylammonium chloride.

Suitable further auxiliaries are: substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures of the substances mentioned.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures.

Suitable wetting agents (dispersants) are the emulsifiers given above.

Other auxiliaries which may be mentioned are those given above.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminium oxides, titanium oxide, silicic acids, argillaceous earths, precipitated or colloidal silica, or phosphates. Organic substances are, for example, sugar, cellulose, foodstuffs and feeds such as milk powder, animal meal, grain meals and shreds, starches.

Suitable auxiliaries are preservatives, antioxidants, and/or colorants which have been mentioned above.

Other suitable auxiliaries are lubricants and glidants such as magnesium stearate, stearic acid, talc, bentonites, disintegration-promoting substances such as starch or crosslinked polyvinylpyrrolidone, binders such as starch, gelatin or linear polyvinylpyrrolidone, and dry binders such as microcrystalline cellulose.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

The compositions which can be used in the invention can comprise generally from about 0.001 to 95% of the compounds of formula I.

Generally, it is favorable to apply the compounds of formula I in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula I against endoparasites in concentrations of 10 ppm to 2 percent by weight, preferably of 0.05 to 0.9 percent by weight, very particularly preferably of 0.005 to 0.25 percent by weight.

In a preferred embodiment of the present invention, the compositions comprising the compounds of formula I are applied dermally/topically.

In a further preferred embodiment, the topical application is conducted in the form of compound-containing shaped articles such as collars, medallions, ear tags, bands for fixing at body parts, and adhesive strips and foils.

Generally, it is favorable to apply solid formulations which release compounds of formula I in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of formula I. A detailed list of plastics and elastomers as well as preparation procedures for the shaped articles is given e.g. in WO 03/086075.

Compositions to be used according to this invention may also contain other active ingredients, for example other pesticides, insecticides, herbicides, fungicides, other pesticides, or bactericides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

These agents can be admixed with the agents used according to the invention in a weight ratio of 1:10 to 10:1. Mixing the compounds of formula I or the compositions comprising them in the use form as pesticides with other pesticides frequently results in a broader pesticidal spectrum of action.

The following list M of pesticides together with which the compounds according to the invention can be used and with which potential synergistic effects might be produced, is intended to illustrate the possible combinations, but not to impose any limitation:

M.1. Organo(thio)phosphate compounds: acephate, azamethiphos, azinphos-ethyl, azinphos-methyl, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, flupyrazophos, fosthiazate, heptenophos, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion;
M.2. Carbamate compounds: aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb, triazamate;
M.3. Pyrethroid compounds: acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zetacypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, metofluthrin, permethrin, phenothrin, prallethrin, profluthrin, pyrethrin (pyrethrum), resmethrin, silafluofen, tefluthrin, tetramethrin, tralomethrin, transfluthrin;
M.4. Juvenile hormone mimics: hydroprene, kinoprene, methoprene, fenoxycarb, pyriproxyfen;
M.5. Nicotinic receptor agonists/antagonists compounds: acetamiprid, bensultap, cartap hydrochloride, clothianidin, dinotefuran, imidacloprid, thiamethoxam, nitenpyram, nicotine, spinosad (allosteric agonist), spinetoram (allosteric agonist), thiacloprid, thiocyclam, thiosultap-sodium and AKD1022.
M.6. GABA gated chloride channel antagonist compounds: chlordane, endosulfan, gamma-HCH (lindane); ethiprole, fipronil, pyrafluprole, pyriprole
M.7. Chloride channel activators: abamectin, emamectin benzoate, milbemectin, lepimectin;
M.8. METI I compounds: fenazaquin, fenpyroximate, pyrimidifen, pyridaben, tebufenpyrad, tolfenpyrad, flufenerim, rotenone;
M.9. METI II and III compounds: acequinocyl, fluacyprim, hydramethylnon;
M.10. Uncouplers of oxidative phosphorylation: chlorfenapyr, DNOC;
M.11. Inhibitors of oxidative phosphorylation: azocyclotin, cyhexatin, diafenthiuron, fenbutatin oxide, propargite, tetradifon;
M.12. Moulting disruptors: cyromazine, chromafenozide, halofenozide, methoxyfenozide, tebufenozide;
M.13. Synergists: piperonyl butoxide, tribufos;
M.14. Sodium channel blocker compounds: indoxacarb, metaflumizone;
M.15. Fumigants: methyl bromide, chloropicrin sulfuryl fluoride;
M.16. Selective feeding blockers: crylotie, pymetrozine, flonicamid;
M.17. Mite growth inhibitors: clofentezine, hexythiazox, etoxazole;
M.18. Chitin synthesis inhibitors: buprofezin, bistrifluoron, chlorfluazuron, diflubenzuron, flucycloxuron, flufenoxuron, hexaflumuron, lufenuron, novaluron, noviflumuron, teflubenzuron, triflumuron;
M.19. Lipid biosynthesis inhibitors: spirodiclofen, spiromesifen, spirotetramat;
M.20. Octapaminergic agonists: amitraz;
M.21. Ryanodine receptor modulators: flubendiamide and the phtalamid compound (R)—,(S)-3-Chlor-N1-{2-methyl-4-[1,2,2,2-tetrafluor-1-(trifluormethyl)ethyl]phenyl}-N2-(1-methyl-2-methylsulfonylethyl)phthalamid (M21.1)
M.22. Isoxazoline compounds: 4-[5-(3,5-Dichloro-phenyl)-5-trifluoromethyl-4,5-dihydroisoxazol-3-yl]-2-methyl-N-pyridin-2-ylmethyl-benzamide (M22.1), 4-[5-(3,5-Dichloro-phenyl)-5-trifluoromethyl-4,5-dihydro-isoxazol-3-yl]-2-methyl-N-(2,2,2-trifluoro-ethyl)-benzamide (M22.2), 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide (M22.3), 4-[5-(3,5-Dichloro-phenyl)-5-trifluoromethyl-4,5-dihydro-isoxazol-3-yl]-naphthalene-1-carboxylic acid [(2,2,2-trifluoro-ethylcarbamoyl)-methyl]-amide (M22.4) 4-[5-(3,5-Dichlorophenyl)-5-trifluoromethyl-4,5-dihydro-isoxazol-3-yl]-N-[(methoxyimino)methyl]-2-methylbenzamide (M22.5), 4-[5-(3-Chloro-5-trifluoromethyl-phenyl)-5-trifluoromethyl-4,5-dihydro-isoxazol-3-yl]-2-methyl-N-[(2,2,2-trifluoroethylcarbamoyl)-methyl]-benzamide (M22.6), 4-[5-(3-Chloro-5-trifluoromethyl-phenyl)-5-trifluoromethyl-4,5-dihydro-isoxazol-3-yl]-naphthalene-1-carboxylic acid [(2,2,2-trifluoro-ethylcarbamoyl)-methyl]-amide (M22.7) and 5-[5-(3,5-Dichloro-4-fluorophenyl)-5-trifluoromethyl-4,5-dihydro-isoxazol-3-yl]-2-[1,2,4]triazol-1-yl-benzonitrile

(M22.8);

M.23. Anthranilamide compounds: chloranthraniliprole, cyantraniliprole,
5-Bromo-2-(3-chloro-pyridin-2-yl)-2H-pyrazole-3-carboxylic acid [4-cyano-2-(1-cyclopropyl-ethylcarbamoyl)-6-methyl-phenyl]-amide (M23.1),
5-Bromo-2-(3-chloro-pyridin-2-yl)-2H-pyrazole-3-carboxylic acid [2-chloro-4-cyano-6-(1-cyclopropyl-ethylcarbamoyl)-phenyl]-amide (M23.2),
5-Bromo-2-(3-chloro-pyridin-2-yl)-2H-pyrazole-3-carboxylic acid [2-bromo-4-cyano-6-(1-cyclopropyl-ethylcarbamoyl)-phenyl]amide (M23.3),
5-Bromo-2-(3-chloro-pyridin-2-yl)-2H-pyrazole-3-carboxylic acid [2-bromo-4-chloro-6-(1-cyclopropyl-ethylcarbamoyl)-phenyl]-amide (M23.4),
5-Bromo-2-(3-chloro-pyridin-2-yl)-2H-pyrazole-3-carboxylic acid [2,4-dichloro-6-(1-cyclopropyl-ethylcarbamoyl)-phenyl]-amide (M23.5),
5-Bromo-2-(3-chloro-pyridin-2-yl)-2H-pyrazole-3-carboxylic acid [4-chloro-2-(1-cyclopropyl-ethylcarbamoyl)-6-methyl-phenyl]-amide (M23.6),
N′-(2-{[5-Bromo-2-(3-chloro-pyridin-2-yl)-2H-pyrazole-3-carbonyl]-amino}-5-chloro-3-methyl-benzoyl)-hydrazinecarboxylic acid methyl ester (M23.7),
N′-(2-{[5-Bromo-2-(3-chloro-pyridin-2-yl)-2H-pyrazole-3-carbonyl]-amino}-5-chloro-3-methyl-benzoyl)-N′-methyl-hydrazinecarboxylic acid methyl ester (M23.8),
N′-(2-{[5-Bromo-2-(3-chloro-pyridin-2-yl)-2H-pyrazole-3-carbonyl]-amino}-5-chloro-3-methyl-benzoyl)-N,N′-dimethyl-hydrazinecarboxylic acid methyl ester (M23.9),
N′-(3,5-Dibromo-2-{[5-bromo-2-(3-chloro-pyridin-2-yl)-2H-pyrazole-3-carbonyl]-amino}-benzoyl)-hydrazinecarboxylic acid methyl ester (M23.10),
N′-(3,5-Dibromo-2-{[5-bromo-2-(3-chloro-pyridin-2-yl)-2H-pyrazole-3-carbonyl]-amino}-benzoyl)-N′-methyl-hydrazinecarboxylic acid methyl ester (M23.11) and
N′-(3,5-Dibromo-2-{[5-bromo-2-(3-chloro-pyridin-2-yl)-2H-pyrazole-3-carbonyl]-amino}-benzoyl)-N,N′-dimethyl-hydrazinecarboxylic acid methyl ester (M23.12);
   M.24. Malononitrile compounds: 2-(2,2,3,3,4,4,5,5-octafluoropentyl)-2-(3,3,3-trifluoropropyl)malononitrile (CF₂H—CF₂—CF₂—CF₂—CH₂—C(CN)₂—CH₂—CH₂—CF₃) (M24.1) and 2-(2,2,3,3,4,4,5,5-octafluoropentyl)-2-(3,3,4,4,4-pentafluorobutyl)-malonodinitrile (CF₂H—CF₂—CF₂—CF₂—CH₂—C(CN)₂—CH₂—CH₂—CF₂—CF₃) (M24.2);
   M.25. Microbial disruptors: Bacillus thuringiensis subsp. Israelensi, Bacillus sphaericus, Bacillus thuringiensis subsp. Aizawai, Bacillus thuringiensis subsp. Kurstaki, Bacillus thuringiensis subsp. Tenebrionis;
   M.26. Aminofuranone compounds:
4-{[(6-Bromopyrid-3-yl)methyl](2-fluoroethyl)amino}furan-2(5H)-on (M26.1),
4-{[(6-Fluoropyrid-3-yl)methyl](2,2-difluoroethyl)amino}furan-2(5H)-on (M26.2),
4-{[(2-Chloro1,3-thiazolo-5-yl)methyl](2-fluoroethyl)amino}furan-2(5H)-on (M26.3),
4-{[(6-Chloropyrid-3-yl)methyl](2-fluoroethyl)amino}furan-2(5H)-on (M26.4),
4-{[(6-Chloropyrid-3-yl)methyl](2,2-difluoroethyl)amino}furan-2(5H)-on (M26.5),
4-{[(6-Chloro-5-fluoropyrid-3-yl)methyl](methyl)amino}furan-2(5H)-on (M26.6),
4-{[(5,6-Dichloropyrid-3-yl)methyl](2-fluoroethyl)amino}furan-2(5H)-on (M26.7),
4-{[(6-Chloro-5-fluoropyrid-3-yl)methyl](cyclopropyl)amino}furan-2(5H)-on (M26.8),
4-{[(6-Chloropyrid-3-yl)methyl](cyclopropyl)amino}furan-2(5H)-on (M26.9) and
4-{[(6-Chloropyrid-3-yl)methyl](methyl)amino}furan-2(5H)-on (M26.10);
   M.27. Various compounds: aluminium phosphide, amidoflumet, benclothiaz, benzoximate, bifenazate, borax, bromopropylate, cyanide, cyenopyrafen, cyflumetofen, chinomethionate, dicofol, fluoroacetate, phosphine, pyridalyl, pyrifluquinazon, sulfur, organic sulfur compounds, tartar emetic, sulfoxaflor, N—R′-2,2-dihalo-1-R″cyclopropanecarboxamide-2-(2,6-dichloro-α,α,α-trifluoro-p-tolyl)hydrazone or N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl, 4-But-2-ynyloxy-6-(3,5-dimethyl-piperidin-1-yl)-2-fluoro-pyrimidine (M27.1), Cyclopropaneacetic acid, 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]ester (M27.2) and
8-(2-Cyclopropylmethoxy-4-trifluoromethyl-phenoxy)-3-(6-trifluoromethyl-pyridazin-3-yl)-3-aza-bicyclo[3.2.1]octane (M27.3).

The commercially available compounds of the group M may be found in The Pesticide Manual, 13th Edition, British Crop Protection Council (2003) among other publications.

Paraoxon and their preparation have been described in Farm Chemicals Handbook, Volume 88, Meister Publishing Company, 2001. Flupyrazofos has been described in Pesticide Science 54, 1988, p. 237-243 and in U.S. Pat. No. 4,822,779. AKD 1022 and its preparation have been described in U.S. Pat. No. 6,300,348. The anthranilamides M23.1 to M23.6 have been described in WO 2008/72743 and WO 200872783, those M23.7 to M23.12 in WO2007/043677. The phthalamide M 21.1 is known from WO 2007/101540. The alkynylether compound M27.1 is described e.g. in JP 2006131529. Organic sulfur compounds have been described in WO 2007060839. The isoxazoline compounds M 22.1 to M 22.8 have been described in e.g. WO2005/085216, WO 2007/079162, WO 2007/026965, WO 2009/126668 and WO2009/051956. The aminofuranone compounds M 26.1 to M 26.10 have been described eg. in WO 2007/115644. The pyripyropene derivative M 27.2 has been described in WO 2008/66153 and WO 2008/108491. The pyridazin compound M 27.3 has been described in JP 2008/115155. Malononitrile compounds as those (M24.1) and (M24.2) have been described in WO 02/089579, WO 02/090320, WO 02/090321, WO 04/006677, WO 05/068423, WO 05/068432 and WO 05/063694.

The following list F of fungicides together with which the compounds according to the invention can be used and with which potential synergistic effects might be produced, is intended to illustrate the possible combinations, but not to impose any limitation:

### F.I) Respiration Inhibitors

F.I-1) Inhibitors of complex III at Qo Site (e.g. strobilurins)
strobilurins: azoxystrobin, coumethoxystrobin, coumoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, trifloxystrobin, 2-[2-(2,5-dimethyl-phenoxymethyl)-phenyl]-3-methoxy-acrylic acid methyl ester and 2 (2-(3-(2,6-dichlorophenyl)-1-methyl-allylideneaminooxymethyl)-phenyl)-2-methoxyimino-N methylacetamide;
oxazolidinediones and imidazolinones: famoxadone, fenamidone;
F.I-2) Inhibitors of complex II (e.g. carboxamides):
carboxanilides: benodanil, bixafen, boscalid, carboxin, fenfuram, fenhexamid, fluopyram, flutolanil, furametpyr, isopyrazam, isotianil, mepronil, oxycarboxin, penflufen, penthiopyrad, sedaxane, tecloftalam, thifluzamide, tiadinil, 2-amino-4 methyl-thiazole-5-carboxanilide, N-(3′,4′,5′trifluorobiphenyl-2yl)-3-difluoromethyl-1-methyl-1H-pyrazole-4carboxamide, N-(4′-trifluoromethylthiobiphenyl-2-yl)-3 difluoromethyl-1-methyl-1H pyrazole-4-carboxamide and N-(2-(1,3,3-trimethyl-butyl)-phenyl)-1,3-dimethyl-5 fluoro-1H-pyrazole-4carboxamide;
F.I-3) Inhibitors of complex III at Qi site: cyazofamid, amisulbrom;
F.I-4) Other respiration inhibitors (complex I, uncouplers)
diflumetorim; tecnazen; ferimzone; ametoctradin; silthiofam;
nitrophenyl derivates: binapacryl, dinobuton, dinocap, fluazinam, nitrthal-isopropyl, organometal compounds: fentin salts, such as fentin-acetate, fentin chloride or fentin hydroxide;

### F.II) Sterol Biosynthesis Inhibitors (SBI Fungicides)

F.II-1) C14 demethylase inhibitors (DMI fungicides, e.g. triazoles, imidazoles)
triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, paclobutrazole, penconazole, propiconazole, prothioconazole, simeconazole, tebuconazole, tetraconazole, triadimefon, triadimenol, triticonazole, uniconazole;
imidazoles: imazalil, pefurazoate, oxpoconazole, prochloraz, triflumizole;
pyrimidines, pyridines and piperazines: fenarimol, nuarimol, pyrifenox, triforine;
F.II-2) Delta14-reductase inhitors (amines, e.g. morpholines, piperidines)
morpholines: aldimorph, dodemorph, dodemorph-acetate, fenpropimorph, tridemorph;
piperidines: fenpropidin, piperalin;
spiroketalamines: spiroxamine;
F.II-3) Inhibitors of 3-keto reductase: hydroxyanilides: fenhexamid;

### F.III) Nucleic Acid Synthesis Inhibitors

F.III-1) RNA, DNA synthesis
phenylamides or acyl amino acid fungicides: benalaxyl, benalaxyl-M, kiralaxyl, metalaxyl, metalaxyl-M (mefenoxam), ofurace, oxadixyl;
isoxazoles and iosothiazolones: hymexazole, octhilinone;
F.III-2) DNA topisomerase inhibitors: oxolinic acid;
F.III-3) Nucleotide metabolism (e.g. adenosin-deaminase)
hydroxy(2-amino)-pyrimidines: bupirimate;

### F.IV) Inhibitors of Cell Division and or Cytoskeleton

F.IV-1) Tubulin inhibitors: benzimidazoles and thiophanates: benomyl, carbendazim, fuberidazole, thiabendazole, thiophanate-methyl;
triazolopyrimidines: 5-chloro-7 (4-methylpiperidin-1-yl)-6-(2,4,6-trifluorophenyl)-[1,2,4]triazolo[1,5a]pyrimidine
F.IV-2) Other cell division inhibitors
benzamides and phenyl acetamides: diethofencarb, ethaboxam, pencycuron, fluopicolide, zoxamide;
F.IV-3) Actin inhibitors: benzophenones: metrafenone;

### F.V) Inhibitors of Amino Acid and Protein Synthesis

F.V-1) Mmethionine synthesis inhibitors (anilino-pyrimidines)
anilino-pyrimidines: cyprodinil, mepanipyrim, nitrapyrin, pyrimethanil;
F.V-2) Protein synthesis inhibitors (anilino-pyrimidines)
antibiotics: blasticidin-S, kasugamycin, kasugamycin hydrochloride-hydrate, mildiomycin, streptomycin, oxytetracyclin, polyoxine, validamycin A;

### F.VI) Signal Transduction Inhibitors

F.VI-1) MAP/Histidine kinase inhibitors (e.g. anilino-pyrimidines)
dicarboximides: fluoroimid, iprodione, procymidone, vinclozolin;
phenylpyrroles: fenpiclonil, fludioxonil;
F.VI-2) G protein inhibitors: quinolines: quinoxyfen;

### F.VII) Lipid and Membrane Synthesis Inhibitors

F.VII-1) Phospholipid biosynthesis inhibitors
organophosphorus compounds: edifenphos, iprobenfos, pyrazophos;
dithiolanes: isoprothiolane;
F.VII-2) Lipid peroxidation
aromatic hydrocarbons: dicloran, quintozene, tecnazene, tolclofos-methyl, biphenyl, chloroneb, etridiazole;
F.VII-3) Carboxyl acid amides (CAA fungicides)
cinnamic or mandelic acid amides: dimethomorph, flumorph, mandiproamid, pyrimorph;
valinamide carbamates: benthiavalicarb, iprovalicarb, pyribencarb, valifenalate and N-(1-(1-(4-cyano-phenyl)ethanesulfonyl)-but-2-yl)carbamic acid-(4-fluorophenyl)ester;
F.VII-4) Compounds affecting cell membrane permeability and fatty acids
carbamates: propamocarb, propamocarb-hydrochlorid
F.VIII) Inhibitors with Multi Site Action
F.VIII-1) Inorganic active substances: Bordeaux mixture, copper acetate, copper hydroxide, copper oxychloride, basic copper sulfate, sulfur;
F.VIII-2) Thio- and dithiocarbamates: ferbam, mancozeb, maneb, metam, methasulphocarb, metiram, propineb, thiram, zineb, ziram;
F.VIII-3) Organochlorine compounds (e.g. phthalimides, sulfamides, chloronitriles):
anilazine, chlorothalonil, captafol, captan, folpet, dichlofluanid, dichlorophen, flusulfamide, hexachlorobenzene, pentachlorphenole and its salts, phthalide, tolylfluanid, N-(4-chloro-2-nitro-phenyl)-N-ethyl-4-methyl-benzenesulfonamide;
F.VIII-4) Guanidines: guanidine, dodine, dodine free base, guazatine, guazatineacetate, iminoctadine, iminoctadine-triacetate, iminoctadine-tris(albesilate);
F.VIII-5) Ahtraquinones: dithianon;

### F.IX) Cell Wall Synthesis Inhibitors

F.IX-1) Inhibitors of glucan synthesis: validamycin, polyoxin B;
F.IX-2) Melanin synthesis inhibitors: pyroquilon, tricyclazole, carpropamide, dicyclomet, fenoxanil;

### F.X) Plant Defence Inducers

F.X-1) Salicylic acid pathway: acibenzolar-S-methyl;
F.X-2) Others: probenazole, isotianil, tiadinil, prohexadione-calcium;
phosphonates: fosetyl, fosetyl-aluminum, phosphorous acid and its salts;

### F.XI) Unknown Mode of Action:

bronopol, chinomethionat, cyflufenamid, cymoxanil, dazomet, debacarb, diclomezine, difenzoquat, difenzoquat-methylsulfate, diphenylamin, flumetover, flusulfamide, flutianil, methasulfocarb, oxin-copper, proquinazid, tebufloquin, tecloftalam, triazoxide, 2-butoxy-6-iodo-3-propylchromen-4-one, N-(cyclopropylmethoxyimino-(6-difluoromethoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N methyl formamidine, N′(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2 methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl-(R)-1,2,3,4-tetrahydro-naphthalen-1-yl-amide, methoxy-acetic acid 6-tert-butyl-8-fluoro-2,3-dimethyl-quinolin-4-yl ester and N-Methyl-2-{1-[(5-methyl-3-trifluoromethyl-1H-pyrazol-1-yl)-acetyl]-piperidin-4-yl}-N-[(1R)-1,2,3,4-tetrahydronaphthalen-1-yl]-4-thiazolecarboxamide, 3-[5-(4-chloro-phenyl)-2,3-dimethyl-isoxazolidin-3-yl]-pyridine (pyrisoxazole), 3-[5-(4-methyl-phenyl)-2,3-dimethyl-isoxazolidin-3-yl]-pyridine, 5-amino-2-isopropyl-3-oxo-4-ortho-tolyl-2,3-dihydro-pyrazole-1 carbothioic acid S-allyl ester, N-(6-methoxy-pyridin-3-yl)cyclopropanecarboxylic acid amide, 5-chloro-1 (4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxy-acetamide;

### F.XII) Growth Regulators:

abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N6benzyladenine, paclobutrazol, prohexadione (prohexadione-calcium), prohydrojasmon, thidiazuron, triapenthenol, tributyl phosphorotrithioate, 2,3,5tri iodobenzoic acid, trinexapac-ethyl and uniconazole;

### F.XIII) Biological Control Agents

antifungal biocontrol agents: Bacillus substilis strain with NRRL No. B-21661 (e.g. RHAPSODY®, SERENADE® MAX and SERENADE® ASO from AgraQuest, Inc., USA.), Bacillus pumilus strain with NRRL No. B-30087 (e.g. SONATA® and BALLAD® Plus from AgraQuest, Inc., USA), Ulocladium oudemansii (e.g. the product BOTRY-ZEN from BotriZen Ltd., New Zealand), Chitosan (e.g. ARMOUR-ZEN from BotriZen Ltd., New Zealand).

The invertebrate pest, i.e. arthropodes and nematodes, the plant, soil or water in which the plant is growing can be contacted with the present compound(s) of formula I or composition(s) containing them by any application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

Moreover, invertebrate pests may be controlled by contacting the target pest, its food supply, habitat, breeding ground or its locus with a pesticidally effective amount of compounds of formula I. As such, the application may be carried out before or after the infection of the locus, growing crops, or harvested crops by the pest.

“Locus” means a habitat, breeding ground, cultivated plants, plant propagation material (such as seed), soil, area, material or environ-ment in which a pest or parasite is growing or may grow.

In general “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of formula I and their compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of are applied not only to the surrounding soil surface or into the under-floor soil in order to protect wooden materials but it can also be applied to lumbered articles such as surfaces of the under-floor concrete, alcove posts, beams, plywood, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

The compounds of formula I can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95% by weight, preferably from 0.1 to 45% by weight, and more preferably from 1 to 25% by weight of at least one repellent and/or insecticide.

For use in bait compositions, the typical content of active ingredient is from 0.001% by weight to 15% by weight, desirably from 0.001% by weight to 5% by weight of active compound.

For use in spray compositions, the content of active ingredient is from 0.001 to 80% by weight, preferably from 0.01 to 50% by weight and most preferably from 0.01 to 15% by weight.

For use in treating crop plants, the rate of application of the active ingredients of this invention may be in the range of 0.1 g to 4000 g per hectare, desirably from 25 g to 600 g per hectare, more desirably from 50 g to 500 g per hectare.

In the treatment of seed, the application rates of the active ingredients are generally from 0.1 g to 10 kg per 100 kg of seed, preferably from 1 g to 5 kg per 100 kg of seed, in particular from 1 g to 200 g per 100 kg of seed.

The present invention is now illustrated in further detail by the following examples.

### I. PREPARATION EXAMPLES

### C. Compound Examples

Compounds were characterized e.g. by coupled High Performance Liquid Chromatography/mass spectrometry (HPLC/MS), by ¹H-NMR and/or by their melting points.

Analytical HPLC column: RP-18 column Chromolith Speed ROD from Merck KgaA, Germany). Elution: acetonitrile+0.1% trifluoroacetic acid (TFA)/water+0.1% trifluoroacetic acid (TFA) in a ratio of from 5:95 to 95:5 in 5 minutes at 40° C.

¹H-NMR, respectively ¹³C-NMR: The signals are characterized by chemical shift (ppm) vs. tetramethylsilane, respectively CDCl₃ for ¹³C-NMR, by their multiplicity and by their integral (relative number of hydrogen atoms given). The following abbreviations are used to characterize the multiplicity of the signals: m=multiplett, q=quartett, t=triplett, d=doublet and s=singulett.

The following examples were synthesized in analogy to Synthesis example S.1.

### C.1 Compound Examples 1

Compound examples 1-1 to 1-10 correspond to compounds of formula C.1:

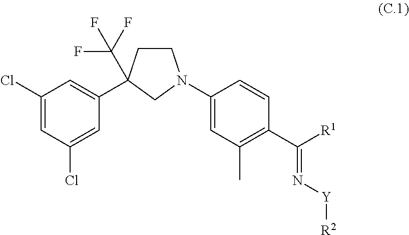

wherein R¹, R² and Y of each synthesized compound is defined in one row of table C.1 below.

TABLE C.1

| Com- | | | | | |
|---|---|---|---|---|---|
| pound | | | | Rₜ | |
| Ex. | R¹ | R² | Y | (min) | [M + H] |
| 1-1 | H | C(═O)NH—CH₂CH₃ | NH | 3.797 | 487.05 |
| 1-2 | H | C(═S)NH—CH₂CH₃ | NH | 3.959 | 503.05 |
| 1-3 | H | C(═O)NH—CH₂CF₃ | NH | 3.844 | 541.05 |
| 1-4 | H | C(═O)NH—CH₃ | NH | 3.681 | 473.05 |
| 1-5 | H | C(═O)—NH-cyclopropyl | NH | 3.817 | 499.05 |
| 1-6 | H | C(═O)—NH—CH₂-cyclopropyl | NH | 3.916 | 513.05 |
| 1-7 | H | C(═O)NH—CH₂CH₂CH₃ | NH | 3.914 | 501.10 |
| 1-8 | H | C(═O)NH—CH₂CH₂CF₃ | NH | 3.925 | 555.10 |
| 1-9 | H | C(═O)NH—CH₂CHF₂ | NH | 3.794 | 523.00 |
| 1-10 | H | C(═O)NH₂ | NH | 3.542 | 459.05 |

### C.2 Compound Examples 2

Compound example 2-1 to 2-10 corresponds to compound formula C.2:

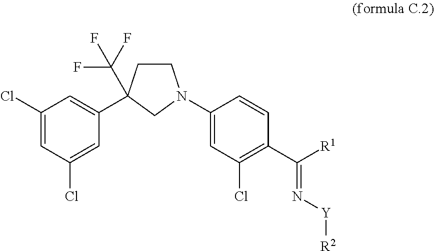

wherein R¹, R², and Y of each synthesized compound is defined in one row of table C.2 below.

TABLE C.2

| Com- | | | | | |
|---|---|---|---|---|---|
| pound | | | | Rₜ | |
| Ex. | R¹ | R² | Y | (min) | [M + H] |
| 2-1 | H | C(═O)NH—CH₂CH₃ | NH | 3.828 | 507.05 |
| 2-2 | H | C(═S)NH—CH₂CH₃ | NH | 3.992 | 522.95 |
| 2-3 | H | C(═O)NH—CH₂CF₃ | NH | 3.876 | 562.95 |
| 2-4 | H | C(═O)NH—CH₃ | NH | 3.720 | 492.95 |
| 2-5 | H | C(═O)—NH-cyclopropyl | NH | 3.841 | 521.05 |
| 2-6 | H | C(═O)—NH—CH₂-cyclopropyl | NH | 3.941 | 533.05 |
| 2-7 | H | C(═O)NH—CH₂CH₂CH₃ | NH | 3.936 | 521.05 |
| 2-8 | H | C(═O)NH—CH₂CH₂CF₃ | NH | 3.920 | 575.05 |
| 2-9 | H | C(═O)NH—CH₂CHF₂ | NH | 3.798 | 542.95 |
| 2-10 | H | C(═O)NH₂ | NH | 4.097 | 478.95 |

### C.3 Compound Examples 3

Compound example 3-1 corresponds to compound formula C.3:

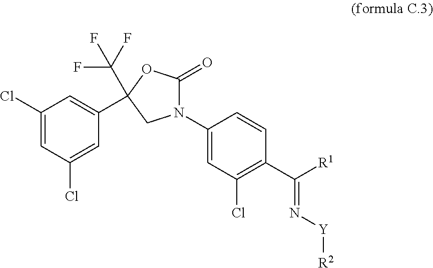

wherein R¹, R², and Y of each synthesized compound is defined in one row of table C.3 below.

TABLE C.3

| Com- | | | | | |
|---|---|---|---|---|---|
| pound | | | | Rₜ | |
| Ex. | R¹ | R² | Y | (min) | [M + H] |
| 3-1 | H | C(═O)NH—CH₂CF₃ | NH | 4.3406 | 576.90 |

### S.1 Synthesis of 4-[3-(3,5-dichloro-phenyl)-3-trifluoromethyl-pyrrolidin-1-yl]-2-methyl-benzaldehyde-ethylsemicarbazone (Compound 1.1 of table C.1)

### Step 1: Synthesis of 1-benzyl-3-(3-chloro-5-methyl-phenyl)-3-trifluoromethyl-pyrrolidine

To a solution of 2-(3,5-dichlorophenyl)-1,1,1-trifluoro-2-propene (25.381 g, 105.30 mmol) in dichloromethane (400 mL) was added N-(methoxymethyl)-N-(trimethylsilyl)benzylamine 25.0 g, 105 mmol) at 0° C. Additional dichloromethane (190 mL) was added and a solution of trifluoroacetic acid (0.6 g, 0.4 mL, 5.3 mmol) in dichloromethane (10 mL) was added dropwise at this temperature. After removal of the cooling bath, the mixture was allowed to reach room temperature over night. A 10% solution of NaHCO₃ was added and the organic layer was separated, dried over Na₂SO₄ and concentrated in vacuum. The residue was triturated with petrol ether to yield the title compound (23.0 g, 58%).

Characterization by HPLC-MS: 3.078 min, M=374.00

Characterization by ¹H-NMR (400 MHz, CDCl₃):

δ [delta]=2.32 (m, 1H), 2.57 (m, 1H), 2.74 (m, 1H), 2.78 (m, 1H), 3.06 (d, 1H), 3.09 (d, 1H), 3.66 (dd, 2H), 7.23-7.38 (m, 8H) ppm.

### Step 2: Synthesis of 3-(3-chloro-5-methyl-phenyl)-3-trifluoromethyl-pyrrolidine

To a solution of 1-benzyl-3-(3-chloro-5-methyl-phenyl)-3-trifluoromethyl-pyrrolidine (23.0 g, 61.5 mmol) in dichloromethane (200 mL) was added 1-chloroethyl chloroformate (128 mL) at 20-25° C. and the mixture was heated to reflux for 2 h. Acter concentration in vacuum, methanol (320 mL) was added and the mixture was refluxed for another 2 h. After concentration in vacuum, water was added and the mixture was extracted with hexanes/ethyl acetate (9:1) mixture twice. The aqueous phase was basified with a saturated solution of NaHCO₃ and extracted with ethyl acetate. Combined ethyl acetate layers were washed with brine, dried over Na₂SO₄ and concentrated in vacuum. The residue contained the title compound (17.0 g, 97%) and was used in the next step without further purification.

Characterization by HPLC-MS: 2.472 min, M=283.95

### Step 3: Synthesis of 4-[3-(3,5-dichloro-phenyl)-3-trifluoromethyl-pyrrolidin-1-yl]-2-methyl-benzonitrile

A solution of 3-(3-chloro-5-methyl-phenyl)-3-trifluoromethyl-pyrrolidine (8.50 g, 29.9 mmol), 4-bromo-2-methoxybenzonitrile (7.04 g, 35.9 mmol), tris(dibenzylideneacetone)dipalladium (1.096 g, 1.20 mmol), NaOtBu (4.255 g, 44.28 mmol) and 4,5-bis(diphenylphosphino)-9,9-dimethylxanthen (xanthphos CAS [161265-03-8], 1.30 g, 2.21 mmol) in degassed toluene (120 mL) was heated at reflux under argon for 16 h. After cooling, water was added and the mixture was filtered. The aqueous phase was extracted with ethyl acetate, dried over Na₂SO₄ and concentrated in vacuum. The residue was purified by flash chromatography on silica gel to obtain the title compound (3.7 g, 31%).

Characterization by HPLC-MS: 4.559 min, M=399.00

Characterization by ¹H-NMR (400 MHz, CDCl₃):

δ=2.48 (s, 3H), 2.56 (m, 1H), 2.87 (m, 1H), 3.50 (m, 1H), 3.60 (m, 1H), 3.79 (d, 1H), 4.10 (d, 1), 6.41 (m, 2H), 7.27 (m, 2H), 7.39 (s, 1H) 7.45 (d, 1H) ppm.

### Step 4: Synthesis of 4-[3-(3,5-dichloro-phenyl)-3-trifluoromethyl-pyrrolidin-1-yl]-2-methyl-benzaldehyde

To a solution of 4-[3-(3,5-dichloro-phenyl)-3-trifluoromethyl-pyrrolidin-1-yl]-2-methyl-benzonitrile (3.70 g, 9.27 mmol) in toluene (25 mL) was added DIBAL (7.5 mL of a 1.49 M solution in toluene, 11.2 mmol) at −40° C. After 15 min at this temperature, the mixture was allowed to gradually reach 0° C. After completion of the reaction, aqueous sulfuric acid (150 mL, 10%) was added and the mixture was stirred vigorously over night. The layers were separated and the organic layer was dried over Na₂SO₄ and concentrated in vacuum which yielded the title compound (3.8 g, 100%) which was used in the next step without further purification.

Characterization by HPLC-MS: 4.402 min, M=402.05

### Step 5: Synthesis of 4-[3-(3,5-dichloro-phenyl)-3-trifluoromethyl-pyrrolidin-1-yl]-2-methyl-benzaldehyde-ethylsemicarbazone

A mixture of 4-[3-(3,5-dichloro-phenyl)-3-trifluoromethyl-pyrrolidin-1-yl]-2-methyl-benzaldehyde (200 mg, 0.50 mml) and ethyl semicarbazide hydrochloride (104 mg 0.75 mmol) in ethanol (5 mL) and glacial acetic acid (0.1 mL) was heated at 70° C. for 4 h. After cooling, the mixture was concentrated in vacuum. The residue was purified by flash chromatography on silica gel to obtain the title compound (0.16 g, 66%).

Characterization by HPLC-MS: 3.797 min, M=487.05

Characterization by ¹H-NMR (400 MHz, DMSO-d⁶):

δ [delta]=1.09 (t, 3H), 2.33 (s, 3H), 2.64 (m, 1H), 2.92 (m, 1H), 3.17 (m, 2H), 3.43 (m, 2H), 3.81 (d, 1H), 4.20 (d, 1H), 6.52 (m, 2H), 6.77 (m, 1H), 7.70 (m, 3H), 7.76 (d, 1H), 8.03 (s, 1H), 9.90 (s, 1H) ppm.

### S.2 1-[(E)-[2-chloro-4-[5-(3,5-dichlorophenyl)-2-oxo-5-(trifluoromethyl)oxazolidin-3-yl]phenyl]methyleneamino]-3-(2,2,2-trifluoroethyl)urea (Compound 3.1 of table C.3)

### Step 1: Synthesis of 2-(3,5-dichlorphenyl)-2-(trifluormethyl)oxirane

Sodium hydride (60%, 5.5 g, 144 mmol) was added to a stirred solution of trimethyl-sulfoxonium iodide (34.1 g, 154 mmol) in dimethylformamide (350 ml) under a nitrogen atmosphere. After stirring for 1 h, the solution was added dropwise over 30 minutes to a solution of 1-(3,5-dichlorophenyl)-2,2,2-trifluoroethanone (25.0 g, 103 mmol) in dimethylformamide (150 ml) at 0° C. The resulting mixture was stirred for 30 minutes at 0° C. and added to an ice/water mixture. The solution was extracted twice with methyl tert-butyl ether (MTBE). The combined MTBE extracts were washed with water and dried (MgSO₄). The solvent was removed under reduced pressure to provide an orange oil (19.9 g). Purification by flash chromatography on silica gel using ethyl acetate-cyclohexane as eluent afforded the title compound (5.2 g, 20%).

Characterization by HPLC: 3.916 min

Characterization by ¹H-NMR (400 MHz, CDCl₃):

δ [delta]=2.91 (m, 1H), 3.43 (d, 1H), 7.42 (m, 3H) ppm.

### Step 2: Synthesis of 3-(4-bromo-3-chloro-anilino)-2-(3,5-dichlorophenyl)-1,1,1-trifluoro-propan-2-ol

A solution of 2-(3,5-dichlorphenyl)-2-(trifluormethyl)oxirane (4.16 g, 20 mmol) and 4-bromo-3-chloro-aniline (3.34 g, 20 mmol) in anhydrous ethanol (40 ml) was heated at 75° C. over night. After cooling, the solvent was concentrated under reduced pressure. The residue was purified by flash chromatography on silica gel using ethyl acetate-cyclohexane as eluent to provide the title compound (6.1 g, 81%).

Characterization by HPLC-MS: 4.317 min, M=463.80

### Step 3: Synthesis of 3-(4-bromo-3-chloro-phenyl)-5-(3,5-dichlorophenyl)-5-(trifluoromethyl)oxazolidin-2-one

A solution of 3-(4-bromo-3-chloro-anilino)-2-(3,5-dichlorophenyl)-1,1,1-trifluoro-propan-2-01 (6.1 g, 10 mmol), N,N-dimethylaminopyridine (6.5 g) and 1,1′-carbonyldiimidazole (10.2 g, 60 mmol) in dichloromethane (60 ml) was stirred at room temperature over night. The solvent was removed under reduced pressure and the residue was purified by flash chromatography on silica gel using ethyl acetate-cyclohexane as eluent to provide the title compound (2.4 g, 37%).

Characterization by HPLC-MS: 4.552 min, M=489.70

### Step 4: Synthesis of 2-chloro-4-[5-(3,5-dichlorophenyl)-2-oxo-5-(trifluoromethyl)oxazolidin-3-yl]benzaldehyde

To a solution of 3-(4-bromo-3-chloro-phenyl)-5-(3,5-dichlorophenyl)-5-(trifluoromethyl)oxazolidin-2-one (0.75 g, 1.5 mmol) in dimethylformamide (DMF) (30 ml) was added [1,1′-bis(diphenylphosphino)ferrocene]dichloropalladium(II) (50 mg), Na₂CO₃ (180 mg) and triethylsilane (0.5 ml). The mixture was purged twice with N₂, then purged three times with CO and stirred under an atmosphere of CO at 90° C. for 48 h. The reaction mixture was poured into water and adjusted with aqueous HCl to pH 4-5. The aqueous phase was extracted twice with methyl tert-butyl ether (MTBE). The combined MTBE extracts were washed with water and dried over MgSO₄. The solvent was removed under reduced pressure and purified by flash chromatography on silica gel using ethyl acetate-cyclohexane as eluent to provide the title compound (153 mg, 23%).

Characterization by HPLC-MS: 4.356 min, M=437.95

### Step 5: Synthesis of 1-[(E)-[2-chloro-4-[5-(3,5-dichlorophenyl)-2-oxo-5-(trifluoromethyl)oxazolidin-3-yl]phenyl]methyleneamino]-3-(2,2,2-trifluoroethyl)urea

A mixture of 2-chloro-4-[5-(3,5-dichlorophenyl)-2-oxo-5-(trifluoromethyl) oxazolidin-3-yl]benzaldehyde (124 mg, 0.28 mmol) and 2,2,2-trifluoroethyl semicarbazide hydrochloride (102 mg 0.42 mmol) in ethanol (10 mL) and glacial acetic acid (0.03 mL) was heated at 70° C. for 4 h. After cooling, the mixture was concentrated in vacuum. The residue was purified by flash chromatography on silica gel to obtain the title compound (0.088 g, 54%).

Characterization by HPLC-MS: 4.346 min, M=576.90

Characterization by ¹H-NMR (400 MHz, CDCl₃):

δ [delta]=4.01 (m, 2H), 4.29 (d, 1H), 4.62 (d, 1H), 6.43 (m, 1H), 7.52 (m, 5H), 7.92 (d, 1H), 8.11 (s, 1H), 8.89 (s, 1H) ppm.

### II. EVALUATION OF PESTICIDAL ACTIVITY

The activity of the compounds of formula I of the present invention can be demonstrated and evaluated by the following biological test.

B.1 Cotton Aphid (Aphis gossypii)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Cotton plants at the cotyledon stage were infested with aphids prior to treatment by placing a heavily infested leaf from the main aphid colony on top of each cotyledon. Aphids were allowed to transfer overnight to accomplish an infestation of 80-100 aphids per plant and the host leaf was removed. The infested plants were then sprayed by an automated electrostatic plant sprayer equipped with an atomizing spray nozzle. The plants were dried in the sprayer fume hood, removed from the sprayer, and then maintained in a growth room under fluorescent lighting in a 24-hr photoperiod at 25° C. and 20-40% relative humidity. Aphid mortality on the treated plants, relative to mortality on untreated control plants, was determined after 5 days.

B.2 Cowpea Aphid (Aphis craccivora)

Potted cowpea plants colonized with approximately 100-150 aphids of various stages were sprayed after the pest population had been recorded. Population reduction was assessed after 24, 72, and 120 hours.

In this test, the compounds 1-8 and 3-1, respectively, at 500 ppm showed a mortality of at least 75% in comparison with untreated controls.

B.3 Diamond Back Moth (Plutella xylostella)

Leaves of Chinese cabbage were dipped in test solution and air-dried. Treated leaves were placed in petri dished lined with moist filter paper. Mortality was recorded 24, 72, and 120 hours after treatment.

In this test, the compounds 1-1, 1-4, 1-6, 1-7, 1-8, 1-9, 2-4, 2-7, 2-8, 2-9 and 3-1, respectively, at 500 ppm showed a mortality of at least 75% in comparison with untreated controls.

B.4 Green Peach Aphid (Myzus persicae)

For evaluating control of green peach aphid (Myzus persicae) through systemic means the test unit consisted of 96-well-microtiter plates containing liquid artificial diet under an artificial membrane.

The compounds were formulated using a solution containing 75% v/v water and 25% v/v DMSO. Different concentrations of formulated compounds were pipetted into the aphid diet, using a custom built pipetter, at two replications.

After application, 5-8 adult aphids were placed on the artificial membrane inside the microtiter plate wells. The aphids were then allowed to suck on the treated aphid diet and incubated at about 23±1° C. and about 50±5% relative humidity for 3 days. Aphid mortality and fecundity was then visually assessed.

In this test, the compounds 1-1, 1-4, 1-5, 1-6, 1-7, 1-8, 1-9, 2-1, 2-2, 2-3, 2-4, 2-5, 2-6, 2-7, 2-8, 2-9 and 3-1, respectively, at 2500 ppm showed a mortality of at least 75% in comparison with untreated controls.

B.5 Mediterranean Fruitfly (Ceratitis capitata)

For evaluating control of Mediterranean fruitfly (Ceratitis capitata) the test unit consisted of microtiter plates containing an insect diet and 50-80 C. capitata eggs.

The compounds were formulated using a solution containing 75% v/v water and 25% v/v DMSO. Different concentrations of formulated compounds were sprayed onto the insect diet at 5 μl, using a custom built micro atomizer, at two replications.

After application, microtiter plates were incubated at about 28±1° C. and about 80±5% relative humidity for 5 days. Egg and larval mortality was then visually assessed.

In this test, the compounds 1-1, 1-3, 1-4, 1-5, 1-6, 1-7, 1-8, 1-9, 2-1, 2-3, 2-4, 2-5, 2-6, 2-7, 2-8, 2-9, 2-10 and 3-1, respectively, at 2500 ppm showed a mortality of at least 75% in comparison with untreated controls.

B.6 Orchid Thrips (dichromothrips corbetti)

Dichromothrips corbetti adults used for bioassay were obtained from a colony maintained continuously under laboratory conditions. For testing purposes, the test compound was diluted to a concentration of 500 ppm (wt compound: vol diluent) in a 1:1 mixture of acetone:water (vol:vol), plus 0.01% vol/vol Kinetic® surfactant.

Thrips potency of each compound was evaluated by using a floral-immersion technique. Plastic petri dishes were used as test arenas. All petals of individual, intact orchid flowers were dipped into treatment solution and allowed to dry. Treated flowers were placed into individual petri dishes along with 10-15 adult thrips. The petri dishes were then covered with lids. All test arenas were held under continuous light and a temperature of about 28° C. for duration of the assay. After 4 days, the numbers of live thrips were counted on each flower, and along inner walls of each petri dish. The level of thrips mortality was extrapolated from pre-treatment thrips numbers.

In this test, the compound 3-1 at 500 ppm showed a mortality of at least 75% in comparison with untreated controls.

B.7 Rice Green Leafhopper (Nephotettix virescens)

Rice seedlings were cleaned and washed 24 hours before spraying. The active compounds were formulated in 50:50 acetone:water (vol:vol), and 0.1% vol/vol surfactant (EL 620) was added. Potted rice seedlings were sprayed with 5 ml test solution, air dried, placed in cages and inoculated with 10 adults. Treated rice plants were kept at about 28-29° C. and relative humidity of about 50-60%. Percent mortality was recorded after 72 hours.

B.8 Silverleaf Whitefly (bemisia argentifolii)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Cotton plants at the cotyledon stage (one plant per pot) were sprayed by an automated electrostatic plant sprayer equipped with an atomizing spray nozzle. The plants were dried in the sprayer fume hood and then removed from the sprayer. Each pot was placed into a plastic cup and about 10 to 12 whitefly adults (approximately 3-5 days old) were introduced. The insects were collected using an aspirator and a nontoxic Tygon® tubing connected to a barrier pipette tip. The tip, containing the collected insects, was then gently inserted into the soil containing the treated plant, allowing insects to crawl out of the tip to reach the foliage for feeding. Cups were covered with a reusable screened lid. Test plants were maintained in a growth room at about 25° C. and about 20-40% relative humidity for 3 days, avoiding direct exposure to fluorescent light (24 hour photoperiod) to prevent trapping of heat inside the cup. Mortality was assessed 3 days after treatment, compared to untreated control plants.

In this test, the compound 2-4, at 300 ppm showed a mortality of at least 75% in comparison with untreated controls.

B.9 Southern Armyworm (Spodoptera eridania)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Lima bean plants (variety Sieva) were grown 2 plants to a pot and selected for treatment at the 1^{st} true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 armyworm larvae were placed into the bag and the bags zipped closed. Test plants were maintained in a growth room at about 25° C. and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, the compounds 1-1, 1-3, 1-4, 1-7, 1-8, 1-9, 1-10, 2-1, 2-3, 2-4, 2-5, 2-7, 2-8 and 2-9 respectively, at 300 ppm showed a mortality of at least 75% in comparison with untreated controls.

B.10 Vetch Aphid (Megoura viciae)

For evaluating control of vetch aphid (Megoura viciae) through contact or systemic means the test unit consisted of 24-well-microtiter plates containing broad bean leaf disks.

The compounds were formulated using a solution containing 75% v/v water and 25% v/v DMSO. Different concentrations of formulated compounds were sprayed onto the leaf disks at 2.5 μl, using a custom built micro atomizer, at two replications.

After application, the leaf disks were air-dried and 5-8 adult aphids placed on the leaf disks inside the microtiter plate wells. The aphids were then allowed to suck on the treated leaf disks and incubated at about 23±1° C. and about 50±5% relative humidity for 5 days. Aphid mortality and fecundity was then visually assessed.

In this test, the compounds 1-1, 1-4, 1-5, 1-6, 1-7, 1-8, 1-9, 2-1, 2-3, 2-4, 2-5, 2-6, 2-7 and 2-9, respectively, at 2500 ppm showed a mortality of at least 75% in comparison with untreated controls.

B.11 Tobacco Budworm (Heliothis virescens) I

For evaluating control of tobacco budworm (Heliothis virescens) the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom built micro atomizer, at two replications.

After application, microtiter plates were incubated at about 28±1° C. and about 80±5% relative humidity for 5 days. Egg and larval mortality was then visually assessed.

In this test, the compounds 1-1, 1-2, 1-3, 1-4, 1-5, 1-6, 1-7, 1-8, 1-9, 2-1, 2-2, 2-3, 2-4, 2-5, 2-6, 2-7, 2-8, 2-9, 2-10 and 3-1, respectively, at 2500 ppm showed a mortality of at least 75% in comparison with untreated controls.

B.12 Boll Weevil (Anthonomus grandis)

For evaluating control of boll weevil (Anthonomus grandis) the test unit consisted of 24-well-microtiter plates containing an insect diet and 20-30 A. grandis eggs.

The compounds were formulated using a solution containing 75% v/v water and 25% v/v DMSO. Different concentrations of formulated compounds were sprayed onto the insect diet at 20 μl, using a custom built micro atomizer, at two replications.

After application, microtiter plates were incubated at about 23±1° C. and about 50±5% relative humidity for 5 days. Egg and larval mortality was then visually assessed.

In this test, the compounds 1-1, 1-3, 1-4, 1-5, 1-6, 1-7, 1-8, 1-9, 2-1, 2-3, 2-4, 2-5, 2-6, 2-7, 2-8, 2-9, 2-10 and 3-1, respectively, at 2500 ppm showed a mortality of at least 75% in comparison with untreated controls.

B.13 Colorado Potato Beetle (Leptinotarsa decemlineata)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Eggplants were grown 2 plants to a pot and were selected for treatment at the 1^{st} true leaf stage. Test solutions were sprayed onto the foliage by an automated electrostatic plant sprayer equipped with an atomizing spray nozzle. The plants were dried in the sprayer fume hood and then removed from the sprayer. The treated foliage was then cut and removed from the pot and placed in a 5-inch Petri dish lined with moistened filter paper. Five beetle larvae were introduced into each Petri dish and the dish was covered by a Petri dish lid. Petri dishes were maintained in a growth room at 25° C. and 20-40% relative humidity for 4 days, avoiding direct exposure to fluorescent light (24 hour photoperiod) to prevent trapping of heat inside the dishes. Mortality and reduced feeding were assessed 4 days after treatment, compared to untreated control plants.

B.14 Red Spider Mite (Tetranychus kanzawai)

The active compound was dissolved at the desired concentration in a mixture of 1:1 (v/v) distilled water:acetone. A surfactant (Alkamuls® EL 620) was added at the rate of 0.1% (v/v).

Potted cowpea beans of 7-10 days of age were cleaned with tap water and sprayed with 5 ml of the test solution using air driven hand atomizer. The treated plants were allowed to air dry and afterwards inculated with 20 or more mites by clipping a cassava leaf section with known mite population. Treated plants were placed inside a holding room at about 25-27° C. and about 50-60% relative humidity.

Mortality was determined by counting the live mites 72 HAT. Percent mortality was assessed after 72 h.

In this test, the compounds 1-9 and 2-9, respectively, at 500 ppm showed a mortality of at least 75% in comparison with untreated controls.

1-38. (canceled)

39. A compound of formula (I) 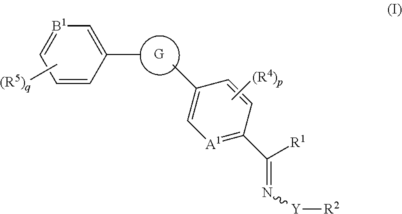
wherein
A¹ is N or CH;
B¹ is N or CH;
G is a bivalent heterocyclic ring selected from the following groups G-1 to G-28 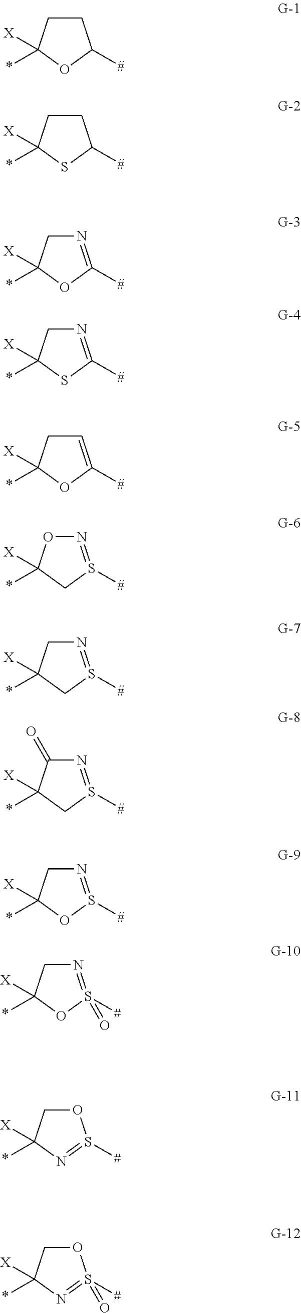 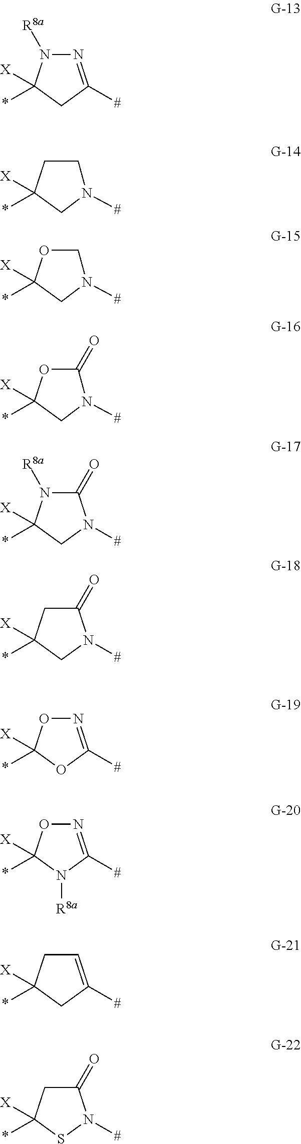 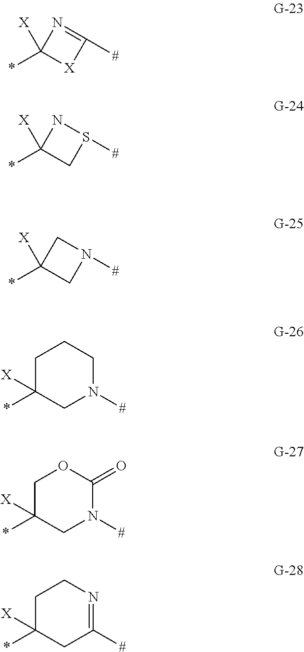
Preliminary Amendment Dated: Mar. 27, 2013
wherein the “*” and “#” in the variables G-1 to G-28 indicate the bonds to the neighbouring phenyl or pyridyl rings;
X is selected from the group consisting of C₁-C₄-alkyl, C₁-C₄-haloalkyl, C₁-C₄-alkoxy-C₁-C₄-alkyl, C₁-C₄-haloalkoxy-C₁-C₄-alkyl, C₂-C₄-alkenyl, C₂-C₄-haloalkenyl, C₂-C₄-alkynyl, C₂-C₄-haloalkynyl, C₃-C₆-cycloalkyl and C₃-C₆-halocycloalkyl;
Y is O, N—R³, S(O)ₙ or a chemical bond;
R¹ is selected from the group consisting of hydrogen; cyano; C₁-C₁₀-alkyl which may be partially or fully halogenated and/or may be substituted by one or more radicals R⁶; C₁-C₁₀-alkoxy; C₁-C₁₀-haloalkoxy; C₁-C₁₀-alkylthio; C₁-C₁₀-haloalkylthio; C₁-C₁₀-alkylsulfinyl; C₁-C₁₀-haloalkylsulfinyl; C₁-C₁₀-alkylsulfonyl; C₁-C₁₀-haloalkylsulfonyl; C₃-C₈-cycloalkyl which may be partially or fully halogenated and/or may be substituted by one or more radicals R⁶; C₂-C₁₀-alkenyl which may be partially or fully halogenated and/or may be substituted by one or more radicals R⁶; C₂-C₁₀-alkynyl which may be partially or fully halogenated and/or may be substituted by one or more radicals R⁶; —C(═O)R⁶; —C(═O)OR⁷; —C(═O)N(R⁸)R⁹; —C(═S)R⁶; —C(═S)OR⁷; —C(═S)N(R⁸)R⁹; phenyl which may be substituted by 1, 2, 3, 4 or 5 radicals R¹⁰; and a C-bound 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
R² is selected from the group consisting of hydrogen; cyano; C₁-C₁₀-alkyl which may be partially or fully halogenated and/or may be substituted by one or more radicals R⁶; C₃-C₈-cycloalkyl which may be partially or fully halogenated and/or may be substituted by one or more radicals R⁶; C₂-C₁₀-alkenyl which may be partially or fully halogenated and/or may be substituted by one or more radicals R⁶; C₂-C₁₀-alkynyl which may be partially or fully halogenated and/or may be substituted by one or more radicals R⁶;
—N(R⁸)R⁹; —N(R⁸)C(═O)R⁶; —Si(R¹⁴)₂R¹³; —OR⁷; —SR⁷; —S(O)ₘR⁷; —S(O)ₙN(R⁸)R⁹; —C(═O)R⁶; —C(═O)OR⁷; —C(═O)N(R⁸)R⁹; —C(═S)R⁶; —C(═S)OR⁷, —C(═S)N(R⁸)R⁹; —C(═NR⁸)R⁶; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
with the proviso that R² is not —OR⁷ if Y is O;
R³ is selected from the group consisting of hydrogen; cyano; C₁-C₁₀-alkyl which may be partially or fully halogenated and/or may be substituted by one or more radicals R⁶; C₃-C₈-cycloalkyl which may be partially or fully halogenated and/or may be substituted by one or more radicals R⁶; C₂-C₁₀-alkenyl which may be partially or fully halogenated and/or may be substituted by one or more radicals R⁶; C₂-C₁₀-alkynyl which may be partially or fully halogenated and/or may be substituted by one or more radicals R⁶;
—N(R⁸)R⁹; —Si(R¹⁴)₂R¹³; —OR⁷; —SR⁷; —S(O)ₘR⁷; —S(O)ₙN(R⁸)R⁹; —C(═O)R⁶; —C(═O)OR⁷; —C(═O)N(R⁸)R⁹; —C(═S)R⁶; —C(═S)OR⁷; —C(═S)N(R⁸)R⁹; —C(═NR⁸)R⁶; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
or R² and R³ together form a group ═CR¹¹R¹²; ═S(O)ₘR⁷; ═S(O)ₘN(R⁸)R⁹; ═NR⁸; or ═NOR⁷;
or R² and R³ together form a C₂-C₇ alkylene chain, thus forming, together with the nitrogen atom to which R² and R³ are bound, a 3-, 4-, 5-, 6-, 7- or 8-membered ring, where the alkylene chain may be interrupted by 1 or 2 O, S or NR¹⁸ and/or 1 or 2 of the CH₂ groups of the alkylene chain may be replaced by a group C═O, C═S or C═NR¹⁸; or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
each R⁴ is independently selected from the group consisting of halogen; cyano; azido; nitro; —SCN; SF₅; C₁-C₆-alkyl which may be partially or fully halogenated and/or may be substituted by one or more radicals R⁶; C₃-C₈-cycloalkyl which may be partially or fully halogenated and/or may be substituted by one or more radicals R⁶; C₂-C₆-alkenyl which may be partially or fully halogenated and/or may be substituted by one or more radicals R⁶; C₂-C₆-alkynyl which may be partially or fully halogenated and/or may be substituted by one or more radicals R⁶; —Si(R¹⁴)₂R¹³; —OR⁷; —OS(O)ₙR⁷; —SR⁷;
—S(O)ₘR⁷; —S(O)ₙN(R⁸)R⁹; —N(R⁸)R⁹; —N(R⁸)C(═O)R⁶; C(═O)R⁶; —C(═O)OR⁷; —C(═NR⁸)H; —C(═NR⁸)R⁶; —C(═O)N(R⁸)R⁹; C(═S)N(R⁸)R⁹; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
or two radicals R⁴ bound on adjacent carbon atoms may be together form a group selected from the group consisting of —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR⁸—, —CH₂CH═N—, —CH═CH—NR⁸—, —OCH═N— and —SCH═N—, thus forming, together with the carbon atoms to which the two radicals R⁴ are bound, a 5- or 6-membered ring, where the hydrogen atoms of the above groups may be replaced by one or more substituents selected from the group consisting of halogen, methyl, halomethyl, hydroxyl, methoxy and halomethoxy or one or more CH₂ groups of the above groups may be replaced by a C═O group;
each R⁵ is independently selected from the group consisting of halogen, cyano, azido, nitro, —SCN, SF₅, C₁-C₆-alkyl which may be partially or fully halogenated and/or may be substituted by one or more radicals R⁶, C₃-C₈-cycloalkyl which may be partially or fully halogenated and/or may be substituted by one or more radicals R⁶, C₂-C₆-alkenyl which may be partially or fully halogenated and/or may be substituted by one or more radicals R⁶, C₂-C₆-alkynyl which may be partially or fully halogenated and/or may be substituted by one or more radicals R⁶, —Si(R¹⁴)₂R¹³, —OS(O)ₙR⁷, —SR⁷, —S(O)ₘR⁷, —S(O)ₙN(R⁸)R⁹, —N(R⁸)R⁹, N(R⁸)C(═O)R⁶, —C(═O)R⁶, —C(═O)OR⁷, —C(═S)R⁶, —C(═S)OR⁷, —C(═NR⁸)R⁶, —C(═O)N(R⁸)R⁹, —C(═S)N(R⁸)R⁹, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
each R⁶ is independently selected from the group consisting of cyano, azido, nitro, —SCN, SF₅, C₃-C₈-cycloalkyl, C₃-C₈-halocycloalkyl, —Si(R¹⁴)₂R¹³, —OR⁷, —OSO₂R⁷, —S(O)ₘR⁷, —S(O)ₙN(R⁸)R⁹, —N(R⁸)R⁹, —C(═O)N(R⁸)R⁹, —C(═S)N(R⁸)R⁹, —C(═O)OR⁷, —C(═O)R¹⁹, —C(═NR⁸)R¹⁹, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
and, in case R⁶ is bound to a cycloalkyl group, R⁶ may additionally be selected from the group consisting of C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, C₂-C₆-haloalkynyl and benzyl which may be substituted by 1, 2, 3, 4 or 5 radicals R¹⁰;
and in groups —C(═O)R⁶, —C(═S)R⁶, —C(═NR⁸)R⁶ and —N(R⁸)C(═O)R⁶, R⁶ may additionally be selected from the group consisting of hydrogen, halogen, C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, C₂-C₆-haloalkynyl and benzyl which may be substituted by 1, 2, 3, 4 or 5 radicals R¹⁰;
or two geminally bound radicals R⁶ together form a group selected from the group consisting of ═CR¹¹R¹², ═S(O)ₘR⁷, ═S(O)ₘN(R⁸)R⁹, ═NR⁸, ═NOR⁷ and ═NNR⁸;
or two radicals R⁶, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from the group consisting of N, O, S, NO, SO and SO₂, as ring members;
each R⁷ is independently selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁴)₂R¹³, —SR⁸, —S(O)ₘR⁷, —S(O)ₙN(R⁸)R⁹, —N(R⁸)R⁹, —N═CR¹⁵R¹⁶, —C(═O)R¹⁷, —C(═O)N(R⁸)R⁹, —C(═S)N(R⁸)R⁹, —C(═O)OR¹⁷, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
with the proviso that R⁷ is not C₁-C₆-alkoxy or C₁-C₆-haloalkoxy if it is bound to an oxygen atom;
each R⁸ is independently selected from the group consisting of hydrogen, cyano, C₁-C₆-alkyl which may be partially or fully halogenated and/or may be substituted by one or more radicals R¹⁹, C₁-C₆-alkoxy, C₁-C₆-haloalkoxy, C₁-C₆-alkylthio, C₁-C₆-haloalkylthio, where the alkyl moiety in the four last-mentioned radicals may be substituted by one or more radicals R¹⁹, C₃-C₈-cycloalkyl which may be partially or fully halogenated and/or may be substituted by one or more radicals R¹⁹, C₃-C₈-cycloalkyl-C₁-C₄-alkyl where the cycloalkyl moiety may be partially or fully halogenated and/or may be substituted by one or more radicals R¹⁹, C₂-C₆-alkenyl which may be partially or fully halogenated and/or may be substituted by one or more radicals R¹⁹, C₂-C₆-alkynyl which may be partially or fully halogenated and/or may be substituted by one or more radicals R¹⁹, —S(O)ₘR²⁰, —S(O)ₙN(R²¹)R²², phenyl which may be substituted by 1, 2, 3, 4 or 5 radicals R¹⁰, benzyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
each R⁹ is independently selected from the group consisting of hydrogen, cyano, C₁-C₆-alkyl which may be partially or fully halogenated and/or may be substituted by one or more radicals R¹⁹, C₁-C₆-alkoxy, C₁-C₆-haloalkoxy, C₁-C₆-alkylthio, C₁-C₆-haloalkylthio, where the alkyl moiety in the four last-mentioned radicals may be substituted by one or more radicals R¹⁹, C₃-C₈-cycloalkyl which may be partially or fully halogenated and/or may be substituted by one or more radicals R¹⁹, C₃-C₈-cycloalkyl-C₁-C₄-alkyl where the cycloalkyl moiety may be partially or fully halogenated and/or may be substituted by one or more radicals R¹⁹, C₂-C₆-alkenyl which may be partially or fully halogenated and/or may be substituted by one or more radicals R¹⁹, C₂-C₆-alkynyl which may be partially or fully halogenated and/or may be substituted by one or more radicals R¹⁹, —S(O)ₘR²⁰, —S(O)ₙN(R²¹)R²², phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
or R⁸ and R⁹ together form a group ═CR¹¹R¹²;
or R⁸ and R⁹, together with the nitrogen atom to which they are bound, may form a 3-, 4-, 5-, 6- or 7-membered saturated, partially unsaturated or aromatic heterocyclic ring which may additionally containing 1 or 2 further heteroatoms or heteroatom groups selected from the group consisting of N, O, S, NO, SO and SO₂, as ring members, where the heterocyclic ring may be substituted by one or more radicals R¹⁰;
each R^{8a} is independently R⁸;
each R¹⁰ is independently selected from the group consisting of halogen, cyano, azido, nitro, —SCN, SF₅, C₁-C₁₀-alkyl which may be partially or fully halogenated and/or may be substituted by one or more radicals R¹⁹, C₃-C₈-cycloalkyl which may be partially or fully halogenated and/or may be substituted by one or more radicals R¹⁹, C₂-C₁₀-alkenyl which may be partially or fully halogenated and/or may be substituted by one or more radicals R¹⁹, C₂-C₁₀-alkynyl which may be partially or fully halogenated and/or may be substituted by one or more radicals R¹⁹, —Si(R¹⁴)₂R¹³, —OR²⁰, —OS(O)ₙR²⁰, —SR²⁰, —S(O)ₘR²⁰, —S(O)ₙN(R²¹)R²², —N(R²¹)R²², C(═O)R¹⁹, —C(═O)OR²⁰, —C(═NR²¹)R²², —C(═O)N(R²¹)R²², —C(═S)N(R²¹)R²², phenyl which may be substituted by 1, 2, 3, 4 or 5 radicals independently selected from the group consisting of halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from the group consisting of N, O, S, NO, SO and SO₂, as ring members, which may be substituted by one or more radicals independently selected from the group consisting of halogen, cyano, nitro, C₁-C₆-alkyl, C₁-C₆-haloalkyl, C₁-C₆-alkoxy and C₁-C₆-haloalkoxy;
or two radicals R¹⁰ bound on adjacent atoms together form a group selected from the group consisting of —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR²¹—, —CH₂CH═N—, —CH═CH—NR²¹—, —OCH═N— and —SCH═N—, thus forming, together with the atoms to which the two radicals R¹⁰ are bound, a 5- or 6-membered ring, where the hydrogen atoms of the above groups may be replaced by one or more substituents selected from the group consisting of halogen, methyl, halomethyl, hydroxyl, methoxy and halomethoxy or one or more CH₂ groups of the above groups may be replaced by a C═O group;
R¹¹, R¹² are, independently of each other and independently of each occurrence, selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, C₁-C₆-alkoxy, C₁-C₆-haloalkoxy, —C(═O)R¹⁹, —C(═O)OR²⁰, —C(═NR²¹)R²², —C(═O)N(R²¹)R²², —C(═S)N(R²¹)R²², phenyl which may be substituted by 1, 2, 3, 4, or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, which may be substituted by one or more radicals R¹⁰;
R¹³, R¹⁴ are, independently of each other and independently of each occurrence, selected from the group consisting of C₁-C₄-alkyl, C₃-C₆-cycloalkyl, C₁-C₄-alkoxy-C₁-C₄-alkyl, phenyl and benzyl;
R¹⁵, R¹⁶ are, independently of each other and independently of each occurrence, selected from the group consisting of C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, phenyl which may be substituted by 1, 2, 3, 4, or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, which may be substituted by one or more radicals R¹⁰;
each R¹⁷ is independently selected from the group consisting of C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, phenyl and benzyl;
each R¹⁸ is independently R³;
each R¹⁹ is independently selected from the group consisting of cyano, azido, nitro, —SCN, SF₅, C₃-C₈-cycloalkyl, C₃-C₈-halocycloalkyl, —Si(R¹⁴)₂R¹³, —OR²⁰, —OSO₂R²⁰, —SR²⁰, —S(O)ₘR²⁰, —S(O)ₙN(R²¹)R²², —N(R²¹)R²², —C(═O)N(R²¹)R²², —C(═S)N(R²¹)R²², —C(═NR²¹)R²⁰, —C(═O)OR²⁰, —C(═O)R²⁰, phenyl which may be substituted by 1, 2, 3, 4 or 5 radicals independently selected from the group consisting of halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals independently selected from the group consisting of halogen, cyano, nitro, C₁-C₆-alkyl, C₁-C₆-haloalkyl, C₁-C₆-alkoxy and C₁-C₆-haloalkoxy;
and, in case R¹⁹ is bound to a cycloalkyl group, R¹⁹ may additionally be selected from the group consisting of C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl and C₂-C₆-haloalkynyl;
and in groups —C(═O)R¹⁹, R¹⁹ may additionally be selected from the group consisting of hydrogen, halogen, C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, and C₂-C₆-haloalkynyl;
or two geminally bound radicals R¹⁹ together form a group selected from the group consisting of ═CR¹¹R¹², ═S(O)ₘR²⁰, ═S(O)ₘN(R²¹)R²², ═NR²¹, ═NOR²⁰ and ═NNR²¹;
or two radicals R¹⁹, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from the group consisting of N, O, S, NO, SO and SO₂, as ring members;
each R²⁰ is independently selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁴)₂R¹³, C₁-C₆-alkylaminosulfonyl, amino, C₁-C₆-alkylamino, di-(C₁-C₆-alkyl)-amino, C₁-C₆-alkylcarbonyl, C₁-C₆-haloalkylcarbonyl, aminocarbonyl, C₁-C₆-alkylaminocarbonyl, di-(C₁-C₆-alkyl)-aminocarbonyl, C₁-C₆-alkoxycarbonyl, C₁-C₆-haloalkoxycarbonyl, phenyl which may be substituted by 1, 2, 3, 4 or 5 radicals independently selected from the group consisting of halogen, cyano, nitro, C₁-C₆-alkyl, C₁-C₆-haloalkyl, C₁-C₆-alkoxy and C₁-C₆-haloalkoxy, benzyl which may be substituted by 1, 2, 3, 4 or 5 radicals independently selected from the group consisting of halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals independently selected from the group consisting of halogen, cyano, nitro, C₁-C₆-alkyl, C₁-C₆-haloalkyl, C₁-C₆-alkoxy and C₁-C₆-haloalkoxy;
with the proviso that R²⁰ is not C₁-C₆-alkoxy or C₁-C₆-haloalkoxy if it is bound to an oxygen atom;
R²¹ and R²² are independently of each other and independently of each occurrence selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₃-C₈-cycloalkyl-C₁-C₄-alkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals independently selected from the group consisting of halogen, cyano, nitro, C₁-C₆-alkyl, C₁-C₆-haloalkyl, C₁-C₆-alkoxy and C₁-C₆-haloalkoxy, benzyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals independently selected from the group consisting of halogen, cyano, nitro, C₁-C₆-alkyl, C₁-C₆-haloalkyl, C₁-C₆-alkoxy and C₁-C₆-haloalkoxy;
or R²¹ and R²², together with the nitrogen atom to which they are bound, may form a 3-, 4-, 5-, 6- or 7-membered saturated, partially unsaturated or aromatic heterocyclic ring which may additionally containing 1 or 2 further heteroatoms or heteroatom groups selected from the group consisting of N, O, S, NO, SO and SO₂, as ring members, where the heterocyclic ring may be substituted by one or more radicals selected from the group consisting of halogen, C₁-C₆-alkyl, C₁-C₆-haloalkyl, C₁-C₆-alkoxy and C₁-C₆-haloalkoxy;
each m is independently 1 or 2;
each n is independently 0, 1 or 2;
p is 0, 1, 2, 3 or 4; and
q is 0, 1, 2, 3, 4 or 5;
or an stereoisomer or agriculturally or veterinarily acceptable salt thereof.

40. The compound according to claim 39, wherein B¹ is CH.

41. The compound according to claim 39, wherein A¹ is CH.

42. The compound according to claim 39, wherein X is selected from the group consisting of C₁-C₄-alkyl, C₁-C₄-haloalkyl, C₁-C₄-alkoxy-C₁-C₄-alkyl, C₁-C₄-haloalkoxy-C₁-C₄-alkyl, C₃-C₆-cycloalkyl and C₃-C₆-halocycloalkyl.

43. The compound according to claim 39, wherein X is selected from the group consisting of CF₃, CHF₂ and CF₂Cl.

44. The compound according to claim 39, wherein R¹ is selected from the group consisting of hydrogen; cyano; C₁-C₁₀-alkyl which may be partially or fully halogenated and/or may be substituted by one or more radicals R⁶; C₁-C₁₀-alkoxy; C₁-C₁₀-haloalkoxy; C₃-C₈-cycloalkyl which may be partially or fully halogenated and/or may be substituted by one or more radicals R⁶; C₂-C₁₀-alkenyl which may be partially or fully halogenated and/or may be substituted by one or more radicals R⁶; C₂-C₁₀-alkynyl which may be partially or fully halogenated and/or may be substituted by one or more radicals R⁶; —C(═O)R⁶; —C(═O)OR⁷; —C(═O)N(R⁸)R⁹; —C(═S)R⁶; —C(═S)OR⁷; —C(═S)N(R⁸)R⁹; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰.

45. The compound according to claim 44, wherein R¹ is selected from the group consisting of hydrogen; cyano; C₁-C₁₀-alkyl which may be partially or fully halogenated and/or may be substituted by one or more radicals R⁶; C₁-C₁₀-alkoxy; C₁-C₁₀-haloalkoxy; C₃-C₈-cycloalkyl which may be partially or fully halogenated and/or may be substituted by one or more radicals R⁶; C₂-C₁₀-alkenyl which may be partially or fully halogenated and/or may be substituted by one or more radicals R⁶; C₂-C₁₀-alkynyl which may be partially or fully halogenated and/or may be substituted by one or more radicals R⁶; —C(═O)R⁶; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰.

46. The compound according to claim 45, wherein R¹ is selected from the group consisting of hydrogen; cyano; C₁-C₆-alkyl; C₁-C₄-haloalkyl; C₁-C₄-alkoxy; C₁-C₄-haloalkoxy; and —C(═O)R⁶.

47. The compound according to claim 39, wherein R³ is selected from the group consisting of hydrogen; cyano; C₁-C₁₀-alkyl which may be partially or fully halogenated and/or may be substituted by one or more radicals R⁶; C₃-C₈-cycloalkyl which may be partially or fully halogenated and/or may be substituted by one or more radicals R⁶; C₂-C₁₀-alkenyl which may be partially or fully halogenated and/or may be substituted by one or more radicals R⁶; C₂-C₁₀-alkynyl which may be partially or fully halogenated and/or may be substituted by one or more radicals R⁶; —N(R⁸)R⁹; —Si(R¹⁴)₂R¹³; —OR⁷; —SR⁷; —S(O)ₘR⁷; —S(O)ₙN(R⁸)R⁹; —C(═O)R⁶; —C(═O)OR⁷; —C(═O)N(R⁸)R⁹; —C(═S)R⁶; —C(═S)OR⁷; —C(═S)N(R⁸)R⁹; —C(═NR⁸)R⁶; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
or R² and R³ together form a group ═CR¹¹R¹²; ═S(O)ₘR⁷; ═S(O)ₘN(R⁸)R⁹; ═NR⁸; or ═NOR⁷;
or R² and R³ together form a C₂-C₇ alkylene chain, thus forming, together with the nitrogen atom to which they are bound, a 3-, 4-, 5-, 6-, 7- or 8-membered ring, where the alkylene chain may be interrupted by 1 or 2 O, S or NR¹⁸ and/or 1 or 2 of the CH₂ groups of the alkylene chain may be replaced by a group C═O, C═S and/or C═NR¹⁸; and/or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰.

48. The compound according claim 47, wherein R³ is selected from the group consisting of hydrogen; C₁-C₁₀-alkyl which may be partially or fully halogenated and/or may be substituted by one or more radicals R⁶; C₃-C₈-cycloalkyl which may be partially or fully halogenated and/or may be substituted by one or more radicals R⁶; C₂-C₁₀-alkenyl which may be partially or fully halogenated and/or may be substituted by one or more radicals R⁶; C₂-C₁₀-alkynyl which may be partially or fully halogenated and/or may be substituted by one or more radicals R⁶; —C(═O)R⁶; —C(═O)OR⁷; —C(═O)N(R⁸)R⁹; —C(═S)R⁶; —C(═S)OR⁷; —C(═S)N(R⁸)R⁹; —C(═NR⁸)R⁶; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰.

49. The compound according claim 48, wherein R³ is selected from the group consisting of hydrogen; C₁-C₆-alkyl and C₁-C₄-haloalkyl.

50. The compounds according to claim 39, wherein each R⁴ is independently selected from halogen; cyano; nitro; —SCN; SF₅; C₁-C₆-alkyl which may be partially or fully halogenated and/or may be substituted by one or more radicals R⁶; C₃-C₈-cycloalkyl which may be partially or fully halogenated and/or may be substituted by one or more radicals R⁶; C₂-C₆-alkenyl which may be partially or fully halogenated and/or may be substituted by one or more radicals R⁶; C₂-C₆-alkynyl which may be partially or fully halogenated and/or may be substituted by one or more radicals R⁶; —Si(R¹⁴)₂R¹³; —OR⁷; —OS(O)ₙR⁷; —SR⁷; —S(O)ₘR⁷; —S(O)ₙN(R⁸)R⁹; —N(R⁸)R⁹; —N(R⁸)C(═O)R⁶; C(═O)R⁶; —C(═O)OR⁷; —C(═NR⁸)H; —C(═NR⁸)R⁶; —C(═O)N(R⁸)R⁹; C(═S)N(R⁸)R⁹; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
or two radicals R⁴ bound on adjacent carbon atoms may be together a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR⁸—, —CH₂CH═N—, —CH═CH—NR⁸—, —OCH═N—, and —SCH═N—, thus forming, together with the carbon atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group.

51. The compounds according to claim 50, wherein each R⁴ is independently selected from halogen; cyano; nitro; —SCN; C₁-C₆-alkyl which may be partially or fully halogenated and/or may be substituted by one or more radicals R⁶; C₃-C₈-cycloalkyl which may be partially or fully halogenated and/or may be substituted by one or more radicals R⁶; —OR⁷; —OS(O)ₙR⁷; —SR⁷; —S(O)ₘR⁷; —S(O)ₙN(R⁸)R⁹; —N(R⁸)R⁹; C(═O)R⁶; —C(═O)OR⁷; —C(═NR⁸)R⁶; —C(═O)N(R⁸)R⁹; and —C(═S)N(R⁸)R⁹.

52. The compound according claim 51, wherein R⁴ is selected from the group consisting of halogen, cyano, C₁-C₄-alkyl and C₁-C₄-haloalkyl.

53. The compound according to claim 39, wherein each R⁵ is independently selected from the group consisting of halogen, cyano, nitro, —SCN, SF₅, C₁-C₆-alkyl, C₁-C₆-alkyl which may be partially or fully halogenated and/or may be substituted by one or more radicals R⁶, C₃-C₈-cycloalkyl which may be partially or fully halogenated and/or may be substituted by one or more radicals R⁶, C₂-C₆-alkenyl which may be partially or fully halogenated and/or may be substituted by one or more radicals R⁶, C₂-C₆-alkynyl which may be partially or fully halogenated and/or may be substituted by one or more radicals R⁶, Si(R¹⁴)₂R¹³, OR⁷, OS(O)ₙR⁷, S(O)ₘR⁷, NR⁸R⁹, N(R⁸)C(═O)R⁶, C(═O)R⁶, C(═O)OR⁷, C(═NR⁸)R⁶, C(═S)NR⁶, phenyl which may be substituted by 1, 2, 3, 4 or 5 radicals le; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰.

54. The compounds according claim 53, wherein R⁵ is selected from the group consisting of halogen and C₁-C₄-haloalkyl.

55. The compound according to claim 39, wherein R² is selected from the group consisting of hydrogen; C₁-C₁₀-alkyl which may be partially or fully halogenated and/or may be substituted by one or more radicals R⁶; C₃-C₈-cycloalkyl which may be partially or fully halogenated and/or may be substituted by one or more radicals R⁶; C₂-C₁₀-alkenyl which may be partially or fully halogenated and/or may be substituted by one or more radicals R⁶; C₂-C₁₀-alkynyl which may be partially or fully halogenated and/or may be substituted by one or more radicals R⁶;
—C(═O)R⁶; —C(═O)OR⁷; —C(═O)N(R⁸)R⁹; —C(═S)R⁶; —C(═S)OR⁷, —C(═S)N(R⁸)R⁹; —C(═NR⁸)R⁶, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰.

56. The compounds according claim 55, wherein R² is selected from the group consisting of C₁-C₄-alkyl; C₁-C₄-haloalkyl; a methyl group substituted by a radical R^{6a}; —C(═O)R^{6c}; —C(═O)N(R⁸)R⁹; —C(═O)OR⁷; —C(═S)R^{6c}; —C(═S)N(R⁸)R⁹; —C(═S)OR⁷; and —C(═NR⁸)R^{6d},
where
R^{6a} is selected from CN, phenyl which may carry 1, 2 or 3 substituents R¹⁰, —C(═O)R^{6b}; —C(═O)N(R⁸)R⁹ and —C(═O)OR⁷;
R^{6b} and R^{6c} are independently selected from C₁-C₄-alkyl, C₁-C₄-haloalkyl, phenyl, benzyl and a 5- or 6-membered saturated, partially unsaturated or aromatic heterocyclic ring containing 1, 2 or 3 heteroatoms or heteroatom groups selected from N, O, S, NO, SO and SO₂, as ring members, where the phenyl or heterocyclyl rings in the three last-mentioned radicals may carry 1, 2 or 3 substituents selected from halogen, CN, C₁-C₄-alkyl, C₁-C₄-haloalkyl, C₁-C₄-alkoxy and C₁-C₄-haloalkoxy;
R^{6d} is selected from N(R⁸)R⁹;
R⁷ is selected from hydrogen, C₁-C₄-alkyl, C₁-C₄-haloalkyl, phenyl, benzyl and a 5- or 6-membered saturated, partially unsaturated or aromatic heterocyclic ring containing 1, 2 or 3 heteroatoms or heteroatom groups selected from N, O, S, NO, SO and SO₂, as ring members, where the phenyl or heterocyclyl rings in the three last-mentioned radicals may carry 1, 2 or 3 substituents selected from halogen, CN, C₁-C₄-alkyl, C₁-C₄-alkoxy and C₁-C₄-haloalkoxy;
each R⁸ is independently selected from hydrogen, cyano, C₁-C₆-alkyl which may be partially or fully halogenated and/or may be substituted by one or more radicals R¹⁹, C₂-C₄-alkenyl which may be partially or fully halogenated and/or may be substituted by one or more radicals R¹⁹, C₃-C₆-cycloalkyl, C₃-C₆-halocycloalkyl, C₃-C₆-cycloalkyl-C₁-C₄-alkyl, C₃-C₆-halocycloalkyl-C₁-C₄-alkyl, —S(O)ₘR²⁰, —S(O)ₙN(R²¹)R²², phenyl, benzyl and a 5- or 6-membered saturated, partially unsaturated or aromatic heterocyclic ring containing 1, 2 or 3 heteroatoms or heteroatom groups selected from N, O, S, NO, SO and SO₂, as ring members, where the phenyl or heterocyclyl rings in the three last-mentioned radicals may carry 1, 2 or 3 substituents selected from halogen, CN, C₁-C₄-haloalkyl, C₁-C₄-alkoxy and C₁-C₄-haloalkoxy;
each R⁹ is independently selected from hydrogen, cyano, C₁-C₆-alkyl which may be partially or fully halogenated and/or may be substituted by one or more radicals R¹⁹, C₂-C₄-alkenyl which may be partially or fully halogenated and/or may be substituted by one or more radicals R¹⁹, C₃-C₆-cycloalkyl, C₃-C₆-halocycloalkyl, C₃-C₆-cycloalkyl-C₁-C₄-alkyl, C₃-C₆-halocycloalkyl-C₁-C₄-alkyl, —S(O)ₘR²⁰, —S(O)ₙN(R²¹)R²², phenyl, benzyl and a 5- or 6-membered saturated, partially unsaturated or aromatic heterocyclic ring containing 1, 2 or 3 heteroatoms or heteroatom groups selected from N, O, S, NO, SO and SO₂, as ring members, where the phenyl or heterocyclyl rings in the three last-mentioned radicals may carry 1, 2 or 3 substituents selected from halogen, CN, C₁-C₄-alkyl, C₁-C₄-haloalkyl, C₁-C₄-alkoxy and C₁-C₄-haloalkoxy; and
R¹⁰ is selected from halogen, CN, C₁-C₄-alkyl, C₁-C₄-haloalkyl, C₁-C₄-alkoxy and C₁-C₄-haloalkoxy;
R⁸ and R⁹ together form a group ═CR¹¹R¹²; or
R⁸ and R⁹, together with the nitrogen atom to which they are bound, form a 3-, 4-, 5-, 6- or 7-membered saturated, partially unsaturated or aromatic heterocyclic ring which may additionally containing 1 or 2 further heteroatoms or heteroatom groups selected from N, O, S, NO, SO and SO₂, as ring members, where the heterocyclic ring may be substituted by one or more radicals R¹⁰;
each R¹⁹ is independently selected from the group consisting of cyano, azido, nitro, —SCN, SF₅, C₃-C₈-cycloalkyl, C₃-C₈-halocycloalkyl, —Si(R¹⁴)₂R¹³, —OR²⁰, —OSO₂R²⁰, —SR²⁰, —S(O)ₘR²⁰, —S(O)ₙN(R²¹)R²², —N(R²¹)R²², —C(═O)N(R²¹)R²², —C(═S)N(R²¹)R²², —C(═NR²¹)R²⁰, —C(═O)OR²⁰, —C(═O)R²⁰, phenyl which may be substituted by 1, 2, 3, 4 or 5 radicals independently selected from the group consisting of halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals independently selected from the group consisting of halogen, cyano, nitro, C₁-C₆-alkyl, C₁-C₆-haloalkyl, C₁-C₆-alkoxy and C₁-C₆-haloalkoxy;
and, in case R¹⁹ is bound to a cycloalkyl group, R¹⁹ may additionally be selected from the group consisting of C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl and C₂-C₆-haloalkynyl;
and in groups —C(═O)R¹⁹, R¹⁹ may additionally be selected from the group consisting of hydrogen, halogen, C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, and C₂-C₆-haloalkynyl;
or two geminally bound radicals R¹⁹ together form a group selected from the group consisting of ═CR¹¹R¹², ═S(O)ₘR²⁰, ═S(O)ₘN(R²¹)R²², ═NR²¹, ═NOR²⁰ and ═NNR²¹;
or two radicals R¹⁹, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from the group consisting of N, O, S, NO, SO and SO₂, as ring members.

57. The compound according claim 56, wherein R² is selected from —C(═O)N(R⁸)R⁹ and —C(═S)N(R⁸)R⁹;
where
R⁸ is selected from hydrogen and C₁-C₆-alkyl which may be partially or fully halogenated and/or may be substituted by one or more radicals R¹⁹;
R⁹ is selected from hydrogen, C₁-C₆-alkyl which may be partially or fully halogenated and/or may be substituted by one or more radicals R¹⁹, C₃-C₆-cycloalkyl, C₃-C₆-halocycloalkyl, C₃-C₆-cycloalkyl-C₁-C₄-alkyl and C₃-C₆-halocycloalkyl-C₁-C₄-alkyl.

58. The compound according claim 57, where
R⁸ is hydrogen; and
R⁹ is selected from hydrogen, C₁-C₆-alkyl; C₁-C₆-haloalkyl, C₃-C₆-cycloalkyl, C₃-C₆-halocycloalkyl, C₃-C₆-cycloalkyl-C₁-C₄-alkyl and C₃-C₆-halocycloalkyl-C₁-C₄-alkyl.

59. The compound according to claim 39, wherein Y is O, NR³ or a chemical bond.

60. The compound according claim 59, wherein Y is NR³.

61. The compound according to claim 39, wherein G is selected from G-3, G-4, G-13, G-14, G-16, G-17, G-18, G-19, G-21, G-26, G-27 and G-28.

62. The compounds according to claim 61, wherein G is selected from G-13, G-14 and G-16.

63. The compound according to claim 39, wherein G is bound via the attachment point “*” to the phenyl or pyridyl group comprising B¹ as ring member and via the attachment point “#” to the phenyl or pyridyl group comprising A¹ as ring member.

64. The compound according to claim 39, wherein R^{8a} is selected from hydrogen, C₁-C₄-alkyl and C₁-C₄-haloalkyl.

65. The compound according to claim 39, wherein p is 0, 1 or 2.

66. The compound according to claim 39, wherein q is 0, 1, 2 or 3.

67. The compound according to claim 39, of formula I-1 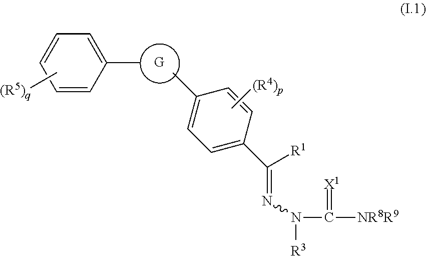
where
G is a bivalent heterocyclic ring selected from the group consisting of G-13, G-14 and G-16X¹ is O or S.

68. An agricultural composition comprising a compound of claim 39 and at least one inert liquid and/or solid agriculturally acceptable carrier.

69. A veterinary composition comprising a compound of claim 39 and at least one inert liquid and/or solid veterinarily acceptable carrier.

70. A method for controlling invertebrate pests which method comprises treating the pests, their food supply, their habitat or their breeding ground or a plant, plant propagation material, soil, area, material or environment in which the pests are growing or may grow, or the materials, plants, plant propagation material, soils, surfaces or spaces to be protected from invertebrate pest attack or infestation with a pesticidally effective amount of a compound of claim 39.

71. The method as claimed in claim 70, for protecting plants from attack or infestation by invertebrate pests, which method comprises treating the plants with a pesticidally effective amount of a compound of claim 39.

72. The method as claimed in claim 70, for protecting plant propagation material and/or the plants which grow therefrom from attack or infestation by invertebrate pests, which method comprises treating the plant propagation material with a pesticidally effective amount of a compound of claim 39.

73. Plant propagation material treated with a compound of claim 39.

74. A method for treating or protecting an animal from infestation or infection by invertebrate pests which comprises bringing the animal in contact with a pesticidally effective amount of a compound of claim 39.
